SEQUENCE LISTING (180 sequences)

SEQ ID NO: 1 - protein, 44 aa, synthetic construct
CEDDGEYFAGLQRLYGGDICYYIKLKFPKVPDLCIKEILDKLGC

SEQ ID NO: 2 - protein, 44 aa, synthetic construct
CEDDEEFFADLKRLRGGDICYYIKLKFDKVPDLCIKEILDKLGC

SEQ ID NO: 3 - protein, 44 aa, synthetic construct
CEYDEEFFAGLKRLRGGDICYYIKKKFDKVPDLCIKEILDKLGC

SEQ ID NO: 4 - protein, 44 aa, synthetic construct
CEDDFQFFADLKRLRGGDICYYIRLKFDKVPDLCIKEILDKLGC

SEQ ID NO: 5 - protein, 44 aa, synthetic construct
CEYDEEFFAGLKRLRGGDICYYIKKKFDKVPDLCIEEILDKLGC

SEQ ID NO: 6 - protein, 44 aa, synthetic construct
CEYDEEFFNGLKRLRGGDICYYIKKKFDKVPDLCIEEILDKLGC

SEQ ID NO: 7 - protein, 44 aa, synthetic construct
CEYDEEFFAGLKRLRRGDICYYIKKKFDKVPDLCIEEILDKLGC

SEQ ID NO: 8 - protein, 44 aa, synthetic construct
CEYDEEFFNGLKRLRRGDICYYIKKKFDKVPDLCIEEILDKLGC

SEQ ID NO: 9 - protein, 44 aa, synthetic construct
CEYDEEFFAGLKRLRGGDICYYIKKKFKKVPDLCIEEILDKLGC

SEQ ID NO: 10 - protein, 44 aa, synthetic construct
CEYDEEFFNGLKRLRGGDICYYIKKKFKKVPDLCIEEILDKLGC

SEQ ID NO: 11 - protein, 44 aa, synthetic construct
CEYDEEFFNGLHRLRRGDICYYIKKKFKKVPDLCIEEILDKLGC

SEQ ID NO: 12 - protein, 44 aa, synthetic construct
CEYDEEFFAGLKRLRGGDICYYIKKKFPKVPDLCIEEILDKLGC

SEQ ID NO: 13 - protein, 44 aa, synthetic construct
CEYDEEFFAGLHRLRGGDICYYIKKKFDKVPDLCIEEILDKLGC

SEQ ID NO: 14 - protein, 44 aa, synthetic construct
CEYDEEFFAGLHRLRGGDICYYIKKKFPKVPDLCIEEILDKLGC

SEQ ID NO: 15 - protein, 44 aa, synthetic construct
CEYDEEFFAGLKRLRGTDICYYIKKKFDKVPDLCIEEILDKLGC

SEQ ID NO: 16 - protein, 44 aa, synthetic construct
CEYDEEFFAGLHRLRGTDICYYIKKKFDKVPDLCIEEILDKLGC

SEQ ID NO: 17 - protein, 44 aa, synthetic construct
CEYDEEFFAGLHRLRGTDICYYIKKKFPKVPDLCIEEILDKLGC

SEQ ID NO: 18 - protein, 44 aa, synthetic construct
CEYDEEFFKGLKRLRGGDICYYIKKKFKKVPDLCIEEILDKLGC

SEQ ID NO: 19 - protein, 44 aa, synthetic construct
CEYDEEFFEGLKRLRGGDICYYIKKKFKKVPDLCIEEILDKLGC

SEQ ID NO: 20 - protein, 44 aa, synthetic construct
CEYDEEFFDGLKRLRGGDICYYIKKKFKKVPDLCIEEILDKLGC

SEQ ID NO: 21 - protein, 44 aa, synthetic construct
CEYDEEFFSGLKRLRGGDICYYIKKKFKKVPDLCIEEILDKLGC

SEQ ID NO: 22 - protein, 44 aa, synthetic construct
CEYDEEFFTGLKRLRGGDICYYIKKKFKKVPDLCIEEILDKLGC

SEQ ID NO: 23 - protein, 44 aa, synthetic construct
CEYDEEFFEGLKKLRGGDICYYIKKKFKKVPDLCIEEILDKLGC

SEQ ID NO: 24 - protein, 44 aa, synthetic construct
CEYDEEFFTGLKRLRGGDICYYIKKKFKKVPKLCIEEILDKLGC

SEQ ID NO: 25 - protein, 44 aa, synthetic construct
CEYDEEFFTGLKRLRGGDICYYIKKKFKKVPELCIEEILDKLGC

SEQ ID NO: 26 - protein, 44 aa, synthetic construct
CEYDEEFFQGLKRLRGGDICYYIKKKFKKVPDLCIEEILDKLGC

SEQ ID NO: 27 - protein, 44 aa, synthetic construct
CEYDEEFFLGLKRLRGGDICYYIKKKFKKVPDLCIEEILDKLGC

SEQ ID NO: 28 - protein, 44 aa, synthetic construct
CEYKEEFFTGLKRLRGGDICYYIKKKFKKVPDLCIEEILDKLGC

SEQ ID NO: 29 - protein, 44 aa, synthetic construct
CEYEEEFFTGLKRLRGGDICYYIKKKFKKVPDLCIEEILDKLGC

SEQ ID NO: 30 - protein, 44 aa, synthetic construct
CEYDEEFFTGLKRLRGGKICYYIKKKFKKVPDLCIEEILDKLGC

SEQ ID NO: 31 - protein, 44 aa, synthetic construct
CEYDEEFFTGLKRLRGGEICYYIKKKFKKVPDLCIEEILDKLGC

SEQ ID NO: 32 - protein, 44 aa, synthetic construct
CEYDEEFFTGLKRLRGGNICYYIKKKFKKVPDLCIEEILDKLGC

SEQ ID NO: 33 - protein, 44 aa, synthetic construct
CEYDEEFFTGLKRLRGGSICYYIKKKFKKVPDLCIEEILDKLGC

SEQ ID NO: 34 - protein, 44 aa, synthetic construct
CEYDEEFFTGLKRLRGGTICYYIKKKFKKVPDLCIEEILDKLGC

SEQ ID NO: 35 - protein, 44 aa, synthetic construct
CEYDEEFFTGLKRLRGGQICYYIKKKFKKVPDLCIEEILDKLGC

SEQ ID NO: 36 - protein, 44 aa, synthetic construct
  VARIANT 9: note = 2,3-Diaminopropionic Acid
CEYDEEFFXGLKRLRGGDICYYIKKKFKKVPDLCIEEILDKLGC

SEQ ID NO: 37 - protein, 44 aa, synthetic construct
CEYDEEFFTGLKRLRGGDICYYIKKKFKKVPDLCIEEILKKLGC

SEQ ID NO: 38 - protein, 44 aa, synthetic construct
CEYDEEFFTGLKRLRGGDICYYIKKKFKKVPDLCIEEILEKLGC

SEQ ID NO: 39 - protein, 44 aa, synthetic construct
CEYDEKFFTGLKRLRGGDICYYIKKKFKKVPDLCIEEILDKLGC

SEQ ID NO: 40 - protein, 44 aa, synthetic construct
CEYDKEFFTGLKRLRGGDICYYIKKKFKKVPDLCIEEILDKLGC

SEQ ID NO: 41 - protein, 44 aa, synthetic construct
CKYDEEFFTGLKRLRGGDICYYIKKKFKKVPDLCIEEILDKLGC

SEQ ID NO: 42 - protein, 44 aa, synthetic construct
CEYDEQFFTGLKRLRGGDICYYIKKKFKKVPDLCIEEILDKLGC

SEQ ID NO: 43 - protein, 44 aa, synthetic construct
CEYDEEFFTDLKRLRGGDICYYIKKKFKKVPKLCIEEILDKLGC

SEQ ID NO: 44 - protein, 44 aa, synthetic construct
CEYDEEFFTELKRLRGGDICYYIKKKFKKVPKLCIEEILDKLGC

SEQ ID NO: 45 - protein, 44 aa, synthetic construct
CEYDEEFFTKLKRLRGGDICYYIKKKFKKVPKLCIEEILDKLGC

SEQ ID NO: 46 - protein, 44 aa, synthetic construct
CEYDEEFFTTLKRLRGGDICYYIKKKFKKVPKLCIEEILDKLGC

SEQ ID NO: 47 - protein, 44 aa, synthetic construct
CEYDEEFFTLLKRLRGGDICYYIKKKFKKVPKLCIEEILDKLGC

SEQ ID NO: 48 - protein, 44 aa, synthetic construct
CEYDEEFFTALKRLRGGDICYYIKKKFKKVPKLCIEEILDKLGC

SEQ ID NO: 49 - protein, 44 aa, synthetic construct
CEYDEEFFLGLKRLRGGDICYYIKKKFKKVPKLCIEEILDKLGC

SEQ ID NO: 50 - protein, 44 aa, synthetic construct
CEYDEEFFTGLKRLRGGDICYYIKKKFKKVPKLCIEEILEKLGC

SEQ ID NO: 51 - protein, 44 aa, synthetic construct
CEYDEEFFLGLKRLRGGDICYYIKKKFKKVPDLCIEEILEKLGC

SEQ ID NO: 52 - protein, 44 aa, synthetic construct
CEYDEEFFLGLKRLRGGDICYYIKKKFKKVPKLCIEEILEKLGC

SEQ ID NO: 53 - protein, 44 aa, synthetic construct
CEYDEEFFTGLKRLRGGDICYYIKKKFKKVPKLCIEEILKKLGC

SEQ ID NO: 54 - protein, 44 aa, synthetic construct
CEYDEEFFLGLKRLRGGDICYYIKKKFKKVPKLCIEEILKKLGC

SEQ ID NO: 55 - protein, 44 aa, synthetic construct
CEYDEEFFLGLKRLRGGDICYYIKKKFKKVPDLCIEEILKKLGC

SEQ ID NO: 56 - protein, 44 aa, synthetic construct
CEYDEEFFTGLKRLRGGDICYYIKKKFKKVPELCIEEILKKLGC

SEQ ID NO: 57 - protein, 44 aa, synthetic construct
CEYDEEFFTGLKRLRGGDICYYIKKKFKKVPELCIEEILEKLGC

SEQ ID NO: 58 - protein, 44 aa, synthetic construct
CEYDEEFFLGLKRLRGGDICYYIKKKFKKVPELCIEEILKKLGC

SEQ ID NO: 59 - protein, 44 aa, synthetic construct
CEYDEEFFLGLKRLRGGDICYYIKKKFKKVPELCIEEILEKLGC

SEQ ID NO: 60 - protein, 44 aa, synthetic construct
CEYDEEFFKGLKRLRGGDICYYIKKKFKKVPKLCIEEILEKLGC

SEQ ID NO: 61 - protein, 44 aa, synthetic construct
CEYDEKFFTGLKRLRGGDICYYIKKKFKKVPKLCIEEILDKLGC

SEQ ID NO: 62 - protein, 44 aa, synthetic construct
CEYDEQFFTGLKRLRGGDICYYIKKKFKKVPKLCIEEILDKLGC

SEQ ID NO: 63 - protein, 44 aa, synthetic construct
CEYDEKFFLGLKRLRGGDICYYIKKKFKKVPKLCIEEILKKLGC

SEQ ID NO: 64 - protein, 44 aa, synthetic construct
CEYDEQFFLGLKRLRGGDICYYIKKKFKKVPKLCIEEILKKLGC

SEQ ID NO: 65 - protein, 44 aa, synthetic construct
CEYDEKFFTGLKRLRGGDICYYIKKKFKKVPKLCIEEILKKLGC

SEQ ID NO: 66 - protein, 44 aa, synthetic construct
CEYDEQFFTGLKRLRGGDICYYIKKKFKKVPKLCIEEILKKLGC

SEQ ID NO: 67 - protein, 44 aa, synthetic construct
CEYDEEFFKALKRLRGGDICYYIKKKFKKVPKLCIEEILEKLGC

SEQ ID NO: 68 - protein, 44 aa, synthetic construct
CEYDEEFFLALKRLRGGDICYYIKKKFKKVPKLCIEEILEKLGC

SEQ ID NO: 69 - protein, 44 aa, synthetic construct
CEYDEEFFLALKRLRGGDICYYIKKKFKKVPKLCIEEILDKLGC

SEQ ID NO: 70 - protein, 44 aa, synthetic construct
CEYDEEFFTALKRLRGGDICYYIKKKFKKVPKLCIEEILEKLGC

SEQ ID NO: 71 - protein, 44 aa, synthetic construct
CEYDEEFFKGLKRLRGGDICYYIKKKFKKVPKLCIEEILDKLGC

SEQ ID NO: 72 - protein, 44 aa, synthetic construct
CEYDEEFFKALKRLRGGDICYYIKKKFKKVPKLCIEEILDKLGC

SEQ ID NO: 73 - protein, 44 aa, synthetic construct
  VARIANT 12: note = Lysine(Ac)
  VARIANT 13: note = Citrulline
  VARIANT 15: note = Citrulline
  VARIANT 41: note = Lysine(Ac)
CEYDEEFFTALXXLXGGDICYYIKKKFKKVPKLCIEEILDXLGC

SEQ ID NO: 74 - protein, 44 aa, synthetic construct
  VARIANT 12: note = Lysine(Ac)
  VARIANT 13: note = Citrulline
  VARIANT 15: note = Citrulline
  VARIANT 24..26: note = Lysine(Ac)
  VARIANT 28..29: note = Lysine(Ac)
  VARIANT 32: note = Lysine(Ac)
  VARIANT 41: note = Lysine(Ac)
CEYDEEFFTALXXLXGGDICYYIXXXFXXVPXLCIEEILDXLGC

SEQ ID NO: 75 - protein, 44 aa, synthetic construct
  VARIANT 24..26: note = Lysine(Ac)
  VARIANT 28..29: note = Lysine(Ac)
  VARIANT 32: note = Lysine(Ac)
CEYDEEFFTALKRLRGGDICYYIXXXFXXVPXLCIEEILDKLGC

SEQ ID NO: 76 - protein, 44 aa, synthetic construct
CEYDEQFFTALKRLRGGDICYYISAQFNTLPDLCIEEILENLGC

SEQ ID NO: 77 - protein, 44 aa, synthetic construct
CEYDEEFFTALKKLRGGDICYYIQQAFNYLPGICIEEILDNLGC

SEQ ID NO: 78 - protein, 45 aa, synthetic construct
CEYDEEFFTALKRLRGGDICYYIQASFQYLPGLCIEEILDNLGCS

SEQ ID NO: 79 - protein, 44 aa, synthetic construct
CEYDEQFFTALKALRGGDICYYIQASFNYLPDLCIEEILDNLGC

SEQ ID NO: 80 - protein, 45 aa, synthetic construct
CEYDEQFFTALKALRGGDICYYIQASFNYLPDLCIEEILDNLGCS

SEQ ID NO: 81 - protein, 45 aa, synthetic construct
CEYDEEFFTALKRLRGGDICYYIQAKFQYLPKLCIEEILDNLGCS

SEQ ID NO: 82 - protein, 45 aa, synthetic construct
CEYDEEFFTALKRLRGGDICYYIQKAFQYLPGLCIEEILDNLGCS

SEQ ID NO: 83 - protein, 45 aa, synthetic construct
CEYDEEFFTALARLRGGDICYYIQAKFQYLPGLCIEEILDNLGCS

SEQ ID NO: 84 - protein, 44 aa, synthetic construct
CEYDEQFFTALARLRGGDICYYIQEQFATVPGLCIEEILDQLGC

SEQ ID NO: 85 - protein, 45 aa, synthetic construct
CEYDEQFFTALARLRGGDICYYIQEQFATVPGLCIEEILDQLGCS

SEQ ID NO: 86 - protein, 44 aa, synthetic construct
CEYDEEFFTALSRLRGGDICYYIQQAFQYLPGLCIEEILDNLGC

SEQ ID NO: 87 - protein, 45 aa, synthetic construct
CEYDEEFFTALSRLRGGDICYYIQQAFQYLPGLCIEEILDNLGCS

SEQ ID NO: 88 - protein, 44 aa, synthetic construct
CEYDEQFFTALSSLRGGDICYYIQEQFANVPGICIEEILDNLGC

SEQ ID NO: 89 - protein, 45 aa, synthetic construct
CEYDEQFFTALSSLRGGDICYYIQEQFANVPGICIEEILDNLGCS

SEQ ID NO: 90 - protein, 45 aa, synthetic construct
CEYDEEFFTALARLRGADICYYIQAKFQYLPGDCIEEILDNLGCS

SEQ ID NO: 91 - protein, 45 aa, synthetic construct
CDYDEEFFTALARLRGGDICEYIQAKFQYLPGLCIEEILDNLGCS

SEQ ID NO: 92 - protein, 45 aa, synthetic construct
CEYDEEFFTALARLRGGDICYYIQAKFQYLPGECIEEILQNLGCS

SEQ ID NO: 93 - protein, 45 aa, synthetic construct
CEYDEEFFTALARLRGDDICSYIQAKFQYLPGLCIEEILDNLGCS

SEQ ID NO: 94 - protein, 45 aa, synthetic construct
CEYDGEFFTALARLRGADICEYIQAKFQYYPGLCIEEILDNLGCS

SEQ ID NO: 95 - protein, 45 aa, synthetic construct
CEYDEEFFTALARLRGGDICYYILAKFQYLPGECIEEILDNLGCS

SEQ ID NO: 96 - protein, 45 aa, synthetic construct
CEYDEQFFTALARLRGGDICEYIQAKFQYLPGLCIEEILDNLGCS

SEQ ID NO: 97 - protein, 45 aa, synthetic construct
CEYDEEFFTALARLRGADICDYIQAKFQYLPGLCIEEILDNLGCS

SEQ ID NO: 98 - protein, 45 aa, synthetic construct
CEYDEEFFTALARLRGGDICEYIQAKFQYLPGLCIQEILDNLGCS

SEQ ID NO: 99 - protein, 45 aa, synthetic construct
CEYDEEFFTALARLRGGDICQYIQAKFQYLPGQCIEEILDNLGCS

SEQ ID NO: 100 - protein, 45 aa, synthetic construct
CEYDEEFFTALARLRGGDICEYIQAKFQYLEGLCIEEILDNLGCS

SEQ ID NO: 101 - protein, 45 aa, synthetic construct
CEYDEAFFTALARLRGGDICQYIQAKFQYLPGLCIEEILDNLGCS

SEQ ID NO: 102 - protein, 45 aa, synthetic construct
CEYDEQFFTALARLRGGDICYYILAKFQYLPQLCIEEILDNLGCS

SEQ ID NO: 103 - protein, 45 aa, synthetic construct
CEYDEEFFTALARLRGGDICQYIQAKFQYLPALCIEEILDNLGCS

SEQ ID NO: 104 - protein, 45 aa, synthetic construct
CEYDEEFFTALARLRGGDICYYIQAKFAYLPALCIEEILDNLGCS

SEQ ID NO: 105 - protein, 45 aa, synthetic construct
CEYDEEFFTALARLRGGDICQYIQAKFAYVPGLCIEEILDNLGCS

SEQ ID NO: 106 - protein, 45 aa, synthetic construct
CEYDEEFFTALARLRGSDICLYIQAKFQYLPGLCIEEILDNLGCS

SEQ ID NO: 107 - protein, 45 aa, synthetic construct
CEYDEEFFTALARLRGGDICDYIQAKFQYLPGLCIAEILDNLGCS

SEQ ID NO: 108 - protein, 45 aa, synthetic construct
CEYDGEFFTALARLRGGDICQYIQAKFQYLPGLCIEEILDNLGCS

SEQ ID NO: 109 - protein, 45 aa, synthetic construct
CDYDEEFFTALARLRGGDICYYIQAKFSYLPGLCIEEILDNLGCS

SEQ ID NO: 110 - protein, 45 aa, synthetic construct
CDYDEEFFTALARLRGGDICQYIQAKFQYLPGLCIEEILDNLGCS

SEQ ID NO: 111 - protein, 45 aa, synthetic construct
CEYDEEFFTALASLRGGDICYYIQAKFQYLPGLCIEEILDNLGCS

SEQ ID NO: 112 - protein, 45 aa, synthetic construct
CEYDEEFFTALAQLRGGDICYYIQAKFQYLPGLCIEEILDNLGCS

SEQ ID NO: 113 - protein, 45 aa, synthetic construct
  VARIANT 13: note = Citrulline
CEYDEEFFTALAXLRGGDICYYIQAKFQYLPGLCIEEILDNLGCS

SEQ ID NO: 114 - protein, 45 aa, synthetic construct
  VARIANT 26: note = hydroxy-norleucine
CEYDEEFFTALARLRGGDICYYIQAXFQYLPGLCIEEILDNLGCS

SEQ ID NO: 115 - protein, 45 aa, synthetic construct
CEYDEEFFTALARLRGGDICYYIQAYFQYLPGLCIEEILDNLGCS

SEQ ID NO: 116 - protein, 45 aa, synthetic construct
CEYDEEFFTALARLRGGDICYYIQAKFQYLPKLCIEEILDNLGCS

SEQ ID NO: 117 - protein, 45 aa, synthetic construct
CEYDEEFFTALARLRGGDICEYIQAKFQYLPKLCIEEILDNLGCS

SEQ ID NO: 118 - protein, 45 aa, synthetic construct
CEYDEEFFTALARLRGGDICSYIQAKFQYLPKLCIEEILDNLGCS

SEQ ID NO: 119 - protein, 45 aa, synthetic construct
CEYDEEFFTALARLRGGDICDYIQAKFQYLPKLCIEEILDNLGCS

SEQ ID NO: 120 - protein, 45 aa, synthetic construct
  VARIANT 13: note = Citrulline
  VARIANT 26: note = hydroxy-norleucine
CEYDEEFFTALAXLRGDDICSYIQAXFQYLPGLCIEEILDNLGCS

SEQ ID NO: 121 - protein, 45 aa, synthetic construct
  VARIANT 13: note = Citrulline
CEYDEEFFTALAXLRGGDICEYIQAKFQYLPGLCIEEILDNLGCS

SEQ ID NO: 122 - protein, 45 aa, synthetic construct
  VARIANT 13: note = Citrulline
CEYDEEFFTALAXLRGDDICSYIQAKFQYLPGLCIEEILDNLGCS

SEQ ID NO: 123 - protein, 45 aa, synthetic construct; identical to SEQ ID NO: 114
  VARIANT 26: note = hydroxy-norleucine

SEQ ID NO: 124 - protein, 45 aa, synthetic construct
  VARIANT 13: note = Citrulline
CEYDEEFFTALAXLRGGDICSYIQAKFQYLPGLCIEEILDNLGCS

SEQ ID NO: 125 - protein, 45 aa, synthetic construct
  VARIANT 13: note = Citrulline
CDYDEEFFTALAXLRGGDICEYIQAKFQYLPGLCIEEILDNLGCS

SEQ ID NO: 126 - protein, 45 aa, synthetic construct
CEYDEEFFTALKRLRGGDICYYIQASFQYLPGECIEEILDNLGCS

SEQ ID NO: 127 - protein, 45 aa, synthetic construct
CEYDEEFFTALKRLRGGDICEYIQASFQYLPGLCIEEILDNLGCS

SEQ ID NO: 128 - protein, 45 aa, synthetic construct
CEYDEEFFTALKRLRGGDICSYIQASFQYLPGLCIEEILDNLGCS

SEQ ID NO: 129 - protein, 45 aa, synthetic construct
CEYDEEFFTALKRLRGDDICYYIQASFQYLPGLCIEEILDNLGCS

SEQ ID NO: 130 - protein, 45 aa, synthetic construct
CEYDEEFFTALKRLRGDDICEYIQASFQYLPGLCIEEILDNLGCS

SEQ ID NO: 131 - protein, 45 aa, synthetic construct
CEYDEEFFTALKRLRGDDICSYIQASFQYLPGLCIEEILDNLGCS

SEQ ID NO: 132 - protein, 45 aa, synthetic construct
CEYDEQFFTALKRLRGADICEYIQASFQYLPGLCIEEILDNLGCS

SEQ ID NO: 133 - protein, 45 aa, synthetic construct
CEYDEQFFTALKRLRGGDICSYIQASFQYLPGLCIEEILDNLGCS

SEQ ID NO: 134 - protein, 45 aa, synthetic construct
CEYDEQFFTALKRLRGADICSYIQASFQYLPGLCIEEILDNLGCS

SEQ ID NO: 135 - protein, 45 aa, synthetic construct
CEYDEQFFTALKRLRGDDICSYIQASFQYLPGLCIEEILDNLGCS

SEQ ID NO: 136 - protein, 45 aa, synthetic construct
CDYDEEFFTALKRLRGGDICEYIQASFQYLPGLCIEEILDNLGCS

SEQ ID NO: 137 - protein, 45 aa, synthetic construct
CDYDEEFFTALKRLRGGDICSYIQASFQYLPGLCIEEILDNLGCS

SEQ ID NO: 138 - protein, 45 aa, synthetic construct
CDYDEQFFTALKRLRGGDICEYIQASFQYLPGLCIEEILDNLGCS

SEQ ID NO: 139 - protein, 45 aa, synthetic construct
CDYDEEFFTALKRLRGDDICEYIQASFQYLPGLCIEEILDNLGCS

SEQ ID NO: 140 - protein, 45 aa, synthetic construct
CDYDEEFFTALKRLRGDDICSYIQASFQYLPGLCIEEILDNLGCS

SEQ ID NO: 141 - protein, 45 aa, synthetic construct
CDYDEQFFTALKRLRGDDICEYIQASFQYLPGLCIEEILDNLGCS

SEQ ID NO: 142 - protein, 45 aa, synthetic construct
CEYDEQFFTALKRLRGADICDYIQASFQYLPGLCIEEILDNLGCS

SEQ ID NO: 143 - protein, 45 aa, synthetic construct
CEYDEEFFTALKRLRGADICDYIQASFQYLPGLCIEEILDNLGCS

SEQ ID NO: 144 - protein, 45 aa, synthetic construct
CEYDEQFFTALKRLRGGDICDYIQASFQYLPGLCIEEILDNLGCS

SEQ ID NO: 145 - protein, 45 aa, synthetic construct
CEYDEQFFTALKRLRGDDICDYIQASFQYLPGLCIEEILDNLGCS

SEQ ID NO: 146 - protein, 45 aa, synthetic construct
CEYDEEFFTALKRLRGGDICEYIQAAFQYLPGLCIEEILDNLGCS

SEQ ID NO: 147 - protein, 45 aa, synthetic construct
  VARIANT 26: note = norleucine
CEYDEEFFTALKRLRGGDICEYIQAXFQYLPGLCIEEILDNLGCS

SEQ ID NO: 148 - protein, 45 aa, synthetic construct
CEYDEEFFTALKRLRGGDICDYIQASFQYLPGLCIEEILDNLGCS

SEQ ID NO: 149 - protein, 45 aa, synthetic construct; identical to SEQ ID NO: 147
  VARIANT 26: note = Lysine(me3)

SEQ ID NO: 150 - protein, 45 aa, synthetic construct
CEYDEEFFTALKRLRGGDICEYIQASFQYLPGECIEEILDNLGCS

SEQ ID NO: 151 - protein, 45 aa, synthetic construct
CEYDEEFFTALKRLRGGDICDYIQASFQYLPGECIEEILDNLGCS

SEQ ID NO: 152 - protein, 45 aa, synthetic construct
CEYDEEFFTALKRLRGGDICEYIQASFQYLPGECIEEILQNLGCS

SEQ ID NO: 153 - protein, 45 aa, synthetic construct
CEYDEEFFTALKRLRGGDICDYIQASFQYLPGECIEEILQNLGCS

SEQ ID NO: 154 - protein, 45 aa, synthetic construct
CDYDEQFFTALKRLRGADICEYIQASFQYLPGLCIEEILDNLGCS

SEQ ID NO: 155 - protein, 45 aa, synthetic construct
CDYDEQFFTALKRLRGADICEYIQASFQYLPGECIEEILDNLGCS

SEQ ID NO: 156 - protein, 45 aa, synthetic construct
CDYDEQFFTALKRLRGADICEYIQASFQYLPGQCIEEILDNLGCS

SEQ ID NO: 157 - protein, 45 aa, synthetic construct
CDYDEQFFTALKRLRGGDICEYIQASFQYLPGECIEEILDNLGCS

SEQ ID NO: 158 - protein, 45 aa, synthetic construct
CDYDEQFFTALKRLRGGDICEYIQASFQYLPGQCIEEILDNLGCS

SEQ ID NO: 159 - protein, 510 aa, Homo sapiens
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 160 - protein, 508 aa, Mus musculus
MPLSLGAEMWGPEAWLRLLFLASFTGQYSAGELETSDVVTVVLGQDAKLPCFYRGDPDEQVGQVAWARVDPNEGIRELALLHSKYGLHVNPAYEDRVEQPPPPRDPLDGSVLLRNAVQADEGEYECRVSTFPAGSFQARMRLRVLVPPLPSLNPGPPLEEGQGLTLAASCTAEGSPAPSVTWDTEVKGTQSSRSFTHPRSAAVTSEFHLVPSRSMNGQPLTCVVSHPGLLQDRRITHTLQVAFLAEASVRGLEDQNLWQVGREGATLKCLSEGQPPPKYNWTRLDGPLPSGVRVKGDTLGFPPLTTEHSGVYVCHVSNELSSRDSQVTVEVLDPEDPGKQVDLVSASVIIVGVIAALLFCLLVVVVVLMSRYHRRKAQQMTQKYEEELTLTRENSIRRLHSHHSDPRSQPEESVGLRAEGHPDSLKDNSSCSVMSEEPEGRSYSTLTTVREIETQTELLSPGSGRTEEDDDQDEGIKQAMNHFVQENGTLRAKPTGNGIYINGRGHLV

SEQ ID NO: 161 - protein, 45 aa, synthetic construct
  VARIANT 2: note = D or E
  VARIANT 5: note = E or G
  VARIANT 6: note = Q or E
  VARIANT 12: note = A, K, or S
  VARIANT 13: note = A, R, Q, K, S, or Citrulline
  VARIANT 17: note = A, D, G, or S
  VARIANT 21: note = D, Q, E, L, S, or Y
  VARIANT 24: note = Q, L, or S
  VARIANT 25: note = A, Q, E, or K
  VARIANT 26: note = A, Q, K, S, Y, OH-Norleucine, or Norleucine
  VARIANT 28: note = A, N, Q, or S
  VARIANT 29: note = N, T, or Y
  VARIANT 30: note = L, Y, or V
  VARIANT 31: note = P or E
  VARIANT 32: note = A, D, Q, G, or K
  VARIANT 33: note = D, Q, E, I, or L
  VARIANT 36: note = Q or E
  VARIANT 40: note = D, Q, or E
  VARIANT 41: note = N or Q
  VARIANT 45: note = Residue may be deleted
CXYDXXFFTALXXLRGXDICXYIXXXFXXXXXXCIXEILXXLGCS

SEQ ID NO: 162 - protein, 45 aa, synthetic construct
  VARIANT 6: note = A, Q, or E
  VARIANT 12: note = A, K, or S
  VARIANT 13: note = A, R, Q, K, or S
  VARIANT 17: note = A, D, G, or S
  VARIANT 21: note = D, Q, E, L, S, or Y
  VARIANT 25: note = A, Q, E, or K
  VARIANT 26: note = A, Q, K, S, Y, OH-Norleucine, or Norleucine
  VARIANT 28: note = A, N, Q, or S
  VARIANT 32: note = A, D, Q, G, or K
CEYDEXFFTALXXLRGXDICXYIQXXFXYLPXLCIEEILDNLGCS

SEQ ID NO: 163 - protein, 45 aa, synthetic construct
  VARIANT 2: note = D, or E
  VARIANT 6: note = A, Q, or E
  VARIANT 12: note = A, K, or S
  VARIANT 13: note = A, R, Q, K, or S
  VARIANT 17: note = A, D, G, or S
  VARIANT 21: note = D, Q, E, L, S, or Y
  VARIANT 25: note = A, Q, E, or K
  VARIANT 26: note = A, Q, K, S, Y, OH-Norleucinecine, or Norleucine
  VARIANT 28: note = A, N, Q, or S
  VARIANT 32: note = A, D, Q, G, or K
CXYDEXFFTALXXLRGXDICXYIQXXFXYLPXLCIEEILDNLGCS

SEQ ID NO: 164 - protein, 45 aa, synthetic construct
  VARIANT 2: note = D, or E
  VARIANT 12: note = A, K, or S
  VARIANT 13: note = A, R, Q, K, or S
  VARIANT 17: note = A, D, G, or S
  VARIANT 21: note = D, Q, E, L, S, or Y
  VARIANT 25: note = A, Q, E, or K
  VARIANT 26: note = A, Q, K, S, Y, OH-Norleucinecine, or Norleucine
CXYDEEFFTALXXLRGXDICXYIQXXFQYLPGLCIEEILDNLGCS

SEQ ID NO: 165 - protein, 45 aa, synthetic construct
  VARIANT 2: note = D, or E
  VARIANT 6: note = A, Q, or E
  VARIANT 12: note = A, K, or S
  VARIANT 17: note = A, D, G, or S
  VARIANT 21: note = D, Q, E, L, S, or Y
  VARIANT 26: note = A, Q, K, S, Y, OH-Norleucinecine, or Norleucine
  VARIANT 32: note = A, D, Q, G, or K
CXYDEXFFTALXRLRGXDICXYIQAXFQYLPXLCIEEILDNLGCS

SEQ ID NO: 166 - protein, 45 aa, synthetic construct
  VARIANT 6: note = A, Q, or E
  VARIANT 12: note = A, K, or S
  VARIANT 13: note = A, R, Q, K, or S
  VARIANT 21: note = D, Q, E, L, S, or Y
  VARIANT 25: note = A, Q, E, or K
  VARIANT 26: note = A, Q, K, S, Y, OH-Norleucinecine, or Norleucine
  VARIANT 28: note = A, N, Q, or S
  VARIANT 32: note = A, D, Q, G, or K
CEYDEXFFTALXXLRGGDICXYIQXXFXYLPXLCIEEILDNLGCS

SEQ ID NO: 167 - protein, 45 aa, synthetic construct; identical to SEQ ID NO: 163
  VARIANT 2: note = D, or E
  VARIANT 6: note = A, Q, or E
  VARIANT 12: note = A, K, or S
  VARIANT 13: note = A, R, Q, K, or S
  VARIANT 17: note = A, D, G, or S
  VARIANT 21: note = D, Q, E, L, S, or Y
  VARIANT 25: note = A, Q, E, or K
  VARIANT 26: note = A, Q, K, S, Y, OH-Norleucinecine, or Norleucine
  VARIANT 28: note = A, N, Q, or S
  VARIANT 32: note = A, D, Q, G, or K

SEQ ID NO: 168 - protein, 45 aa, synthetic construct
  VARIANT 2: note = D, or E
  VARIANT 6: note = A, Q, or E
  VARIANT 12: note = A, K, or S
  VARIANT 17: note = A, D, G, or S
  VARIANT 21: note = D, Q, E, L, S, or Y
  VARIANT 26: note = A, Q, K, S, Y, OH-Norleucinecine, or Norleucine
CXYDEXFFTALXRLRGXDICXYIQAXFQYLPGLCIEEILDNLGCS

SEQ ID NO: 169 - protein, 45 aa, synthetic construct
  VARIANT 2: note = D, or E
  VARIANT 6: note = A, Q, or E
  VARIANT 12: note = A, K, or S
  VARIANT 13: note = A, R, Q, K, or S
  VARIANT 17: note = A, D, G, or S
  VARIANT 21: note = D, Q, E, L, S, or Y
  VARIANT 26: note = A, Q, K, S, Y, OH-Norleucinecine, or Norleucine
  VARIANT 32: note = A, D, Q, G, or K
CXYDEXFFTALXXLRGXDICXYIQAXFQYLPXLCIEEILDNLGCS

SEQ ID NO: 170 - protein, 45 aa, synthetic construct; identical to SEQ ID NO: 169
  VARIANT 2: note = D, or E
  VARIANT 6: note = A, Q, or E
  VARIANT 12: note = A, K, or S
  VARIANT 13: note = A, R, Q, K, or S
  VARIANT 17: note = A, D, G, or S
  VARIANT 21: note = D, Q, E, L, S, or Y
  VARIANT 26: note = A, Q, K, S, Y, OH-Norleucinecine, or Norleucine
  VARIANT 32: note = A, D, Q, G, or K

SEQ ID NO: 171 - protein, 45 aa, synthetic construct; identical to SEQ ID NO: 163
  VARIANT 2: note = D, or E
  VARIANT 6: note = A, Q, or E
  VARIANT 12: note = A, K, or S
  VARIANT 13: note = A, R, Q, K, or S
  VARIANT 17: note = A, D, G, or S
  VARIANT 21: note = D, Q, E, L, S, or Y
  VARIANT 25: note = A, Q, E, or K
  VARIANT 26: note = A, Q, K, S, Y, OH-Norleucinecine, or Norleucine
  VARIANT 28: note = A, N, Q, or S
  VARIANT 32: note = A, D, Q, G, or K

SEQ ID NO: 172 - protein, 45 aa, synthetic construct
  VARIANT 2: note = D, or E
  VARIANT 6: note = A, Q, or E
  VARIANT 12: note = A, K, or S
  VARIANT 13: note = A, R, Q, K, or S
  VARIANT 17: note = A, D, G, or S
  VARIANT 21: note = D, Q, E, L, S, or Y
  VARIANT 25: note = A, Q, E, or K
  VARIANT 26: note = A, Q, K, S, Y, OH-Norleucinecine, or Norleucine
  VARIANT 28: note = A, N, Q, or S
CXYDEXFFTALXXLRGXDICXYIQXXFXYLPGLCIEEILDNLGCS

SEQ ID NO: 173 - protein, 45 aa, synthetic construct
  VARIANT 2: note = D, or E
  VARIANT 6: note = A, Q, or E
  VARIANT 12: note = A, K, or S
  VARIANT 17: note = A, D, G, or S
  VARIANT 21: note = D, Q, E, L, S, or Y
  VARIANT 26: note = A, Q, K, S, Y, OH-Norleucinecine, or Norleucine
  VARIANT 28: note = A, N, Q, or S
  VARIANT 32: note = A, D, Q, G, or K
CXYDEXFFTALXRLRGXDICXYIQAXFXYLPXLCIEEILDNLGCS

SEQ ID NO: 174 - protein, 45 aa, synthetic construct; identical to SEQ ID NO: 163
  VARIANT 2: note = D, or E
  VARIANT 6: note = A, Q, or E
  VARIANT 12: note = A, K, or S
  VARIANT 13: note = A, R, Q, K, or S
  VARIANT 17: note = A, D, G, or S
  VARIANT 21: note = D, Q, E, L, S, or Y
  VARIANT 25: note = A, Q, E, or K
  VARIANT 26: note = A, Q, K, S, Y, OH-Norleucinecine, or Norleucine
  VARIANT 28: note = A, N, Q, or S
  VARIANT 32: note = A, D, Q, G, or K

SEQ ID NO: 175 - protein, 45 aa, synthetic construct; identical to SEQ ID NO: 172
  VARIANT 2: note = D, or E
  VARIANT 6: note = A, Q, or E
  VARIANT 12: note = A, K, or S
  VARIANT 13: note = A, R, Q, K, or S
  VARIANT 17: note = A, D, G, or S
  VARIANT 21: note = D, Q, E, L, S, or Y
  VARIANT 25: note = A, Q, E, or K
  VARIANT 26: note = A, Q, K, S, Y, OH-Norleucinecine, or Norleucine
  VARIANT 28: note = A, N, Q, or S

SEQ ID NO: 176 - protein, 45 aa, synthetic construct; identical to SEQ ID NO: 163
  VARIANT 2: note = D, or E
  VARIANT 6: note = A, Q, or E
  VARIANT 12: note = A, K, or S
  VARIANT 13: note = A, R, Q, K, or S
  VARIANT 17: note = A, D, G, or S
  VARIANT 21: note = D, Q, E, L, S, or Y
  VARIANT 25: note = A, Q, E, or K
  VARIANT 26: note = A, Q, K, S, Y, OH-Norleucinecine, or Norleucine
  VARIANT 28: note = A, N, Q, or S
  VARIANT 32: note = A, D, Q, G, or K

SEQ ID NO: 177 - protein, 44 aa, synthetic construct
CEYDEEFFTALKRLRGGDICYYIKKKFDYLPKLCIEEILDNLGC

SEQ ID NO: 178 - protein, 20 aa, synthetic construct
  VARIANT 2..10: note = Residue may be deleted
  VARIANT 11..20: note = gamma-glutamine
  VARIANT 12..20: note = Residue may be deleted
GGGGGGGGGGEEEEEEEEEE

SEQ ID NO: 179 - protein, 10 aa, synthetic construct
  VARIANT 2..10: note = Residue may be deleted
GGGGGGGGGG

SEQ ID NO: 180 - protein, 81 aa, synthetic construct
  VARIANT 2..16: note = Any amino acid or deleted
  VARIANT 18..32: note = Any amino acid or deleted
  VARIANT 34..48: note = Any amino acid or deleted
  VARIANT 50..64: note = Any amino acid or deleted
  VARIANT 66..80: note = Any amino acid or deleted
CXXXXXXXXXXXXXXXCXXXXXXXXXXXXXXXCXXXXXXXXXXXXXXXCXXXXXXXXXXXXXXXCXXXXXXXXXXXXXXXC

(19) United States
(12) Patent Application Publication
(10) Pub. No.: US 2026/0034253 A1
(43) Pub. Date: Feb. 5, 2026

(54) NECTIN-4 MINIPROTEIN CONJUGATES

(71) Applicant: Aktis Oncology, Inc., Boston, MA (US)

(72) Inventors: Matthew Roden, Princeton, NJ (US); Brian Goodman, Boston, MA (US); Ved Srivastava, Cary, NC (US); Paul L. Feldman, Durham, NC (US); William C. Blackwell, III, Raleigh, NC (US); Isaiah Nathaniel Gober, Durham, NC (US); Hyun Joo Kil, Cary, NC (US)

(21) Appl. No.: 18/881,668

(22) PCT Filed: Jul. 7, 2023
(86) PCT No.: PCT/US2023/027173

§ 371 Date: Jan. 6, 2025

Related U.S. Application Data
(60) Provisional application No. 63/359,146, filed on Jul. 7, 2022.

(51) Int. Cl. A61K 51/08; A61K 47/64; A61P 35/00; C07K 14/47; G01N 33/574

(52) U.S. Cl. CPC A61K 51/088; A61K 47/6425; A61P 35/00; C07K 14/47; G01N 33/57484; A61K 2121/00

(57) ABSTRACT

Provided herein are peptides and conjugates, including radionuclide conjugates, useful in compositions and methods of treating, diagnosing, monitoring, and/or imaging a disease, disorder, or condition associated with expression of one or more targets, including Nectin-4.

### CROSS-REFERENCE TO RELATED APPLICATIONS

This application is a national phase entry of international patent application PCT/US2023/027173, filed on Jul. 7, 2023, which claims priority to and the benefit of U.S. Provisional Patent Application No. 63/359,146, filed on Jul. 7, 2022, the entire contents of which is incorporated by reference.

### BACKGROUND

Cancer is a leading cause of death worldwide. Classical cancer therapies such as radiotherapy, chemotherapy, and surgical procedures can be accompanied by severe side effects including those due to killing of healthy non-cancerous cells. Newer therapeutics enhance the targeting of cytotoxic drugs to tumor cells, relative to earlier therapies, including those that use biologics conjugates.

### SUMMARY

The present disclosure provides technologies such as compositions and methods of use and manufacture thereof to addresses needs in the field of cancer. For example, in contrast to classical cancer diagnostics or therapies, targeted molecules can be designed to increase specificity and decrease toxicity of, e.g., imaging or therapeutic modalities. For instance, delivery of a therapeutic such as a chelator and/or radionuclide (e.g., alpha emitter) using a polypeptide to specifically target the therapeutic to the tumor microenvironment provides focused treatment to tumor cells and avoids or reduces risk of toxicity to surrounding healthy tissues by the therapeutic targeted to, e.g., a tumor.

In contrast to classical cancer therapies, radionuclide therapies are more targeted and less toxic. For instance, delivery of a radionuclide specifically to a tumor microenvironment allows for selective direction of radiation towards tumor tissue, effectively killing malignant cells while preserving the surrounding healthy tissue. For example, a radionuclide can employ a targeting molecule that specifically binds to an antigen expressed at an increased level and/or density on the surface of tumor cells relative to non-tumor cells. Binding of the radionuclide to the antigen-positive tumor cells targets radiation to those cells without targeting healthy tissue. Full-length antibodies have previously been evaluated as targeting moieties; however, due to considerations such as their large size, full-length antibodies, or even antibody fragments (e.g., fragments larger than polypeptides provided herein) can have several challenges such as having poor tumor tissue penetration, as well as challenges such as surrounding administration, manufacturing, and storage, among other things. Accordingly, a need remains for new approaches to specifically target tumors, and particularly solid tumors. The present disclosure provides technologies that meet this and other needs. Among other things, the present disclosure provides compositions comprising polypeptides and conjugates thereof with improved tumor penetration, decreased off-target toxicity and/or accumulation, and improved affinity for Nectin-4, as compared to currently existing technologies (e.g., antibodies, e.g., antibody-drug conjugates).

In some aspects, the present disclosure provides polypeptides comprising certain amino acid sequences. In some embodiments, these polypeptides bind to Nectin-4 with certain affinities. In some embodiments, the present disclosure provides polypeptides comprising an amino acid sequence, wherein the amino acid sequence comprises Formula I: CX1YDX2X3FFTALX4X5LRGX6DICX7YIX8X9X10FX11X12X13X14X15X16CIX17EI LX18X19LGCX20 (SEQ ID NO: 161), wherein X20 is an optional amino acid or carboxy terminus comprising an —OH and wherein X1 is D or E; X2 is E or G; X3 is Q or E; X4 is A, K, or S; X5 is A, R, Q, K, S, or Cit; X6 is A, D, G, or S; X7 is D, Q, E, L, S, or Y; X8 is Q, L, or S; X9 is A, Q, E, K; X10 Is A, Q, K, S, Y, OH-Norleu, or Norleu; X11 is A, N, Q, or S; X12 is N, T, or Y; X13 is L, Y, or V; X14 is P or E; X15 is A, D, Q, G, or K; X16 is D, Q, E, I, or L; X17 is Q or E; X18 is D, Q, or E; X19 is N or Q; and X20, when present as an amino acid is S.

In some embodiments, X1 is E; X2 is E; X3 is E; X4 is K; X5 is R; X6 is G; X7 is Y; X8 is Q; X9 is A; X10 is S; X11 is Q; X12 is Y; X13 is L; X14 is P; X15 is G; X16 is L; X17 is E; X18 is D; X19 is N; and X20 is S.

In some embodiments, the polypeptide further comprises one or more of a linker, chelator, and radionuclide. In some embodiments, the linker comprises or consists of a polyethylene glycol (PEG) linker of PEG4, PEG, PEG2, PEG6, PEG8, PEG12, PEG24, an ester linker, an amide linker, a maleimide linker, a succinimidyl-4-(N-maleimidomethyl) cyclohexane-1-carboxylate (SMCC) linker, a propanoic acid linker, a caproleic acid linker, or (Gly)n-(gGlu)n- or (PEG)n, wherein n is from 1 to 10, (Gly)1-10, or any fragment or combination via covalent bond thereof. In some embodiments, the chelator comprises or consists of DOTA, DOPA, Macropa, or Crown. In some embodiments, the radionuclide is selected from Ac-225, Ga-68, In-111, Pb-212, Lu-177, Cu-67, Cu-64, La-132, La-135, or Ce-134. In some embodiments, when present, the linker is attached to the C-terminal end of the polypeptide. In some embodiments, when present, the chelator is attached to either the polypeptide or the linker. In some embodiments, when present, the radionuclide is attached to the chelator. In some embodiments, the polypeptide comprises one or more additional N-terminal amino acids. In some embodiments, the polypeptide comprises one or more amino acids on the N-terminal and/or the C-terminal end of the polypeptide. In some embodiments, the polypeptide is a monomer. In some embodiments, the polypeptide is a dimer, trimer, or tetramer. In some embodiments, the polypeptide comprises an amino acid sequence with at least 80% sequence identity to that of SEQ ID NO: 78. In some embodiments, the polypeptide comprises an amino acid sequence with at least 80% sequence identity to that of any of SEQ ID NOs: 1-158 or 177. In some embodiments, the polypeptide comprises or consists of a consensus sequence according to any of Formulas IIA-IIO (SEQ ID NOs: 161-176) according to Table 1B, further comprising a substitution in accordance with those set forth in Table 1C.

In some embodiments, the present disclosure provides compositions comprising a miniprotein (M), an optional linker (L), and one or both of a chelator (C) and a radionuclide (R), represented by a formula selected from one or more of (M)ₓ-L-C-R, (M)ₓ-L-C, (M)ₓ-C-R, (M)ₓ-L-R, (M)ₓ-C, (M)ₓ-L, and (M)ₓ-R, wherein x is 1, 2, 3, or 4. In some embodiments, M comprises or consists of a sequence selected from SEQ ID NO: 78, or any of SEQ ID NOS: 1-77, 79-158, 177, 161-176 (including amino acid substitutions as set forth in Table 1C) or a functional variant or portion thereof. In some embodiments, when L is present, L comprises or consists of a polyethylene glycol (PEG) linker of PEG4, PEG, PEG2, PEG6, PEG8, PEG12, PEG24, an ester linker, an amide linker, a maleimide linker a valine-citrulline linker, a hydrazone linker, a N-succinimidyl-4-(2-pyridyldithio)butyrate (SPDB) linker, a succinimidyl-4-(N-maleimidomethyl)cyclohexane-1-carboxylate (SMCC) linker, a vinylsulfone-based linker, a propanoic acid linker, a caproleic acid linker, or (Gly)n-(gGlu)n- or (PEG)n, wherein n is from 1 to 10, (Gly)1-10, or any fragment or combination via covalent bond thereof. In some embodiments, when C is present, C comprises or consists of:

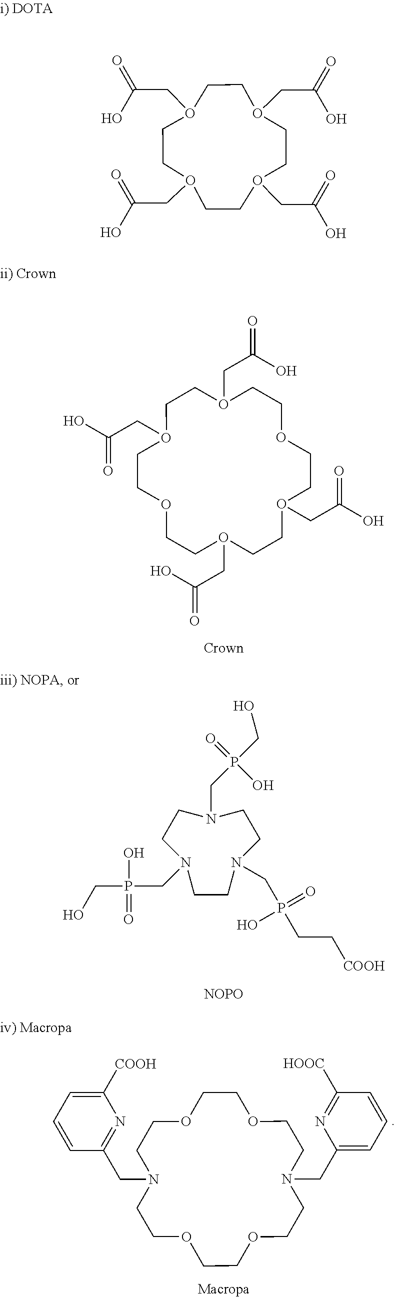

Among other things, the present disclosure provides conjugates comprising polypeptides (e.g., miniproteins) that target tumor microenvironments and/or tumor cells conjugated to one or more additional components including, for example, a linker, chelator, and/or radionuclide (e.g., an alpha emitter). In some embodiments, such conjugates are used in treatment of cells expressing a target (e.g., Nectin-4). In some such embodiments, the cells are cancer cells.

In some embodiments, a composition comprises a linker and a chelator.

In some embodiments, a composition comprises a linker, chelator, and radionuclide.

In some embodiments, a composition comprises a miniprotein, an optional linker, a chelator and/or a radionuclide.

In some embodiments, a composition is represented by the formula selected from one or more of (M)ₓ-L-C-R, (M)ₓ-L-C, (M)ₓ-C-R, (M)ₓ-L-R, (M)ₓ-C, (M)ₓ-L, and (M)ₓ-R, wherein M comprises a miniprotein (M), L comprises a linker (L), C comprises a chelator (C), R comprises a radionuclide (R), and x is 1, 2, 3, or 4, wherein M comprises a miniprotein that binds to Nectin-4 or a functional variant or portion thereof.

In some embodiments, a polypeptide in accordance with the present disclosure is manufactured using solid phase peptide synthesis methods. In some embodiments, the polypeptide is recombinant. In some embodiments, the polypeptide comprises or consists of a miniprotein. In some embodiments, the miniprotein comprises or consists of a cysteine-dense polypeptide, a knottin-protein, a binder, an affibody, an engineered Kunitz domain, a monobody, an anticalin, a designed ankyrin repeat domain (DARPin), or an avimer. In some embodiments, the miniprotein comprises or consists of approximately 100 amino acids or less. In some embodiments, the miniprotein is a cysteine-dense protein. In some embodiments, the miniprotein comprises at least one cysteine-dense region. In some embodiments, the amino acid sequence of the miniprotein comprises at least two cysteines (which may form at least one disulfide bridge). In some embodiments, the miniprotein comprises one or more disulfide bridges. In some embodiments, the amino acid sequence of the miniprotein comprises at least three cysteines. In some embodiments, the miniprotein comprises or consists of at least two disulfide bridges. In some embodiments, the miniprotein comprises or consists of a non-disulfide amino acid sequence. In some embodiments, the miniprotein comprises three alpha helices with 44-45 amino acids. In some such embodiments, the miniprotein has a molar mass of about 6 kDa or less. In some embodiments, the miniprotein is stable at high temperatures and under acidic or alkaline conditions. In some embodiments, the miniprotein comprises an engineered protein derived from a lipocalin. In some embodiments, the miniprotein comprises an eight-stranded β-barrel. In some embodiments, the miniprotein displays (i) high structural plasticity as a consequence of sequence variation and (ii) elevated conformational flexibility, allowing induced fit to targets with differing shape. In some embodiments, the miniprotein comprises a class of antibody mimetics which consist of two or more peptide sequences of 30 to 35 amino acids each, which are derived from A-domains of various membrane receptors and which are connected by linker peptides. In some embodiments, the miniprotein comprises a peptide derived from Ankyrin. In some embodiments, the miniprotein comprises an ankyrin repeat, a 33-residue motif consisting of two alpha-helices and a beta-turn. In some embodiments, the miniprotein comprises a peptide derived from the Kunitz domain of a Kunitz-type protease inhibitor such as bovine pancreatic trypsin inhibitor (BPTI), amyloid precursor protein (APP) or tissue factor pathway inhibitor (TFPI). In some embodiments, the miniprotein comprises a molecule based on the 10th extracellular domain of human fibronectin III (1° fn3), which adopts an Ig-like b-sandwich fold of 94 residues with 2 to 3 exposed loops, but lacks the central disulfide bridge.

In some embodiments, the present disclosure provides compositions that have various advantages over compositions comprising a full-length protein or a protein that is not a miniprotein. For example, in some embodiments, a composition of the present disclosure comprises one or more improved properties selected from increased protein expression, increased thermoactivity, increased thermostability, increased pH activity, increased stability, increased activity, increased receptor binding specificity and/or affinity, increased specific activity, increased resistance to substrate and/or end-product inhibition, increased chemical stability, improved chemoselectivity, improved solvent stability, increased tolerance to acidic pH, increased tolerance to proteolytic activity (e.g.., reduced sensitivity to proteolysis), reduced aggregation, increased solubility, and/or reduced immunogenicity.

In some embodiments, a composition of the present disclosure specifically binds to at least one epitope of a target in, on, or near a cell. In some embodiments, the target is Nectin-4. In some embodiments, the cell is a tumor cell (which may or may not have properties of having cancer, but is derived from a tumor sample). In some embodiments, the cell is a cancer cell. In some embodiments, the cell is a mammalian cell. In some embodiments, the cell is a primary cell. In some embodiments, the primary cell is taken from a sample from a subject (e.g., from a biopsy, e.g., from a tumor). In some embodiments, the cell is from a cell line.

In some embodiments, a miniprotein or conjugate thereof selectively binds to a target. In some embodiments, the target is a protein or portion thereof expressed on the surface of a cell. In some embodiments, the cell is a mammalian cell. In some embodiments, the cell is a human cell. In some embodiments, the cell is a cancer cell. In some such embodiments, the cancer cell is in (i.e., part of) or near a tumor. In some embodiments, the cancer cell is a circulating cell.

In some embodiments, a target is a cell adhesion receptor, a cytokine receptor, a chemokine receptor, a growth factor receptor, an immune cell receptor, or a tumor associated extracellular matrix polypeptide. In some such embodiments the target comprises or consists of Nectin-4 or a portion thereof.

In some embodiments, a miniprotein or fragment thereof can be used to generate a binding protein such as an antibody (e.g., a recombinant antibody, polyclonal, monoclonal, monospecific, multispecific (e.g., bispecific, trispecific, tetraspecific, etc.), chimeric, synthetic, natural, humanized, grafted, a full-length antibody, an antigen-binding region of an antibody such as a Fab, Fab′ F(ab′)2, single chain (Fv, sFv, scFv, bispecific scFv), diabody, single chain (e.g., single heavy chain, single light chain), nanobody, VHH, etc.) that specifically binds to at least one epitope on a target. In some embodiments, the target is Nectin-4. In some embodiments, the Nectin-4 is human Nectin-4. In some such embodiments, such an antibody may be used, for example, in diagnostic or blocking applications, etc.

In related aspects, the present disclosure provides pharmaceutical compositions comprising a conjugate.

In some embodiments, a conjugate comprises one or more of a miniprotein, linker, chelator, and radionuclide.

In some embodiments, one or more components of a conjugate are provided using a vector, e.g., a recombinant expression vector comprising a nucleic acid encoding a polypeptide.

In some embodiments, the present disclosure provides a vector comprising the isolated polynucleotide as provided herein.

In some embodiments the present disclosure provides a host cell comprising a vector encoding one or more components of a conjugate as provided herein.

In some embodiments, the present disclosure provides a composition that is capable of binding to, modulating, and/or inhibiting Nectin-4. In some embodiments, the Nectin-4 is human Nectin-4. In some embodiments, a composition of the present disclosure is or comprises monoclonal antibody therapy, peptide therapy, peptide receptor radionuclide therapy, immunotherapy, radioimmunotherapy, gene therapy, or RNA therapy.

In some embodiments a composition of the present disclosure is formulated for administration to a subject in need thereof (e.g., a pharmaceutical composition). In some embodiments, a composition is administered for the treatment of one or more diseases, disorders, or conditions. In some embodiments, a disease, disorder or condition is cancer. In some embodiments, the treatment comprises providing a composition (e.g., by administering, e.g., by contacting a cell or population of cells, e.g., a tumor), wherein the composition comprises a component that specifically binds to Nectin-4. In some embodiments, the Nectin-4 is expressed on a cell or population of cells. In some embodiments, providing the composition treats the disease, disorder, or condition.

In some embodiments, the present disclosure provides methods for modulating Nectin-4. That is, in some embodiments, a method of modulating biological activity of Nectin-4 comprises providing (e.g., by administering, e.g., by contacting a cell or population of cells) a composition comprising one or more components in an amount effective to modulate Nectin-4 and its activity. For example, in some embodiments, a composition comprises a miniprotein that specifically binds to Nectin-4 and one or more of a linker, chelator, and/or radionuclide. In some such embodiments, the composition binds to cells expressing Nectin-4 and targets one or more therapies (e.g., a chelator, e.g., a radionuclide) to a cell expressing the Nectin-4 such as, for example, by internalization of the composition or a component thereof into the cell.

In some embodiments, the present disclosure provides methods and compositions for use therein that are capable of activating or inhibiting immune cell response. In some embodiments, provided compositions are administered for the treatment of cancer. In some embodiments, the cancer is associated with expression or overexpression of Nectin-4. In some embodiments, the cancer is selected from non-small-cell lung cancer (NSCLC), cutaneous squamous cell carcinoma, pancreatic cancer, primary hepatocellular carcinoma, colorectal carcinoma, clear cell renal carcinoma, breast cancer and prostate cancer. See, e.g., Yang, Shuo et al “Nectin-4, a checkpoint molecule, as a target for cancer innmunotherapy” “International journal of biological sciences vol. 16,11 1767-1773. 25 Mar. 2020, doi:10.7150/ijbs.41105.

In some embodiments, the present disclosure provides methods and compositions for use in detecting the presence or extent of a disease, disorder, or condition in a subject. In some embodiments, the disease, disorder, or condition is cancer. In some embodiments, the subject has been diagnosed as having cancer. In some embodiments, the subject is suspected of having or being at risk of having cancer. In some embodiments, the subject diagnosed with cancer has been treated with one or more treatments, such as a composition as provided herein. In some embodiments, a method of detecting (e.g., monitoring/determining prognosis, diagnosing, etc.) comprises measuring a level of a target or a miniprotein that specifically binds to a target in a sample comprising one or more cells from a subject (e.g., using a cell-based assay) or in a subject (e.g., using an in vivo scan or measurement). In some such embodiments, a level of the target or of the miniprotein is used to determine presence and/or extent of cancer in the subject by comparing a level to a control level or to a level from the same patient at a different point in time.

In some embodiments, the present disclosure provides kits. In some such embodiments, a kit comprises one or more components such as a miniprotein, linker, chelator, and/or radionuclide that may be combined in one or more methods for use in binding to a target (e.g., Nectin-4).

In some embodiments, the present disclosure provides compositions comprising a miniprotein (M), an optional linker (L), and one or both of a chelator (C) and a radionuclide (R), represented by a formula selected from one or more of (M)ₓ-L-C-R, (M)ₓ-L-C, (M)ₓ-C-R, (M)ₓ-L-R, (M)ₓ-C, (M)ₓ-L, and (M)ₓ-R, wherein x is 1, 2, 3, or 4. In some embodiments, M comprises or consists of a cysteine-dense peptide, a knottin peptide, a binder, an affibody, an engineered Kunitz domain, a monobody, an anticalin, a designed ankyrin repeat domain (DARPin), or an avimer. In some embodiments, the binder comprises or consists of a linear polypeptide and/or a non-disulfide sequence. In some embodiments, M is characterized in that it comprises (i) no more than 1,000 amino acids, 900 amino acids, 800 amino acids, 700 amino acids, 600 amino acids, 500 amino acids, 400 amino acids, 300 amino acids, 200 amino acids, or 100 amino acids. In some preferred embodiments, M is characterized in that it comprises (i) no more than 100 amino acids and/or 12 kDa; (ii) at least one secondary structure elements; (iii) a sequestered hydrophobic core; and/or displays cooperative folding. In some embodiments, M comprises no more than about 100 amino acids or less, 90 amino acids, 85 amino acids, 80 amino acids, 75 amino acids, 70 amino acids, 65 amino acids, 60 amino acids, 55 amino acids, 50 amino acids, 45 amino acids, 40 amino acids, 35 amino acids, 30 amino acids, 25 amino acids, 20 amino acids, 15 amino acids, 10 amino acids, or 5 amino acids.

In some embodiments, the miniprotein comprises at least one disulfide bridge. In some embodiments, the miniprotein comprises zero, one, two or more disulfide bonds. In some embodiments, the miniprotein comprises at least one disulfide bridge. In some embodiments, the amino acid sequence of the miniprotein comprises at least three cysteine (C) amino acids. In some embodiments, the miniprotein comprises at least two disulfide bridges.

In some embodiments, the present disclosure provides compositions set forth as L-C, wherein L comprises or consists of a linker, C comprises or consists of a chelator, and wherein the linker is designed to be conjugated to a polypeptide.

In some embodiments, the present disclosure provides compositions set forth as L-C-R, wherein L comprises or consists of a linker, C comprises or consists of a chelator, and R comprises or consists of a radionuclide, and wherein the composition is capable of being conjugated to a miniprotein. In some embodiments, L, when present, comprises or consists of a polyethylene glycol (PEG) linker of PEG4, PEG, PEG2, PEG6, PEG8, PEG12, PEG24, an ester linker, an amide linker, a maleimide linker, a succinimidyl-4-(N-maleimidomethyl) cyclohexane-1-carboxylate (SMCC) linker, a propanoic acid linker, a caproleic acid linker, or (Gly)n-(yGlu)n- or (PEG)n, wherein n is from 1 to 10, (Gly)1__{I}o, or any fragment or combination via covalent bond thereof. In some embodiments, C when present, comprises or consists of:

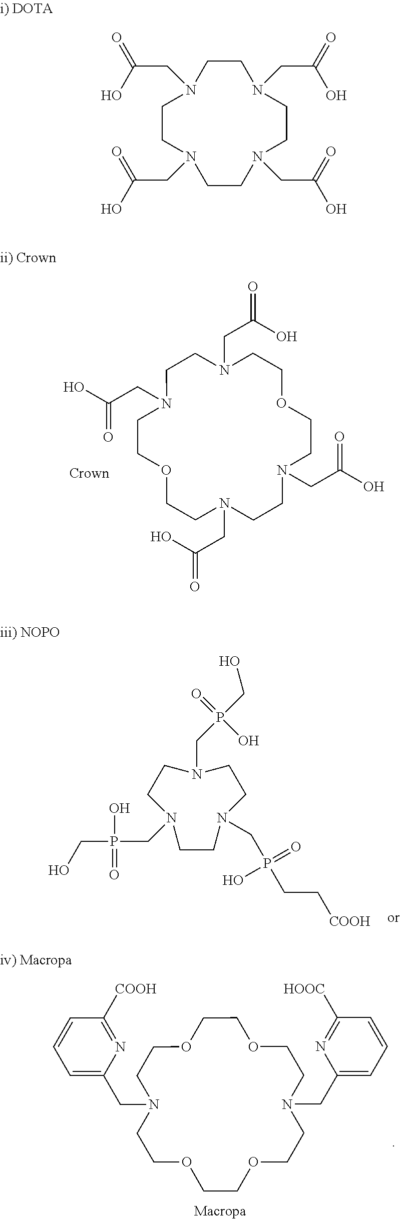 [SMILES: O=C(O)CN1CCN(CC(=O)O)CCN(CC(=O)O)CCN(CC(=O)O)CC1 ; O=C(O)CN1CCOCCN(CC(=O)O)CCN(CC(=O)O)CCOCCN(CC(=O)O)CC1 ; O=C(O)CCP(=O)(O)CN1CCN(CP(=O)(O)CO)CCN(CP(=O)(O)CO)CC1 ; O=C(O)c1cccc(CN2CCOCCOCCN(Cc3cccc(C(=O)O)n3)CCOCCOCC2)n1]

In some embodiments, C, when present, comprises or consists of derivative of tetraazacyclododecane-1,4,7,10-tetraacetic acid (DOTA), Crown, NOPO, or Macropa. In some embodiments, when L is present and C is absent, L is covalently attached to M. In some embodiments, R, when present, comprises or consists of Ac-225, In-111, Ga-68, Pb-212, Lu-177, Cu-67, Cu-64, La-132, La-135, or Ce-134. In some embodiments, a composition binds to the target with an affinity of 1 μM to 100 nM as measured by an in vitro binding assay. In some embodiments, when M is present the miniprotein binding to the target modulates biological function. In some embodiments, when M is present, M selectively binds to Nectin-4 or a portion thereof.

In some embodiments, the present disclosure provides a composition comprising a miniprotein that binds to Nectin-4 with an affinity of about that set forth in Table 1C.

In some embodiments, a composition provided herein comprises a miniprotein (M), which miniprotein comprises an amino acid sequence selected from any one of SEQ ID NOs: 1-158 or 177. In some embodiments, the composition comprises a miniprotein (M), which miniprotein comprises an amino acid sequence selected from any one of SEQ ID NOs: 161-176. In some embodiments, M comprises amino acid substitutions at any of SEQ ID NOs: 161-176 as set forth in Table 1C.

In some embodiments, the present disclosure provides a composition comprising isolated compounds or pharmaceutically acceptable salts thereof comprising an optional linker (L), and one or more of a miniprotein (M), a chelator (C) or radionuclide (R). In some embodiments, the miniprotein comprises an amino acid sequence selected from any one of SEQ ID NOs: 1-158 or 177 or an amino acid sequence according to any of SEQ ID NOs: 161-176 and/or Table 1C.

In some embodiments, the present disclosure provides pharmaceutical compositions comprising a miniprotein (M), wherein the miniprotein selectively binds to a target.

In some embodiments, the present disclosure provides pharmaceutical compositions comprising one or more miniproteins (M)x, wherein the miniprotein selectively binds to Nectin-4, and x is 1, 2, 3, or 4.

In some embodiments, the composition displays adherence in a tumor. In some embodiments, the composition displays passage through a kidney, a liver, a bone marrow, or a spleen.

In some embodiments, the present disclosure provides pharmaceutical compositions comprising miniproteins (M)x, an optional linker (L), and one or both of a chelator (C) and radionuclide (R), wherein x is 1, 2, 3, or 4. In some embodiments, when C is present, C covalently attaches to M. In some embodiments, the chelation efficiency is >90%. In some embodiments, the pharmaceutical composition further comprises a radionuclide R. In some embodiments, R, when present, is an alpha-emitter. In some embodiments, R, when present, is Ac-225, In-111. Ga-68, Pb-212, Lu-177, Cu-67, Cu-64, La-132, La-135, or Ce-134. In some embodiments, M specifically binds to a target. In some embodiments, the target is Nectin-4. In some embodiments, the Nectin-4 is expressed on a cell. In some embodiments, the cell is a human cell. In some embodiments, the human cell is a tumor cell. In some embodiments, the tumor cell is a solid tumor cell. In some embodiments, the miniprotein comprises or consists of a cysteine-dense peptide, a knottin peptide, a binder, an affibody, an engineered Kunitz domain, a monobody, an anticalin, a designed ankyrin repeat domain (DARPin), or an avimer. In some embodiments, the miniprotein comprises one or more disulfide bonds. In some embodiments, the miniprotein is characterized in that it has nM or sub-nM binding affinity on the target in vivo or in a cell-based assay. In some embodiments, the miniprotein a binding affinity of 1 pM to 100 nM to Nectin-4 on a cell line expressing human Nectin-4. In some embodiments, the miniprotein has a binding affinity of about that disclosed for a compound in Table 2. In some embodiments, the miniprotein has an amino acid sequence no more than about 100 amino acids or less, 90 amino acids, 85 amino acids, 80 amino acids, 75 amino acids, 70 amino acids, 65 amino acids, 60 amino acids, 55 amino acids, 50 amino acids, 45 amino acids, 40 amino acids, 35 amino acids, 30 amino acids, 25 amino acids, 20 amino acids, 15 amino acids, 10 amino acids, or 5 amino acids.

In some embodiments, administration of the pharmaceutical composition to a subject in need thereof does not elicit an immune response or wherein the immune response elicited is tolerable to the subject. In some embodiments, the composition comprises high tumor tissue penetration. In some embodiments, the composition displays adherence in a tumor. In some embodiments, the composition is not taken up and/or retained in the kidney or liver. In some embodiments, the composition is internalized in a cell expressing human Nectin-4. In some embodiments, the miniprotein comprises an amino acid sequence selected from any one of SEQ ID NOs: 1-158 or 177. In some embodiments, the miniprotein comprises an amino acid sequence according to any of SEQ ID NOs: 161-176. In some embodiments, the amino acid sequence of the miniprotein further comprises one or more substitutions as set forth in Table 1C.

In some embodiments, a composition comprises a miniprotein-drug conjugate, comprising a miniprotein and at least one drug moiety. In some embodiments, a pharmaceutical composition comprises a miniprotein-drug conjugate, comprising a miniprotein and at least one drug moiety. In certain embodiments, the drug moiety includes but is not limited to a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder, a topoisomerase inhibitor, an immunotoxin, and/or a DHFR inhibitor or functional derivatives, variants, or portions thereof.

In other embodiments, the drug moiety includes but is not limited to alkylating agents, such a thioTEPA and cyclosphosphamide (CYTOXAN™); alkyl sulfonates such as busulfan, improsulfan, and piposulfan; aziridines such as benzodopa, carboquone, meturedopa, and uredopa; ethylenimines and methylamelamines including altretamine, triethylenemelamine, triethylenephosphoramide, triethylenethiophosphoramide, and trimethylolomelamine; acetogenins (especially bullatacin and bullatacinone); camptothecin (including the synthetic analogue topotecan); bryostatin; callystatin; CC-1065 (including its adozelesin, carzelesin, and bizelesin synthetic analogues) and derivatives thereof; cryptophycines (particularly cryptophycin 1 and cryptophycin 8); dolastatin, auristatins (including analogues monomethyl-auristatin E and monomethyl-auristatin F (see, e.g., U.S. Published Application No. 2005-0238649, published Oct. 27, 2005, incorporated herein in its entirety); duocarmycin (including the synthetic analogues, KW-2189 and CBI-TMI); eleutherobin; pancratistatin; sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine; trofosfamide, uracil mustard; nitrosureas such as carmustine, chlorozotocin, fotemustine, lomustine, nimustine, ranimustine; antibiotics such as the enediyne antibiotics (e.g., calicheamicin, especially calichemicin gammalI and calicheamicin phill, see for example, Agnew, Chem. Intl. Ed. Engl. 33:183-186; dynemicin, including dynemicin A; bisphosphonates, such as clodronate; esperamicin; as well as neocarzinostatin chromophore and related chromoprotein enediyne antibiotic chromomophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (Adriamycin™) (including morpholino-doxorubicin, cyanomorpholino-doxorubicin, 2-pyrrolino-doxorubicin, and deoxydoxorubicin), epirubucin, esorubicin, idarubicin, marcellomycin, mitomycins such as mitomycin C, mycophenolic acid, nogalamycin, olivomycins, peplomycin, potfiromycin, puromycine, quelamycin, rodorubicin, streptonigrin, streptozocin, tubercidin, ubenimex, zinostatin, zorubicin; anti-metabolites such a methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adranals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; democolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone, mitoxantrone; mopidamol; nitracrine; pentostatin; phenamet; pirarubicin; losoxantrone; podophyllinic acid; 2-ethylhydrazide; procarbazine; PSK®; razoxane; rhizoxin; sizofiran; spirogermanium; tenuazonic acid; triaziquone; 2,2′,2@-trichlorotriethylamine; trichothecenes (especially T-2 toxin, verracurin A, roridin A and anguidine); urethan; vindesine; dacarbazine; mannomustine; mitabronitol; mitolactol; pipobroman; gacytosine; arabinoside (“Ara-C”); cyclophosphamide; thiotepa; taxoids, e.g., paclitaxel (TAXOL™, Bristol-Myers Squibb Oncology, Princeton, N.J.) and doxetaxel (TAXOTERE®, Rhone-Poulenc Rorer, Antony, France); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitoxantrone; vincristine; vinorelbine (Navelbine™); novantrone; teniposide; edatrexate; daunomycin; aminopterin; xeloda; ibandronate; CPT-11; difluoromethylornithine (DMFO); retinoids such as retinoic acid; capecitabine; and pharmaceutically acceptable salts, acids, or derivatives of any of the above.

In some embodiments, the drug moiety is or comprises an auristatin. In some such embodiments, the auristatin includes analogues such as monomethyl-auristatin E and monomethyl-auristatin F (see, e.g., U.S. Published Application No. 2005-0238649, published Oct. 27, 2005, incorporated herein in its entirety.

In some embodiments, a drug moiety includes but is not limited to anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); aromatase inhibitors that inhibit the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprolide, and goserelin; and pharmaceutically acceptable salts, acids, or derivatives of any of the above.

In some embodiments, a drug moiety is or comprises a topoisomerase inhibitor including but not limited to irinotecan, topotecan, camptothecin, etoposide, etoposide phosphate, doxorubicin, epirubicin, indolocarbazole and derivatives, phenanthridine and derivatives, indenoisoquinoline and derivatives, amsacrine, teniposide, daunomycin, mitoxantrone, idarubicin, indotecan, indimitecan, lamellarin D, SN-38, exatecan, lurtotecan, diflomotecan, gimatecan, lurtotecan, belotecan, edotecarin, rosettacin, cositecan, etirinotecan pegol, HOCPT, silatecan, elmotecan, topoisomerase inhibitor RFS 2000; or functional derivatives or portions thereof.

In some embodiments, a drug moiety is or comprises an immunotoxin or a functional derivative, variant, or portion thereof. In some embodiments, an immunotoxin is or comprises Erb38, scFv(FRP5)-ETA, SSIP (SSI(dsFv)-PE38, LMB-I, LMB-7, LMB-9, SGN-10, OvB3-PE, TP40, denileukin diftitox, moxetumomab pasudotox, LMB-2, UCHTI, D2219ARL, SSIP, MOC31PE, oportuzumab monatox, HuMI95-gelonin, RG7787, VB8-845 hSGZ, D2c7-(scdsfv)-PE38KDEI or a functional derivative, variant, or portion thereof.

In some embodiments, the pharmaceutical composition comprises one or more of an antioxidant molecule, wherein the antioxidant molecule neutralizes a free radical. In some embodiments, the pharmaceutical composition comprises a stabilizer. In further embodiments, the stabilizer comprises gentisic acid or salts thereof, ascorbic acid or salts thereof, methionine, N-acetyl cysteine, histidine, melatonin, ethanol, Se-methionine, or combinations thereof. In some embodiments, the pharmaceutical composition is characterized to exhibit an improved resistance to a peptidase, a protease, or heat.

In some embodiments, a method of producing a composition represented by the formula selected from one or more of (M)x-L-C-R, (M)x-L-C, (M)x-C-R, (M)x-L-R, (M)x-C, (M)x-L, and (M)x-R, wherein M comprises a miniprotein (M), L comprises a linker (L), C comprises a chelator (C), R comprises a radionuclide (R), and x is 1, 2, 3, or 4, comprises synthesizing a miniprotein (M) and/or linker (L), and optionally reacting a chelator (C) and/or a radionuclide (R), and conjugating one or more of the miniprotein (M) to the linker (L), one or more of the miniprotein (M) to the chelator (C), one or more of the miniprotein (M) to the radionuclide (R), one or more of the miniprotein (M) to the linker (L) to the chelator (C), one or more of the miniprotein (M) to the linker (L) to the chelator (C) and the radionuclide (R), one or more of the miniprotein (M) to the chelator (C) to the radionuclide (R), or one or more of the miniprotein (M) to the linker (L) to the radionuclide (R).

In some embodiments, the method involves reacting a chelator (C) and a radionuclide (R) at a temperature of between about 10° C. and 65° C. during an incubation period. In some embodiments, reacting a chelator (C) and a radionuclide (R) is performed during an incubation period of about 5 minutes to about 30 minutes. In further embodiments, reacting a chelator (C) and a radionuclide (R) is performed at a pH in the range of about 5.0 to 7.4. In some embodiments, reacting a chelator (C) and a radionuclide (R) is performed in an aqueous solution that is substantially free of alcohol.

In some embodiments, a method of delivering a radionuclide to a selected location within a patient involves administering a composition represented by the formula selected from one or more of (M)x-L-C-R, (M)x-L-C, (M)x-C-R, (M)x-L-R, (M)x-C, (M)x-L, and (M)x-R, wherein M comprises a miniprotein (M), L comprises a linker (L), C comprises a chelator (C), R comprises a radionuclide (R), and x is 1, 2, 3, or 4. In some embodiments, the radionuclide is selected from Ac-225, In-111, Ga-68, Pb-212, Lu-177, Cu-67, Cu-64, La-132, La-135, or Ce-134. In some embodiments, the method further comprises carrying out an imaging procedure to evaluate the localization of the radionuclide within the body, wherein the imaging procedure optionally comprises positron emission tomography (PET) imaging or single-photon emission computerized tomography (SPECT) imaging. In further embodiments, the imaging procedure allows for selecting patients. In certain embodiments, the imaging procedure allows for monitoring patients. In some embodiments, the imaging procedure allows for determining an appropriate dose for treating a patient in need of a pharmaceutical composition comprising one or more miniprotein.

In some embodiments, the present disclosure provides compositions comprising a miniprotein (M), an optional linker (L), and/or one or both of a chelator (C) and a radionuclide (R), represented by a formula selected from one or more of (M)x-L-C-R, (M)x-L-C, (M)x-C-R, (M)x-L-R, (M)x-C, (M)x-L, and (M)x-R, wherein x is 1, 2, 3, or 4. In some embodiments, M comprises or consists of a sequence selected from any of SEQ ID NOS: 1-158 or 177 or a functional variant or portion thereof or SEQ ID NOs: 161-176 and/or in accordance with Tables 1B and/or 1C or a functional variant or portion thereof. In some embodiments, L, when present, comprises or consists of a polyethylene glycol (PEG) linker of PEG4, PEG, PEG2, PEG6, PEG8, PEG12, PEG24, an ester linker, an amide linker, a maleimide linker a valine-citrulline linker, a hydrazone linker, a N-succinimidyl-4-(2-pyridyldithio)butyrate (SPDB) linker, a succinimidyl-4-(N-maleimidomethyl)cyclohexane-1-carboxylate (SMCC) linker, a vinylsulfone-based linker, a propanoic acid linker, a caproleic acid linker, or (Gly)n-(γGlu)n- or (PEG)n, wherein n is from 1 to 10, (Gly)1-10, or any fragment or combination via covalent bond thereof. In some embodiments, C, when present comprises or consists of:

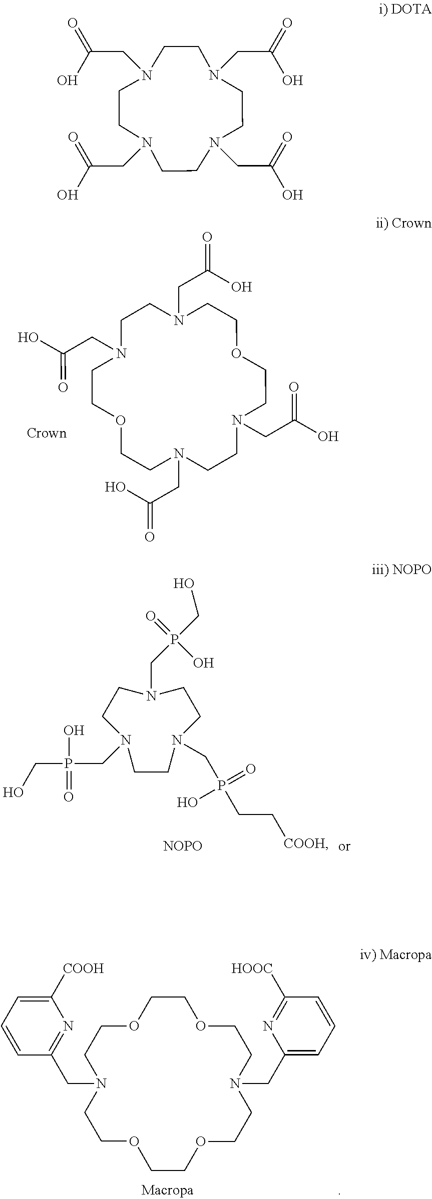 [SMILES: O=C(O)CN1CCN(CC(=O)O)CCN(CC(=O)O)CCN(CC(=O)O)CC1 ; O=C(O)CN1CCOCCN(CC(=O)O)CCN(CC(=O)O)CCOCCN(CC(=O)O)CC1 ; O=C(O)CCP(=O)(O)CN1CCN(CP(=O)(O)CO)CCN(CP(=O)(O)CO)CC1 ; "NOPO" ; "or" ; O=C(O)c1cccc(CN2CCOCCOCCN(Cc3cccc(C(=O)O)n3)CCOCCOCC2)n1]

In some embodiments, R, when present, comprises or consists of Ac-225, In-111, Ga-68, Pb-212, Lu-177, Cu-67, Cu-64, La-132, La-135, or Ce-134.

In some embodiments, the present disclosure provides methods of treating a subject in need thereof comprising administering a composition comprising a miniprotein (M), an optional linker (L), and one or both of a chelator (C) and a radionuclide (R).

In some embodiments, L comprises or consists of a polyethylene glycol (PEG) linker, an ester linker, an amide linker, a maleimide linker, a valine-citrulline linker, a hydrazone linker, a N-succinimidyl-4-(2-pyridyldithio)butyrate (SPDB) linker, a succinimidyl-4-(N-maleimidomethyl)cyclohexane-1-carboxylate (SMCC) linker, a vinylsulfone-based linker, a propanoic acid linker, a caproleic acid linker, or any fragment or combination thereof. In some embodiments, C comprises or consists of:

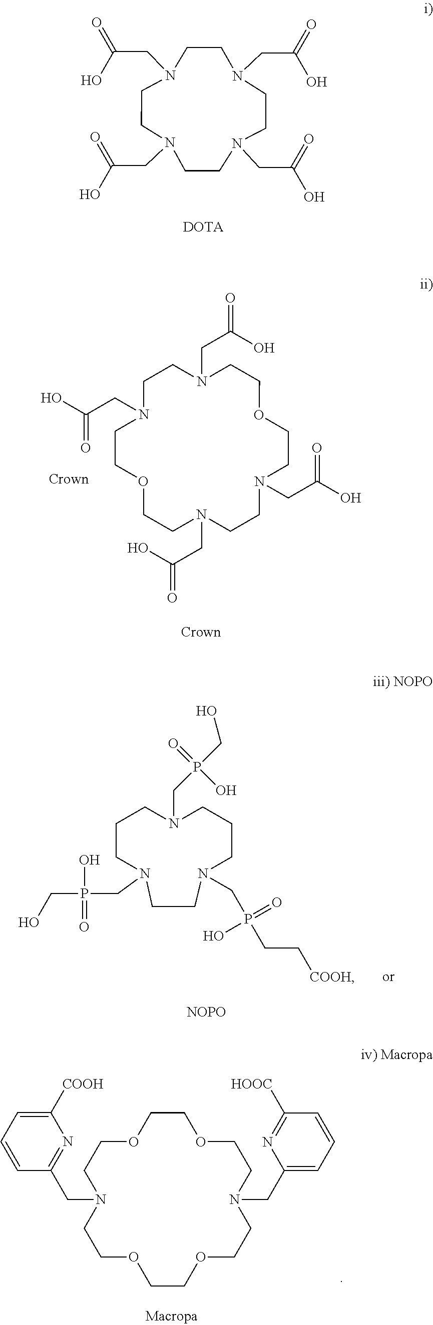 [SMILES: O=C(O)CN1CCN(CC(=O)O)CCN(CC(=O)O)CCN(CC(=O)O)CC1 ; "DOTA" ; O=C(O)CN1CCOCCN(CC(=O)O)CCN(CC(=O)O)CCOCCN(CC(=O)O)CC1 ; O=C(O)CCP(=O)(O)CN1CCCN(CP(=O)(O)CO)CCCN(CP(=O)(O)CO)CC1 ; "NOPO" ; O=C(O)c1cccc(CN2CCOCCOCCN(Cc3cccc(C(=O)O)n3)CCOCCOCC2)n1]

In some embodiments, R comprises or consists of Ac-225, In-111, Ga-68, Pb-212, Lu-177, Cu-67, Cu-64, La-132, La-135, or Ce-134. In some embodiments, M comprises or consists of a cysteine-dense polypeptide, a knottin-protein, a binder, an affibody, an engineered Kunitz domain, a monobody, an anticalin, a designed ankyrin repeat domain (DARPin), or an avimer. In some embodiments, M is characterized in that it comprises (i) no more than 1,000 amino acids, 900 amino acids, 800 amino acids, 700 amino acids, 600 amino acids, 500 amino acids, 400 amino acids, 300 amino acids, 200 amino acids, or 100 amino acids. In some preferred embodiments, M is characterized in that it comprises (i) no more than 100 amino acids and/or 12 kDa; (ii) at least two secondary structure elements; (iii) a sequestered hydrophobic core; and/or displays cooperative folding. In some embodiments, the composition comprises at least one additional component. In some embodiments, the composition can penetrate tumor tissue. In some embodiments, the miniprotein comprises no more than about 100 amino acids or less, 90 amino acids, 85 amino acids, 80 amino acids, 75 amino acids, 70 amino acids, 65 amino acids, 60 amino acids, 55 amino acids, 50 amino acids, 45 amino acids, 40 amino acids, 35 amino acids, 30 amino acids, 25 amino acids, 20 amino acids, 15 amino acids, 10 amino acids, or 5 amino acids. In some embodiments, the miniprotein comprises at least one disulfide bridge. In some embodiments, the miniprotein specifically binds to a target. In some embodiments, the composition displays mm or nM binding affinity to the target in an in vitro assay.

In some embodiments, the composition binds to the target with an affinity of 100 pM to 100 nM as measured by an in vitro binding assay. In some embodiments, the composition binds to Nectin-4 with an affinity of about that set forth for any of the compounds of Table 1C.

In some embodiments, the composition is characterized in that it has high tissue penetrating properties relative to a composition comprising a full-size protein that binds to the same target. In some embodiments, the miniprotein binding to the target modulates biological function.

In some embodiments, administration of the composition to a subject in need thereof does not elicit an immune response or wherein the immune response elicited is tolerable to the subject. In some embodiments, the tolerable immune response includes a systemic immune response or a local immune response.

In some embodiments, the subject is diagnosed as having cancer. In some embodiments, a cancer cell from the subject expresses Nectin-4, or a portion thereof. In some embodiments, the expression of the target is higher in the cancer cell than in a non-cancer cell.

In some embodiments, the cancer is selected from breast cancer, ovarian cancer, melanoma, pancreatic cancer, peripheral neuroma, glioblastoma, adrenocortical carcinoma, AIDS-related lymphoma, anal cancer, bladder cancer, meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, male breast cancer, malignant mesothelioma, medulloblastoma, Merkel cell carcinoma, metastatic squamous neck cell carcinoma, multiple myeloma and other plasma cell neoplasms, mycosis fimgoides and the Sezary syndrome, myelodysplastic syndromes, nasopharyngeal cancer, neuroblastoma, non-small cell lung cancer, small cell lung cancer, head and neck cancer, skin cancer, oropharyngeal cancer, bone cancers, including osteosarcoma and malignant fibrous histiocytoma of bone, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

In some embodiments, the composition is administered intravenously or subcutaneously. In some embodiments, the cancer is treated after administration of the composition.

In some embodiments, the present disclosure provides methods of targeting a population of cancer cells expressing Nectin-4, the method comprising: (i) determining a level of expression of a target in a population of cancer cells; (ii) administering to a subject in need thereof a composition comprising a polypeptide as provided herein, a composition as provided herein, or a pharmaceutical composition as provided herein, wherein the composition comprises at least one component that specifically binds Nectin-4, wherein the composition targets the Nectin-4-expressing cells and is internalized into the Nectin-4 expressing cells; (iii) wherein the patient is treated after the administering as compared to prior to the administering; and the treatment does not damage cells not expressing Nectin-4. In some embodiments, the polypeptide comprises or consists of a miniprotein (M) comprising an amino acid sequence selected from any one of SEQ ID NOs: 1-158 or 177 or 161-176 and/or Table 1C.

In some embodiments, the present disclosure provides miniprotein conjugates comprising: (i) miniprotein (M) that specifically binds to Nectin-4; (ii) a chelator (C) conjugated to (M) through an optional linker (L), wherein (C) comprises DOTA, and (L), when present, comprises PEG; and (iii) a radionuclide (R) chelated to (C), wherein (R) is Actinium-225.

In some embodiments, the present disclosure provides miniprotein conjugates comprising: (i) miniprotein (M) that specifically binds to Nectin-4; (ii) a chelator (C) conjugated to (M) through an optional linker (L), wherein (C) comprises DOTA, and (L), when present, comprises PEG; and (iii) a radionuclide (R) chelated to (C), wherein (R) is Indium-111.

In some embodiments, the present disclosure provides miniprotein conjugates comprising: (i) a miniprotein (M) that specifically binds to Nectin-4; (ii) a chelator (C) conjugated to (M) through an optional linker (L), wherein (C) comprises DOTA, and (L), when present, comprises PEG; and (iii) a radionuclide (R) chelated to (C), wherein (R) is Gallium-68.

In some embodiments, the present disclosure provides miniprotein conjugates comprising: (i) a miniprotein (M) that specifically binds to Nectin-4; (ii) a chelator (C) conjugated to (M) through an optional linker (L), wherein (C) comprises DOTA, and (L), when present, comprises PEG; and (iii) a radionuclide (R) chelated to (C), wherein (R) is Copper-64.

In some embodiments, the miniprotein conjugate as provided herein comprises a miniprotein (M), which miniprotein comprises an amino acid sequence selected from any one of SEQ ID NOs: 1-158 or 177 or 161-176 and/or Table 1C.

In some embodiments, a target is a cell adhesion receptor, a cytokine receptor, a chemokine receptor, a growth factor receptor, an immune cell receptor, or a tumor associated extracellular matrix polypeptide. In some such embodiments the target comprises or consists of Nectin-4 or a portion thereof. In some embodiments, the target comprises an amino acid sequence at least about 90%, 91%, 92%, 93%, 94%, 95%, 96%, 97%, 98%, 99%, or 100% identical to the amino acid sequence set forth in SEQ ID NO: 159 or SEQ ID NO: 160.

In some embodiments, the miniprotein specifically binds to a target. In some embodiments, a miniprotein binds a protein that comprises or consists of an amino acid sequence at least about 90%, 91%, 92%, 93%, 94%, 95%, 96%, 97%, 98%, 99%, or 100% identical to the amino acid sequence set forth in any of SEQ ID NOs: 1-158 or 177 or 161-176 and/or in accordance with Tables 1B and/or 1C or any functional variants or portions thereof.

In some embodiments, the miniprotein comprises an amino acid sequence at least about 90%, 91%, 92%, 93%, 94%, 95%, 96%, 97%, 98%, 99%, or 100% identical to the amino acid sequence set forth in any of SEQ ID NOs: 1-158 or 177 or 161-176 and/or in accordance with Tables 1B and/or 1C or any functional variants or portions thereof.

In some embodiments, the target is Nectin-4. In some embodiments, the miniprotein selectively binds to Nectin-4 or a portion thereof. In some embodiments, the miniprotein comprises an amino acid sequence selected from any one of SEQ ID NOs: 1-158 or 177 or 161-176 and/or in accordance with Tables 1B and/or 1C or any functional variants or portions thereof. In some embodiments, at least 2 amino acids in the amino acid sequence bind to a location on the target. In some embodiments, the miniprotein displays μm or nM binding affinity to the target in an in vitro assay. In some embodiments, a miniprotein has an amino acid sequence at least 98% identical to that set forth in any one of SEQ ID NOs: 1-158 or 177 or 161-176 and/or in accordance with Tables 1B and/or 1C or any functional variants or portions thereof. In some embodiments, a miniprotein has an amino acid sequence at least 99% identical to that set forth in any one of SEQ ID NOs: 1-1-158 or 177 or 161-176 and/or in accordance with Tables 1B and/or 1C or any functional variants or portions thereof. In some embodiments, a composition consists of any one of SEQ ID NOs: 1-158 or 177 or 161-176 and/or in accordance with Tables 1B and/or 1C or any functional variants or portions thereof. In some embodiments, the miniprotein binds to the target with an affinity of about 1 pM to about 500 nM. In some embodiments, the miniprotein binds to the target with an affinity of about 1 pM to about 500 nM, e.g., as measured by an in vitro binding assay. In some embodiments, the affinity is about 100 pM to about 10 nM. In some embodiments, the miniprotein is characterized in that it has high tissue penetrating properties relative to a full-size protein corresponding to the same target. In some embodiments, the miniprotein is stable under ambient conditions. In some embodiments, the miniprotein is stable at 80 degrees Celsius.

In some embodiments, the miniprotein selectively binds to Nectin-4, or a portion thereof. In some embodiments, the miniprotein comprises an amino acid sequence selected from any one of SEQ ID NOs: 1-158 or 177 or 161-176 and/or in accordance with Tables 1B and/or 1C or any functional variants or portions thereof. In some embodiments, at least 2 amino acids in the amino acid sequence bind to a location on the target. In some embodiments, the miniprotein binds to the target with an affinity of about 1 pM to about 500 nM, e.g., as measured by an in vitro binding assay. In some embodiments, the miniprotein is characterized in that it has high tissue penetrating properties relative to a full-size protein corresponding to the same target.

In some embodiments, the present disclosure provides polynucleotides encoding a polypeptide, which polypeptide comprises or consists of an amino acid sequence of any one of SEQ ID NOs: 1-158 or 177 or 161-176 or a functional fragment or portion thereof. In some embodiments, the sequence is codon optimized.

In some embodiments, the present disclosure provides one or more vectors comprising one or more polynucleotides, wherein the one or more polynucleotides encodes one or more polypeptides, which one or more polypeptides comprises or consists of an amino acid sequence comprising or consisting of any one of SEQ ID NOs: 1-158 or 177 or 161-176 and/or in accordance with Tables 1B and/or 1C or any functional variants or portions thereof.

In some embodiments, the present disclosure provides an isolated polynucleotide comprising one or more nucleic acid sequences encoding one or more of SEQ ID NO: 1-158 or 177 or 161-176 and/or in accordance with Tables 1B and/or 1C or any functional variants or portions thereof. In some such embodiments, such a nucleic acid encodes a polypeptide comprising at least 90%, 95%, 96%, 97%, 98%, 99% or greater identity to SEQ ID NO: 1-158 or 177 or 161-176 and/or in accordance with Tables 1B and/or 1C or any functional variants or portions thereof.

In some embodiments, the present disclosure provides a host cell comprising a vector encoding a polypeptide comprising or consisting of any one of SEQ ID NOs: 1-158 or 177 or 161-176 and/or in accordance with Tables 1B and/or 1C or any functional variants or portions thereof.

In some embodiments, the present disclosure provides an isolated polypeptide having an amino acid sequence comprising or consisting of: one or more amino acid sequences selected from SEQ ID NO: 161-176, in accordance with Tables 1B and 1C; or one or more amino acid sequences comprising at least 80%, 85%, 90%, 95%, 96%, 97%, 98%, 99% or greater identity to SEQ ID NO: 161-176, in accordance with Tables 1B and 1C. In some embodiments, the polypeptide comprises no more than 100 amino acids. In some embodiments, the polypeptide specifically binds to Nectin-4 or a portion thereof.

In some embodiments, the present disclosure provides isolated polynucleotides comprising one or more nucleic acid sequences encoding a polypeptide selected from SEQ ID NO: 161-176 and/or in accordance with Tables 1B and 1C; or a nucleic acid sequence encoding a polypeptide comprising at least 90%, 95%, 96%, 97%, 98%, 99% or greater identity to SEQ ID NO: 161-176 and/or in accordance with Tables 1B and/or 1C. In some embodiments, a polypeptide does not bind to a non-target (e.g., a non-target protein) at an affinity greater than 10 nM. In some embodiments, the non-target does not include Nectin-4 or a functional variant or portion thereof.

### BRIEF DESCRIPTION OF FIGURES

FIGS. 1A and 1B show binding affinities (BIACORE) of an exemplary Nectin-4 peptide.

FIGS. 2A and 2B show binding affinities (BIACORE) of an exemplary Nectin-4 peptide.

FIGS. 3A and 3B show binding affinities (BIACORE) of an exemplary conjugate comprising an exemplary Nectin-4 peptide.

FIGS. 4A and 4B show association (4A) and dissociation (4B) kinetics for exemplary compounds comprising a miniprotein provided herein.

FIGS. 5A and 5B show association (5A) and dissociation (5B) exemplary compounds comprising a miniprotein provided herein.

FIGs. 6A and 6B show internalization of an exemplary radioconjugate, C215, in HT1376 (6A) and MCF7 (6B) cell lines. TB=total binding, NSB=non-specific binding.

FIG. 7 shows a graph depicting thermostability showing percent of exemplary compound, C215, over 60 minutes while heated to 75 degrees Celsius.

### DETAILED DESCRIPTION

Among other things, the present disclosure provides compositions and methods of use thereof. In some embodiments, a composition selectively binds to a target (e.g., Nectin-4). In some embodiments the composition comprises one or more therapeutic agents (e.g., a chelator, a radionuclide), wherein the therapeutic agent is selectively targeted to a cell expressing Nectin-4 such that the Nectin-4-expressing cell is treated and cells not expressing Nectin-4 are not treated. The present disclosure recognizes that a source of a problem in treating cells expressing a target (e.g., cancer cells) is that traditional therapies are not selective enough to specifically target cells and to deliver a therapeutic in a way that minimizes damage to surrounding cells. The present disclosure provides the insight that a combination of selective targeting with a specific therapeutic such as a chelator and/or radionuclide (e.g., an alpha emitter) provides an advantage over previously used therapeutics (e.g., antibodies, beta-emitters, etc.)

Unless otherwise defined herein, scientific and technical terms used in connection with the present disclosure shall have the meanings that are commonly understood by those of ordinary skill in the art. Furthermore, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well-known and commonly used in the art.

The methods and techniques of the present disclosure are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Wittrup and VanAntwerp, Fine Affinity Discrimination by Yeast Surface Display and Flow Cytometry, Biotechnol. Prog. 2002, (16) 31-37; C. Queen et al., A humanized antibody that binds to the interleukin 2 receptor, Proc. Natl. Acad. Sci. USA 1989, 86 (24) 10029-10033; Scheinberg D A and McDevitt M R. Actinium-225 in targeted alpha-particle therapeutic applications. Curr Radiopharm. 2011; 4(4):306-320.

All publications, patents, and other references mentioned herein are hereby incorporated by reference in their entireties. In case of conflict, the present specification, including definitions, will control. Materials, methods, and examples as disclosed herein are illustrative only and not intended to be limiting.

### Definitions

Unless otherwise defined, all technical and scientific terms used herein have the same meaning as commonly understood by one of ordinary skill in the art to which this present disclosure pertains. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well-known and commonly used in the art.

Throughout this specification and claims, the word “comprise” or variations such as “comprises” or “comprising,” will be understood to imply the inclusion of a stated integer or group of integers but not the exclusion of any other integer or group of integers.

As used herein, ranges and amounts can be expressed as “about” a particular value or range. About also includes the exact amount. Hence “about 100 nucleotides” means “about 100 nucleotides” and also “100 nucleotides.” Generally, the term “about” as used herein includes an amount that would be expected to be within experimental error. If “about” appears before a quantitative value, the present disclosure also includes the specific quantitative value itself, unless specifically stated otherwise. In such instances “about” can also refer to a ±10% variation from the nominal value unless otherwise indicated or inferred.

Unless otherwise indicated, and as an example for all sequences described herein under the general format “SEQ ID NO:”, “nucleic acid comprising SEQ ID NO: 1” refers to a nucleic acid, at least a portion of which has either (i) the sequence of SEQ ID NO: 1, or (ii) a sequence complementary to SEQ ID NO: 1. The choice between the two is dictated by the context. For instance, if the nucleic acid is used as a probe, the choice between the two is dictated by the requirement that the probe be complementary to the desired target.

As used herein, the term “administration” refers providing a composition to a subject or system. Administration to a subject may be by any appropriate route, dose and/or dose schedule.

As used herein, the term “affibody” refers to a subgenus of miniproteins. An affibody is a molecule derived from the Z-domain of staphylococcal protein A that consists of three alpha helices with 58 amino acids and has a molar mass of about 6 kDa. See, for exemplary details of affibody structures and uses, Orlova, A; Magnusson, M; Eriksson, T L; Nilsson, M; Larsson, B; H6id6n-Guthenberg, I; Widstrom, C; Carlsson, J et al. (2006). “Tumor imaging using a picomolar affinity HER2 binding affibody molecule”, Cancer Res. 66 (8): 4339-48. Exemplary Affibody® Molecules are commercially available from Abcam Corp. Cambridge Mass. An affibody is stable at high temperatures and under acidic or alkaline conditions. Target specificity is obtained by randomization of 13 amino acids located in two alpha-helices involved in the binding activity of the parent protein domain (Feldwisch J, Tolmachev V.; (2012) Methods Mol Biol. 899:103-26).

As used herein, the term “affinity maturation” generally refers to a process whereby successive changes to a sequence (e.g., successive mutations) are made and selection of the polypeptide sequences are performed to choose one or more sequences with increased affinity relative to the “starting” sequence or another sequence with less affinity as compared to one with greater affinity.

As used herein, the terms “amino acid sequence” and “polypeptide” refer to a polymer of amino acids connected by one or more peptide bonds. A polypeptide of the present disclosure encompasses both naturally-occurring and non-naturally-occurring proteins, and any fragments, portions, peptides, mutants, derivatives, and analogs thereof. A polypeptide may be monomeric or polymeric. Further, a polypeptide may comprise a number of different domains each of which has one or more distinct activities. A polypeptide may be fully or partially synthetic or otherwise modified (i.e., comprising one or more synthetically-produced amino acids and/or modifications thereof). The term “peptide” may be used to refer to a short polypeptide, such as one comprising fewer than about 70 amino acids (e.g., between about 5, 10, 15, 20, 25, 30, 35, 40, 45, 50, 55, 60, 65, or 70 amino acids).

As used herein, the term “anticalin” refers to a subgenus of miniproteins. An anticalin is an engineered protein derived from a lipocalin (Beste G, Schmidt F S, Stibora T, Skerra A. (1999) Proc Natl Acad Sci USA. 96(5): 1898-903; Gebauer and Skerra (2009) Curr Opinion in Chemical Biology 13:245-255). Anticalins possess an eight-stranded b-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

As used herein, the term “attenuate” as used herein generally refers to a functional deletion, including a mutation, partial or complete deletion, insertion, or other variation made to a gene sequence or a sequence controlling the transcription of a gene sequence, which reduces or inhibits production of the gene product, or renders the gene product non-functional. In some instances, a functional deletion is described as a knockout mutation. Attenuation also includes amino acid sequence changes by altering the nucleic acid sequence, placing the gene under the control of a less active promoter, down-regulation, expressing interfering RNA, ribozymes or antisense sequences that target the gene of interest, or through any other technique known in the art. In one example, the sensitivity of a particular enzyme to feedback inhibition or inhibition caused by a composition that is not a product or a reactant (non-pathway specific feedback) is lessened such that the enzyme activity is not impacted by the presence of a compound. In other instances, an enzyme that has been altered to be less active can be referred to as attenuated.

As used herein, the term “avimer” refers to a subgenus of miniproteins. An avimer is a class of antibody mimetics which consist of two or more peptide sequences of preferably 30 to 35 amino acids each, which are derived from A-domains of various membrane receptors and which are connected by linker peptides. Binding of target molecules occurs via the A-domain and domains with the desired binding specificity can be selected, for example, by phage display techniques. The binding specificity of the different A-domains contained in an avimer may, but does not have to be identical (Weidle U H, et al., (2013), Cancer Genomics Proteomics; 10(4): 155-68). For further details see Nature Biotechnology 23(12), 1556-1561 (2005) and Expert Opinion on Investigational Drugs 16(6), 909-917 (June 2007).

As used herein, the term “binder” refers to a subgenus of miniprotein. A binder is characterized in that it comprises or consists of a polypeptide (e.g., peptide) that is capable of binding or has known ability to engage and associate a target or a portion thereof. Binders generally comprise a cysteine-containing peptide comprising one or more disulfide bonds, though some binders do not comprise cysteine-residues and/or disulfide bonds. Binders are preferably cleared rapidly from circulation when administered systemically to a mammalian subject. As will be understood, given context, reference to a binder may be or include its nucleic acid sequence or amino acid sequence encoding it. A binder may be provided, for instance, as a polynucleotide, polypeptide, using a vector, host cell, etc., and/or any combination of modalities. A binder may be derived or manufactured using any method known to those of skill in the art. For instance, in some embodiments, a binder can be recombinant (i.e., produced using recombinant nucleic acids encoding a polypeptide). In some embodiments, a binder can be synthetic (e.g., synthesized such as using standard solid phase synthesis methods, such as solid phase peptide synthesis, known to those of skill in the art (see, e.g., Palomo, J. RSC Adv., 2014,4, 32658-32672) and described herein).

The term “chelator” as used herein refers to any molecule or moiety that is capable of forming a complex (i.e., “chelates”) with a metal ion. Chelators generally have two or more unshared electron pairs that can be used to donate to a metal ion. Metal ions are usually coordinated to the chelator by two or more pairs of electrons.

As used herein, the term “conjugated” refers to the joining by covalent or noncovalent means of two compounds or agents.

As used herein, a “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference). The following six groups each contain amino acids that are conservative substitutions for one another: 1) Serine (S), Threonine (T); 2) Aspartic Acid (D), Glutamic Acid I; 3) Asparagine (N), Glutamine (Q); 4) ArginiI(R), Lysine (K); 5) Isoleucine (I), Leucine (L), Methionine (M), Alanine (A), Valine (V), and 6) Phenylalanine (F), Tyrosine (Y), Tryptophan (W).

As used herein the terms “cysteine-dense peptide” and “CDP” are used interchangeably and refer to a subgenus of miniproteins that generally comprise a high density of cysteines (e.g., at least one, two, three, four, or more cysteines in a span of about 10 to about 90 amino acids, or about 13 to 80 amino acids in a polypeptide). In some embodiments, such a CDP may comprise at least two independent folding domains. In some embodiments, the CDP comprises at least one, two, three, four, or more cysteine residues in a span of from about 10 to about 90 amino acid residues, preferably 13 to 80 amino acid residues. (pubmed.ncbi.nlm.nih.gov/29483648/) In some embodiments, the CDP comprises a constrained distribution of cysteines, Cys-_{X[0-15]}-Cys-_{X[0-15]}-Cys-_{X[0-15]}-Cys-_{X[0-15]}-Cys-X_{[0-15]}-Cys (wherein X represents any amino acid).

As used herein, the term “deletion” generally refers to the removal of one or more nucleotides from a nucleic acid molecule or one or more amino acids from a protein, the regions on either side being joined together.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

As used herein, the term “derived from,” with reference to a nucleic acid sequence refers to a nucleic acid sequence that has at least 85% sequence identity to a reference naturally occurring nucleic acid sequence from which it is derived. The term “derived from,” with reference to an amino acid sequence refers to an amino acid sequence that has at least 85% sequence identity to a reference naturally occurring amino acid sequence from which it is derived. The term “derived from” as used herein does not denote any specific process or method for obtaining the nucleic acid or amino acid sequence. For example, the nucleic acid or amino acid sequence can be chemically synthesized.

As used herein, the term “designed ankyrin repeat domain (DARPin)” refers to a subgenus of miniproteins. A DARPin is a peptide derived from Ankyrin which is a family of proteins that mediate attachment of integral membrane proteins to the cytoskeleton. A single ankyrin repeat is preferably a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1. DARPins typically provide a rigid interface and lack structural flexibility (Gebauer and Skerra, 2009).

As used herein, the term “domain” as used herein refers to a structure of a biomolecule that contributes to a known or suspected function of the biomolecule. Domains may be co-extensive with regions or portions thereof; domains may also include distinct, non-contiguous regions of a biomolecule. Examples of protein domains include, but are not limited to, an Ig domain, an extracellular domain, a transmembrane domain, and a cytoplasmic domain.

As used herein, the term “engineered Kunitz domain” refers to a subgenus of miniproteins. An engineered Kunitz domain is preferably a peptide derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6 kDa and domains with the required target specificity can be selected by display techniques such as phage display (Weidle et al., (2013), Cancer Genomics Proteomics; 10(4): 155-68).

As used herein, the term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

As used herein, the term “functional variant” refers to a polypeptide that comprises or consists of a portion of a sequence of a polypeptide provided herein, and still retains one or more functions of a polypeptide comprising or consisting of an entire amino acid sequence as provided herein (e.g., still binds to a target, e.g., Nectin-4).

As used herein, the term “fusion protein” refers to a polypeptide comprising a polypeptide or fragment coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusions that include the entirety of the proteins of the present disclosure have particular utility. The heterologous polypeptide included within the fusion protein of the present disclosure is at least 6 amino acids in length, often at least 8 amino acids in length, and usefully at least 15, 20, and 25 amino acids in length. Fusions that include larger polypeptides, such as an IgG Fe region, and even entire proteins, such as the green fluorescent protein (“GFP”) chromophore-containing proteins, have particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

As used herein, when referring to a protein, “homology” to a second protein can exist if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) Homology between two regions of amino acid sequences (especially with respect to predicted structural similarities) can be interpreted as implying similarity in function. Homologous proteins or peptides with residue positions that are not identical are often recognized to differ by conservative amino acid substitutions.

As used herein the term “identical” refers to a nucleic acid sequence or amino acid sequence refers to at least two nucleic acid or at least two amino acid sequences or subsequences that have a specified percentage of nucleotides or amino acids, respectively, that are the same, when compared and aligned for maximum correspondence, as measured using a sequence comparison algorithm or by visual inspection. For sequence comparison, typically one sequence acts as a reference sequence, to which test sequences are compared. A length of sequence identity comparison may be over a stretch of any number of nucleotides or amino acids. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. A number of algorithms are known in the art. Non-limiting examples of algorithms that are suitable for determining percent sequence identity and sequence similarity are the BLAST and BLAST 2.0 algorithms, which are described in, e.g., Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information. Additionally or alternatively sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity can be determined using FASTA with its default parameters (a word size of 6 and the NOPAM factor for the scoring matrix) or using Gap with its default parameters as provided in GCG Version 6.1, herein incorporated by reference.

As used herein, the term “isolated” polynucleotide or polypeptide is one which is substantially separated from other cellular components that naturally accompany the native polynucleotide in its natural host cell, e.g., ribosomes, polymerases and genomic sequences with which it is naturally associated. For instance, an isolated molecule is one that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polynucleotide or polypeptide found in nature or it includes amino acid analogs or derivatives not found in nature or linkages other than standard peptide bonds). Thus, a polynucleotide or polypeptide that is chemically synthesized or synthesized in a cellular system different from the cell from which it naturally originates will be “isolated” from its naturally associated components. A polynucleotide or polypeptide may also be rendered substantially free of naturally associated components by isolation, using protein purification techniques well known in the art. As thus defined, “isolated” does not necessarily require that any molecule so described has been physically removed from its native environment. In some embodiments, as used in reference to an isolated construct, isolated means in the absence of a pharmaceutically acceptable salt.

As used herein, the term “K_{D}” or “Kd” refers to the dissociation equilibrium constant for a particular antibody-antigen interaction. Typically, the antibody of the present disclosure binds to Nectin-4 with a dissociation equilibrium constant (K_{D}) of less than about 10⁻⁷ M, such as less than about 10⁻⁸ M, 10⁻⁹ M, or 10⁻¹⁰ M or less, for example, as determined using surface plasmon resonance (SPR) techniques in a BIACORE instrument.

As used herein, the term “knock out” generally refers to a gene whose level of expression or activity has been reduced to zero. In some examples, a gene is knocked out via deletion of some or all of its coding sequence. In other examples, a gene is knocked out via introduction of one or more nucleotides into its open reading frame, which results in translation of a nonsense or otherwise nonfunctional protein product.

As used herein, the term “knottin” refers to a structural motif of a miniprotein containing three disulfide bridges.

As used herein, the term “knottin peptide” refers to a subgenus of miniproteins that comprises at least one knottin.

As used herein, the term “linker” refers to a moiety that is used to conjugate a miniprotein to a chelator.

As used herein, the term “miniprotein” refers to short proteins of less than or equal to 100 amino acids with well-defined folds comprising two or more secondary structure elements, a sequestered hydrophobic core, and/or cooperative folding. CDPs, knottins, affibodies, engineered Kunitz domains, monobodies (adnectins), anticalins, designed ankyrin repeat domains (DARPins), avimers, and binders as disclosed herein are all examples of miniproteins.

As used herein, the term “modification,” with reference to a nucleic acid sequence, refers to a nucleic acid sequence that comprises at least one substitution, alteration, inversion, addition, or deletion of nucleotide compared to a reference nucleic acid sequence. As used herein, the term “modification,” with reference to an amino acid sequence refers to an amino acid sequence that comprises at least one substitution, alteration, inversion, addition, or deletion of an amino acid residue compared to a reference nucleic acid sequence.

As used herein, the term “modified derivative” refers to polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as 125I, 32P, 35S, and 3H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002) (hereby incorporated by reference).

As used herein, the term “molecule” means any compound, including, but not limited to, a small molecule, peptide, protein, sugar, nucleotide, nucleic acid, lipid, etc., and such a compound can be natural or synthetic.

As used herein, the term “monobody” or “adnectin” are used interchangeably and refer to a subgenus of miniproteins. A monobody relates to a molecule, preferably based on ^{th}e 10th extracellular domain of human fibronectin III (1° fn3), which adopts an Ig-like b-sandwich fold of preferably 94 residues with 2 to 3 exposed loops, but lacks the central disulfide bridge (Gebauer and Skerra (2009) Curr Opinion in Chemical Biology 13:245-255). Adnectins with the desired target specificity can be genetically engineered by introducing modifications in specific loops of the protein.

As used herein, the term “mutein” or “mutant protein” or “variant” means a protein comprising an amino acid sequence with at least one variation (e.g., an insertion, a deletion, or a substitution, which can be a conservative or non-conservative substitution) compared to a reference sequence. When applied to sequences (e.g., nucleic acid sequences, amino acid sequences) “mutated” means that nucleotides in a nucleic acid sequence or amino acids in an amino acid sequences may be inserted, deleted or changed compared to a reference sequence. A single alteration may be made at a locus (a point mutation) or multiple nucleotides or amino acids may be inserted, deleted or changed at a single locus. In addition, one or more alterations may be made at any number of loci within a nucleic acid or amino acid sequence. A nucleic acid or amino acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); “oligonucleotide-directed mutagenesis” (a process which enables the generation of site-specific mutations in any cloned DNA segment of interest; see, e.g., Reidhaar-Olson and Sauer, Science 241:53-57 (1988)); directed evolution (e.g., exposing a polypeptide to differing sets of conditions resulting in production of different polypeptides with one or more amino acid changes that may or may not confer greater fitness upon the polypeptide); and site-directed mutagenesis (e.g., specifically directed changes in a sequence).

As used herein, the term “non-disulfide sequence” refers to an amino acid sequence encoding a polypeptide that does not comprise more than one cysteine residue and/or disulfide bonds in its folded and active form. In some embodiments, miniprotein may comprise or consist of a non-disulfide sequence.

As used herein, the term “non-peptide analog” refers to a compound with properties that are analogous to those of a reference polypeptide. A non-peptide compound may also be termed a “peptide mimetic” or a “peptidomimetic.” See, e.g., Jones, Amino Acid and Peptide Synthesis, Oxford University Press (1992); Jung, Combinatorial Peptide and Nonpeptide Libraries: A Handbook, John Wiley (1997); Bodanszky et al., Peptide Che-stry--A Practical Textbook, Springer Verlag (1993); Synthetic Peptides: A Users Guide, (Grant, ed., W. H. Freeman and Co., 1992); Evans et al., J. Med. Chem. 30:1229 (1987); Fauchere, J. Adv. Drug Res. 15:29 (1986); Veber and Freidinger, Trends Neurosci., 8:392-396 (1985); and references sited in each of the above, which are incorporated herein by reference. Such compounds are often developed with the aid of computerized molecular modeling. Peptide mimetics that are structurally similar to useful peptides of the present disclosure may be used to produce an equivalent effect and are therefore envisioned to be part of the present disclosure.

As used herein, the terms “nucleic acid sequence” and “polynucleotide” are used interchangeably to refer to a polymer of nucleotides. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation. The nucleic acid sequence can contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid sequence. Nucleic acid sequences include, but are not limited to, all nucleic acid sequences which are obtained by any means available in the art, including, without limitation, recombinant means, e.g., the cloning of nucleic acid sequences from a recombinant library or a cell genome, using ordinary cloning technology and polymerase chain reaction, and the like, and by synthetic means. Polynucleotides of the present disclosure may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

As used herein, the terms “operatively linked” or “operably linked” expression control sequences refers to a linkage in which the expression control sequence is contiguous with the gene of interest to control the gene of interest, as well as expression control sequences that act in trans or at a distance to control the gene of interest.

As used herein, the terms “polypeptide mutant” or “mutein” refers to a polypeptide whose sequence contains an insertion, duplication, deletion, rearrangement or substitution of one or more amino acids compared to the amino acid sequence of a native or wild-type protein. A mutein may have one or more amino acid point substitutions, in which a single amino acid at a position has been changed to another amino acid, one or more insertions and/or deletions, in which one or more amino acids are inserted or deleted, respectively, in the sequence of the naturally-occurring protein, and/or truncations of the amino acid sequence at either or both the amino or carboxy termini. A mutein may have the same but preferably has a different biological activity compared to the naturally-occurring protein. A mutein has at least 85% overall sequence homology to its wild-type counterpart. Even more preferred are muteins having at least 90% overall sequence homology to the wild-type protein. In an even more preferred embodiment, a mutein exhibits at least 95% sequence identity, even more preferably 98%, even more preferably 99% and even more preferably 99.9% overall sequence identity. Sequence homology may be measured by any common sequence analysis algorithm, such as Gap or Bestfit. Amino acid substitutions can include those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such analogs.

As used herein, the term “polypeptide fragment” as used herein refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

The term “radionuclide” as used herein refers to an atom capable of undergoing radioactive decay.

As used herein, the term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, and/or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it. A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, the term “recombinant host cell” (or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

As used herein, the term “region” as used herein refers to a physically contiguous portion of the primary structure of a biomolecule. In the case of proteins, a region is defined by a contiguous portion of the amino acid sequence of that protein.

As used herein the phrase “secondary structure elements” refers to local folded structures that form within a polypeptide due to interactions between atoms of its backbone. Examples of secondary structure elements can include an alpha helix, a beta sheet, a 310 helix, a pi helix, and a random coil. A miniprotein of the present disclosure may comprise one or more of any of such secondary structures (e.g., one or more alpha helix, one or more alpha helices and one or more beta sheets). It will be understood by those of skill in the art that secondary structure elements may be joined by loop regions, which may or may not be modified to change the interactions of secondary structure elements of the polypeptide. As will be understood to those of skill in the art, in some embodiments, loops may be secondary structural elements. In some embodiments, loops may be interstructural elements that are not necessarily considered secondary structural elements.

As used herein, “sequence homology” for polypeptides, also referred to as “percent sequence identity,” is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1. A preferred algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)). Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOSUM62. The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

As used herein, the term “specificity” generally refers to a sequence that, when in a conformation that can bind, selectively or “specifically” binds to a specific target. As used herein, “specifically binds” means that the binding of a polynucleotide, polypeptide, or protein is selective for a specified antigen and can be discriminated from unwanted or non-specific interactions. For example, the ability of a protein (e.g., cysteine-dense peptides) to bind to a specific antigenic determinant can be measured techniques familiar to one of skill in the art, for example through an enzyme-linked immunosorbent assay (ELISA) or surface plasmon resonance. Between two molecules, “specific binding” refers to the ability of two molecules to bind to each other in preference to binding to other molecules in the environment. Typically, “specific binding” discriminates over adventitious binding in a reaction by at least two-fold, more typically by at least 10-fold, often at least 100-fold. Typically, the affinity or avidity of a specific binding reaction, as quantified by a dissociation constant, is about 10⁻⁷ M or stronger (e.g., about 10⁻⁸ M, 10⁻⁹ M or even stronger). Specific-binding requires specificity of a particular first entity (e.g., a polypeptide) for a particular second entity (e.g., an antigen binding sequence).

As used herein, the term “stabilizer” in the context of a pharmaceutical composition refers to an agent, molecule, or compound that may act to impact the active pharmaceutical ingredient or ingredients to maintain desirable properties (e.g., therapeutic properties or properties that allow therapeutic effect to be achieved) until it is administered to a subject.

As used herein “stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization. In general, “stringent hybridization” is performed at about 25° C. below the thermal melting It (Tm) for the specific DNA hybrid under a particular set of conditions. “Stringent washing” is performed at temperatures about 5° C. lower than the Tm for the specific DNA hybrid under a particular set of conditions. The Tm is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51, hereby incorporated by reference. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

As used herein, the term “synthetic” is used to refer to an entity that is made is lab-created and not naturally produced or isolated, without modification, from a naturally occurring source. A recombinant polymer, such as a recombinant polynucleotide or polypeptide, may be synthetic. Synthetic polymers such as polynucleotides or polypeptides may be produced by any method known to those of skill in the art, including but not limited to solid phase synthesis, solution phase synthesis, biological synthesis by, e.g., host cells, etc.

As used herein, the term “subject” is a mammal. A subject may be a human or non-human mammal. Given context, a subject may be used interchangeably with patient, individual, donor, etc.

As used herein, the terms “substantial homology” or “substantial similarity,” when referring to a polynucleotide or polypeptide, indicate that, when optimally aligned with appropriate nucleotide or amino acid insertions or deletions with another reference molecule (or its complementary strand when appropriate), there is sequence identity in at least about 70%, 75%, 80%, 85%, preferably at least about 90%, and more preferably at least about 95%, 96%, 97%, 98% or 99% or more of the nucleic acid or amino acid residues, as measured by any well-known algorithm of sequence identity, such as, e.g., FASTA, BLAST, Gap, etc.. Alternatively or additionally, substantial homology or similarity exists when, for example, a nucleic acid or fragment thereof hybridizes to another nucleic acid, to a strand of another nucleic acid, or to the complementary strand thereof, under stringent hybridization conditions.

As used herein, the term “target” refers to a protein or functional portion or variant thereof. A target may be expressed on the surface of a particular cell (a “target cell”) or expressed within (e.g., on the surfaces of) cells in a population of cells. A target may have a certain percent identity to a reference protein and still be referred to as a target by a particular name (e.g., Nectin-4). A target may also refer to a protein in a pathway related to another protein. For example, if a target is Nectin-4, a target may also be a protein in a pathway that is necessary for Nectin-4 activity. A target may be or comprise a binding region, such as an epitope, to which a miniprotein (e.g., CDP, knottin, binder, affibody, engineered Kunitz domain, monobody, anticalin, designed ankyrin repeat domain (DARPin), avimer) of the present disclosure binds.

As used herein “thermal stability” refers to the ability of a miniprotein to remain stable (e.g., not unfolded, e.g., structurally intact) over a period of time. In some embodiments, a miniprotein of the present disclosure retains at least 95% of its stability for at least one hour.

As used herein, a treatment that is “tolerable” to a subject refers to a therapeutic administration and/or regimen that is not terminated because of dose-limiting toxicity.

As used herein, the term “treatment” (as well as “treat” or “treating”) refers to partial or complete alleviation, amelioration, mitigation, prevention, reduction in risk of onset, relief, inhibition, delay in onset of, reduction in severity of, reduction in frequency or incidence of one or more causes, features, and/or symptoms of or associated with a particular disease, disorder, and/or condition.

As used herein, the term “vector” is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply “expression vectors”).

### Compositions

Provided herein are novel compositions comprising one or more of a polypeptide, linker, chelator, and/or radionuclide. In some embodiments, a composition comprises a linker and a chelator. In some embodiments, a composition comprises a linker, chelator, and radionuclide. In some embodiments, a composition comprises or consists of a polypeptide (i.e., miniprotein), an optional linker, and a chelator and/or radionuclide. In some embodiments, a chelator and/or radionuclide are conjugated to a miniprotein via a linker. In some embodiments, a miniprotein of the present disclosure comprises or consists of a CDP, a knottin, a binder, an affibody, an engineered Kunitz domain, a monobody, an anticalin, a designed ankyrin repeat domain (DARPin), and/or an avimer. In some embodiments, the miniprotein comprises or consists of a CDP. In some such embodiments, the miniprotein comprises or consists of a knottin. In some such embodiments, the miniprotein comprises or consists of a binder. In some such embodiments, the miniprotein comprises or consists of an affibody. In some such embodiments, the miniprotein comprises or consists of an engineered Kunitz domain. In some such embodiments, the miniprotein comprises or consists of a monobody. In some such embodiments, the miniprotein comprises or consists of an anticalin. In some such embodiments, the miniprotein comprises or consists of a designed ankyrin repeat domain (DARPin). In some such embodiments, the miniprotein comprises or consists of an avimer. In some embodiments the miniprotein (e.g., CDP, knottin, binder, affibody, engineered Kunitz domain, monobody, anticalin, designed ankyrin repeat domain (DARPin), avimer) is designed to be linked to one or more other components. For example, in some embodiments, a miniprotein (e.g., CDP, knottin, binder, affibody, engineered Kunitz domain, monobody, anticalin, designed ankyrin repeat domain (DARPin), avimer) may be linked (conjugated) to another component such as a chelator and/or a radionuclide. In some embodiments, a radionuclide of the present disclosure is an alpha emitter. In some such embodiments, a chelator and/or radionuclide are conjugated to a miniprotein via a linker.

Without wishing to be bound by any particular theory, the present disclosure contemplates that compositions of the present disclosure are more effective than previously described compositions (e.g., such as those comprising antibodies and/or beta-emitter radionuclides). Such miniproteins or compositions comprising miniproteins can be used to treat subjects in need thereof with improved target specificity, increased speed of clearance, and decreased off-target effects (e.g., as compared to conjugates with non-alpha emitter radionuclides, e.g., as compared to compositions comprising antibodies or antibody-drug-conjugates, etc.) For example, while miniproteins (e.g., to be used in compositions as provided herein) have several key features of antibody-based therapeutics (e.g., affinity, potency, specificity, and ability to disrupt protein:protein interactions), they can avoid undesirable limitations such as, e.g., large size, expensive manufacturing, and the necessity of chimerization or humanization. For instance, in some embodiments, a miniprotein (e.g., CDP, knottin, binder, affibody, engineered Kunitz domain, monobody, anticalin, designed ankyrin repeat domain (DARPin), avimer) of the present disclosure is no more than about 100 amino acids in length. In some embodiments, such a miniprotein (e.g., CDP, knottin, binder, affibody, engineered Kunitz domain, monobody, anticalin, designed ankyrin repeat domain (DARPin), avimer) may be or comprise a cysteine dense peptide. In some embodiments, a miniprotein (e.g., CDP, knottin, binder, affibody, engineered Kunitz domain, monobody, anticalin, designed ankyrin repeat domain (DARPin), avimer) comprises one or more disulfide bridges. In some embodiments, a miniprotein (e.g., CDP, knottin, binder, affibody, engineered Kunitz domain, monobody, anticalin, designed ankyrin repeat domain (DARPin), avimer) comprises multiple cysteine residues that crosslink to maintain a very stable, folded state for a peptide of its length (e.g., relative to a peptide of the same length without as many cysteine residues). Without being bound by any particular theory, the present disclosure contemplates that in some embodiments, a miniprotein does not comprise multiple cysteine residues such as, for example, a miniprotein comprising a single cysteine residue. In some such embodiments, the miniprotein may form a dimer, such as with another miniprotein (e.g., self-dimerization). In some embodiments, two miniproteins are linked together to form a dimer. In other embodiments, two miniproteins are each linked to a linker to form a dimer. In other embodiments, two different miniproteins are each linked to a linker to form a dimer. The present disclosure contemplates that stability conferred by crosslinked cysteines contributes to reduced immunogenicity of miniproteins or comprising such miniproteins. In some embodiments, such stability may also confer resistance to harsher conditions provided for efficient chelation (e.g., high temperature, low pH incubations, etc.), while continuing to retain biological activity (e.g., capability of binding a target).

In some embodiments, miniproteins as provided herein function as targeting moieties, e.g., specifically binding to a target expressed on the surface of a tumor cell. In some such embodiments, a miniprotein is designed such that it may be joined to one or more additional components. For example, without being bound by any particular theory, miniproteins of the present disclosure may be formulated such that they are combined with other components such as a therapeutic molecule (e.g., chelator compositions and/or radionuclide) and/or a detectable agent (e.g., a visualizable agent, e.g., a metabolizable and visualizable agent). In some such embodiments, such miniproteins conjugated to one or more additional components may be used, for example, in diagnosis, prognosis, monitoring, and/or treatment of one or more diseases, disorders or conditions such as those with expression of particular targets on particular populations of cells.

In some embodiments a miniprotein (e.g., CDP, knottin, binder, affibody, engineered Kunitz domain, monobody, anticalin, designed ankyrin repeat domain (DARPin), avimer) has low immunogenicity relative to a larger protein. In some such embodiments, the lower immunogenicity increases amenability to harsher environmental conditions (e.g., high temperature and low pH incubations) while retaining biological activity. Thus, in some embodiments, a conjugate comprising a miniprotein has lower immunogenicity than a composition comprising a larger protein or different targeting moiety (i.e., other than a miniprotein).

In some embodiments, a composition comprising a linker, chelator, and/or radionuclide can efficiently penetrate a tumor.

In some embodiments, miniproteins (e.g., CDP, knottin, binder, affibody, engineered Kunitz domain, monobody, anticalin, designed ankyrin repeat domain (DARPin), avimer) have superior penetration efficiency relative to larger proteins. That is, in some embodiments, a miniprotein or composition comprising a miniprotein can penetrate a solid tumor better than a larger protein or composition comprising a protein larger than a miniprotein. For example, in some embodiments, a binder has superior tumor penetration efficiency with a hydrodynamic radius on the order of about 1 nm -25 nm. In some embodiments, the hydrodynamic radius is between about 1 nm −5 nm. In some embodiments, the hydrodynamic radius is between about 1 nm -3 nm. In some embodiments, the hydrodynamic radius is about 1, 2, 3, 4, 5, 6, 7, 8, 9, 10, 11, 12, 13, 14, 15, 16, 17, 18, 19, 20, 21, 22, 23, 24, or 25 nm.

As described herein, miniproteins (e.g., CDP, knottin, binder, affibody, engineered Kunitz domain, monobody, anticalin, designed ankyrin repeat domain (DARPin), avimer), are conjugated to a chelator. In some embodiments, the chelator binds a radionuclide (e.g., an alpha-emitter radionuclide, e.g., actinium). In some such embodiments, such radionuclide conjugates combine specific-binding capabilities and properties of a miniprotein (e.g., CPD, knotting, binder, affibody, engineered Kunitz domain, monobody, anticalin, designed ankyrin repeat domain (DARPin), avimer) with a radionuclide. That is, without being bound by any particular theory, the present disclosure provides a conjugate wherein, in some embodiments, a miniprotein (e.g., CDP, knottin, binder, affibody, engineered Kunitz domain, monobody, anticalin, designed ankyrin repeat domain (DARPin), avimer) targets a radioisotope to which it's conjugated to a cell expressing a target. In some embodiments, the target is expressed on the surface of a cell. In some embodiments, the target is Nectin-4. In some embodiments, the cell is a tumor cell. In some embodiments, the conjugate binds to the Nectin-4 on the surface of the tumor cell. In some such embodiments, the radionuclide is targeted to the tumor cell. In some embodiments, the radionuclide is an alpha-emitter radionuclide and when internalized, serves to specifically target (e.g., without damaging surrounding tissue/cells) the tumor cell.

### Targets

Any cell expressing a target may be targeted by a miniprotein (e.g., CDP, knottin, binder, affibody, engineered Kunitz domain, monobody, anticalin, designed ankyrin repeat domain (DARPin), avimer) as provided herein.

In some embodiments, a cell is a mammalian cell. In some embodiments, a cell is a human cell. In some embodiments, a cell is from a cell line. In some embodiments, a cell is a primary cell. In some embodiments, a primary cell is from a sample from a subject such as from a tumor or from corresponding tissue without a tumor (e.g., from another area of an organ or from a healthy donor). In some embodiments, a cell is in vitro (e.g., a primary cell, a cell line, etc.). In some embodiments, a cell is in vivo (e.g., in a subject, e.g., in a human subject, e.g., in a tumor of a human subject.) In some embodiments, a cell expresses or has been induced to express (e.g., via recombinant technology) a target. In some embodiments, the target is expressed on the surface of a cell. In some embodiments, a cell is contacted by a composition binding to a target expressed on its surface. In some embodiments, upon binding (e.g., upon binding of a miniprotein provided by the present disclosure), a target and any bound proteins and/or payloads is/are internalized into the cell. In some embodiments, a cell is killed by a payload (e.g., a radionuclide and/or chelator, etc.) after internalization.

In some embodiments, a target is a protein or portion thereof that is upregulated or overexpressed on cancer cells as compared to non-cancer cells. That is, in some embodiments, a target is expressed or overexpressed in a tumor or in a tumor microenvironment relative to a level of the target in non-diseased tissue (e.g., tissue without a tumor or tumor microenvironment). In some such embodiments, the target is absent or non-detectable in non-diseased (e.g., healthy) tissue. In some embodiments, a target is a biomarker for cancer (e.g., for cancer cells, for a tumor).

In some embodiments, a target may be related to a protein such as, for example, a protein in a pathway activated or acted upon by another protein. For instance, in some embodiments, a protein may be expressed on the surface of a cancer cell and a target may be a pathway that the surface-cell protein acts upon. In some embodiments, a protein may be expressed on a cancer cell and a target may be a protein on a different cell that causing a cancer cell to proliferate or otherwise be refractory to a treatment. In some embodiments, a tumor-associated cell surface molecule or tumor-specific cell surface molecule may be targeted by a miniprotein or composition comprising a miniprotein as provided herein.

In some embodiments, the miniprotein or composition comprising a miniprotein specifically binds a target expressed on the surface of a cell. In some embodiments, a target is cleaved from a cell surface. In some such embodiments, if the target is in an organism, cleavage of the target results in circulation of the target throughout the system of the organism. In some such embodiments, a target is found at a particular level in, e.g., blood, serum, plasma. In some embodiments, however, a substantial portion of expressed target is localized to cell surfaces; thus, in some embodiments, measurements of a level of a target may not accurately reflect the amount of target in a population of cells (e.g., a tumor). In some embodiments, a target is a secreted protein. In some such embodiments, a target is found at a particular level in, e.g., blood, serum, plasma. In some such embodiments, the miniprotein binds to a region of a target such as, for example, an epitope. In some embodiments, a miniprotein or composition comprising a miniprotein specifically binds a target expressed on the surface of a cancer cell. In some embodiments, the cancer cell is in, on, or near a solid tumor. In some embodiments, the cancer cell is a circulating cancer cell. In some embodiments, a miniprotein or composition comprising a miniprotein specifically binds a target or expressed at a higher level on a cancer cell than a reference cell. In some embodiments, the cell is a mammalian cell. In some embodiments, the cell is a human cell.

In some embodiments, the miniprotein or composition comprising a miniprotein specifically binds to Nectin-4. In some embodiments, the target comprises or consists of Nectin-4. In some embodiments, the miniprotein specifically binds to a target comprising an amino acid sequence or portion thereof as set forth in Table 1A.

### Nectin-4

Nectin proteins are involved in cellular adhesion, migration, and polarization. Nectin-4 in most human organs was found to be homogenously expressed at weak to moderate levels, but specifically overexpressed in a majority of samples from metastatic urothelial tumors. One ADC, enfortumab vedotin (“EV”;Padcev; Astellas; Tokyo, Japan; and Seattle Genetics; Bothell, WA, USA), specifically targets Nectin-4 that is overexpressed on the surface of bladder tumor cells. EV is conjugated to a microtubule inhibitor (monomethyl auristatin E), which causes G2/M cell cycle arrest and apoptosis. In some embodiments, a miniprotein of the present disclosure targets Nectin-4 on tumor cells. (Bednova O. & Leyton J V. Int J Mol Sci. 2020 Oct. 1; 21(19): 7268).

Clinical trials with EV suggest that, in some embodiments, depending on context (e.g., type of cancer), targeting of Nectin-4 can be all or part of a successful therapeutic strategy for treatment of cancer. For example, EV approval in the United States followed PhI and II clinical trial results. In the PhI study, patients who had previously been treated with ICI therapy had an overall tumor objective response rate (ORR) of 42% and patients with particularly high tumor burden (e.g., liver metastases) has a 36% ORR. Furthermore, in the EV PhII trial, patients with locally advanced or metastatic bladder cancer who had been previously treated with platinum-containing chemotherapy or ICI therapy were treated with EV and all tumors were positive for Nectin-4 and all characterized as having a “strong” level of expression. PD-L1 expression was also evaluated, but results of EV therapy showed that at 10.2 months (median follow up time), the ORR was 44%, with a 12% complete response rate (CRR); PD-L1 status had no impact on ORR or CRR. This study showed that Nectin-4 is a relevant target in bladder cancer, PD-L1 status or therapy does not negatively impact efficacy of Nectin-4-based therapy and provides a targeted alternative or addition to ICI-based therapy.

EV is currently being explored in a Phase III study, as well as developed for a PhII combination study with ICI therapy in cisplatin-ineligible patients. (Bednova O. & Leyton J V. Int J Mol Sci. 2020 Oct. 1; 21(19): 7268).

In some embodiments, Nectin-4 is an important target, alone or in conjunction with one or more therapies, for use with a miniprotein of the present disclosure.

In some embodiments, compositions provided by the present disclosure more specifically and effectively target a cell overexpressing Nectin-4 while minimizing or eliminating damage to surrounding cells not expressing or overexpressing Nectin-4 by providing a targeted composition including, in some embodiments, a chelator and/or alpha-emitter, which when combined with a miniprotein as provided herein provide specific, efficient and effective approaches to target cells overexpressing Nectin-4.

Importantly, novel compositions provided by the present disclosure are capable of specifically, efficiently, and effectively targeting Nectin-4 overexpressing cells with reduced toxicity as compared to presently available treatments. That is, in some embodiments, a composition targeting Nectin-4 as provided by the present disclosure provides improved treatment as compared to presently available treatments.

In some embodiments, a target of compositions of the present disclosure comprises or consists of Nectin-4. In some embodiments, Nectin-4 is expressed on the surface of a cell. In some embodiments, the cell is a cancer cell. In some such embodiments, the cancer cell is a tumor cell and the tumor is a solid tumor. In some embodiments, a level of Nectin-4 expressed in a tumor cell or population of tumor cells is higher than that expressed in non-tumor cells. In some embodiments, targeting of Nectin-4 by a miniprotein or composition comprising a miniprotein as provided by the present disclosure specifically targets a composition or one or more components thereof (e.g., a chelator and/or radionuclide) to a cancer cell or a tumor microenvironment (e.g., a location comprising a population of cancer cells or cells at risk of becoming cancer cells).

In some embodiments, miniproteins in accordance with the present disclosure specifically bind to Nectin-4. Without limitation, exemplary Nectin-4 miniproteins are provided in Table 2A and exemplary Nectin-4 sequences are shown in Table 1A.

In some embodiments, a miniprotein of the present disclosure comprises or consists of a polypeptide sequence corresponding to a polypeptide sequence shown in the Tables 1B, 1C, and/or 2A or binding to a polypeptide as shown in Table 1A. In some embodiments, a miniprotein comprises or consists of an amino acid sequence having sequence at least 85% identical to a polypeptide sequence shown in the Table(s). In some embodiments, a miniprotein of the present disclosure has at least 70%, 80%, 85%, 90%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, or higher identity to a polypeptide sequence according to Tables 2A, 1B and/or 1C.

In some embodiments, compositions in accordance with the present disclosure specifically bind to Nectin-4 (e.g., through a miniprotein that specifically binds to Nectin-4).

In some embodiments, a composition comprising a miniprotein comprises or consists of a protein comprising a specific an amino acid sequence that binds to Nectin-4 or a portion thereof. In some such embodiments, such a Nectin-4 miniprotein comprises or consists of an amino acid sequence selected from any of SEQ ID NOs: 1-158 or 177 or a functional variant or portion thereof (e.g., a functional fragment, e.g., a miniprotein that folds and binds to Nectin-4 or a portion thereof). In some embodiments, such a Nectin-4 miniprotein is a binding protein or part of a conjugate comprising such a binding protein as set forth in Table 2A.

In some embodiments a Nectin-4 miniprotein comprises or consists of an amino acid sequence that is 80%, 85%, 90%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or more identical to any one of SEQ ID NOs: 1-158 or 177 or a functional variant or portion thereof.

In some embodiments, a Nectin-4 miniprotein comprises or consists of an amino acid sequence according to SEQ ID NO: 1. In some embodiments, a Nectin-4 miniprotein comprises or consists of an amino acid sequence according to SEQ ID NO: 2. In some embodiments, a Nectin-4 miniprotein comprises or consists of an amino acid sequence according to SEQ ID NO: 3. In some embodiments, a Nectin-4 miniprotein comprises or consists of an amino acid sequence according to SEQ ID NO: 4. In some embodiments, a Nectin-4 miniprotein comprises or consists of an amino acid sequence according to SEQ ID NO: 5. In some embodiments, a Nectin-4 miniprotein comprises or consists of an amino acid sequence according to SEQ ID NO: 6. In some embodiments, a Nectin-4 miniprotein comprises or consists of an amino acid sequence according to SEQ ID NO: 7. In some embodiments, a Nectin-4 miniprotein comprises or consists of an amino acid sequence according to SEQ ID NO: 8. In some embodiments, a Nectin-4 miniprotein comprises or consists of an amino acid sequence according to SEQ ID NO: 9. In some embodiments, a Nectin-4 miniprotein comprises or consists of an amino acid sequence according to SEQ ID NO: 10. In some embodiments, a Nectin-4 miniprotein comprises or consists of an amino acid sequence according to SEQ ID NO: 11. In some embodiments, a Nectin-4 miniprotein comprises or consists of an amino acid sequence according to SEQ ID NO: 12. In some embodiments, a Nectin-4 miniprotein comprises or consists of an amino acid sequence according to SEQ ID NO: 13. In some embodiments, a Nectin-4 miniprotein comprises or consists of an amino acid sequence according to SEQ ID NO: 14. In some embodiments, a Nectin-4 miniprotein comprises or consists of an amino acid sequence according to SEQ ID NO: 15. In some embodiments, a Nectin-4 miniprotein comprises or consists of an amino acid sequence according to SEQ ID NO: 16. In some embodiments, a Nectin-4 miniprotein comprises or consists of an amino acid sequence according to SEQ ID NO: 17. In some embodiments, a Nectin-4 miniprotein comprises or consists of an amino acid sequence according to SEQ ID NO: 18. In some embodiments, a Nectin-4 miniprotein comprises or consists of an amino acid sequence according to SEQ ID NO: 19. In some embodiments, a Nectin-4 miniprotein comprises or consists of an amino acid sequence according to SEQ ID NO: 20. In some embodiments, a Nectin-4 miniprotein comprises or consists of an amino acid sequence according to SEQ ID NO: 21. In some embodiments, a Nectin-4 miniprotein comprises or consists of an amino acid sequence according to SEQ ID NO: 22. In some embodiments, a Nectin-4 miniprotein comprises or consists of an amino acid sequence according to SEQ ID NO: 23. In some embodiments, a Nectin-4 miniprotein comprises or consists of an amino acid sequence according to SEQ ID NO: 24. In some embodiments, a Nectin-4 miniprotein comprises or consists of an amino acid sequence according to SEQ ID NO: 25. In some embodiments, a Nectin-4 miniprotein comprises or consists of an amino acid sequence according to SEQ ID NO: 26. In some embodiments, a Nectin-4 miniprotein comprises or consists of an amino acid sequence according to SEQ ID NO: 27. In some embodiments, a Nectin-4 miniprotein comprises or consists of an amino acid sequence according to SEQ ID NO: 28. In some embodiments, a Nectin-4 miniprotein comprises or consists of an amino acid sequence according to SEQ ID NO: 29. In some embodiments, a Nectin-4 miniprotein comprises or consists of an amino acid sequence according to SEQ ID NO: 30. In some embodiments, a Nectin-4 miniprotein comprises or consists of an amino acid sequence according to SEQ ID NO: 31. In some embodiments, a Nectin-4 miniprotein comprises or consists of an amino acid sequence according to SEQ ID NO: 32. In some embodiments, a Nectin-4 miniprotein comprises or consists of an amino acid sequence according to SEQ ID NO: 33. In some embodiments, a Nectin-4 miniprotein comprises or consists of an amino acid sequence according to SEQ ID NO: 34. In some embodiments, a Nectin-4 miniprotein comprises or consists of an amino acid sequence according to SEQ ID NO: 35. In some embodiments, a Nectin-4 miniprotein comprises or consists of an amino acid sequence according to SEQ ID NO: 36. In some embodiments, a Nectin-4 miniprotein comprises or consists of an amino acid sequence according to SEQ ID NO: 37. In some embodiments, a Nectin-4 miniprotein comprises or consists of an amino acid sequence according to SEQ ID NO: 38. In some embodiments, a Nectin-4 miniprotein comprises or consists of an amino acid sequence according to SEQ ID NO: 39. In some embodiments, a Nectin-4 miniprotein comprises or consists of an amino acid sequence according to SEQ ID NO: 40. In some embodiments, a Nectin-4 miniprotein comprises or consists of an amino acid sequence according to SEQ ID NO: 41. In some embodiments, a Nectin-4 miniprotein comprises or consists of an amino acid sequence according to SEQ ID NO: 42. In some embodiments, a Nectin-4 miniprotein comprises or consists of an amino acid sequence according to SEQ ID NO: 43. In some embodiments, a Nectin-4 miniprotein comprises or consists of an amino acid sequence according to SEQ ID NO: 44. In some embodiments, a Nectin-4 miniprotein comprises or consists of an amino acid sequence according to SEQ ID NO: 45. In some embodiments, a Nectin-4 miniprotein comprises or consists of an amino acid sequence according to SEQ ID NO: 46. In some embodiments, a Nectin-4 miniprotein comprises or consists of an amino acid sequence according to SEQ ID NO: 47. In some embodiments, a Nectin-4 miniprotein comprises or consists of an amino acid sequence according to SEQ ID NO: 48. In some embodiments, a Nectin-4 miniprotein comprises or consists of an amino acid sequence according to SEQ ID NO: 49. In some embodiments, a Nectin-4 miniprotein comprises or consists of an amino acid sequence according to SEQ ID NO: 50. In some embodiments, a Nectin-4 miniprotein comprises or consists of an amino acid sequence according to SEQ ID NO: 51. In some embodiments, a Nectin-4 miniprotein comprises or consists of an amino acid sequence according to SEQ ID NO: 52. In some embodiments, a Nectin-4 miniprotein comprises or consists of an amino acid sequence according to SEQ ID NO: 53. In some embodiments, a Nectin-4 miniprotein comprises or consists of an amino acid sequence according to SEQ ID NO: 54. In some embodiments, a Nectin-4 miniprotein comprises or consists of an amino acid sequence according to SEQ ID NO: 55. In some embodiments, a Nectin-4 miniprotein comprises or consists of an amino acid sequence according to SEQ ID NO: 56. In some embodiments, a Nectin-4 miniprotein comprises or consists of an amino acid sequence according to SEQ ID NO: 57. In some embodiments, a Nectin-4 miniprotein comprises or consists of an amino acid sequence according to SEQ ID NO: 58. In some embodiments, a Nectin-4 miniprotein comprises or consists of an amino acid sequence according to SEQ ID NO: 59. In some embodiments, a Nectin-4 miniprotein comprises or consists of an amino acid sequence according to SEQ ID NO: 60. In some embodiments, a Nectin-4 miniprotein comprises or consists of an amino acid sequence according to SEQ ID NO: 61. In some embodiments, a Nectin-4 miniprotein comprises or consists of an amino acid sequence according to SEQ ID NO: 62. In some embodiments, a Nectin-4 miniprotein comprises or consists of an amino acid sequence according to SEQ ID NO: 63. In some embodiments, a Nectin-4 miniprotein comprises or consists of an amino acid sequence according to SEQ ID NO: 64. In some embodiments, a Nectin-4 miniprotein comprises or consists of an amino acid sequence according to SEQ ID NO: 65. In some embodiments, a Nectin-4 miniprotein comprises or consists of an amino acid sequence according to SEQ ID NO: 66. In some embodiments, a Nectin-4 miniprotein comprises or consists of an amino acid sequence according to SEQ ID NO: 67. In some embodiments, a Nectin-4 miniprotein comprises or consists of an amino acid sequence according to SEQ ID NO: 68. In some embodiments, a Nectin-4 miniprotein comprises or consists of an amino acid sequence according to SEQ ID NO: 69. In some embodiments, a Nectin-4 miniprotein comprises or consists of an amino acid sequence according to SEQ ID NO: 70. In some embodiments, a Nectin-4 miniprotein comprises or consists of an amino acid sequence according to SEQ ID NO: 71. In some embodiments, a Nectin-4 miniprotein comprises or consists of an amino acid sequence according to SEQ ID NO: 72. In some embodiments, a Nectin-4 miniprotein comprises or consists of an amino acid sequence according to SEQ ID NO: 73. In some embodiments, a Nectin-4 miniprotein comprises or consists of an amino acid sequence according to SEQ ID NO: 74. In some embodiments, a Nectin-4 miniprotein comprises or consists of an amino acid sequence according to SEQ ID NO: 75. In some embodiments, a Nectin-4 miniprotein comprises or consists of an amino acid sequence according to SEQ ID NO: 76. In some embodiments, a Nectin-4 miniprotein comprises or consists of an amino acid sequence according to SEQ ID NO: 77. In some embodiments, a Nectin-4 miniprotein comprises or consists of an amino acid sequence according to SEQ ID NO: 78. In some embodiments, a Nectin-4 miniprotein comprises or consists of an amino acid sequence according to SEQ ID NO: 79. In some embodiments, a Nectin-4 miniprotein comprises or consists of an amino acid sequence according to SEQ ID NO: 80. In some embodiments, a Nectin-4 miniprotein comprises or consists of an amino acid sequence according to SEQ ID NO: 81. In some embodiments, a Nectin-4 miniprotein comprises or consists of an amino acid sequence according to SEQ ID NO: 82. In some embodiments, a Nectin-4 miniprotein comprises or consists of an amino acid sequence according to SEQ ID NO: 83. In some embodiments, a Nectin-4 miniprotein comprises or consists of an amino acid sequence according to SEQ ID NO: 84. In some embodiments, a Nectin-4 miniprotein comprises or consists of an amino acid sequence according to SEQ ID NO: 85. In some embodiments, a Nectin-4 miniprotein comprises or consists of an amino acid sequence according to SEQ ID NO: 86. In some embodiments, a Nectin-4 miniprotein comprises or consists of an amino acid sequence according to SEQ ID NO: 87. In some embodiments, a Nectin-4 miniprotein comprises or consists of an amino acid sequence according to SEQ ID NO: 88. In some embodiments, a Nectin-4 miniprotein comprises or consists of an amino acid sequence according to SEQ ID NO: 89. In some embodiments, a Nectin-4 miniprotein comprises or consists of an amino acid sequence according to SEQ ID NO: 90. In some embodiments, a Nectin-4 miniprotein comprises or consists of an amino acid sequence according to SEQ ID NO: 91. In some embodiments, a Nectin-4 miniprotein comprises or consists of an amino acid sequence according to SEQ ID NO: 92. In some embodiments, a Nectin-4 miniprotein comprises or consists of an amino acid sequence according to SEQ ID NO: 93. In some embodiments, a Nectin-4 miniprotein comprises or consists of an amino acid sequence according to SEQ ID NO: 94. In some embodiments, a Nectin-4 miniprotein comprises or consists of an amino acid sequence according to SEQ ID NO: 95. In some embodiments, a Nectin-4 miniprotein comprises or consists of an amino acid sequence according to SEQ ID NO: 96. In some embodiments, a Nectin-4 miniprotein comprises or consists of an amino acid sequence according to SEQ ID NO: 97. In some embodiments, a Nectin-4 miniprotein comprises or consists of an amino acid sequence according to SEQ ID NO: 98. In some embodiments, a Nectin-4 miniprotein comprises or consists of an amino acid sequence according to SEQ ID NO: 99. In some embodiments, a Nectin-4 miniprotein comprises or consists of an amino acid sequence according to SEQ ID NO: 100. In some embodiments, a Nectin-4 miniprotein comprises or consists of an amino acid sequence according to SEQ ID NO: 101. In some embodiments, a Nectin-4 miniprotein comprises or consists of an amino acid sequence according to SEQ ID NO: 102. In some embodiments, a Nectin-4 miniprotein comprises or consists of an amino acid sequence according to SEQ ID NO: 103. In some embodiments, a Nectin-4 miniprotein comprises or consists of an amino acid sequence according to SEQ ID NO: 104. In some embodiments, a Nectin-4 miniprotein comprises or consists of an amino acid sequence according to SEQ ID NO: 105. In some embodiments, a Nectin-4 miniprotein comprises or consists of an amino acid sequence according to SEQ ID NO: 106. In some embodiments, a Nectin-4 miniprotein comprises or consists of an amino acid sequence according to SEQ ID NO: 107. In some embodiments, a Nectin-4 miniprotein comprises or consists of an amino acid sequence according to SEQ ID NO: 108. In some embodiments, a Nectin-4 miniprotein comprises or consists of an amino acid sequence according to SEQ ID NO: 109. In some embodiments, a Nectin-4 miniprotein comprises or consists of an amino acid sequence according to SEQ ID NO: 110. In some embodiments, a Nectin-4 miniprotein comprises or consists of an amino acid sequence according to SEQ ID NO: 111. In some embodiments, a Nectin-4 miniprotein comprises or consists of an amino acid sequence according to SEQ ID NO: 112. In some embodiments, a Nectin-4 miniprotein comprises or consists of an amino acid sequence according to SEQ ID NO: 113. In some embodiments, a Nectin-4 miniprotein comprises or consists of an amino acid sequence according to SEQ ID NO: 114. In some embodiments, a Nectin-4 miniprotein comprises or consists of an amino acid sequence according to SEQ ID NO: 115. In some embodiments, a Nectin-4 miniprotein comprises or consists of an amino acid sequence according to SEQ ID NO: 116. In some embodiments, a Nectin-4 miniprotein comprises or consists of an amino acid sequence according to SEQ ID NO: 117. In some embodiments, a Nectin-4 miniprotein comprises or consists of an amino acid sequence according to SEQ ID NO: 118. In some embodiments, a Nectin-4 miniprotein comprises or consists of an amino acid sequence according to SEQ ID NO: 119. In some embodiments, a Nectin-4 miniprotein comprises or consists of an amino acid sequence according to SEQ ID NO: 120. In some embodiments, a Nectin-4 miniprotein comprises or consists of an amino acid sequence according to SEQ ID NO: 121. In some embodiments, a Nectin-4 miniprotein comprises or consists of an amino acid sequence according to SEQ ID NO: 122. In some embodiments, a Nectin-4 miniprotein comprises or consists of an amino acid sequence according to SEQ ID NO: 123. In some embodiments, a Nectin-4 miniprotein comprises or consists of an amino acid sequence according to SEQ ID NO: 124. In some embodiments, a Nectin-4 miniprotein comprises or consists of an amino acid sequence according to SEQ ID NO: 125. In some embodiments, a Nectin-4 miniprotein comprises or consists of an amino acid sequence according to SEQ ID NO: 126. In some embodiments, a Nectin-4 miniprotein comprises or consists of an amino acid sequence according to SEQ ID NO: 127. In some embodiments, a Nectin-4 miniprotein comprises or consists of an amino acid sequence according to SEQ ID NO: 128. In some embodiments, a Nectin-4 miniprotein comprises or consists of an amino acid sequence according to SEQ ID NO: 129. In some embodiments, a Nectin-4 miniprotein comprises or consists of an amino acid sequence according to SEQ ID NO: 130. In some embodiments, a Nectin-4 miniprotein comprises or consists of an amino acid sequence according to SEQ ID NO: 131. In some embodiments, a Nectin-4 miniprotein comprises or consists of an amino acid sequence according to SEQ ID NO: 132. In some embodiments, a Nectin-4 miniprotein comprises or consists of an amino acid sequence according to SEQ ID NO: 133. In some embodiments, a Nectin-4 miniprotein comprises or consists of an amino acid sequence according to SEQ ID NO: 134. In some embodiments, a Nectin-4 miniprotein comprises or consists of an amino acid sequence according to SEQ ID NO: 135. In some embodiments, a Nectin-4 miniprotein comprises or consists of an amino acid sequence according to SEQ ID NO: 136. In some embodiments, a Nectin-4 miniprotein comprises or consists of an amino acid sequence according to SEQ ID NO: 137. In some embodiments, a Nectin-4 miniprotein comprises or consists of an amino acid sequence according to SEQ ID NO: 138. In some embodiments, a Nectin-4 miniprotein comprises or consists of an amino acid sequence according to SEQ ID NO: 139. In some embodiments, a Nectin-4 miniprotein comprises or consists of an amino acid sequence according to SEQ ID NO: 140. In some embodiments, a Nectin-4 miniprotein comprises or consists of an amino acid sequence according to SEQ ID NO: 141. In some embodiments, a Nectin-4 miniprotein comprises or consists of an amino acid sequence according to SEQ ID NO: 142. In some embodiments, a Nectin-4 miniprotein comprises or consists of an amino acid sequence according to SEQ ID NO: 143. In some embodiments, a Nectin-4 miniprotein comprises or consists of an amino acid sequence according to SEQ ID NO: 144. In some embodiments, a Nectin-4 miniprotein comprises or consists of an amino acid sequence according to SEQ ID NO: 145. In some embodiments, a Nectin-4 miniprotein comprises or consists of an amino acid sequence according to SEQ ID NO: 146. In some embodiments, a Nectin-4 miniprotein comprises or consists of an amino acid sequence according to SEQ ID NO: 147. In some embodiments, a Nectin-4 miniprotein comprises or consists of an amino acid sequence according to SEQ ID NO: 148. In some embodiments, a Nectin-4 miniprotein comprises or consists of an amino acid sequence according to SEQ ID NO: 149. In some embodiments, a Nectin-4 miniprotein comprises or consists of an amino acid sequence according to SEQ ID NO: 150. In some embodiments, a Nectin-4 miniprotein comprises or consists of an amino acid sequence according to SEQ ID NO: 151. In some embodiments, a Nectin-4 miniprotein comprises or consists of an amino acid sequence according to SEQ ID NO: 152. In some embodiments, a Nectin-4 miniprotein comprises or consists of an amino acid sequence according to SEQ ID NO: 153. In some embodiments, a Nectin-4 miniprotein comprises or consists of an amino acid sequence according to SEQ ID NO: 154. In some embodiments, a Nectin-4 miniprotein comprises or consists of an amino acid sequence according to SEQ ID NO: 155. In some embodiments, a Nectin-4 miniprotein comprises or consists of an amino acid sequence according to SEQ ID NO: 156. In some embodiments, a Nectin-4 miniprotein comprises or consists of an amino acid sequence according to SEQ ID NO: 157. In some embodiments, a Nectin-4 miniprotein comprises or consists of an amino acid sequence according to SEQ ID NO: 158. In some embodiments, a Nectin-4 miniprotein comprises or consists of an amino acid sequence according to SEQ ID NO: 177.

In some embodiments a miniprotein of the present disclosure binds to a Nectin-4 protein as set forth in SEQ ID NO: 159 and/or SEQ ID NO: 160. In some embodiments a miniprotein of the present disclosure binds to a portion of Nectin-4 as set forth in SEQ ID NO: 159 and/or SEQ ID NO: 160.

In some embodiments a Nectin-4 miniprotein comprises or consists of an amino acid sequence that is 80%, 85%, 90%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or more identical to any one of SEQ ID NOs: 161-176 or a functional variant or portion thereof. In some embodiments, a miniprotein of SEQ ID NOs: 161-176 has one or more substitutions as set forth in Table 1C. In some embodiments, a Nectin-4 miniprotein comprises or consists of an amino acid sequence that is 80%, 85%, 90%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or more identical to any one of SEQ ID NOs: 161-176 or a functional variant or portion thereof having one or more substitutions as set forth in Table 1C.

In some embodiments, the present disclosure provides a polynucleotide encoding a polypeptide that comprises or consists of one or more portions of a composition as provided herein. In some embodiments, the present disclosure provides a vector and/or host cell comprising a sequence encoding one or more components of a composition as provided herein. In some embodiments, the present disclosure provides methods of detecting a target. In some embodiments, a method as provided herein comprises detecting presence of a target for, e.g., imaging, e.g., diagnostic, prognostic, and/or monitoring purposes, e.g., treatment. In some embodiments, the present disclosure provides methods of treatment and/or methods of manufacturing using composition as provided herein (e.g., a miniprotein, e.g., a linker-chelator, e.g., a miniprotein comprising one or more of a linker, chelator, and radionuclide, etc.). In some embodiments, a method of treatment comprises administering a composition as provided herein to a subject in need thereof.

### Miniproteins

Provided herein are novel polypeptides (i.e., miniproteins) and methods of use thereof. In some embodiments, a polypeptide comprises or consists of a miniprotein. In some such embodiments, the miniprotein comprises or consists of a CDP, knottin, and/or binder. In some embodiments the miniprotein is designed to be linked to one or more other components. For example, in some embodiments, a miniprotein may be linked (conjugated) to another component such as a chelator and/or a radionuclide. In some embodiments, conjugation is via a lysine or cysteine residue. For example, in some embodiments, a miniprotein is engineered to remove all lysine residues except for one, which is, in some embodiments, used for conjugation. In some embodiments, conjugation occurs via an optional linker. In some embodiments, conjugation between a miniprotein and a chelator and/or radionuclide is direct.

Without wishing to be bound by any particular theory, the present disclosure contemplates that therapeutics comprising compositions provided by the present disclosure are characterized by several features relative to other (e.g., antibody-based) therapeutics. For example, in some embodiments, miniproteins display several key features of antibody-based therapeutics (e.g., affinity, potency, specificity, and ability to disrupt protein:protein interactions) but also have several advantages as compared to antibody-based therapeutics such as smaller size, cheaper manufacturing, and elimination of need to chimerize or humanize the proteins. In addition, the size and specificity of binding increases tumor penetrance and uptake into cells expressing the target of the miniprotein or composition (e.g., conjugate) comprising a miniprotein.

In some embodiments, a miniprotein of the present disclosure is no more than about 100 amino acids in length. In some embodiments, a miniprotein is about 20, 25, 30, 35, 40, 45, 50, 55, 60, 65, 70, 75, 80, 85, 90, 95 or more amino acids in lengths. In some such embodiments, however, a miniprotein of the present disclosure does not exceed about 100 amino acids in length. In some embodiments a miniprotein is between about 20 to about 40, about 30 to about 50, about 40 to about 60, about 45 to about 65, about 50 to about 70, about 55 to about 75, about 65 to about 85 or more amino acids in length, but not exceeding about 100 amino acids in length. In some preferred embodiments, a miniprotein is about 65 amino acids or less. In some preferred embodiments, a miniprotein is about 50 amino acids or less.

In some embodiments, a miniprotein of the present disclosure is not larger than about 12 kDa. In some embodiments, a miniprotein of the present disclosure is about 2, 2.5, 3, 3.5, 4, 4.5, 5, 5.5, 6, 6.5, 7, 7.5, 8, 8.5, 9, 9.5, 10, 10.5, 11, 11.5 or more kDa. In some such embodiments, however, a miniprotein of the present disclosure does not exceed about 12 kDa.

In some embodiments, a miniprotein of the present disclosure comprises or consists of a cysteine-dense peptide, knottin, binder, affibody, engineered Kunitz domain, monobody, anticalin, designed ankyrin repeat domain (DARPin), and/or avimer.

In some embodiments, a miniprotein comprises one or more disulfide bridges. In some embodiments, a miniprotein comprises at least two disulfide bridges. In some embodiments, a miniprotein comprises at least three cysteine residues. In some embodiments, a miniprotein comprises multiple (e.g., more than three) cysteine residues. In some such embodiments, cysteine residues crosslink to maintain a very stable, folded state for a peptide of its length (e.g., relative to a peptide of the same length without as many cysteine residues). The present disclosure contemplates that such crosslinking confers improved stability with reduced (i.e., very low to no) immunogenicity and/or sustains or improves ability to maintain biological activity in harsh but efficient chelation conditions (e.g., high temperature and low pH).

In some embodiments a miniprotein or composition comprising a miniprotein (e.g., a radionuclide conjugate) has low immunogenicity relative to a larger protein or composition comprising or consisting of a larger protein (e.g., an antibody).

In some embodiments, miniproteins (e.g., CDP, knottin, binder, affibody, engineered Kunitz domain, monobody, anticalin, designed ankyrin repeat domain (DARPin), avimer) have superior penetration efficiency relative to larger proteins. That is, in some embodiments, a miniprotein or composition comprising a miniprotein can penetrate a solid tumor better than a larger protein or composition comprising a protein larger than a miniprotein. For example, in some such embodiments, a miniprotein or composition comprising a miniprotein has a hydrodynamic radius of about 1 to about 25 nm. In some embodiments, a hydrodynamic radius is in a range of about 1-25 nm, 10⁻²⁰ nm, 5-15 nm, 1-5 nm, 2-4 nm, or 1-3 nm. In some embodiments, hydrodynamic radius is measured using light scatter methods known to those of skill in the art.

In some embodiments, a miniprotein of the present disclosure is characterized in that it has one or more properties relative to a protein larger than 100 amino acids like an antibody, antibody fragment, VHH domain, single chain antibody, or other protein or binder greater than 12 kDa. In some embodiments a property is selected from increased protein expression, increased thermoactivity, increased thermostability, increased pH activity, increased stability, increased activity, increased receptor binding specificity and/or affinity, increased specific activity, increased resistance to substrate and/or end-product inhibition, increased chemical stability, improved chemoselectivity, improved solvent stability, increased tolerance to acidic pH, increased tolerance to proteolytic activity (i.e., reduced sensitivity to proteolysis), reduced aggregation, increased solubility, reduced immunogenicity, and altered temperature profile, increased resistance to liver uptake, kidney uptake or healthy tissue binding, increased tumor penetration, and/or increased volume of distribution.

In some embodiments, a miniprotein or composition comprising a miniprotein (e.g., conjugate, e.g., radionuclide conjugate) provided by the present disclosure exhibits binding affinity to Nectin-4. In some embodiments, the Nectin-4 is human Nectin-4. In some embodiments, the human Nectin-4 is on a cell. In some embodiments, the cell is a cell line, a primary cell, or a cell in a human (e.g., in a tumor).

In some embodiments, a miniprotein or composition comprising a miniprotein (e.g., conjugate, e.g., radionuclide conjugate) displays nM or sub-nM binding affinity to Nectin-4. In some embodiments, the affinity is measured in an in vitro assay. In some embodiments, the in vitro assay is a cell-based assay. In some embodiments, affinity is measured in an in vivo assay (e.g., a PET scan) or using a sample from a subject (e.g., an in vitro assay using a biological specimen such as blood or a cell biopsy from a subject).

In some embodiments, a miniprotein or conjugate thereof displays a binding affinity to Nectin-4. In some embodiments, the binding affinity of a miniprotein or conjugate thereof to human Nectin-4 is about 500 nM In some embodiments, the miniprotein comprises picomolar binding affinity. In some embodiments, the miniprotein or conjugate thereof comprises a binding affinity characterized by a dissociation constant ranging from about 900 nM to about 1 nM, e.g., 900, 800, 700, 600, 500, 400, 300. 200, 100, 90, 80, 70, 60, 50, 40, 30, 20, 10, 9, 8, 7, 6, 5, 4, 3, 2, 1, 0.9, 0.8, 0.7, 0.6, 0.5, 0.4 nM or less binding affinity to human Nectin-4. In some embodiments, the binding is selective to human Nectin-4 and, not, e.g., non-human Nectin-4.

In some embodiments, a miniprotein or conjugate thereof provided by the present disclosure has high affinity for Nectin-4. In some such embodiments, the Nectin-4 is human Nectin-4. In some embodiments, a miniprotein of the present disclosure is stable, including in the presence of one or more additional molecules (e.g., a cytotoxic molecule, e.g., radiation).

In some embodiments, binding ability of a miniprotein or conjugate thereof to a target is improved by one or more modifications. For example, in some embodiments, binding ability of a miniprotein or conjugate thereof as provided herein to Nectin-4, is improved using chemical crosslinking. In some embodiments, binding can be enhanced by using one or more of lysine residues, fusion proteins, non-natural amino acids, or other chemical moieties to enhance binding and/or functional activity.

In some embodiments, to ensure proper folding and connectivity, selected cysteine pairs can be replaced with selenocysteines. It is contemplated that, in some embodiments, diselenide crosslinks can form more readily than disulfide crosslinks due to their lower redox potential and such a replacement may cross-couple remaining cysteine residues.

In some embodiments, a miniproteins or conjugates thereof provided by the present disclosure comprises or consists of monomers that make up a dimer, polymer or a multimer. In some such embodiments, the monomers all bind to the same target. For example, in some embodiments, where more than one miniprotein is present, each miniprotein is no greater than about 30-40 amino acids in length or a total of about 8 kDa in size (with both miniproteins). In some embodiments, the monomers each bind to a different target. In some embodiments, some monomers bind to one target and others bind to one or more additional targets.

In some embodiments, a miniprotein of the present disclosure comprises or consists of an antigen for use in generating an antibody that specifically binds to at least one epitope on Nectin-4. In some embodiments, such an antibody may be used for, e.g., diagnostic purposes, blocking (e.g., antagonism), etc.

In some embodiments, the miniprotein comprises one or more disulfide bridges. In some embodiments, the miniprotein comprises at least two disulfide bridges.

In some embodiments, a miniprotein or conjugate thereof as provided herein does not comprise one or more cysteine residues. In some embodiments, the miniprotein does not comprise one or more disulfide bridges.

In some embodiments, a miniprotein or conjugate thereof as provided herein is specific for a target. In some embodiments, a miniprotein is specific for Nectin-4 or a fragment thereof.

In some embodiments, a miniprotein or conjugate thereof as provided herein comprises or consists of a specific amino acid sequence.

In some embodiments, miniproteins or compositions comprising miniproteins (e.g., radionuclide conjugates) are conjugated to a chelator that optionally binds a radionuclide (e.g., actinium). In some embodiments, the conjugation is via a linker. In some embodiments, conjugation is direct conjugation. In some embodiments, such radionuclide conjugates combine and synergize to provide target specificity (e.g., via the miniprotein) and superior treatment (e.g., via directed radioisotope delivery to the cell expressing the target).

As used herein and known to those of skill in the art, the twenty conventional amino acids and their abbreviations follow conventional usage. See Immunology-A Synthesis (Golub and Gren eds., Sinauer Associates, Sunderland-Mass., 2nd ed. 1991), which is incorporated herein by reference. In some embodiments, an amino acid of the present disclosure may be a stereoisomer (e.g., D-amino acids) of the twenty conventional amino acids. In some embodiments, an amino acid in a polypeptide of the present disclosure may be a non-natural amino acid. For example, amino acids such as α-, α-disubstituted amino acids, N-alkyl amino acids, and other unconventional amino acids may also be suitable components for polypeptides of the present disclosure. Examples of unconventional amino acids include: 4-hydroxyproline, γ-carboxyglutamate, F-N,N,N-trimethyllysine, &-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). Arrangements of polypeptide sequence notations used herein have a left-side end corresponding to the amino terminal and a right-side end corresponding to the carboxy-terminal end, in accordance with standard usage and convention.

In some embodiments, a miniprotein as provided herein is specific for a polypeptide or portion thereof having an amino acid sequence or portion or functional variant thereof as set forth in Table 1A.

TABLE 1A
Exemplary Target Protein Amino Acid Sequences

| Target Protein | | SEQ ID |
|---|---|---|
| (Uniprot Acc. No.) | Amino Acid Sequence | NO. |
| Human Nectin-4 | MPLSLGAEMWGPEAWLLLLLLLASFTGRCPAGELETSDVVTVVLGQ | 159 |
| (Q96NY8) | DAKLPCFYRGDSGEQVGQVAWARVDAGEGAQELALLHSKYGLHVSP | |
| | AYEGRVEQPPPPRNPLDGSVLLRNAVQADEGEYECRVSTFPAGSFQ | |
| | ARLRLRVLVPPLPSLNPGPALEEGQGLTLAASCTAEGSPAPSVTWD | |
| | TEVKGTTSSRSFKHSRSAAVTSEFHLVPSRSMNGQPLTCVVSHPGL | |
| | LQDQRITHILHVSFLAEASVRGLEDQNLWHIGREGAMLKCLSEGQP | |
| | PPSYNWIRLDGPLPSGVRVDGDILGFPPLITEHSGIYVCHVSNEFS | |
| | SRDSQVTVDVLDPQEDSGKQVDLVSASVVVVGVIAALLFCLLVVVV | |
| | VLMSRYHRRKAQQMTQKYEEELILTRENSIRRLHSHHTDPRSQPEE | |
| | SVGLRAEGHPDSLKDNSSCSVMSEEPEGRSYSTLTTVREIETQTEL | |
| | LSPGSGRAEEEEDQDEGIKQAMNHFVQENGTLRAKPIGNGIYINGR | |
| | GHLV | |
| Murine Nectin-4 | MPLSLGAEMWGPEAWLRLLFLASFTGQYSAGELETSDVVIVVLGQD | 160 |
| (Q8R007) | AKLPCFYRGDPDEQVGQVAWARVDPNEGIRELALLHSKYGLHVNPA | |
| | YEDRVEQPPPPRDPLDGSVLLRNAVQADEGEYECRVSTFPAGSFQA | |
| | RMRLRVLVPPLPSLNPGPPLEEGQGLTLAASCTAEGSPAPSVTWDT | |
| | EVKGTQSSRSFTHPRSAAVTSEFHLVPSRSMNGQPLTCVVSHPGLL | |
| | QDRRITHTLQVAFLAEASVRGLEDQNLWQVGREGATLKCLSEGQPP | |
| | PKYNWIRLDGPLPSGVRVKGDILGFPPLITEHSGVYVCHVSNELSS | |
| | RDSQVTVEVLDPEDPGKQVDLVSASVIIVGVIAALLFCLLVVVVVL | |
| | MSRYHRRKAQQMTQKYEEELTLTRENSIRRLHSHHSDPRSQPEESV | |
| | GLRAEGHPDSLKDNSSCSVMSEEPEGRSYSTLTTVREIETQTELLS | |
| | PGSGRTEEDDDQDEGIKQAMNHFVQENGTLRAKPIGNGIYINGRGH | |
| | LV | |

In some embodiments, a miniprotein comprises or consists of a specific amino acid sequence. In some embodiments, a miniprotein has an amino acid sequence that is 70%, 75%, 80%, 85%, 90%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, or 100% identical to the amino acid sequence set forth in any of SEQ ID NOs: 1-158 or 177.

In some embodiments, a miniprotein comprises or consists of a specific amino acid sequence. In some embodiments, a miniprotein has an amino acid sequence that is 70%, 75%, 80%, 85%, 90%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, or 100% identical to the amino acid sequence set forth in any of SEQ ID NOs: 161-176.

In some embodiments, a miniprotein comprises or consists of a specific amino acid sequence. In some embodiments, a miniprotein has an amino acid sequence that is 70%, 75%, 80%, 85%, 90%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, or 100% identical to the amino acid sequence set forth in any of Tables 1B, 1C, and/or 2A.

As used herein and known to those of skill in the art, the twenty conventional amino acids and their abbreviations follow conventional usage. See Immunology-A Synthesis (Golub and Gren eds., Sinauer Associates, Sunderland-Mass., 2nd ed. 1991), which is incorporated herein by reference. In some embodiments, an amino acid of the present disclosure may be a stereoisomer (e.g., D-amino acids) of the twenty conventional amino acids. In some embodiments, an amino acid in a polypeptide of the present disclosure may be a non-natural amino acid. For example, amino acids such as α-, α-disubstituted amino acids, N-alkyl amino acids, and other unconventional amino acids may also be suitable components for polypeptides of the present disclosure. Examples of unconventional amino acids include: 4-hydroxyproline, γ-carboxyglutamate, F-N,N,N-trimethyllysine, &-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). Arrangements of polypeptide sequence notations used herein have a left-side end corresponding to the amino terminal and a right-side end corresponding to the carboxy-terminal end, in accordance with standard usage and convention.

In some embodiments, a miniprotein provided by the present disclosure is set forth in one or more consensus sequences provided in Table 1B. In some embodiments, a miniprotein with a sequence set forth in Table 1B has amino acid substitutions as provided in Table 1C.

TABLE 1B
Consensus Sequences of Exemplary Nectin-4 Miniproteins

| SEQ ID | | |
|---|---|---|
| NO | Formula | Consensus Sequence |
| 161 | I | CX1YDX2X3FFTALX4X5LRGX6DICX7YIX8X9X10FX11X12X13X14X15 |
| | | X16CIX17EILX18X19LGCX20 |
| 162 | II-A | CEYDEX1FFTALX2X3LRGX4DICX5YIQX6X7FX8YLPX9LCIEEILDNLGCS |
| 163 | II-B | CX1YDEX2FFTALX3X4LRGX5DICX6YIQX7X8FX9YLPX10LCIEEILDNLGCS |
| 164 | II-C | CX1YDEEFFTALX2X3LRGX4DICX5YIQX6X7FQYLPGLCIEEILDNLGCS |
| 165 | II-D | CX1YDEX2FFTALX3RLRGX4DICX5YIQAX6FQYLPX7LCIEEILDNLGCS |
| 166 | II-E | CEYDEX1FFTALX2X3LRGGDICX4YIQX5X6FX7YLPX8LCIEEILDNLGCS |
| 167 | II-F | CX1YDEX2FFTALX3X4LRGX5DICX6YIQX7X8FX9YLPX10LCIEEILDNLGCS |
| 168 | II-G | CX1YDEX2FFTALX3RLRGX4DICX5YIQAX6FQYLPGLCIEEILDNLGCS |
| 169 | II-H | CX1YDEX2FFTALX3X4LRGX5DICX6YIQAX7FQYLPX8LCIEEILDNLGCS |
| 170 | II-I | CX1YDEX2FFTALX3X4LRGX5DICX6YIQAX7FQYLPX8LCIEEILDNLGCS |
| 171 | II-J | CX1YDEX2FFTALX3X4LRGX5DICX6YIQX7X8FX9YLPX10LCIEEILDNLGCS |
| 172 | II-K | CX1YDEX2FFTALX3X4LRGX5DICX6YIQX7X8FX9YLPGLCIEEILDNLGCS |
| 173 | II-L | CX1YDEX2FFTALX3RLRGX4DICX5YIQAX6FX7YLPX8LCIEEILDNLGCS |
| 174 | II-M | CX1YDEX2FFTALX3X4LRGX5DICX6YIQX7X8FX9YLPX10LCIEEILDNLGCS |
| 175 | II-N | CX1YDEX2FFTALX3X4LRGX5DICX6YIQX7X8FX9YLPGLCIEEILDNLGCS |
| 176 | II-O | CX1YDEX2FFTALX3X4LRGX5DICX6YIQX7X8FX9YLPX10LCIEEILDNLGCS |

TABLE 1C
Consensus Sequences Substitutions

| SEQ ID NO | Position | Amino Acids |
|---|---|---|
| 161 | X1 | D, E |
| 161 | X2 | E, G |
| 161 | X3 | A, Q, E |
| 161 | X4 | A, K, S |
| 161 | X5 | A, R, Q, K, S |
| 161 | X6 | A, D, G, S |
| 161 | X7 | D, Q, E, L, S, Y |
| 161 | X8 | Q, L, S |
| 161 | X9 | A, Q, E, K |
| 161 | X10 | A, Q, K, S, Y, OH-Norleu, Norleu |
| 161 | X11 | A, N, Q, S |
| 161 | X12 | N, T, Y |
| 161 | X13 | L, Y, V |
| 161 | X14 | P, E |
| 161 | X15 | A, D, Q, G, K |
| 161 | X16 | D, Q, E, I, L |
| 161 | X17 | Q, E |
| 161 | X18 | D, Q, E |
| 161 | X19 | N, Q |
| 161 | X20 | Absent or S |
| 162 | X1 | A, Q, E |
| 162 | X2 | A, K, S |
| 162 | X3 | A, R, Q, K, S |
| 162 | X4 | A, D, G, S |
| 162 | X5 | D, Q, E, L, S, Y |
| 162 | X6 | A, Q, E, K |
| 162 | X7 | A, Q, K, S, Y, OH-Norleu, Norleu |
| 162 | X8 | A, N, Q, S |
| 162 | X9 | A, D, Q, G, K |
| 163 | X1 | D, E |
| 163 | X2 | A, Q, E |
| 163 | X3 | A, K, S |
| 163 | X4 | A, R, Q, K, S |
| 163 | X5 | A, D, G, S |
| 163 | X6 | D, Q, E, L, S, Y |
| 163 | X7 | A, Q, E, K |
| 163 | X8 | A, Q, K, S, Y, OH-Norleu, Norleu |
| 163 | X9 | A, N, Q, S |
| 163 | X10 | A, D, Q, G, K |
| 164 | X1 | D, E |
| 164 | X2 | A, K, S |
| 164 | X3 | A, R, Q, K, S |
| 164 | X4 | A, D, G, S |
| 164 | X5 | D, Q, E, L, S, Y |
| 164 | X6 | A, Q, E, K |
| 164 | X7 | A, Q, K, S, Y, OH-Norleu, Norleu |
| 165 | X1 | D, E |
| 165 | X2 | A, Q, E |
| 165 | X3 | A, K, S |
| 165 | X4 | A, D, G, S |
| 165 | X5 | D, Q, E, L, S, Y |
| 165 | X6 | A, Q, K, S, Y, OH-Norleu, Norleu |
| 165 | X7 | A, D, Q, G, K |
| 166 | X1 | A, Q, E |
| 166 | X2 | A, K, S |
| 166 | X3 | A, R, Q, K, S |
| 166 | X4 | D, Q, E, L, S, Y |
| 166 | X5 | A, Q, E, K |
| 166 | X6 | A, Q, K, S, Y, OH-Norleu, Norleu |
| 166 | X7 | A, N, Q, S |
| 166 | X8 | A, D, Q, G, K |
| 167 | X1 | D, E |
| 167 | X2 | A, Q, E |
| 167 | X3 | A, K, S |
| 167 | X4 | A, R, Q, K, S |
| 167 | X5 | A, D, G, S |
| 167 | X6 | D, Q, E, L, S, Y |
| 167 | X7 | A, Q, E, K |
| 167 | X8 | A, Q, K, S, Y, OH-Norleu, Norleu |
| 167 | X9 | A, N, Q, S |
| 167 | X10 | A, D, Q, G, K |
| 168 | X1 | D, E |
| 168 | X2 | A, Q, E |
| 168 | X3 | A, K, S |
| 168 | X4 | A, D, G, S |
| 168 | X5 | D, Q, E, L, S, Y |
| 168 | X6 | A, Q, K, S, Y, OH-Norleu, Norleu |
| 169 | X1 | D, E |
| 169 | X2 | A, Q, E |
| 169 | X3 | A, K, S |
| 169 | X4 | A, R, Q, K, S |
| 169 | X5 | A, D, G, S |
| 169 | X6 | D, Q, E, L, S, Y |
| 169 | X7 | A, Q, K, S, Y, OH-Norleu, Norleu |
| 169 | X8 | A, D, Q, G, K |
| 170 | X1 | D, E |
| 170 | X2 | A, Q, E |
| 170 | X3 | A, K, S |
| 170 | X4 | A, R, Q, K, S |
| 170 | X5 | A, D, G, S |
| 170 | X6 | D, Q, E, L, S, Y |
| 170 | X7 | A, Q, K, S, Y, OH-Norleu, Norleu |
| 170 | X8 | A, D, Q, G, K |
| 171 | X1 | D, E |
| 171 | X2 | A, Q, E |
| 171 | X3 | A, K, S |
| 171 | X4 | A, R, Q, K, S |
| 171 | X5 | A, D, G, S |
| 171 | X6 | D, Q, E, L, S, Y |
| 171 | X7 | A, Q, E, K |
| 171 | X8 | A, Q, K, S, Y, OH-Norleu, Norleu |
| 171 | X9 | A, N, Q, S |
| 171 | X10 | A, D, Q, G, K |
| 172 | X1 | D, E |
| 172 | X2 | A, Q, E |
| 172 | X3 | A, K, S |
| 172 | X4 | A, R, Q, K, S |
| 172 | X5 | A, D, G, S |
| 172 | X6 | D, Q, E, L, S, Y |
| 172 | X7 | A, Q, E, K |
| 172 | X8 | A, Q, K, S, Y, OH-Norleu, Norleu |
| 172 | X9 | A, N, Q, S |
| 173 | X1 | D, E |
| 173 | X2 | A, Q, E |
| 173 | X3 | A, K, S |
| 173 | X4 | A, D, G, S |
| 173 | X5 | D, Q, E, L, S, Y |
| 173 | X6 | A, Q, K, S, Y, OH-Norleu, Norleu |
| 173 | X7 | A, N, Q, S |
| 173 | X8 | A, D, Q, G, K |
| 174 | X1 | D, E |
| 174 | X2 | A, Q, E |
| 174 | X3 | A, K, S |
| 174 | X4 | A, R, Q, K, S |
| 174 | X5 | A, D, G, S |
| 174 | X6 | D, Q, E, L, S, Y |
| 174 | X7 | A, Q, E, K |
| 174 | X8 | A, Q, K, S, Y, OH-Norleu, Norleu |
| 174 | X9 | A, N, Q, S |
| 174 | X10 | A, D, Q, G, K |
| 175 | X1 | D, E |
| 175 | X2 | A, Q, E |
| 175 | X3 | A, K, S |
| 175 | X4 | A, R, Q, K, S |
| 175 | X5 | A, D, G, S |
| 175 | X6 | D, Q, E, L, S, Y |
| 175 | X7 | A, Q, E, K |
| 175 | X8 | A, Q, K, S, Y, OH-Norleu, Norleu |
| 175 | X9 | A, N, Q, S |
| 176 | X1 | D, E |
| 176 | X2 | A, Q, E |
| 176 | X3 | A, K, S |
| 176 | X4 | A, R, Q, K, S |
| 176 | X5 | A, D, G, S |
| 176 | X6 | D, Q, E, L, S, Y |
| 176 | X7 | A, Q, E, K |
| 176 | X8 | A, Q, K, S, Y, OH-Norleu, Norleu |
| 176 | X9 | A, N, Q, S |
| 176 | X10 | A, D, Q, G, K |

In some embodiments, the present disclosure provides a miniprotein. In some embodiments, the miniprotein is or comprises a polypeptide, comprising: an amino acid sequence, wherein the amino acid sequence comprises Formula I:

(SEQ ID NO: 161)
CX1YDX2X3FFTALX4X5LRGX6DICX7YIX8X9X10FX11
X12X13X14X15X16CIX17EILX18X19LGCX20

wherein X20 is an optional amino acid or carboxy terminus comprising an —OH and wherein X1 is D or E; X2 is E or G; X3 is Q or E; X4 is A, K, or S; X5 is A, R, Q, K, S, or Cit; X6 is A, D, G, or S; X7 is D, Q, E, L, S, or Y; X8 is Q, L, or S; X9 is A, Q, E, K; X10 is A, Q, K, S, Y, OH-Norleu, or Norleu; X1I is A, N, Q, or S; X12 is N, T, or Y; X13 is L, Y, or V; X14 is P or E; X15 is A, D, Q, G, or K; X16 is D, Q, E, I, or L; X17 is Q or E; X18 is D, Q, or E; X19 is N or Q; and X20, when present as an amino acid is S.

In some embodiments, a miniprotein comprises or consists of a polypeptide according to the amino acid sequence of SEQ ID NO: 161, wherein X1 is E; X2 is E; X3 is E; X4 is K; X5 is R; X6 is G; X7 is Y; X8 is Q; X9 is A; X10 is S; X11 is Q; X12 is Y; X13 is L; X14 is P; X15 is G; X16 is L; X17 is E; X18 is D; X19 is N; and X20 is S.

In some embodiments, the present disclosure provides a miniprotein. In some embodiments, the miniprotein is or comprises a polypeptide, comprising: an amino acid sequence, wherein the amino acid sequence comprises Formula IIA:

(SEQ ID NO: 162)
CEYDEX1FFTALX2X3LRGX4DICX5YIQX6X7FX8YLP
X9LCIEEILDNLGCS

wherein X1 is A, Q, or E; X2 is A, K, or S; X3 is A, R, Q, K, or S; X4 is A, D, G, or 5; X5 is D, Q, E, L, S, or Y; X6 is A, Q, E, or K; X7 is A, Q, K, S, Y, OH-Norleu, or Norleu; X8 is A, N, Q, or S; and X9 is A, D, Q, G, or K.

In some embodiments, the present disclosure provides a miniprotein. In some embodiments, the miniprotein is or comprises a polypeptide, comprising: an amino acid sequence, wherein the amino acid sequence comprises Formula II-B:

(SEQ ID NO: 163)
CX1YDEX2FFTALX3X4LRGX5DICX6YIQX7X8FX9YLPX10LCIEEI
LDNLGCS

wherein X1 is D or E; X2 is A, Q, or E; X3 is A, K, or S; X4 is A, R, Q, K, or S; X5 is A, D, G, or S; X6 is D, Q, E, L, S, or Y; X7 is A, Q, E, or K; X8 is A, Q, K, S, Y, OH-Norleu, or Norleu; X9 is A, N, Q, or S; and X10 is A, D, Q, G, or K.

In some embodiments, the present disclosure provides a miniprotein. In some embodiments, the miniprotein is or comprises a polypeptide, comprising: an amino acid sequence, wherein the amino acid sequence comprises Formula II-C:

(SEQ ID NO: 164)
CX1YDEEFFTALX2X3LRGX4DICX5YIQX6X7FQYLPGLCIEEILDNL
GCS

wherein X1 is D or E; X2 is A, K, or S; X3 is A, R, Q, K, or S; X4 is A, D, G, or S; X5 is D, Q, E, L, S, or Y; X6 is A, Q, E, or K; and X7 is A, Q, K, S, Y, Norleu, or OH-Norleu.

In some embodiments, the present disclosure provides a miniprotein. In some embodiments, the miniprotein is or comprises a polypeptide, comprising: an amino acid sequence, wherein the amino acid sequence comprises Formula II-D:

(SEQ ID NO: 165)
CX1YDEX2FFTALX3RLRGX4DICX5YIQAX6FQYLPX7LCIEEILDNL
GCS

wherein X1 is D or E; X2 is A, Q, or E; X3 is A, K, or S; X4 is A, D, G, or S; X5 is D, Q, E, L, S, or Y; X6 is A, Q, K, S, Y, OH-Norleu, or Norleu; and X7 is A, N, Q, or S.

In some embodiments, the present disclosure provides a miniprotein. In some embodiments, the miniprotein is or comprises a polypeptide, comprising: an amino acid sequence, wherein the amino acid sequence comprises Formula II-E:

(SEQ ID NO: 166)
CEYDEX1FFTALX2X3LRGGDICX4YIQX5X6FX7YLPX8LCIEEILDN
LGCS

wherein X1 is A, Q, or E; X2 is A, L, or S; X3 is A, R, Q, K, or S; X4 is D, Q, E, L, S, or Y; X5 is A, Q, E, or K; X6 is A, Q, K, S, Y, OH-Norleu, or Norleu; X7 is A, N, Q, S; and X8 is A, D, Q, G, or K.

In some embodiments, the present disclosure provides a miniprotein. In some embodiments, the miniprotein is or comprises a polypeptide, comprising: an amino acid sequence, wherein the amino acid sequence comprises Formula II-F:

(SEQ ID NO: 167)
CX1YDEX2FFTALX3X4LRGX5DICX6YIQX7X8FX9YLPX10LCIEEI
LDNLGCS

wherein X1 is D or E; X2 is A, Q, or E; X3 is A, K, or S; X4 is A, R, Q, K, or S; X5 is A, D, G, or S; X6 is D, Q, E, L, S, or Y; X7 is A, Q, E, or K; X8 is A, Q, K, S, Y, OH-Norleu, or Norleu; X9 is A, N, Q, or S; and X10 is A, D, Q, G, or K.

In some embodiments, the present disclosure provides a miniprotein. In some embodiments, the miniprotein is or comprises a polypeptide, comprising: an amino acid sequence, wherein the amino acid sequence comprises Formula II-G:

(SEQ ID NO: 168)
CX1YDEX2FFTALX3RLRGX4DICX5YIQAX6FQYLPGLCIEEILDNLG
CS

wherein X1 is D or E; X2 is A, Q, or E; X3 is A, K, or S; X4 is A, D, G, or S; X5 is D, Q, E, L, S, or Y; and X6 is A, Q, K, S, Y, OH-Norleu, or Norleu.

In some embodiments, the present disclosure provides a miniprotein. In some embodiments, the miniprotein is or comprises a polypeptide, comprising: an amino acid sequence, wherein the amino acid sequence comprises Formula II-H:

(SEQ ID NO: 169)
CX1YDEX2FFTALX3X4LRGX5DICX6YIQAX7FQYLPX8LCIEEILDN
LGCS

wherein X1 is D or E; X2 is A, Q, or E; X3 is A, K, or S; X4 is A, R, Q, K, or S; X5 is A, D, G, or S; X6 is D, Q, E, L, S, or Y; and X7 is A, Q, K, S, Y, OH-Norleu, or Norleu; and X8 is A, D, Q, G, or K.

In some embodiments, the present disclosure provides a miniprotein. In some embodiments, the miniprotein is or comprises a polypeptide, comprising: an amino acid sequence, wherein the amino acid sequence comprises Formula II-I:

(SEQ ID NO: 170)
CX1YDEX2FFTALX3X4LRGX5DICX6YIQAX7FQYLPX8LCIEEILDN
LGCS

wherein X1 is D or E; X2 is A, Q, or E; X3 is A, K, or S; X4 is A, R, Q, K, or S; X5 is A, D, G, or S; X6 is D, Q, E, L, S, or Y; X7 is A, Q, K, S, Y, OH-Norleu, or Norleu; and X8 is A, D, Q, G, or K.

In some embodiments, the present disclosure provides a miniprotein. In some embodiments, the miniprotein is or comprises a polypeptide, comprising: an amino acid sequence, wherein the amino acid sequence comprises Formula II-J:

(SEQ ID NO: 171)
CX1YDEX2FFTALX3X4LRGX5DICX6YIQX7X8FX9YLPX10LCIEEI
LDNLGCS

wherein X1 is D or E; X2 is A, Q, or E; X3 is A, K, or S; X4 is A, R, Q, K, or S; X5 is A, D, G, or S; X6 is D, Q, E, L, S, or Y; X7 is A, Q, E, or K; X8 is A, Q, K, S, Y, OH-Norleu, Norleu; X9 is A, N, Q, or S; and X10 is A, D, Q, G, or K.

In some embodiments, the present disclosure provides a miniprotein. In some embodiments, the miniprotein is or comprises a polypeptide, comprising: an amino acid sequence, wherein the amino acid sequence comprises Formula II-K:

(SEQ ID NO: 172)
CX1YDEX2FFTALX3X4LRGX5DICX6YIQX7X8FX9YLPGLCIEEILD
NLGCS

wherein X1 is D or E; X2 is A, Q, or E; X3 is A, K, or S; X4 is A, R, Q, K, or S; X5 is A, D, G, or S; X6 is D, Q, E, L, S, or Y; X7 is A, Q, E, or K; X8 is A, Q, K, S, Y, OH-Norleu, or Norleu; and X9 is A, N, Q, or S.

In some embodiments, the present disclosure provides a miniprotein. In some embodiments, the miniprotein is or comprises a polypeptide, comprising: an amino acid sequence, wherein the amino acid sequence comprises Formula II-L:

(SEQ ID NO: 173)
CX1YDEX2FFTALX3RLRGX4DICX5YIQAX6FX7YLPX8LCIEEILDN
LGCS

wherein X1 is D or E; X2 is A, Q, or E; X3 is A, K, or S; X4 is A, D, G, or S; X5 is D, Q, E, L, S, or Y; X6 is A, Q, K, S, Y, OH-Norleu, or Norleu; X7 is A, N, Q, or S; and X8 is A, D, Q, G, or K.

In some embodiments, the present disclosure provides a miniprotein. In some embodiments, the miniprotein is or comprises a polypeptide, comprising: an amino acid sequence, wherein the amino acid sequence comprises Formula II-M:

(SEQ ID NO: 174)
CX1YDEX2FFTALX3X4LRGX5DICX6YIQX7X8FX9YLPX10LCIEEI
LDNLGCS

wherein X1 is D or E; X2 is A, Q, or E; X3 is A, K, or S; X4 is A, R, Q, K, or S; X5 is A, D, G, or S; X6 is D, Q, E, L, S, or Y; X7 is A, Q, E, or K; X8 is A, Q, K, S, Y, OH-Norleu, or Norleu; X9 is A, N, Q, or S; and X10 is A, D, Q, G, or K.

In some embodiments, the present disclosure provides a miniprotein. In some embodiments, the miniprotein is or comprises a polypeptide, comprising: an amino acid sequence, wherein the amino acid sequence comprises Formula II-N:

(SEQ ID NO: 175)
CX1YDEX2FFTALX3X4LRGX5DICX6YIQX7X8FX9YLPGLCIEEILD
NLGCS

wherein X1 is D or E; X2 is A, Q, or E; X3 is A, K, or S; X4 is A, R, Q, K, or S; X5 is A, D, G, or S; X6 is D, Q, E, L, S, or Y; X7 is A, Q, E, or K; X8 is A, Q, K, S, Y, OH-Norleu, or Norleu; and X9 is A, N, Q, or S.

In some embodiments, the present disclosure provides a miniprotein. In some embodiments, the miniprotein is or comprises a polypeptide, comprising: an amino acid sequence, wherein the amino acid sequence comprises Formula II-O:

(SEQ ID NO: 176)
CX1YDEX2FFTALX3X4LRGX5DICX6YIQX7X8FX9YLPX10LCIEEI
LDNLGCS

wherein X1 is D or E; X2 is A, Q, or E; X3 is A, K, or S; X4 is A, R, Q, K, or S; X5 is A, D, G, or S; X6 is D, Q, E, L, S, or Y; X7 is A, Q, E, or K; X8 is A, Q, K, S, Y, OH-Norleu, or Norleu; X9 is A, N, Q, or S; and X10 is A, D, Q, G, or K.

In some embodiments, a polypeptide according to Formula I or any of Formula IIA-IIO further comprises one or more of a linker, chelator, and radionuclide. In some embodiments, the linker can be or comprise linker comprises or consists of a polyethylene glycol (PEG) linker of PEG4, PEG, PEG2, PEG6, PEG8, PEG12, PEG24, an ester linker, an amide linker, a maleimide linker, a, a succinimidyl-4-(N-maleimidomethyl) cyclohexane-1-carboxylate (SMCC) linker, a propanoic acid linker, a caproleic acid linker, or (Gly)n-(gGlu)n- or (PEG)n, wherein n is from 1 to 10, (Gly)1-10, or any fragment or combination via covalent bond thereof. In some embodiments, the chelator comprises or consists of DOTA, DOPA, Macropa, and Crown. In some embodiments, the radionuclide is Ac-225, In-111, Ga-68, Pb-212, Lu-177, Cu-67, Cu-64, La-132, La-135, or Ce-134.

In some embodiments, when present, the linker is attached to the C-terminal end of the polypeptide. In some embodiments, when present, the chelator is attached to either the polypeptide or the linker. In some embodiments, when present, the radionuclide is attached to the chelator. In some embodiments, the polypeptide comprises one or more additional N-terminal amino acids. In some embodiments, the one or more amino acids on the N-terminal and/or the C-terminal end of the polypeptide.

In some embodiments, a miniprotein comprising an amino acid sequence of any of SEQ ID NOs: 161-176 and/or according to any of Tables 1B, 1C, and/or 2A has at least one disulfide bridge.

In some embodiments, the miniprotein has at least two disulfide bridges.

In some embodiments, disulfide bridges are formed between cysteines 1 and 34 and between cysteines 20 and 44. In some embodiments, a miniprotein comprising four cysteines has two disulfide bridges and Cys1 is connected to Cys34 and Cys 20 is connected to Cys 44.

In some embodiments, the miniprotein has one or more additional amino acids inserted into amino acids within CDP loop regions (amino acids in loops of secondary structures between cysteines connected to one another, e.g., amino acids in structures formed between Cys 1 and Cys 34 and Cys 20 and Cys 44). As will be understood to those in the art, retention of conformation is key to binding affinity and behavior; accordingly, in some embodiments, any additions within CDP loop regions can be made in a way that preserves proper conformation and stability such that binding affinity is not reduced and/or conformation or stability is not impaired or destroyed.

In some embodiments, the polypeptide is a monomer. In some embodiments, the polypeptide is a dimer, trimer, or tetramer. In some embodiments, the polypeptide comprises an amino acid sequence with at least 80% sequence identity to that of SEQ ID NO: 78. In some embodiments, the polypeptide comprises an amino acid sequence with at least 80% sequence identity to that of any of SEQ ID NOs: 1-158 or 177. In some embodiments, the polypeptide comprises a consensus sequence according to any of Formulas IIA-IIO (SEQ ID NOs: 161-176) according to Table 1B, further comprising a substitution in accordance with those set forth in Table 1C.

In some embodiments, a sequence comprising a variable position (e.g., as in Table 1B, can also include a substitution with any cognate amino acid. As used herein, a cognate amino acid is one with one or more similar characteristics to another amino acid, such as, for example, a similar charge (e.g., a negatively charged amino acid, in reference to charge at physiologic pH), or, in some embodiments, a set of substitutions (e.g., more than one amino acid) that creates a similar charge profile to the polypeptide as prior to the substitution or set of substitutions, etc..

TABLE 2A
Miniprotein Sequences and Compound Structures

| | | | | | | Calcu- | Ob- |
|---|---|---|---|---|---|---|---|
| | | | | | | lated | served |
| SEQ | Com- | | | C- | | Mass | Mass |
| ID | pound | N- | | termi- | Parent | (M + | (M + |
| NO | Name | terminus | Sequence | nus | MW | 4/4) | 4/4) |
| 1 | C1 | NH2 | CEDDGEYFAGLQRLYGGDICYYIKLKFPKVPDLCIKEILDKLGC | OH | 5042.88 | 1261.72 | Not |
| | | | | | | | observed |
| 2 | C2 | Biotin-PEG4 | CEDDEEFFADLKRLRGGDICYYIKLKFDKVPDLCIKEILDKLGC | OH | 5641.6 | 1411.40 | 1412.5 |
| 3 | C3 | NH2 | CEYDEEFFAGLKRLRGGDICYYIKKKFDKVPDLCIKEILDKLGC | OH | 5173.08 | 1294.27 | 1295.2 |
| 4 | C4 | NH2 | CEDDFQFFADLKRLRGGDICYYIRLKFDKVPDLCIKEILDKLGC | OH | 5213.1 | 1304.28 | 1305.1 |
| 3 | C5 | ACETYL | CEYDEEFFAGLKRLRGGDICYYIKKKFDKVPDLCIKEILDKLGC | OH | 5215.12 | 1304.78 | 1305.6 |
| 4 | C6 | ACETYL | CEDDFQFFADLKRLRGGDICYYIRLKFDKVPDLCIKEILDKLGC | OH | 5255.14 | 1314.79 | 1315.9 |
| 5 | C7 | — | CEYDEEFFAGLKRLRGGDICYYIKKKFDKVPDLCIEEILDKLGC | OH | 5174.02 | 1294.51 | 1296.0 |
| 5 | C8 | Biotinylated | CEYDEEFFAGLKRLRGGDICYYIKKKFDKVPDLCIEEILDKLGC | OH | 5647.6 | 1412.90 | 1413.2 |
| 5 | C9 | ACETYL | CEYDEEFFAGLKRLRGGDICYYIKKKFDKVPDLCIEEILDKLGC | OH | 5216.06 | 1305.02 | 1305.5 |
| 5 | C10 | FITC | CEYDEEFFAGLKRLRGGDICYYIKKKFDKVPDLCIEEILDKLGC | OH | 5809.75 | 1453.44 | 1454.1 |
| 5 | C11 | DTPA-PEG4 | CEYDEEFFAGLKRLRGGDICYYIKKKFDKVPDLCIEEILDKLGC | OH | 5796.64 | 1450.16 | 1450.8 |
| 5 | C12 | DOTA-PEG4 | CEYDEEFFAGLKRLRGGDICYYIKKKFDKVPDLCIEEILDKLGC | OH | 5806.7 | 1452.68 | 1454.1 |
| 6 | C13 | ACETYL | CEYDEEFFNGLKRLRGGDICYYIKKKFDKVPDLCIEEILDKLGC | OH | 5259.08 | 1315.77 | 1316.6 |
| 7 | C14 | ACETYL | CEYDEEFFAGLKRLRRGDICYYIKKKFDKVPDLCIEEILDKLGC | OH | 5315.19 | 1329.80 | 1330.5 |
| 8 | C15 | ACETYL | CEYDEEFFNGLKRLRRGDICYYIKKKFDKVPDLCIEEILDKLGC | OH | 5358.22 | 1340.56 | 1341.4 |
| 9 | C16 | ACETYL | CEYDEEFFAGLKRLRGGDICYYIKKKFKKVPDLCIEEILDKLGC | OH | 5233.17 | 1309.29 | 1309.3 |
| 10 | C17 | ACETYL | CEYDEEFFNGLKRLRGGDICYYIKKKFKKVPDLCIEEILDKLGC | OH | 5272.17 | 1319.04 | 1320.2 |
| 11 | C18 | ACETYL | CEYDEEFFNGLHRLRRGDICYYIKKKFKKVPDLCIEEILDKLGC | OH | 5384.3 | 1347.08 | 1347.0 |
| 12 | C19 | ACETYL | CEYDEEFFAGLKRLRGGDICYYIKKKRPKVPDLCIEEILDKLGC | OH | 5198.08 | 1300.52 | 1301.2 |
| 13 | C20 | ACETYL | CEYDEEFFAGLHRLRGGDICYYIKKKFDKVPDLCIEEILDKLGC | OH | 5225.02 | 1307.26 | 1307.9 |
| 14 | C21 | ACETYL | CEYDEEFFAGLHRLRGGDICYYIKKKFPKVPDLCIEEILDKLGC | OH | 5207.05 | 1302.76 | 1303.2 |
| 15 | C22 | ACETYL | CEYDEEFFAGLKRLRGTDICYYIKKKFDKVPDLCIEEILDKLGC | OH | 5260.11 | 1316.03 | 1317.1 |
| 16 | C23 | ACETYL | CEYDEEFFAGLHRLRGTDICYYIKKKFDKVPDLCIEEILDKLGC | OH | 5269.08 | 1318.27 | 1319.3 |
| 17 | C24 | ACETYL | CEYDEEFFAGLHRLRGTDICYYIKKKFPKVPDLCIEEILDKLGC | OH | 5251.1 | 1313.78 | 1315.0 |
| 18 | C25 | ACETYL | CEYDEEFFKGLKRLRGGDICYYIKKKFKKVPDLCIEEILDKLGC | OH | 5286.24 | 1322.56 | 1323.2 |
| 19 | C26 | ACETYL | CEYDEEFFEGLKRLRGGDICYYIKKKFKKVPDLCIEEILDKLGC | OH | 5287.18 | 1322.80 | 1324.1 |
| 20 | C27 | ACETYL | CEYDEEFFDGLKRLRGGDICYYIKKKFKKVPDLCIEEILDKLGC | OH | 5273.15 | 1319.29 | 1320.1 |
| 21 | C28 | ACETYL | CEYDEEFFSGLKRLRGGDICYYIKKKFKKVPDLCIEEILDKLGC | OH | 5245.14 | 1312.29 | 1314.5 |
| 22 | C29 | ACETYL | CEYDEEFFTGLKRLRGGDICYYIKKKFKKVPDLCIEEILDKLGC | OH | 5259.17 | 1315.79 | 1316.4 |
| 23 | C30 | ACETYL | CEYDEEFFEGLKKLRGGDICYYIKKKFKKVPDLCIEEILDKLGC | OH | 5228.06 | 1308.02 | 1308.8 |
| 24 | C31 | ACETYL | CEYDEEFFTGLKRLRGGDICYYIKKKFKKVPKLCIEEILDKLGC | OH | 5272.26 | 1319.07 | 1320.3 |
| 25 | C32 | ACETYL | CEYDEEFFTGLKRLRGGDICYYIKKKFKKVPELCIEEILDKLGC | OH | 5273.2 | 1319.30 | 1320.3 |
| 26 | C33 | ACETYL | CEYDEEFFQGLKRLRGGDICYYIKKKFKKVPDLCIEEILDKLGC | OH | 5286.19 | 1322.55 | 1323.9 |
| 27 | C34 | ACETYL | CEYDEEFFLGLKRLRGGDICYYIKKKFKKVPDLCIEEILDKLGC | OH | 5271.22 | 1318.81 | 1319.6 |
| 28 | C35 | ACETYL | CEYKEEFFTGLKRLRGGDICYYIKKKFKKVPDLCIEEILDKLGC | OH | 5272.26 | 1319.07 | 1320.3 |
| 29 | C36 | ACETYL | CEYEEEFFTGLKRLRGGDICYYIKKKFKKVPDLCIEEILDKLGC | OH | 5273.20 | 1319.30 | 1320.3 |
| 30 | C37 | ACETYL | CEYDEEFFTGLKRLRGGKICYYIKKKFKKVPDLCIEEILDKLGC | OH | 5272.26 | 1319.07 | 1320.3 |
| 31 | C38 | ACETYL | CEYDEEFFTGLKRLRGGEICYYIKKKFKKVPDLCIEEILDKLGC | OH | 5273.2 | 1319.30 | 1320.3 |
| 32 | C39 | ACETYL | CEYDEEFFTGLKRLRGGNICYYIKKKFKKVPDLCIEEILDKLGC | OH | 5258.18 | 1315.55 | 1316.5 |
| 33 | C40 | ACETYL | CEYDEEFFTGLKRLRGGSICYYIKKKFKKVPDLCIEEILDKLGC | OH | 5231.16 | 1308.79 | 1309.8 |
| 34 | C41 | ACETYL | CEYDEEFFTGLKRLRGGQICYYIKKKFKKVPDLCIEEILDKLGC | OH | 5248.22 | 1313.06 | 1313.7 |
| 35 | C42 | ACETYL | CEYDEEFFTGLKRLRGGQICYYIKKKFKKVPDLCIEEILDKLGC | OH | 5276.24 | 1320.06 | 1320.2 |
| 36 | C43 | ACETYL | CEYDEEFFDapGLKRLRGGDICYYIKKKFKKVPDLCIEEILDKLGC | OH | 5249.17 | 1313.29 | 1313.2 |
| 37 | C44 | ACETYL | CEYDEEFFTGLKRLRGGDICYYIKKKFKKVPDLCIEEILKKLGC | OH | 5258.27 | 1315.57 | 1316.7 |
| 38 | C45 | ACETYL | CEYDEEFFTGLKRLRGGDICYYIKKKFKKVPDLCIEEILEKLGC | OH | 5273.2 | 1319.30 | 1316.7 |
| 39 | C46 | ACETYL | CEYDEKFFTGLKRLRGGDICYYIKKKFKKVPDLCIEEILDKLGC | OH | 5258.23 | 1315.56 | 1316.6 |
| 40 | C47 | ACETYL | CEYDKEFFTGLKRLRGGDICYYIKKKFKKVPDLCIEEILDKLGC | OH | 5258.23 | 1315.56 | 1316.5 |
| 41 | C48 | ACETYL | CKYDEEFFTGLKRLRGGDICYYIKKKFKKVPDLCIEEILDKLGC | OH | 5258.23 | 1315.56 | 1316.7 |
| 42 | C49 | ACETYL | CEYDEQFFTGLKRLRGGDICYYIKKKFKKVPDLCIEEILDKLGC | OH | 5258.18 | 1315.55 | 1316.5 |
| 5 | C50 | DOTA-PEG8 | CEYDEEFFAGLKRLRGGDICYYIKKKFDKVPDLCIEEILDKLGC | OH | 5983.93 | 1496.98 | 1498.4 |
| 5 | C51 | DOTA- | CEYDEEFFAGLKRLRGGDICYYIKKKFDKVPDLCIEEILDKLGC | OH | 6160.14 | 1541.04 | 1542.1 |
| | | PEG12 | | | | | |
| 43 | C52 | ACETYL | CEYDEEFFTDLKRLRGGDICYYIKKKFKKVPKLCIEEILDKLGC | OH | 5330.29 | 1333.57 | 1334.6 |
| 44 | C53 | ACETYL | CEYDEEFFTELKRLRGGDICYYIKKKFKKVPKLCIEEILDKLGC | OH | 5344.32 | 1337.08 | 1338.3 |
| 45 | C54 | ACETYL | CEYDEEFFTKLKRLRGGDICYYIKKKFKKVPKLCIEEILDKLGC | OH | 5343.38 | 1336.85 | 1338.2 |
| 46 | C55 | ACETYL | CEYDEEFFTTLKRLRGGDICYYIKKKFKKVPKLCIEEILDKLGC | OH | 5316.31 | 1330.08 | 1331.2 |
| 47 | C56 | ACETYL | CEYDEEFFTLLKRLRGGDICYYIKKKFKKVPKLCIEEILDKLGC | OH | 5328.36 | 1333.09 | 1334.0 |
| 48 | C57 | ACETYL | CEYDEEFFTALKRLRGGDICYYIKKKFKKVPKLCIEEILDKLGC | OH | 5286.28 | 1322.57 | 1324.0 |
| 49 | C58 | ACETYL | CEYDEEFFLGLKRLRGGDICYYIKKKFKKVPKLCIEEILDKLGC | OH | 5284.31 | 1322.08 | 1323.2 |
| 50 | C59 | ACETYL | CEYDEEFFTGLKRLRGGDICYYIKKKFKKVPKLCIEEILEKLGC | OH | 5286.28 | 1322.57 | 1323.7 |
| 51 | C60 | ACETYL | CEYDEEFFLGLKRLRGGDICYYIKKKFKKVPDLCIEEILEKLGC | OH | 5285.25 | 1322.31 | 1323.2 |
| 52 | C61 | ACETYL | CEYDEEFFLGLKRLRGGDICYYIKKKFKKVPKLCIEEILEKLGC | OH | 5298.34 | 1325.59 | 1326.7 |
| 53 | C62 | ACETYL | CEYDEEFFTGLKRLRGGDICYYIKKKFKKVPKLCIEEILKKLGC | OH | 5285.34 | 1322.34 | 1323.2 |
| 54 | C63 | ACETYL | CEYDEEFFLGLKRLRGGDICYYIKKKFKKVPKLCIEEILKKLGC | OH | 5297.4 | 1325.35 | 1326.6 |
| 55 | C64 | ACETYL | CEYDEEFFLGLKRLRGGDICYYIKKKFKKVPDLCIEEILKKLGC | OH | 5284.31 | 1322.08 | 1323.2 |
| 56 | C65 | ACETYL | CEYDEEFFTGLKRLRGGDICYYIKKKFKKVPELCIEEILKKLGC | OH | 5286.28 | 1322.57 | 1323.7 |
| 57 | C66 | ACETYL | CEYDEEFFTGLKRLRGGDICYYIKKKFKKVPELCIEEILEKLGC | OH | 5287.22 | 1322.81 | 1324.1 |
| 58 | C67 | ACETYL | CEYDEEFFLGLKRLRGGDICYYIKKKFKKVPELCIEEILKKLGC | OH | 5284.31 | 1322.08 | 1326.8 |
| 59 | C68 | ACETYL | CEYDEEFFLGLKRLRGGDICYYIKKKFKKVPELCIEEILEKLGC | OH | 5299.28 | 1325.82 | 1327.2 |
| 60 | C69 | ACETYL | CEYDEEFFKGLKRLRGGDICYYIKKKFKKVPKLCIEEILEKLGC | OH | 5313.35 | 1329.34 | 1329.8 |
| 61 | C70 | ACETYL | CEYDEKFFTGLKRLRGGDICYYIKKKFKKVPKLCIEEILDKLGC | OH | 5271.32 | 1318.83 | 1319.8 |
| 62 | C71 | ACETYL | CEYDEQFFTGLKRLRGGDICYYIKKKFKKVPKLCIEEILDKLGC | OH | 5271.27 | 1318.82 | 1319.6 |
| 63 | C72 | ACETYL | CEYDEKFFLGLKRLRGGDICYYIKKKFKKVPKLCIEEILKKLGC | OH | 5296.46 | 1325.12 | 1326.5 |
| 64 | C73 | ACETYL | CEYDEQFFLGLKRLRGGDICYYIKKKFKKVPKLCIEEILKKLGC | OH | 5296.41 | 1325.10 | 1326.6 |
| 65 | C74 | ACETYL | CEYDEKFFTGLKRLRGGDICYYIKKKFKKVPKLCIEEILKKLGC | OH | 5284.4 | 1322.10 | 1323.1 |
| 66 | C75 | ACETYL | CEYDEQFFTGLKRLRGGDICYYIKKKFKKVPKLCIEEILKKLGC | OH | 5284.36 | 1322.09 | 1323.1 |
| 67 | C76 | ACETYL | CEYDEEFFKALKRLRGGDICYYIKKKFKKVPKLCIEEILEKLGC | OH | 5327.38 | 1332.85 | 1333.9 |
| 68 | C77 | ACETYL | CEYDEEFFLALKRLRGGDICYYIKKKFKKVPKLCIEEILEKLGC | OH | 5312.36 | 1329.09 | 1329.9 |
| 69 | C78 | ACETYL | CEYDEEFFLALKRLRGGDICYYIKKKFKKVPKLCIEEILDKLGC | OH | 5298.34 | 1325.59 | 1327.1 |
| 70 | C79 | ACETYL | CEYDEEFFTALKRLRGGDICYYIKKKFKKVPKLCIEEILEKLGC | OH | 5300.31 | 1326.08 | 1327.7 |
| 71 | C80 | ACETYL | CEYDEEFFKGLKRLRGGDICYYIKKKFKKVPKLCIEEILDKLGC | OH | 5299.33 | 1325.83 | 1327.4 |
| 72 | C81 | ACETYL | CEYDEEFFKALKRLRGGDICYYIKKKFKKVPKLCIEEILDKLGC | OH | 5313.35 | 1329.34 | 1329.9 |
| 48 | C82 | DOTA-PEG4 | CEYDEEFFTALKRLRGGDICYYIKKKFKKVPKLCIEEILDKLGC | NH2 | 5880.99 | 1471.25 | 1471.6 |
| 48 | C83 | ACETYL | CEYDEEFFTALKRLRGGDICYYIKKKFKKVPKLCIEEILDKLGC | NH2 | 5286.28 | 1322.57 | 1323.4 |
| 73 | C84 | DOTA-PEG4 | CEYDEEFFTALLys(Ac)CitrullineLCitrullineGGDICYYIKKKFKK- | NH2 | 5963 | 1491.75 | 1492.3 |
| | | | VPKLCIEEILDLys(Ac)LGC | | | | |
| 73 | C85 | FITC-PEG4 | CEYDEEFFTALLys(Ac)CitrullineLCitrullineGGDICYYIKKKFKK- | NH2 | 5965.98 | 1492.495 | 1493.7 |
| | | | VPKLCIEEILDLys(Ac)LGC | | | | |
| 74 | C86 | DOTA-PEG4 | CEYDEEFFTALLys(Ac)CitrullineLCitrullineGGDICYYILys(Ac)Lys- | NH2 | 6215.22 | 1554.805 | 1556.5 |
| | | | (Ac)Lys(Ac)FLys(Ac)Lys(Ac)VPLys(Ac)LCIEEILDLys(Ac)LGC | | | | |
| 74 | C87 | FITC-PEG4 | CEYDEEFFTALLys(Ac)CitrullineLCitrullineGGDICYYILys(Ac)Lys- | NH2 | 6218.2 | 1555.55 | 1556.8 |
| | | | (Ac)Lys(Ac)FLys(Ac)Lys(Ac)VPLys(Ac)LCIEEILDLys(Ac)LGC | | | | |
| 75 | C88 | DOTA-PEG4 | CEYDEEFFTALKRLRGGDICYYILys(Ac)Lys(Ac)Lys(Ac)FLys(Ac)- | NH2 | 6129.18 | 1533.295 | 1534.2 |
| | | | Lys(Ac)VPLys(Ac)LCIEEILDKLGC | | | | |
| 75 | C89 | FITC-PEG4 | CEYDEEFFTALKRLRGGDICYYILys(Ac)Lys(Ac)Lys(Ac)FLys(Ac)- | NH2 | 6132.16 | 1534.04 | 1534.7 |
| | | | Lys(Ac)VPLys(Ac)LCIEEILDKLGC | | | | |
| 5 | C90 | ¹⁵¹Eu-DOTA- | CEYDEEFFAGLKRLRGGDICYYIKKKFDKVPDLCIEEILDKLGC | OH | 5956.65 | — | 1193.6 |
| | | PEG4 | | | | | |
| 5 | C91 | ¹³⁸La-DOTA- | CEYDEEFFAGLKRLRGGDICYYIKKKFDKVPDLCIEEILDKLGC | OH | 5943.6 | — | 1191.3 |
| | | PEG4 | | | | | |
| 5 | C92 | ^{nat}In-DOTA- | CEYDEEFFAGLKRLRGGDICYYIKKKFDKVPDLCIEEILDKLGC | OH | 5919.51 | — | 1185.9 |
| | | PEG4 | | | | | |
| 5 | C93 | ⁶⁹Ga-DOTA- | CEYDEEFFAGLKRLRGGDICYYIKKKFDKVPDLCIEEILDKLGC | OH | 5874.41 | — | 1176.8 |
| | | PEG4 | | | | | |
| 5 | C94 | ⁶³Cu-DOTA- | CEYDEEFFAGLKRLRGGDICYYIKKKFDKVPDLCIEEILDKLGC | OH | 5869.24 | — | 1175.8 |
| | | PEG4 | | | | | |
| 48 | C95 | ¹⁵¹Eu-DOTA- | CEYDEEFFTALKRLRGGDICYYIKKKFKKVPKLCIEEILDKLGC | OH | 6027.84 | — | 1207.6 |
| | | PEG4 | | | | | |
| 48 | C96 | ¹³⁸La-DOTA- | CEYDEEFFTALKRLRGGDICYYIKKKFKKVPKLCIEEILDKLGC | OH | 6013.82 | — | 1205.1 |
| | | PEG4 | | | | | |
| 48 | C97 | ¹³⁸La-DOTA- | CEYDEEFFTALKRLRGGDICYYIKKKFKKVPKLCIEEILDKLGC | NH2 | 6012.83 | — | 1204.9 |
| | | PEG4 | | | | | |
| 48 | C98 | ^{nat}In-DOTA- | CEYDEEFFTALKRLRGGDICYYIKKKFKKVPKLCIEEILDKLGC | NH2 | 5988.75 | — | 1199.3 |
| | | PEG4 | | | | | |
| 5 | C99 | ²²⁵Ac-DOTA- | CEYDEEFFAGLKRLRGGDICYYIKKKFDKVPDLCIEEILDKLGC | OH | — | — | — |
| | | PEG4 | | | | | |
| 76 | C100 | Acetyl | CEYDEQFFTALKRLRGGDICYYISAQFNTLPDLCIEEILENLGC | OH | 5146.84 | 1287.71 | 1287.35 |
| 76 | C101 | DOTA-PEG4 | CEYDEQFFTALKRLRGGDICYYISAQFNTLPDLCIEEILENLGC | OH | 5738.50 | 1435.63 | 1435.68 |
| 76 | C102* | In:DOTA- | CEYDEQFFTALKRLRGGDICYYISAQFNTLPDLCIEEILENLGC | OH | 5850.30 | 1463.57 | 1463.02 |
| | | PEG4 | | | | | |
| 76 | C103 | Biotin-PEG4 | CEYDEQFFTALKRLRGGDICYYISAQFNTLPDLCIEEILENLGC | OH | 5578.409 | 1395.60 | 1395.17 |
| 77 | C104 | Acetyl | CEYDEEFFTALKKLRGGDICYYIQQAFNYLPGICIEEILDNLGC | OH | 5150.88 | 1288.72 | 1288.26 |
| 77 | C105 | DOTA-PEG4 | CEYDEEFFTALKKLRGGDICYYIQQAFNYLPGICIEEILDNLGC | OH | 5742.53 | 1436.63 | 1436.6 |
| 77 | C106* | In:DOTA- | CEYDEEFFTALKKLRGGDICYYIQQAFNYLPGICIEEILDNLGC | OH | 5854.33 | 1464.58 | 1464.01 |
| | | PEG4 | | | | | |
| 77 | C107 | Biotin-PEG4 | CEYDEEFFTALKRLRGGDICYYIQASFQYLPGLCIEEILDNLGCS | OH | 5582.43 | 1396.61 | 1395.56 |
| 78 | C108 | Acetyl | CEYDEEFFTALKRLRGGDICYYIQASFQYLPGLCIEEILDNLGCS | OH | 5238.94 | 1310.74 | 1309.96 |
| 78 | C109 | DOTA-PEG4 | CEYDEEFFTALKRLRGGDICYYIQASFQYLPGLCIEEILDNLGCS | OH | 5830.6 | 1458.65 | 1458.12 |
| 78 | C110* | In:DOTA- | CEYDEEFFTALKRLRGGDICYYIQASFQYLPGLCIEEILDNLGCS | OH | 5942.39 | 1486.60 | 1486.06 |
| | | PEG4 | | | | | |
| 78 | C111 | Biotin-PEG4 | CEYDEEFFTALKRLRGGDICYYIQASFQYLPGLCIEEILDNLGCS | OH | 5670.50 | 1418.62 | 1417.98 |
| 79 | C112 | Acetyl | CEYDEQFFTALKALRGGDICYYIQASFNYLPDLCIEEILDNLGC | NH2 | 5109.78 | 1278.44 | — |
| 80 | C113 | DOTA-PEG4 | CEYDEQFFTALKALRGGDICYYIQASFNYLPDLCIEEILDNLGCS | OH | 5788.52 | 1448.13 | 1447.66 |
| 80 | C114* | In:DOTA- | CEYDEQFFTALKALRGGDICYYIQASFNYLPDLCIEEILDNLGCS | OH | 5904.34 | 1477.09 | 1475.39 |
| | | PEG4 | | | | | |
| 80 | C115 | Biotin-PEG4 | CEYDEQFFTALKALRGGDICYYIQASFNYLPDLCIEEILDNLGCS | OH | 5628.41 | 1408.10 | 1407.69 |
| 81 | C116 | Acetyl | CEYDEEFFTALKRLRGGDICYYIQAKFQYLPKLCIEEILDNLGCS | OH | 5351.16 | 1338.79 | 1337.78 |
| 81 | C117 | DOTA-PEG4 | CEYDEEFFTALKRLRGGDICYYIQAKFQYLPKLCIEEILDNLGCS | OH | 5942.82 | 1486.71 | 1486.25 |
| 81 | C118* | In:DOTA- | CEYDEEFFTALKRLRGGDICYYIQAKFQYLPKLCIEEILDNLGCS | OH | 6050.738 | 1513.68 | 1514.03 |
| | | PEG4 | | | | | |
| 81 | C119 | Biotin-PEG4 | CEYDEEFFTALKRLRGGDICYYIQAKFQYLPKLCIEEILDNLGCS | OH | 5782.72 | 1446.68 | 1446.55 |
| 82 | C120 | Acetyl | CEYDEEFFTALKRLRGGDICYYIQKAFQYLPGLCIEEILDNLGCS | OH | 5280.04 | 1321.01 | 1320.52 |
| 82 | C121 | DOTA-PEG4 | CEYDEEFFTALKRLRGGDICYYIQKAFQYLPGLCIEEILDNLGCS | OH | 5871.70 | 1468.92 | 1468.34 |
| 82 | C122* | In:DOTA- | CEYDEEFFTALKRLRGGDICYYIQKAFQYLPGLCIEEILDNLGCS | OH | 5983.69 | 1496.92 | 1496.26 |
| | | PEG4 | | | | | |
| 82 | C123 | Biotin-PEG4 | CEYDEEFFTALKRLRGGDICYYIQKAFQYLPGLCIEEILDNLGCS | OH | 5711.59 | 1428.90 | 1428.33 |
| 83 | C124 | Acetyl | CEYDEEFFTALARLRGGDICYYIQAKFQYLPGLCIEEILDNLGCS | OH | 5222.94 | 1306.74 | 1306.24 |
| 83 | C125 | DOTA-PEG4 | CEYDEEFFTALARLRGGDICYYIQAKFQYLPGLCIEEILDNLGCS | OH | 5814.6 | 1454.65 | 1454.19 |
| 83 | C126* | In:DOTA- | CEYDEEFFTALARLRGGDICYYIQAKFQYLPGLCIEEILDNLGCS | OH | 5926.39 | 1482.60 | 1482.09 |
| | | PEG4 | | | | | |
| 83 | C127 | Biotin-PEG4 | CEYDEEFFTALARLRGGDICYYIQAKFQYLPGLCIEEILDNLGCS | OH | 5654.50 | 1414.62 | 1414.15 |
| 84 | C128 | Acetyl | CEYDEQFFTALARLRGGDICYYIQEQFATVPGLCIEEILDQLGC | NH2 | 5073.75 | 1269.44 | — |
| 85 | C129 | DOTA-PEG4 | CEYDEQFFTALARLRGGDICYYIQEQFATVPGLCIEEILDQLGCS | OH | 5752.49 | 1439.12 | 1438.7 |
| 85 | C130* | In:DOTA- | CEYDEQFFTALARLRGGDICYYIQEQFATVPGLCIEEILDQLGCS | OH | 5864.28 | 1467.07 | 1466.58 |
| | | PEG4 | | | | | |
| 85 | C131 | Biotin-PEG4 | CEYDEQFFTALARLRGGDICYYIQEQFATVPGLCIEEILDQLGCS | OH | 5592.38 | 1399.10 | 1398.69 |
| 86 | C132 | Acetyl | CEYDEEFFTALSRLRGGDICYYIQQAFQYLPGLCIEEILDNLGC | NH2 | 5150.84 | 1288.71 | 1288.35 |
| 87 | C133 | DOTA-PEG4 | CEYDEEFFTALSRLRGGDICYYIQQAFQYLPGLCIEEILDNLGCS | OH | 5,830.56 | 1458.64 | 1457.92 |
| 87 | C134* | In:DOTA- | CEYDEEFFTALSRLRGGDICYYIQQAFQYLPGLCIEEILDNLGCS | OH | 5942.35 | 1486.59 | 1486.11 |
| | | PEG4 | | | | | |
| 87 | C135 | Biotin-PEG4 | CEYDEEFFTALSRLRGGDICYYIQQAFQYLPGLCIEEILDNLGCS | OH | 5670.45 | 1418.61 | 1417.98 |
| 88 | C136 | Acetyl | CEYDEQFFTALSSLRGGDICYYIQEQFANVPGICIEEILDNLGC | OH | 5019.61 | 1255.90 | — |
| 89 | C137 | DOTA-PEG4 | CEYDEQFFTALSSLRGGDICYYIQEQFANVPGICIEEILDNLGCS | OH | 5698.35 | 1425.59 | 1425.09 |
| 89 | C138* | In:DOTA- | CEYDEQFFTALSSLRGGDICYYIQEQFANVPGICIEEILDNLGCS | OH | 5810.14 | 1453.54 | 1453.08 |
| | | PEG4 | | | | | |
| 89 | C139 | Biotin-PEG4 | CEYDEQFFTALSSLRGGDICYYIQEQFANVPGICIEEILDNLGCS | OH | 5538.24 | 1385.56 | — |
| 90 | C140 | Biotin-PEG4 | CEYDEEFFTALARLRGADICYYIQAKFQYLPGDCIEEILDNLGCS | OH | 5,670.45 | 1418.61 | 1418.11 |
| 91 | C141 | Biotin-PEG4 | CDYDEEFFTALARLRGGDICEYIQAKFQYLPGLCIEEILDNLGCS | OH | 5606.4 | 1402.60 | 1402.2 |
| 92 | C142 | Biotin-PEG4 | CEYDEEFFTALARLRGGDICYYIQAKFQYLPGECIEEILQNLGCS | OH | 5683.49 | 1421.87 | 1421.36 |
| 93 | C143 | Biotin-PEG4 | CEYDEEFFTALARLRGDDICSYIQAKFQYLPGLCIEEILDNLGCS | OH | 5636.43 | 1410.11 | 1409.7 |
| 94 | C144 | Biotin-PEG4 | CEYDGEFFTALARLRGADICEYIQAKFQYYPGLCIEEILDNLGCS | OH | 5612.41 | 1404.10 | 1403.7 |
| 95 | C145 | Biotin-PEG4 | CEYDEEFFTALARLRGGDICYYILAKFQYLPGECIEEILDNLGCS | OH | 5655.48 | 1414.87 | 1414.35 |
| 96 | C146 | Biotin-PEG4 | CEYDEQFFTALARLRGGDICEYIQAKFQYLPGLCIEEILDNLGCS | OH | 5619.45 | 1405.86 | 1405.29 |
| 97 | C147 | Biotin-PEG4 | CEYDEEFFTALARLRGADICDYIQAKFQYLPGLCIEEILDNLGCS | OH | 5620.44 | 1406.11 | 1405.7 |
| 98 | C148 | Biotin-PEG4 | CEYDEEFFTALARLRGGDICEYIQAKFQYLPGLCIQEILDNLGCS | OH | 5619.45 | 1405.86 | 1405.48 |
| 99 | C149 | Biotin-PEG4 | CEYDEEFFTALARLRGGDICQYIQAKFQYLPGQCIEEILDNLGCS | OH | 5634.42 | 1409.61 | 1408.83 |
| 100 | C150 | Biotin-PEG4 | CEYDEEFFTALARLRGGDICEYIQAKFQYLEGLCIEEILDNLGCS | OH | 5652.4 | 1414.10 | 1412.93 |
| 101 | C151 | Biotin-PEG4 | CEYDEAFFTALARLRGGDICQYIQAKFQYLPGLCIEEILDNLGCS | OH | 5561.38 | 1391.35 | 1390.75 |
| 102 | C152 | Biotin-PEG4 | CEYDEQFFTALARLRGGDICYYILAKFQYLPQLCIEEILDNLGCS | OH | 5709.59 | 1428.40 | 1428.28 |
| 103 | C153 | Biotin-PEG4 | CEYDEEFFTALARLRGGDICQYIQAKFQYLPALCIEEILDNLGCS | OH | 5633.44 | 1409.36 | 1409 |
| 104 | C154 | Biotin-PEG4 | CEYDEEFFTALARLRGGDICYYIQAKFAYLPALCIEEILDNLGCS | OH | 5611.44 | 1403.86 | 1403.55 |
| 105 | C155 | Biotin-PEG4 | CEYDEEFFTALARLRGGDICQYIQAKFAYVPGLCIEEILDNLGCS | OH | 5548.34 | 1388.09 | 1387.71 |
| 106 | C156 | Biotin-PEG4 | CEYDEEFFTALARLRGSDICLYIQAKFQYLPGLCIEEILDNLGCS | OH | 5634.47 | 1409.62 | 1409.2 |
| 107 | C157 | Biotin-PEG4 | CEYDEEFFTALARLRGGDICDYIQAKFQYLPGLCIAEILDNLGCS | OH | 5548.34 | 1388.09 | 1387.65 |
| 108 | C158 | Biotin-PEG4 | CEYDGEFFTALARLRGGDICQYIQAKFQYLPGLCIEEILDNLGCS | OH | 5547.35 | 1387.84 | 1387.45 |
| 109 | C159 | Biotin-PEG4 | CDYDEEFFTALARLRGGDICYYIQAKFSYLPGLCIEEILDNLGCS | OH | 5599.41 | 1400.85 | 1400.36 |
| 110 | C160 | Biotin-PEG4 | CYDEEEFFTALARLRGGDICQYIQAKFQYLPGLCIEEILDNLGCS | OH | 5605.42 | 1402.36 | 1402 |
| 111 | C161 | Biotin-PEG4 | CEYDEEFFTALASLRGGDICYYIQAKFQYLPGLCIEEILDNLGCS | OH | 5585.38 | 1397.35 | 1396.9 |
| 112 | C162 | Biotin-PEG4 | CEYDEEFFTALAQLRGGDICYYIQAKFQYLPGLCIEEILDNLGCS | OH | 5626.43 | 1407.61 | 1406.98 |
| 113 | C163 | Biotin-PEG4 | CEYDEEFFTALA(Cit)LRGGDICYYIQAKFQYLPGLCIEEILDNLGCS | OH | 5661.48 | 1416.37 | 1414.47 |
| 114 | C164 | Biotin-PEG4 | CEYDEEFFTALARLRGGDICYYIQA(hydroxy)- | OH | 5655.48 | 1414.87 | 5655.481 |
| | | | norleucinne)FQYLPGLCIEEILDNLGCS | | | | |
| 115 | C165 | Biotin-PEG4 | CEYDEEFFTALARLRGGDICYYIQAYFQYLPGLCIEEILDNLGCS | OH | 5,689.50 | 1423.37 | 1422.94 |
| 116 | C166 | Biotin-PEG4 | CEYDEEFFTALARLRGGDICYYIQAKFQYLPKLCIEEILDNLGCS | OH | 5725.62 | 1432.41 | 1432.17 |
| 117 | C167 | Biotin-PEG4 | CEYDEEFFTALARLRGGDICEYIQAKFQYLPKLCIEEILDNLGCS | OH | 5691.55 | 1423.89 | 1424.3 |
| 118 | C168 | Biotin-PEG4 | CEYDEEFFTALARLRGGDICSYIQAKFQYLPKLCIEEILDNLGCS | OH | 5649.52 | 1413.38 | 1413.82 |
| 119 | C169 | Biotin-PEG4 | CEYDEEFFTALARLRGGDICDYIQAKFQYLPKLCIEEILDNLGCS | OH | 5677.53 | 1420.38 | 1419.95 |
| 120 | C170 | Biotin-PEG4 | CEYDEEFFTALACitLRGDDICSYIQA(hydroxy- | OH | 5638.40 | 1410.60 | 1410.24 |
| | | | norleucine)FQYLPGLCIEEILDNLGCS | | | | |
| 121 | C171 | Biotin-PEG4 | CEYDEEFFTALA(Cit)LRGGDICEYIQAKFQYLPGLCIEEILDNLGCS | OH | 5621.42 | 1406.36 | 1410.24 |
| 122 | C172 | Biotin-PEG4 | CEYDEEFFTALA(Cit)LRGDDICSYIQAKFQYLPGLCIEEILDNLGCS | OH | 5637.41 | 1410.35 | 1409.93 |
| 123 | C173 | Biotin-PEG4 | CEYDEEFFTALARLRGGDICYYIQA(hydroxy- | OH | 5655.48 | 1414.87 | 1414.46 |
| | | | norleucine)FQYLPGLCIEEILDNLGCS | | | | |
| 115 | C174 | Biotin-PEG4 | DEYDEEFFTALARLRGGDICYYIQAYFQYLPGLCIEEILDNLGCS | OH | 5689.49 | 1423.37 | 1422.94 |
| 124 | C175 | Biotin-PEG4 | CEYDEEFFTALA(Cit)LRGGDICSYIQAKFQYLPGLCIEEILDNLGCS | OH | 5579.38 | 1395.85 | 1395.34 |
| 125 | C176 | Biotin-PEG4 | CDYDEEFFTALA(Cit)LRGGDICEYIQAKFQYLPGLCIEEILDNLGCS | OH | 5607.39 | 1402.85 | 1402.35 |
| 126 | C177 | Biotin-PEG4 | CEYDEEFFTALKRLRGGDICYYIQASFQYLPGECIEEILDNLGCS | OH | 5686.45 | 1422.61 | 1422.15 |
| 127 | C178 | Biotin-PEG4 | CEYDEEFFTALKRLRGGDICEYIQASFQYLPGLCIEEILDNLGCS | OH | 5,636.43 | 1410.11 | 1409.9 |
| 128 | C179 | Biotin-PEG4 | CEYDEEFFTALKRLRGGDICSYIQASFQYLPGLCIEEILDNLGCS | OH | 5,594.39 | 1399.60 | 1399.24 |
| 129 | C180 | Biotin-PEG4 | CEYDEEFFTALKRLRGDDICYYIQASFQYLPGLCIEEILDNLGCS | OH | 5,728.53 | 1433.13 | 1432.7 |
| 130 | C181 | Biotin-PEG4 | CEYDEEFFTALKRLRGDDICEYIQASFQYLPGLCIEEILDNLGCS | OH | 5,694.47 | 1424.62 | 1424.07 |
| 131 | C182 | Biotin-PEG4 | CEYDEEFFTALKRLRGDDICSYIQASFQYLPGLCIEEILDNLGCS | OH | 5,652.43 | 1414.11 | 1413.13 |
| 132 | C183 | Biotin-PEG4 | CEYDEQFFTALKRLRGADICEYIQASFQYLPGLCIEEILDNLGCS | OH | 5,649.47 | 1413.37 | 1413.07 |
| 133 | C184 | Biotin-PEG4 | CEYDEQFFTALKRLRGGDICSYIQASFQYLPGLCIEEILDNLGCS | OH | 5,593.41 | 1399.35 | 1398.93 |
| 134 | C185 | Biotin-PEG4 | CEYDEQFFTALKRLRGADICSYIQASFQYLPGLCIEEILDNLGCS | OH | 5,607.44 | 1402.86 | 1402.32 |
| 135 | C186 | Biotin-PEG4 | CEYDEQFFTALKRLRGDDICSYIQASFQYLPGLCIEEILDNLGCS | OH | 5,651.45 | 1413.86 | 1413.43 |
| 93 | C187 | ^{nat}In:DOTA- | CEYDEEFFTALARLRGDDICSYIQAKFQYLPGLCIEEILDNLGCS | OH | 5908.33 | 1478.08 | 1477.66 |
| | | PEG4 | | | | | |
| 136 | C188 | Biotin-PEG4 | CDYDEEFFTALKRLRGGDICEYIQASFQYLPGLCIEEILDNLGCS | OH | 5,622.40 | 1406.60 | 1406.18 |
| 137 | C189 | Biotin-PEG4 | CDYDEEFFTALKRLRGGDICSYIQASFQYLPGLCIEEILDNLGCS | OH | 5,580.37 | 1396.09 | 1395.63 |
| 138 | C190 | Biotin-PEG4 | CDYDEQFFTALKRLRGGDICEYIQASFQYLPGLCIEEILDNLGCS | OH | 5,621.42 | 1406.36 | 1405.57 |
| 139 | C191 | Biotin-PEG4 | CDYDEEFFTALKRLRGDDICEYIQASFQYLPGLCIEEILDNLGCS | OH | 5,680.44 | 1421.11 | 1420.73 |
| 140 | C192 | Biotin-PEG4 | CDYDEEFFTALKRLRGDDICSYIQASFQYLPGLCIEEILDNLGCS | OH | 5,638.40 | 1410.60 | 1410.18 |
| 141 | C193 | Biotin-PEG4 | CDYDEQFFTALKRLRGDDICEYIQASFQYLPGLCIEEILDNLGCS | OH | 5,679.46 | 1420.87 | 1420.48 |
| 142 | C194 | Biotin-PEG4 | CEYDEQFFTALKRLRGADICDYIQASFQYLPGLCIEEILDNLGCS | OH | 5635.45 | 1409.86 | 1409.74 |
| 143 | C195 | Biotin-PEG4 | CEYDEEFFTALKRLRGADICDYIQASFQYLPGLCIEEILDNLGCS | OH | 5636.44 | 1410.11 | 1409.68 |
| 144 | C196 | Biotin-PEG4 | CEYDEQFFTALKRLRGGDICDYIQASFQYLPGLCIEEILDNLGCS | OH | 5621.42 | 1406.36 | 1405.95 |
| 145 | C197 | Biotin-PEG4 | CEYDEQFFTALKRLRGDDICDYIQASFQYLPGLCIEEILDNLGCS | OH | 5679.46 | 1420.87 | 1420.52 |
| 146 | C198 | Biotin-PEG4 | CEYDEEFFTALKRLRGGDICEYIQAAFQYLPGLCIEEILDNLGCS | OH | 5620.44 | 1406.11 | 1405.45 |
| 147 | C199 | Biotin-PEG4 | CEYDEEFFTALKRLRGGDICEYIQANleFQYLPGLCIEEILDNLGCS | OH | 5662.52 | 1416.63 | 1416.22 |
| 148 | C200 | Biotin-PEG4 | CEYDEEFFTALKRLRGGDICDYIQASFQYLPGLCIEEILDNLGCS | OH | 5622.41 | 1406.60 | 1406.12 |
| 149 | C201 | Biotin-PEG4 | CEYDEEFFTALKRLRGGDICEYIQAKme3FQYLPGLCIEEILDNLGCS | OH | 5720.62 | 1431.16 | 1430.4 |
| 150 | C202 | Biotin-PEG4 | CEYDEEFFTALKRLRGGDICEYIQASSFQYLPGECIEEILDNLGCS | OH | 5652.39 | 1414.10 | 1413.65 |
| 151 | C203 | Biotin-PEG4 | CEYDEEFFTALKRLRGGDICDYIQASFQYLPGECIEEILDNLGCS | OH | 5638.36 | 1410.59 | 1410.22 |
| 152 | C204 | Biotin-PEG4 | CEYDEEFFTALKRLRGGDICEYIQASFQYLPGECIEEILQNLGCS | OH | 5665.43 | 1417.36 | 1417.02 |
| 153 | C205 | Biotin-PEG4 | CEYDEEFFTALKRLRGGDICDYIQASFQYLPGECIEEILQNLGCS | OH | 5651.4 | 1413.85 | 1413.71 |
| 154 | C207 | Biotin-PEG4 | CDYDEQFFTALKRLRGADICEYIQASFQYLPGLCIEEILDNLGCS | OH | 5635.45 | 1409.86 | 1409.62 |
| 155 | C208 | Biotin-PEG4 | CDYDEQFFTALKRLRGADICEYIQASFQYLPGECIEEILDNLGCS | OH | 5651.41 | 1413.85 | 1413.38 |
| 156 | C209 | Biotin-PEG4 | CDYDEQFFTALKRLRGADICEYIQASFQYLPGQCIEEILDNLGCS | OH | 5650.42 | 1413.61 | 1413.25 |
| 157 | C210 | Biotin-PEG4 | CDYDEQFFTALKRLRGGDICEYIQASFQYLPGECIEEILDNLGCS | OH | 5637.38 | 1410.34 | 1409.92 |
| 158 | C211 | Biotin-PEG4 | CDYDEQFFTALKRLRGGDICEYIQASFQYLPGQCIEEILDNLGCS | OH | 5636.40 | 1410.10 | 1409.69 |
| 177 | C212 | ACETYL | CEYDEEFFTALKRLRGGDICYYIKKKFDYLPKLCIEEILDNLGC | NH2 | — | — | — |
| 177 | C213 | ACETYL | CEYDEEFFTALKRLRGGDICYYIKKKFDYLPKLCIEEILDNLGC | OH | — | — | — |
| 177 | C214 | DOTA-PEG4 | CEYDEEFFTALKRLRGGDICYYIKKKFDYLPKLCIEEILDNLGC | NH2 | — | — | — |
| 48 | C215 | DOTA-PEG4 | CEYDEEFFTALKRLRGGDICYYIKKKFKKVPKLCIEEILDKLGC | OH | — | — | — |
| 75 | C216 | ^{nat}In-DOTA- | CEYDEEFFTALKRLRGGDICYYILys(Ac)Lys(Ac)Lys(Ac)FLys(Ac)- | NH2 | — | — | — |
| | | PEG4 | (Ac)VPLys(Ac)LCIEEILDKLGC | | | | |
| 73 | C217 | ^{nat}In-DOTA- | CEYDEEFFTALLys(Ac)CitrullineLCitrullineGGDICYYIKKKFKKVP- | NH2 | — | — | — |
| | | PEG4 | KLCIEEILDLys(Ac)LGC | | | | |

#NOTE: In all sequences shown, Cys1 is connected to Cys34 and Cys 20 is connected to Cys 44.
*Compounds designated In-labeled in Table 2A are cold-metal labeled, “using natural abundance” Indium, also known as ^{nat}In. This Indium-may
comprise a combination of 113-In and 115-In, and distinguished from 111-In, which can be used as a radiolabel for conjugates provided herein.
DOTA-PEG4: alpha-(1,4,7,10-tetraazacyclododecan-1,4,7,10-tetraacetate)-4(ethylene glycol)
DOTA-PEG8: alpha-(1,4,7,10-tetraazacyclododecan-1,4,7,10-tetraacetate)-8(ethylene glycol)
DOTA-PEG12: alpha-(1,4,7,10-tetraazacyclododecan-1,4,7,10-tetraacetate)-12(ethylene glycol)
Biotin-PEG4: 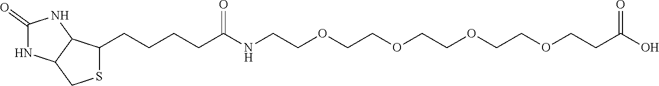 [SMILES: O=C(O)CCOCCOCCOCCOCCNC(=O)CCCCC1SCC2NC(=O)NC21]
FITCl-PEG4: Fluorescein isothiocyanate-4(ethylene glycol)

TABLE 2B
Binding Affinities of Exemplary Compounds to Nectin-4

| SEQ ID NO OF | Compound | Binding Affinity |
|---|---|---|
| POLYPEPTIDE TESTED | Name | (nM) |
| 1 | C1 | 184.20 |
| 2 | C2 | 58.92 |
| 3 | C3 | 9.68 |
| 4 | C4 | 14.40 |
| 3 | C5 | 18.93 |
| 4 | C6 | 17.09 |
| 5 | C7 | 5.50 |
| 5 | C8 | 9.77 |
| 5 | C9 | 7.50 |
| 5 | C10 | 37.58 |
| 5 | C11 | 6.86 |
| 5 | C12 | 10.50 |
| 6 | C13 | 6.01 |
| 7 | C14 | 14.11 |
| 8 | C15 | 12.17 |
| 9 | C16 | 4.34 |
| 10 | C17 | 4.16 |
| 11 | C18 | 38.16 |
| 12 | C19 | 10.81 |
| 13 | C20 | 22.08 |
| 14 | C21 | 49.39 |
| 15 | C22 | 12.83 |
| 16 | C23 | 18.51 |
| 17 | C24 | 84.39 |
| 18 | C25 | 3.745 |
| 19 | C26 | 5.04 |
| 20 | C27 | 9.85 |
| 21 | C28 | 5.29 |
| 22 | C29 | 3.91 |
| 23 | C30 | 24.92 |
| 24 | C31 | 1.31 |
| 25 | C32 | 3.26 |
| 26 | C33 | 3.47 |
| 27 | C34 | 3.17 |
| 34 | C41 | 5.60 |
| 36 | C43 | 3.97 |
| 37 | C44 | 3.14 |
| 38 | C45 | 3.29 |
| 39 | C46 | 2.28 |
| 42 | C49 | 1.93 |
| 5 | C51 | 8.37 |
| 43 | C52 | 5.24 |
| 46 | C55 | 3.39 |
| 47 | C56 | 3.88 |
| 48 | C57 | 0.29 |
| 49 | C58 | 1.81 |
| 51 | C60 | 3.17 |
| 52 | C61 | 1.79 |
| 54 | C63 | 1.97 |
| 55 | C64 | 3.80 |
| 56 | C65 | 2.21 |
| 57 | C66 | 2.63 |
| 58 | C67 | 2.64 |
| 59 | C68 | 2.66 |
| 60 | C69 | 1.86 |
| 61 | C70 | 2.20 |
| 62 | C71 | 2.85 |
| 67 | C76 | 0.52 |
| 68 | C77 | 0.58 |
| 69 | C78 | 0.49 |
| 70 | C79 | 0.38 |
| 71 | C80 | 2.70 |
| 72 | C81 | 0.43 |
| 76 | C100 | 4.80 |
| 76 | C102 | 6.40 |
| 76 | C103 | 6.60 |
| 77 | C104 | 3.70 |
| 77 | C106 | 4.38 |
| 77 | C107 | 3.38 |
| 78 | C108 | 5.78 |
| 78 | C111 | 4.32 |
| 79 | C112 | 6.24 |
| 80 | C115 | 6.46 |
| 81 | C116 | 0.78 |
| 81 | C119 | 0.95 |
| 82 | C120 | 2.11 |
| 82 | C123 | 1.43 |
| 83 | C124 | 1.10 |
| 83 | C127 | 1.27 |
| 84 | C128 | 10.10 |
| 85 | C131 | 12.40 |
| 86 | C132 | 30.10 |
| 87 | C135 | 5.42 |
| 88 | C136 | 36.50 |
| 89 | C139 | 25.20 |
| 90 | C140 | 1.10 |
| 91 | C141 | 0.64 |
| 92 | C142 | 0.81 |
| 93 | C143 | 0.55 |
| 94 | C144 | 3.70 |
| 95 | C145 | 1.30 |
| 96 | C146 | 0.54 |
| 97 | C147 | 0.69 |
| 98 | C148 | 0.82 |
| 99 | C149 | 0.75 |
| 100 | C150 | 1.00 |
| 101 | C151 | 0.68 |
| 102 | C152 | 0.85 |
| 103 | C153 | 0.56 |
| 104 | C154 | 0.94 |
| 105 | C155 | 1.10 |
| 106 | C156 | 1.10 |
| 107 | C157 | 1.10 |
| 108 | C158 | 1.10 |
| 111 | C161 | 7.50 |
| 112 | C162 | 8.80 |
| 113 | C163 | 1.90 |
| 114 | C164 | 5.00 |
| 115 | C165 | 4.10 |
| 116 | C166 | 0.75 |
| 117 | C167 | 1.20 |
| 118 | C168 | 0.90 |
| 119 | C169 | 0.74 |
| 120 | C170 | 8.80 |
| 121 | C171 | 2.20 |
| 122 | C172 | 1.80 |
| 123 | C173 | 5.00 |
| 115 | C174 | 4.10 |
| 124 | C175 | 2.30 |
| 125 | C176 | 1.30 |
| 126 | C177 | 2.00 |
| 127 | C178 | 1.40 |
| 128 | C179 | 1.80 |
| 129 | C180 | 2.70 |
| 130 | C181 | 3.00 |
| 131 | C182 | 3.00 |
| 132 | C183 | 1.20 |
| 133 | C184 | 2.50 |
| 134 | C185 | 1.00 |
| 135 | C186 | 1.70 |
| 136 | C188 | 1.60 |
| 137 | C189 | 1.10 |
| 138 | C190 | 0.71 |
| 139 | C191 | 1.40 |
| 140 | C192 | 1.30 |
| 141 | C193 | 1.00 |
| 142 | C194 | 1.10 |
| 143 | C195 | 2.30 |
| 144 | C196 | 1.60 |
| 145 | C197 | 0.97 |
| 146 | C198 | 2.30 |
| 147 | C199 | 3.50 |
| 148 | C200 | 2.00 |
| 149 | C201 | 0.68 |
| 150 | C202 | 2.20 |
| 151 | C203 | 2.30 |
| 152 | C204 | 1.98 |
| 153 | C205 | 2.10 |
| 154 | C207 | 1.08 |
| 155 | C208 | 1.02 |
| 156 | C209 | 1.25 |
| 157 | C210 | 1.54 |
| 158 | C211 | 1.19 |

In some embodiments, a miniprotein of the present disclosure exhibits binding specificity to human Nectin-4. For example, in some embodiments a miniprotein provided by the present disclosure, such as, for example, those represented by any one of SEQ ID NOs: 1-158 or 177 or 161-176, demonstrates binding when expressed on the surface of yeast and binding to Nectin-4 tested by flow cytometry. In some embodiments, a miniprotein provided by the present disclosure, such as, for example, those represented by any one of SEQ ID NOs: 1-158 or 177 or 161-176 or in accordance with Tables 1B, 1C, and/or 2A, demonstrates binding specificity via flow cytometry when, for example, such a Nectin-4 miniprotein (e.g., as represented by any of SEQ ID NOs: 1-158, 177, 161-176, or in accordance with any of Tables 1B, 1C, and/or 2A) only binds to Nectin-4 and not to other antigens.

In some embodiments, a miniprotein of the present disclosure such as, for example, any of those represented by SEQ ID NOs 1-158 or 177 or 161-176 or in accordance with Tables 1B, 1C, and/or 2A, shows greater than 10 nM potency. In some embodiments, a miniprotein shows potency greater than 1, 2, 3, 4, 5, 6, 7, 8, 9 nM or more.

In some embodiments, a miniprotein is part of a conjugate comprising one or more modifications or components, for example, as provided herein (see, e.g., Table 2A).

In some embodiments, a miniprotein in accordance with the present disclosure displays a binding specificity to human Nectin-4. In some embodiments, the miniprotein comprises a binding affinity characterized by a dissociation constant ranging from about 500 nM to about 1 pM, e.g., 500, 400, 300. 200, 100, 90, 80, 70, 60, 50, 40, 30 20, 10, 9, 8, 7, 6, 5, 4, 3, 2, 1 nM, 100, 90, 80, 70, 60, 50, 40, 30, 20, 10, 9, 8, 7, 6, 5, 4, 3, 2, or 1 pM binding affinity to human Nectin-4. Without being bound by theory, the present disclosure contemplates that, in some embodiments, a preferred dissociation constant of a miniprotein is about 10 nM or less, about 7.5 nM, about 5 nM or less, about 2.5 nM or less, about 1 nM or less (i.e., in the picomolar range).

In some embodiments, a miniprotein of the present disclosure binds to Nectin-4 with a binding affinity of about 1 pM to 100 nM. In some embodiments, a miniprotein in accordance with the present disclosure binds to Nectin-4 with a binding affinity of about 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 pM; 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, or 1000 nM. In some embodiments, a miniprotein in accordance with the present disclosure binds to Nectin-4 with a binding affinity of about 1 pM to 100 pM, 10 pM to 1 nM, 100 pM to 10 nM, or 1 nM to 100 nM.

### CDPs

In some embodiments, miniproteins of the present disclosure comprise or consist of a cysteine-dense peptides (CDPs). In some embodiments, conjugates provided herein comprise a CDP. In some embodiments, a CDP functions as a targeting moiety, e.g., specifically binding to a protein target or antigen expressed on the surface of a target tumor cell. In some embodiments, a CDP comprises or consists of at least two independent folding domains and a high density of cysteines. In some embodiments, the CDP comprises at least one, two, three, four, five, six, or more than six cysteine residues in a span of from about 10 to about 90 amino acid residues, preferably 13 to 80 amino acid residues. (See, e.g., Correnti et al., Nat Struct Mol Biol. 2018 March; 25(3):270-278, for exemplary CDPs and characteristics thereof). In some embodiments, the CDP comprises a constrained distribution of cysteines, Cys-_{X[0-15]}-Cys-_{X[0-15]}-Cys-_{X[0-15]}-Cys-_{X[0-15]}-Cys-_{X[0-15]}-Cys (wherein X represents any amino acid). In some embodiments, a CDP comprises one or more cysteine dense regions comprising at least one cysteine residue, preferably at least two, three, four, or more cysteine residues in a span of from about 10 to 80 amino acid residues. In some embodiments, a CDP can be further engineered to modify binding, folding, and/or related properties.

In some embodiments, a CDP specifically binds to a target. In some embodiments, the target is located in, on, or near a cell. In some embodiments, the CDP specifically binds to Nectin-4 or a fragment thereof. In some embodiments, a CDP is conjugated to a chelator and/or radionuclide. In some embodiments, conjugation is via a linker. It will be understood by those of skill in the art, that in some embodiments, the particular CDP employed in a conjugate of the present disclosure may vary depending on the target protein or antigen of interest.

In some embodiments, in miniproteins having cysteine residues, to ensure proper folding and connectivity, selected cysteine pairs can be replaced with selenocysteines. In some embodiments, diselenide crosslinks may form more readily than disulfide crosslinks due to their lower redox potential. In some such embodiments, such replacement can lead to cross-coupling of remaining cysteines.

### Knottins

In some a embodiments, miniproteins of the present disclosure comprise or consist of knottin peptides. In some embodiments, conjugates provided herein comprise a knottin peptide. In some embodiments, a knottin peptide functions as a targeting moiety, e.g., specifically binding to an antigen expressed on the surface of a target tumor cell. In some embodiments, a knottin comprises at least three disulfide bonds connected in an arrangement that generates the so-called “cysteine-knot” for which knottins are named. (See, e.g., Kintzing & Cochran et al., Curr Opin Chem Biol. 2016 October; 34:143-150.). In some embodiments, knottins have high stability (e.g., thermal, proteolytic, chemical, etc.). In some embodiments, a knottin can be further engineered to modify binding, folding, and/or related properties.

In some embodiments, a given knottin is highly specific for a given target. In some embodiments, a knottin specifically binds to a target. In some embodiments, the target is located in, on, or near a cell. In some embodiments, the knottin specifically binds to Nectin-4 or a fragment thereof. In some embodiments, a knottin is conjugated to a chelator and/or radionuclide. In some embodiments, conjugation is via a linker. It will be understood by those of skill in the art, that in some embodiments, the particular knottin employed in a conjugate of the present disclosure may vary depending on the target protein or antigen of interest.

In some embodiments, folded structures of miniproteins (e.g., CDPs, knottins, binders, affibodies, engineered Kunitz domains, monobodies, anticalins, designed ankyrin repeat domains (DARPins), avimers) make them rigid, providing for very tight and potent binding to the target protein or antigen (relative to less structured peptides). In some such embodiments, a miniprotein (e.g., CDP, knottin, binder, affibody, engineered Kunitz domain, monobody, anticalin, designed ankyrin repeat domain (DARPin), avimer) exhibits extraordinary stability with resistance to heat, peptidase cleavage, and pH.

### Binders

In some embodiments, a miniprotein of the present disclosure comprises or consists of a binder. In some embodiments, the binder functions as a targeting moiety, e.g., specifically binding to a target expressed on the surface of a tumor cell.

In some embodiments, a binder has certain structural features; for example, in some embodiments, a binder may be rich in alpha-helices, such as a helix-helix-helix structure (see, e.g., Crook et al., Nat Commun. (2017) 8, 2244; Berger et al, Elife (2016) 5, e20352; and Procko et al., Cell (2014), 157, 1644-1656). In some embodiments, a binder comprises sufficient surface to functionalize the molecule on a disparate surface to a binding surface. In some embodiments, a binder comprises a sequestered hydrophobic core. In some embodiments, a binder displays cooperative folding. In some embodiments, a binder has two or more of the following features: (i) represented by an amino acid sequence of 100 amino acids or fewer; (ii) at least two secondary structure elements; (iii) a sequestered hydrophobic core; and/or (iv) cooperative folding.

In some embodiments, a given binder is highly specific for a given target. In some embodiments, a binder specifically binds to a target. In some embodiments, the target is located in, on, or near a cell. In some embodiments, the binder specifically binds to Nectin-4 or a fragment thereof. In some embodiments, a binder is conjugated to a chelator and/or radionuclide. In some embodiments, conjugation is via a linker. It will be understood by those of skill in the art, that in some embodiments, the particular binder employed in a conjugate of the present disclosure may vary depending on the target protein or antigen of interest.

### Affibodies

In some embodiments, miniproteins of the present disclosure comprise or consist of affibodies. In some embodiments, conjugates provided herein comprise an affibody. In some embodiments, an affibody functions as a targeting moiety, e.g., specifically binding to a protein target or antigen expressed on the surface of a target tumor cell. In some embodiments, an affibody comprises or consists of no more than 100 amino acids, 90 amino acids, 80 amino acids, 70 amino acids, 60 amino acids, 50 amino acids, 40 amino acids, 30 amino acids, 20 amino acids, or 10 amino acids. In some embodiments, an affibody comprises or consists of at least three alpha helices with 58 amino acids. In some embodiments, the affibody comprises target specificity that is obtained by randomization of 13 amino acids located in two alpha-helices involved in the binding activity of the parent protein domain (Feldwisch J, Tolmachev V.; (2012) Methods Mol Biol. 899:103-26). In some embodiments, an affibody can be further engineered to modify binding, folding, and/or related properties.

In some embodiments, an affibody specifically binds to a target. In some embodiments, the target is located in, on, or near a cell. In some embodiments, the affibody specifically binds to Nectin-4 or a fragment thereof. In some embodiments, an affibody is conjugated to a chelator and/or radionuclide. In some embodiments, conjugation is via a linker. It will be understood by those of skill in the art, that in some embodiments, the particular affibody employed in a conjugate of the present disclosure may vary depending on the target protein or antigen of interest.

### Engineered Kunitz Domains

In some embodiments, miniproteins of the present disclosure comprise or consist of engineered Kunitz domains. In some embodiments, conjugates provided herein comprise an engineered Kunitz domain. In some embodiments, an engineered Kunitz domain functions as a targeting moiety, e.g., specifically binding to a protein target or antigen expressed on the surface of a target tumor cell. In some embodiments, an engineered Kunitz domain comprises or consists of at least one peptide derived from the Kunitz domain of a Kunitz-type protease inhibitor such as bovine pancreatic trypsin inhibitor (BPTI), amyloid precursor protein (APP) or tissue factor pathway inhibitor (TFPI). In some embodiments, an engineered Kunitz domain can be further engineered to modify binding, folding, and/or related properties.

In some embodiments, an engineered Kunitz domain specifically binds to a target. In some embodiments, the target is located in, on, or near a cell. In some embodiments, the engineered Kunitz domain specifically binds to Nectin-4 or a fragment thereof. In some embodiments, an engineered Kunitz domain is conjugated to a chelator and/or radionuclide. In some embodiments, conjugation is via a linker. It will be understood by those of skill in the art, that in some embodiments, the particular engineered Kunitz domain employed in a conjugate of the present disclosure may vary depending on the target protein or antigen of interest.

### Monobodies

In some embodiments, miniproteins of the present disclosure comprise or consist of monobodies. In some embodiments, conjugates provided herein comprise a monobody. In some embodiments, a monobody functions as a targeting moiety, e.g., specifically binding to a protein target or antigen expressed on the surface of a target tumor cell. In some embodiments, a monobody comprises or consists of a molecule based ° n the 10th extracellular domain of human fibronectin III (1° fn3), which adopts an Ig-like b-sandwich fold of about 94 residues with 2 to 3 exposed loops, but lacks the central disulfide bridge. In some embodiments, a monobody can be further engineered to modify binding, folding, and/or related properties.

In some embodiments, a monobody specifically binds to a target. In some embodiments, the target is located in, on, or near a cell. In some embodiments, the monobody specifically binds to Nectin-4 or a fragment thereof. In some embodiments, a monobody is conjugated to a chelator and/or radionuclide. In some embodiments, conjugation is via a linker. It will be understood by those of skill in the art, that in some embodiments, the particular monobody employed in a conjugate of the present disclosure may vary depending on the target protein or antigen of interest.

### Anticalins

In some embodiments, miniproteins of the present disclosure comprise or consist of anticalins. In some embodiments, conjugates provided herein comprise an anticalin. In some embodiments, an anticalin functions as a targeting moiety, e.g., specifically binding to a protein target or antigen expressed on the surface of a target tumor cell. In some embodiments, an anticalin comprises or consists of an eight-stranded 0-barrel which forms a highly conserved core unit among the lipocalins and naturally forms binding sites for ligands by means of four structurally variable loops at the open end. In some embodiments, an anticalin can be further engineered to modify binding, folding, and/or related properties.

In some embodiments, an anticalin specifically binds to a target. In some embodiments, the target is located in, on, or near a cell. In some embodiments, the anticalin specifically binds to Nectin-4 or a fragment thereof. In some embodiments, an anticalin is conjugated to a chelator and/or radionuclide. In some embodiments, conjugation is via a linker. It will be understood by those of skill in the art, that in some embodiments, the particular anticalin employed in a conjugate of the present disclosure may vary depending on the target protein or antigen of interest.

### Designed Ankyrin Repeat Domains

In some embodiments, miniproteins of the present disclosure comprise or consist of designed Ankyrin repeat domains. In some embodiments, conjugates provided herein comprise a designed Ankyrin repeat domain. In some embodiments, a designed Ankyrin repeat domain functions as a targeting moiety, e.g., specifically binding to a protein target or antigen expressed on the surface of a target tumor cell. In some embodiments, a designed Ankyrin repeat domain comprises a peptide derived from Ankyrin. In some embodiments, a designed Ankyrin repeat domain comprises a single ankyrin repeat, preferably comprising a 33-residue motif comprising two alpha-helices and a beta-turn. In some embodiments a designed Ankyrin repeat domain provides a rigid interface and lacks structural flexibility. In some embodiments, a designed Ankyrin repeat domain can be further engineered to modify binding, folding, and/or related properties.

In some embodiments, a designed Ankyrin repeat domain specifically binds to a target. In some embodiments, the target is located in, on, or near a cell. In some embodiments, the designed Ankyrin repeat domain specifically binds to Nectin-4 or a fragment thereof. In some embodiments, a designed Ankyrin repeat domain is conjugated to a chelator and/or radionuclide. In some embodiments, conjugation is via a linker. It will be understood by those of skill in the art, that in some embodiments, the particular designed Ankyrin repeat domain employed in a conjugate of the present disclosure may vary depending on the target protein or antigen of interest.

### Avimers

In some embodiments, miniproteins of the present disclosure comprise or consist of avimers. In some embodiments, conjugates provided herein comprise an avimer. In some embodiments, an avimer functions as a targeting moiety, e.g., specifically binding to a protein target or antigen expressed on the surface of a target tumor cell. In some embodiments, an avimer comprises a peptide of about 10 amino acids, 20 amino acids, 30 amino acids, 40 amino acids, 50 amino acids, 60 amino acids, 70 amino acids, 80 amino acids, 90 amino acids, or 100 amino acids. In some embodiments, an avimer comprises at least one peptide sequence of about 30 to 35 amino acids. In some embodiments, an avimer comprises two or more of two peptide sequences of about 30 to 35 amino acids. In some embodiments, an avimer comprises one or more peptide sequences derived from A-domains of various membrane receptors. (Weidle U H, et al., (2013), Cancer Genomics Proteomics; 10(4): 155-68). For further details see Nature Biotechnology 23(-2), 1556-1561 (2005) and Expert Opinion on Investigational Drugs 16(6), 909-917 (June 2007). In some embodiments, an avimer can be further engineered to modify binding, folding, and/or related properties.

In some embodiments, an avimer specifically binds to a target. In some embodiments, the target is located in, on, or near a cell. In some embodiments, the avimer specifically binds to Nectin-4 or a fragment thereof. In some embodiments, an avimer is conjugated to a chelator and/or radionuclide. In some embodiments, conjugation is via a linker. It will be understood by those of skill in the art, that in some embodiments, the particular avimer employed in a conjugate of the present disclosure may vary depending on the target protein or antigen of interest.

### Linkers

In some embodiments the present disclosure provides linkers for use in one or more conjugates. For example, in some embodiments, a linker is linked to a chelator. In some embodiments, a linker is linked to a chelator, which itself is coupled to a radionuclide. In some embodiments, a miniprotein is conjugated to a chelator and/or radionuclide. In some embodiments, a miniprotein is conjugated to a chelator, optionally, through a linker. In some embodiments, a composition as provided herein comprises one or more linkers.

As described herein, in some embodiments, a miniprotein conjugate comprises a linker. In some embodiments, the linker functions to connect the chelator to miniprotein. In some embodiments, a linker is non-cleavable. In some embodiments, a linker is cleavable. In some embodiments, selection and placement of one or more linkers and chelators on a miniprotein aids to maintain desired potency and receptor engagement profile, enhance binder affinity and optimize physicochemical and pharmacokinetic properties of a miniprotein or conjugate thereof. Any suitable linker known in the art can be utilized. Exemplary linkers include, but are not limited to polyethylene glycol (PEG) linkers, an ester linker, an amide linker, a maleimide linker, a valine-citrulline linker, a hydrazone linker, a N-succinimidyl-4-(2-pyridyldithio)butyrate (SPDB) linker, a succinimidyl-4-(N-maleimidomethyl)cyclohexane-1-carboxylate (SMCC) linker, a vinylsulfone-based linker, a propanoic acid linker, a caproleic acid linker, or a linker including any combination thereof. One or more additional linkers may be contemplated as will be known to those of skill in the art and chosen given the context and components of a given composition. In some embodiments, the linker is a PEG linker. In some embodiments, the linker is a non-cleavable PEG linker. In some embodiments, the PEG linker is any of PEGs (2-24).

In some embodiments, linkers are used to assess lead polypeptide sequences binding to a target, a target expressed on cells, and target selectivity and/or affinity. For instance, in some embodiments, confirmation of in vitro on-target binding and affinity for lead polypeptide sequences and lead polypeptide sequences-linker-fluorophore reagent can be assessed using Biacore. In some embodiments, other linkers such as a fast clear linker or a halogen linker are also contemplated.

### Chelators

In some embodiments, a composition (e.g., conjugate) as provided herein comprises a linker. In some embodiments, a composition comprises a linker and a chelator. In some embodiments, a composition comprises a linker, a chelator, and a radionuclide. In some embodiments, a composition comprises a miniprotein, optional linker, chelator, and/or radionuclide. In some embodiments, a chelator is covalently attached to a miniprotein. In some embodiments, a chelator binds to a radionuclide. In some embodiments, a chelator refers to any molecule or moiety that “binds” to a metal ion, in solution (effectively collecting/binding up metal ions so that they may, e.g., no longer participate in one or more cellular activities or processes). In some embodiments a chelator chelates one or more components of a metabolic pathway in a cell (e.g., metal ions, e.g., copper, iron, zinc, etc.). In some such embodiments, a chelator disrupts a life-cycle of a cancer cell and may, in some embodiments, reduce its viability, function, and/or ability to grow or proliferate. In some embodiments, a chelator chelates one or more toxins that are produced as a result of targeted radiotherapy (e.g., to reduce toxicity of the therapy).

In some embodiments, a chelator comprises or consists of, but is not limited to tetrazacyclododecane-1,4,7,10-tetraacetic acid (DOTA), diethylenetriamine pentaacetic acid (DTPA), ethylenediaminetetraacetic acid (EDTA), 1,4,7-triazacyclo′on″ne-N,N′,N”-triacetic acid (NOTA), ethylenediaminetetraacetic acid (EDTA), diethylenetriaminepentaacetic acid (DTPA), 1,4,7- triazacyclo′on″ne-N,N′,N”-triacetic acid (NOTA), ({4-[2- (bis-carboxymethyl-amino)-ethyl]-7-carboxymethyl-[1,4,7]triazonan-l-yl}acetic acid (NETA), Macropa, and p-bromoacetamidobenzyl-tetraethylaminetetraacetic acid (TETA), porphyrins, polyamines, crown ethers, bis-thiosemicarbazones, polyoximes. In some embodiments, the chelator is 1,4,7,10-tetraazacyclododecane-1,4,7,10-tetraacetic acid (DOTA). In some embodiments, the chelator is Macropa. In some embodiments, a chelator comprises or consists of:

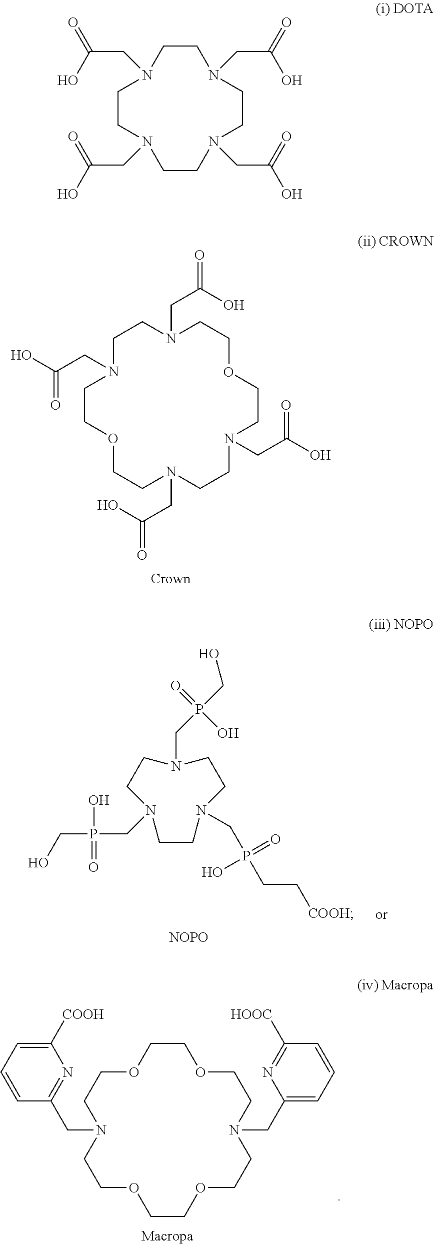 [SMILES: O=C(O)CN1CCN(CC(=O)O)CCN(CC(=O)O)CCN(CC(=O)O)CC1 ; O=C(O)CN1CCOCCN(CC(=O)O)CCN(CC(=O)O)CCOCCN(CC(=O)O)CC1 ; O=C(O)CCP(=O)(O)CN1CCN(CP(=O)(O)CO)CCN(CP(=O)(O)CO)CC1 ; "NOPO" ; O=C(O)c1cccc(CN2CCOCCOCCN(Cc3cccc(C(=O)O)n3)CCOCCOCC2)n1]

In some embodiments, a chelator comprises or consists of, but is not limited to diethylenetriamine pentaacetic acid (DTPA), tetrazacyclododecane-1,4,7,10-tetraacetic acid (DOTA), ethylenediaminetetraacetic acid (EDTA), 1,4,7-triazacyclo′on″ne-N,N′,N “-tri acetic acid (NOTA), ethylenediaminetetraacetic acid (EDTA), diethylenetriaminepentaacetic acid (DTPA), 1,4,7- triazacyclo′on″ne-N,N′,N”-triacetic acid (NOTA), ({4-[2- (bis-carboxymethyl-amino)-ethyl]-7-carboxymethyl-[1,4,7]triazonan-l-yl}acetic acid (NETA), Macropa, and p-bromoacetamidobenzyl-tetraethylaminetetraacetic acid (TETA), porphyrins, polyamines, crown ethers, bis-thiosemicarbazones, polyoximes. In some embodiments, the chelator is 1,4,7,10-tetraazacyclododecane-1,4,7,10-tetraacetic acid (DOTA). In some embodiments, the chelator is Macropa. In some embodiments, a chelator comprises or consists of:

In additional embodiments, the chelation conditions are optimized using methods known to those of skill in the art (see, e.g., J Nucl Med. 1998 December; 39(12):2105-10). In some embodiments, chelation efficiency is about >99%, >98%, >97%, >96%, >95%, >94%, >93%, >92%, >91%, >90%,>89%, >88%, >87%, >86%, >85%, >84%, >83%, >82%, >81%, or >80%.

In some embodiments, a chelator for use in a composition as described herein is chosen based on if and which radionuclide is present. As provided herein, in some embodiments, a chelator is DOTA, NOPO, Crown, or Macropa. In some embodiments, DOTA is the chelator and the radionuclide is Ac-225, In-111, Ga-68, Pb-212, Lu-177, Cu-67, Cu-64, La-132, La-135, or Ce-134. In some embodiments, Crown is the chelator and the radionuclide is Ac-225, In-111, Ga-68, Pb-212, Lu-177, Cu-67, Cu-64, La-132, La-135, or Ce-134. In some embodiments, NOPO is the chelator, and the radionuclide is Ac-225, In-111, Ga-68, Pb-212, Lu-177, Cu-67, Cu-64, La-132, La-135, or Ce-134. In some embodiments, Macropa is the chelator, and the radionuclide is Ac-225, In-111, Ga-68, Pb-212, Lu-177, Cu-67, Cu-64, La-132, La-135, or Ce-134.

In some embodiments, a particular chelator or type of chelator may be chosen for certain applications. In some embodiments, DOTA is used for diagnostic, theranostic, and/or therapeutic applications. For instance, in some embodiments, NOPO is used in diagnostic or theranostic applications. In some embodiments, Crown is used for therapeutic applications. In some embodiments, Macropa is used for diagnostic, theranostic, and/or therapeutic applications.

It is recognized that screening chelators for certain characteristics is within the scope of this disclosure and methods for such screening are known to those of skill in the art. For example, in some embodiments, chelators are screened for their ability to bind radionuclides (e.g., Ac225 and daughter(s) of Ac225 (Bi213), In-111, Ga68) and display serum stability.

In some embodiments, a miniprotein conjugate described herein comprises a chelator. Any suitable chelator known in the art can be utilized. In some embodiments the chelator is directly conjugated to the miniprotein. In some embodiments, the chelator is indirectly connected to the miniprotein through a linker. In some embodiments, the chelator is indirectly connected to the miniprotein through a linker (e.g., a linker described herein).

### Radionuclides

In some embodiments the present disclosure provides one or more radionuclides for use in a composition (e.g., conjugate).

In some embodiments, miniprotein conjugates comprise a radionuclide bound to a chelator. As will be understood to those of skill in the art, any suitable radionuclide known in the art may be used. In some embodiments, a radionuclide is selected for imaging of a tumor within a human having cancer. In some embodiments, a radionuclide is selected for its inability to kill cells in vivo. In some embodiments, the radionuclide is selected for its ability to kill cells in vivo.

In some embodiments, a composition of the present disclosure comprises one or more cytotoxic payloads including particle-emitting isotopes such as alpha-, beta-particles, and Auger electrons in radiotherapeutic applications. In some embodiments, a radionuclide of the present disclosure is an alpha emitter. As will be known to those of skill in the art, in some embodiments, an alpha emitter has a more localized area of impact such that when internalized into a cell it will act to, e.g., kill a cancer cell, but will spare surrounding tissue from extensive damage such as could occur with use of a beta or gamma emitter.

Studies have evaluated alpha nuclide therapy versus beta nuclide therapy with the stronger clinical results pointing to alpha nuclides. In some embodiments, a benefit of alpha therapy is that the short path length means patients do not have to physically distance themselves from family and health care providers making treatment more tolerable. Further, in some embodiments, alpha therapy exhibits better cell killing potency due to its ability to induce double stranded DNA breaks.

In some embodiments, a composition comprises a linker, chelator, and radionuclide. In some embodiments, a composition comprises a miniprotein, optional linker, chelator, and a radionuclide. Without being bound by any particular theory, the present disclosure contemplates that a wide variety of radionuclides can be used in the pharmaceutical composition or as a diagnostic. Exemplary radionuclides, include but are not limited to, Actinium-225, Indium-111, Astatine-211, Bismuth-212, Bismuth-213, Cesium-137, Chromium-51, Cobalt-60, Copper-64 Dysprosium-165, Erbium-169, Fermium-255, Fluor-18, Gallium-67, Gallium-68, Gold-198, Holmium-166, Iodine-123, Iodine-124, Iodine-125, Iodine-131, Iridium-192, Iron-59, Lead-212, Lutetium-177, Molybdenum-99, Palladium-103, Phosphorus-32, Potassium-42, Rhenium-186, Rhenium-188, Samarium-153, Technetium-99m, Radium-223, Ruthenium-106, Sodium-24, Strontium-89, Terbium-149, Thorium-227, Xenon-133, Ytterbium-169, Ytterbium-177, Yttrium-90, and Zirconium-89. Accordingly, in some embodiments, a radionuclide is selected from: actinium (225Ac), indium (111In), iodine (131I or 125I), yttrium (90Y), lutetium (177Lu), praseodymium, astatine (211At), rhenium (186Re), bismuth (212Bi or 213Bi), technetium (99Tc), phosphorus (32P), rhodium (188Rh), sulfur (35S), carbon (14C), tritium (3H), chromium (51Cr), chlorine (36C1), cobalt (57Co or 58Co), iron (59Fe), selenium (75Se), or gallium (67Ga) or (68Ga). In some embodiments, the present disclosure contemplates that certain radioisotopes may be useful in or as therapeutic agents including but not limited to yttrium (90Y), lutetium (177Lu), actinium (225Ac), praseodymium, astatine (211At), rhenium (186Re), bismuth (212 Bi or 213Bi), and rhodium (188Rh). In some embodiments, radioisotopes are useful as labels, e.g., for use in diagnostics. In some such embodiments, such radioisotopes may include but are not limited to iodine (131I or 125I), technetium (99Tc), phosphorus (32P), carbon (14C), lead (212Pb) or tritium (3H). See, e.g., U.S. Pat. No. 7,514,078.

In some embodiments, radionuclides are conjugated to different complexing agents and chelators. In some embodiments, chelators are identified and attached/bound to miniproteins through a linker or by acyclic, cyclic and macrocyclic chelates such as, for example, 1,4,7,10,13,16-hexaazacyclohexadecane-N,N′,N”,N″′,N” ”,N″″′-hexaacetic acid (HEHA), 1,4,7,10-tetraazacyclododecane-N,N′,N”,N″′-tetraacetic acid (DOTA), NOPO, Crown, etc. In some embodiments, certain chelators may be preferred for certain radionuclides such as, for example, Ac-225 with DOTA or Crown, Ga-68 with NOPO, etc. In some embodiments, preferred combinations of chelators and radionuclides comprise one or more of the following: DOTA and Ac-225, In-111, Ga-68, Pb-212, Lu-177, Cu-67, Cu-64, La-132, La-135, or Ce-134; Crown and Ac-225, In-111, Ga-68, Pb-212, Lu-177, Cu-67, Cu-64, La-132, La-135, or Ce-134; NOPO and Ac-225, In-111, Ga-68, Pb-212, Lu-177, Cu-67, Cu-64, La-132, La-135, or Ce-134; and/or Macropa and Ac-225, In-111, Ga-68, Pb-212, Lu-177, Cu-67, Cu-64, La-132, La-135, or Ce-134.

Preferably, in some embodiments, a preferred radionuclide complex comprises Ac-225, In-111, Ga-68, Pb-212, Lu-177, Cu-67, Cu-64, La-132, La-135, or Ce-134. In some such embodiments, such a complex with desired stability is selected. That is, in some embodiments, a complex comprising Ac-225, In-111, Ga-68, Pb-212, Lu-177, Cu-67, Cu-64, La-132, La-135, or Ce-134 is characterized as having better stability in vivo in comparison to other complexes. Without being bound by any particular theory, the present disclosure contemplates that, in some embodiments, a radionuclide complex comprising a miniprotein forms with the miniprotein target (e.g., Nectin-4 or a fragment thereof). In some such embodiments, such a complex is internalized in the target cell.

In some embodiments, a radionuclide complex forms with a chelator (e.g., DOTA, NOPO, Crown, Macropa, etc.) and is considerably more stable in vivo. In some embodiments, a miniprotein forms internalizing complexes with targets (e.g., Nectin-4).

In some embodiments, a composition provided by the present disclosure comprises Actinium-225 (Ac-225). In some embodiments, a composition provided by the present disclosure comprises indium (In-111). In some embodiments, a composition provided by the present disclosure comprises gallium (Ga-68). In some embodiments, a composition provided by the present disclosure comprises copper (Cu-64). In some embodiments, a composition provided by the present disclosure comprises lutetium (Lu-177). In some embodiments, a composition provided by the present disclosure comprises lead (Pb-212) In some embodiments, a composition provided by the present disclosure comprises copper (Cu-67). In some embodiments, a composition provided by the present disclosure comprises lanthanum (La-132). In some embodiments, a composition provided by the present disclosure comprises lanthanum (La-135). In some embodiments, a composition provided by the present disclosure comprises cerium (Ce-134). For example, in some embodiments, radioimmunotherapy comprising Ac-225 may provide i) limited range in tissue of a few cell diameters; ii) high linear energy transfer leading to dense radiation damage along each alpha track; iii) a 10 day half-life; and/or iv) four net alpha particles emitted per decay (see, e.g., as described in Scheinberg, David A, and Michael R McDevitt. “Actinium-225 in targeted alpha-particle therapeutic applications.” Current radiopharmaceuticals vol. 4,4 (2011): 306-20).

In some embodiments, targeting constructs (e.g., 225-Ac-drug constructs, e.g., 68-Ga-constructs) have potential for use in cancer. For example, in some such embodiments, such constructs may be used in the treatment of cancer, such as, for example 225-Ac-drug constructs. In some embodiments, such constructs may be used in imaging, such as for prognostics, diagnostics, and/or monitoring, such as Ga-68 or Cu-64-based constructs.

In some embodiments, Ac-225 is conjugated to a miniprotein as provided herein. In some embodiments, the actinium is conjugated onto a chelator and may include an optional linker to link it to a miniprotein, which miniprotein targets the conjugate to a cell expressing the target (e.g., Nectin-4).

In some embodiments, Ga-68 is conjugated to a miniprotein as provided herein. In some embodiments, the gallium is conjugated onto a chelator and may include an optional linker to link it to a miniprotein, which miniprotein targets the conjugate to a cell expressing the target (e.g., Nectin-4).

In some embodiments, Cu-64 is conjugated to a miniprotein as provided herein. In some embodiments, the copper is conjugated onto a chelator and may include an optional linker to link it to a miniprotein, which miniprotein targets the conjugate to a cell expressing the target (e.g., Nectin-4).

In some embodiments, In-111 is conjugated to a miniprotein as provided herein. In some embodiments, the indium is conjugated onto a chelator and may include an optional linker to link it to a miniprotein, which miniprotein targets the conjugate to a cell expressing the target (e.g., Nectin-4).

In some embodiments, Lu-177 is conjugated to a miniprotein as provided herein. In some embodiments, the lutetium is conjugated onto a chelator and may include an optional linker to link it to a miniprotein, which miniprotein targets the conjugate to a cell expressing the target (e.g., Nectin-4).

In some embodiments, Pb-212 is conjugated to a miniprotein as provided herein. In some embodiments, the lead is conjugated onto a chelator and may include an optional linker to link it to a miniprotein, which miniprotein targets the conjugate to a cell expressing the target (e.g., Nectin-4).

In some embodiments, Cu-67 is conjugated to a miniprotein as provided herein. In some embodiments, the copper is conjugated onto a chelator and may include an optional linker to link it to a miniprotein, which miniprotein targets the conjugate to a cell expressing the target (e.g., Nectin-4).

In some embodiments, La-132 is conjugated to a miniprotein as provided herein. In some embodiments, the lanthanum is conjugated onto a chelator and may include an optional linker to link it to a miniprotein, which miniprotein targets the conjugate to a cell expressing the target (e.g., Nectin-4).

In some embodiments, La-135 is conjugated to a miniprotein as provided herein. In some embodiments, the lanthanum is conjugated onto a chelator and may include an optional linker to link it to a miniprotein, which miniprotein targets the conjugate to a cell expressing the target (e.g., Nectin-4).

In some embodiments, Ce-134 is conjugated to a miniprotein as provided herein. In some embodiments, the cerium is conjugated onto a chelator and may include an optional linker to link it to a miniprotein, which miniprotein targets the conjugate to a cell expressing the target (e.g., Nectin-4).

In some embodiments, alpha particles (e.g., of Actinium-225, etc.) are positively charged. In some such embodiments, the range of penetration in tissue varies between 5 and 10 cell diameters (40 to 100 μm) depending on their energy (Radiobiologic principles in radionuclide therapy. Kassis Al, Adelstein S J J Nucl Med. 2005 January; 46 Suppl 1( ):4S-12S). In some such embodiments, such penetration allows for localized irradiation of target cells with minimal toxicity on surrounding normal cells, and internalization by cancer cells with as few as 1-3 tracks across the cell nucleus resulting in cell death (Humm 1987; Macklis et al 1988; Humm and Chin 1993; Couturier et al 2005) causing single- and double-stranded DNA breaks. See, e.g., Sofou S. Radionuclide carriers for targeting of cancer. Int J Nanomedicine. 2008; 3(2):181-199. doi:10.2147/ijn.s2736.

### Dose Calculation

In some embodiments, a dose of a radiotherapeutic is calculated. In some such embodiments, calculation of an absorbed dose (D) is necessary to quantitatively correlate tumor response to a particular radiotherapeutic modality and to project on the potential effect of other radiotherapeutic modalities or administration strategies. That is, in some embodiments, the absorbed dose from a target site is defined as the energy (E) absorbed by a particular mass of tissue, normalized by the tissue mass (M): D=E/M (Sgouros 2005). The absorbed energy is defined as a function of three parameters: the number of disintegrations within the particular volume of interest (δ), the energy emitted per disintegration (s), and the fraction of emitted energy that is absorbed by the particular volume of interest (the target mass) (f): E=δ×ε×f. For the relatively long range beta emitters, the dose evaluation at a target site includes not only the energy emitted by radionuclides localized within the target volume, but also the energy emitted by radionuclides accumulated in neighboring organs or areas whose emissions cross along their path the target volume of interest (Kolbert et al 2003). In other words, in some embodiments, the calculated total absorbed dose is the sum of the dose contributions from all regions containing radionuclides that act as secondary sources. In some embodiments, the adsorbed dose due to photon emissions is usually calculated separately and added to the dose due to alpha or beta particles. In some embodiments, where a composition comprises an alpha particle emitter, such cross organ absorbed doses may be of no significance due to their short recoil distances. In some embodiments, given appropriate context, at the micron-scale and at distances comparable to a few cells, microdosimetric evaluations are used to evaluate dose or ‘hits‘ acquired by cancer cells within micrometastatic clusters (Palm et al 2002).

In some embodiments, a miniprotein conjugate comprising a radionuclide displays binding specificity to human Nectin-4. In some embodiments, the miniprotein comprises a binding affinity characterized by a dissociation constant ranging from about 500 nM to about 1 pM, e.g., 500, 400, 300. 200, 100, 90, 80, 70, 60, 50, 40, 30 20, 10, 9, 8, 7, 6, 5, 4, 3, 2, 1 nM, 100, 90, 80, 70, 60, 50, 40, 30, 20, 10, 9, 8, 7, 6, 5, 4, 3, 2, or 1 pM binding affinity to human Nectin-4. Without being bound by any particular theory, the present disclosure contemplates that, in some embodiments, a preferred dissociation constant of a miniprotein is about 10 nM or less, about 7.5 nM, about 5 nM or less, about 2.5 nM or less, about 1 nM or less (i.e., in the picomolar range).

In some embodiments, a miniprotein comprising a radionuclide in accordance with the present disclosure binds to Nectin-4 with a binding affinity of about 1 pM to 100 nM. In some embodiments, a miniprotein in accordance with the present disclosure binds to Nectin-4 with a binding affinity of about 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 pM; 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, or 1000 nM. In some embodiments, a miniprotein in accordance with the present disclosure binds to Nectin-4 with a binding affinity of about 1 pM to 100 pM, 10 pM to 1 nM, 100 pM to 10 nM, or 1 nM to 100 nM.

In some embodiments, compositions as provided herein are characterized for one or more of absorbed dose, dose rate, tumor penetration profile of radionuclides, intracellular localization profiles of radionuclides of shorter range, and tumor radiosensitivity (see, e.g., Sofou S. Radionuclide carriers for targeting of cancer. Int J Nanomedicine. 2008; 3(2):181-199).

As is known to those of skill in the art, due to toxicity of radionuclides, dose needs to be carefully controlled and considered. Accordingly, in some embodiments, compositions comprising radionuclides of the present disclosure address dose-limiting toxicity of compositions such that radionuclides do not accumulate significantly (e.g., in a toxicity-limiting manner) in vital organs.

In some embodiments, alpha particle-emitting isotopes engage in on-target cell killing while minimizing toxic effects (e.g., to surrounding tissue, e.g., as compared to, e.g., beta emitters, etc.).

In some embodiments, compositions provided herein (comprising a radionuclide) are administered in a single step such as, e.g., using a ligand, e.g., a miniprotein resulting in improved biodistributions (e.g., specific targeting), pK with partial and acceptable damage or no damage to normal tissues, enhanced penetration of the pharmaceutical composition into the tumor heterogeneous interstitial space.

In some embodiments, one or more radionuclides is conjugated to a miniprotein. Relatedly, in some embodiments, radiolabeling efficiency of a miniprotein is optimized to radiolabel a desired number of radionuclides. In some embodiments, a ratio of radionuclides conjugated to a miniprotein is 1:1, 2:1, 3:1, 4:1, 5:1, 6:1, 7:1, 8:1, 9:1, 10:1. In some embodiments, radionuclides conjugated to a miniprotein does not present toxicity. In some embodiments, a composition comprising a miniprotein and radionuclide does not accumulate in the liver, spleen, and/or pancreas and is cleared rapidly when administered to a subject. For instance, in some embodiments, after administration to a subject, biodistribution in the kidney is >10% of the injected dose (ID) at 24 hrs and in tumors is >3% ID at 24 hrs.

In some embodiments, after administration to a subject, t1/2 is shorter than that of, e.g., a Nectin-4 antibody, e.g., enfortumab vedotin.

### Radionuclides and Chelation

A radionuclide can be bound to a chelator through any method known in the art. In some embodiments, chelation methods may differ based on the radionuclide and chelator selected. For example, in some embodiments, chelation can be carried out in one step by incubating the miniprotein-chelator conjugate with the radionuclide for a predetermined period at a predetermined temperature to achieve a sufficient amount of chelation. In some embodiments, a miniprotein-chelator conjugate comprises a chelator or variant thereof as provided herein (e.g., DOTA, e.g., NOPO, e.g., Crown, e.g., Macropa, etc.). In some embodiments, miniprotein-chelator conjugates can be chelated to a radionuclide (e.g., Actinium-225, Indium-111, Gallium-68, Copper-64, Lutetium-177, Lead-212, etc.) by incubation with the radionuclide for about 1 hour at 70° C. In some embodiments, miniprotein-chelator conjugates can be chelated to a radionuclide (e.g., Actinium-225, Indium-111, Gallium-68, Copper-64, Lutetium-177, Lead-212, etc.) by incubation with the radionuclide for about 1 hour at 70° C.

In some embodiments, the chelation process yields a preparation in which at least 90%, 91%, 92%, 93%, 94%, 95%, 96%, 97%, 98%, or 99% of the miniprotein-chelator is bound to a radionuclide. In some embodiments, the chelation process yields a preparation in which more than 90%, 91%, 92%, 93%, 94%, 95%, 96%, 97%, 98%, or 99% of the miniprotein-chelator is bound to a radionuclide. Excess radionuclide can be removed from the preparation by purification methods known in the art.

### Polypeptides

Among other things, the present disclosure provides polypeptides. In some embodiments a polypeptide is assembled using solid phase synthesis methods. In some embodiments, a polypeptide is recombinant. In some embodiments, a polypeptide comprises or consists of a miniprotein. In some such embodiments, a miniprotein comprises or consists of a binder. In some embodiments, polypeptides of the present disclosure (including muteins, allelic variants, fragments, derivatives, and analogs) are encoded by polynucleotides as described and provided herein.

In some embodiments, a miniprotein of the present disclosure comprises or consists of a polypeptide capable of binding to target as shown in Table 1A.

In some embodiments, the present disclosure provides binders comprising or consisting of a fragment of a polypeptide as provided herein. In some such embodiments, fragments include at least 20 contiguous amino acids, more preferably at least 25, 30, 35, 40, 45, 50, 55, 60, 65, 70, 75, 80, 85, 90, 95 or more contiguous amino acids.

In some embodiments, miniproteins of the present disclosure can also include fusions or conjugates with one or more other components, such as heterologous polypeptides. For example, in some embodiments, heterologous sequences can comprise or consist of sequences designed to facilitate purification, e.g., histidine tags, and/or visualization of recombinantly-expressed proteins. Other non-limiting examples of such fusions or conjugates include those that permit display of the encoded protein on the surface of a phage or a cell, including any detectable or visualizable component such as, e.g., green fluorescent protein (GFP), and fusions to the IgG Fc region.

In some embodiments, a miniprotein comprises or consists of a specific amino acid sequence. In some embodiments, a miniprotein has an amino acid sequence that is 70%, 75%, 80%, 85%, 90%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, or 100% identical to the amino acid sequence set forth in any of SEQ ID NOs: 1-158 or 177 or 161-176 and/or according to Tables 1B and/or 1C. In some embodiments, a miniprotein has an amino acid sequence that is 70%, 75%, 80%, 85%, 90%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, or 100% identical to the amino acid sequence set forth in SEQ ID NO: 78. In some embodiments, a miniprotein comprises or consists of an amino acid sequence as set forth in SEQ ID NO: 78.

In some embodiments, a miniprotein has an amino acid sequence that is 70%, 75%, 80%, 85%, 90%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, or 100% identical to the amino acid sequence set forth in any amino acid sequences set forth in Table 2A.

As used herein and known to those of skill in the art, the twenty conventional amino acids and their abbreviations follow conventional usage. See Immunology-A Synthesis (Golub and Gren eds., Sinauer Associates, Sunderl'd, Mass., 2nd ed. 1991), which is incorporated herein by reference. In some embodiments, an amino acid of the present disclosure may be a stereoisomer (e.g., D-amino acids) of the twenty conventional amino acids. In some embodiments, an amino acid in a polypeptide of the present disclosure may be a non-natural amino acid. For example, amino acids such as α-, μ-disubstituted amino acids, N-alkyl amino acids, and other unconventional amino acids may also be suitable components for polypeptides of the present disclosure. Examples of unconventional amino acids include: 4-hydroxyproline, γ-carboxyglutamate, F-N,N,N-trimethyllysine, &-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). Arrangements of polypeptide sequence notations used herein have a left-side end corresponding to the amino terminal and a right-side end corresponding to the carboxy-terminal end, in accordance with standard usage and convention.

In some embodiments, miniproteins of the present disclosure comprising two or more cysteine residues, such as those set forth in SEQ ID NOs: 1-158 or 177 or 161-176, have cysteine residues connected via disulfide bridges (e.g., via natural folding).

In some embodiments, cysteine connections are between Cys1 and Cys34 and Cys20 and Cys44.

In some embodiments, the present disclosure provides a miniprotein comprising or consisting of an amino acid sequence set forth in SEQ ID NO: 1-158 or 177 or 161-176 and/or as set forth in Tables 1B, 1C, and/or 2A or a portion or functional variant thereof. In some embodiments, a miniprotein comprises or consists of an amino acid sequence having at least 70%, 75%, 80%, 85%, 90%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, or greater sequence identity to SEQ ID NO: 1-158 or 177 or 161-176 and/or as set forth in Tables 1B, 1C, and/or 2A or a portion or functional variant thereof. In some embodiments, the miniprotein comprises or consists of an amino acid sequence having at least 2, at least 3, at least 4, at least 5, at least 6, at least 7, at least 8, at least 9, at least 10, at least 15, at least 20 or more amino acid residue differences from SEQ ID NO: 1-158 or 177 or 161-176 and/or as set forth in Tables 1B, 1C, and/or 2A or a portion or functional variant thereof. In some embodiments, the miniprotein comprising or consisting of SEQ ID NO: 1-158 or 177 or 161-176 and/or as set forth in Tables 1B, 1C, and/or 2A or a portion or functional variant thereof selectively binds to the target Nectin-4.

### Nucleic Acids

Among other things, the present disclosure provides herein polynucleotides and methods of use thereof. In some embodiments, all or a portion of the polynucleotides encode a polypeptide (e.g., a miniprotein) that specifically binds to Nectin-4. In some embodiments, the Nectin-4 is murine or human Nectin-4. In some embodiments, the nucleic acid sequence has a specific sequence. In some embodiments, a polynucleotide of the present disclosure is codon-optimized (i.e., the nucleic acid sequence is codon optimized).

In some embodiments, a polynucleotide of the present disclosure comprises or consists of a nucleic acid sequence encoding a polypeptide that is or comprises a miniprotein that specifically binds To Nectin-4 or any portion, fragment, or variant thereof.

In some embodiments, a miniprotein is represented by a nucleic acid molecule encoding an amino acid that, when folded, comprises one or more disulfide bridges.

In some embodiments, for example, a nucleic acid molecule (i.e., a polynucleotide) may be non-identical to a reference sequence as provided herein, but still encode a binder as provided by the present disclosure. In some such embodiments, such as provided polynucleotide (i.e., encoding a miniprotein or analog thereto) hybridizes under stringent conditions as disclosed herein.

In some embodiments, the present disclosure provides nucleic acid molecules comprising a fragment of any polynucleotide as provided herein. In some embodiments, a polynucleotide fragment comprises or consists of a portion of contiguous nucleic acid residues. For instance, in some embodiments, a polynucleotide fragment comprises or consists of 20, 25, 30, 35, 40, 45, 50, 55, 60, 65, 70, 75, 80, 85, 100 or more nucleic acid residues.

In some embodiments, fragments of the present disclosure display utility in a variety of systems and methods. For example, the fragments may be used as probes in various assays. For instance, in some embodiments, fragments may be used in hybridization techniques. Depending on the method, the target nucleic acid sequences may be either DNA or RNA. The target nucleic acid sequences may be fractionated (e.g., by gel electrophoresis) prior to the hybridization, or the hybridization may be performed on samples in situ. One of skill in the art will appreciate that nucleic acid probes of known sequence find utility in determining chromosomal structure (e.g., by Southern blotting) and in measuring gene expression (e.g., by Northern blotting). In such experiments, the sequence fragments are preferably detectably labeled, so that their specific hybridization to target sequences can be detected and optionally quantified. In some embodiments, fragments may be used as probes, e.g., such as when immobilized on a microarray. Methods for creating microarrays by deposition and fixation of nucleic acids onto support substrates are well known in the art. Reviewed in DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip: Tools and Technology, Schena (ed.), Eaton Publishing Company/BioTechniques Books Division (2000) (ISBN: 1881299376), the disclosures of which are incorporated herein by reference in their entireties. Analysis of, for example, gene expression using microarrays comprising nucleic acid sequence fragments, such as the nucleic acid sequence fragments disclosed herein, is a well-established utility for sequence fragments in the field of cell and molecular biology. Other uses for sequence fragments immobilized on microarrays are described in Gerhold et al., Trends Biochem. Sci. 24:168-173 (1999) and Zweiger, Trends Biotechnol. 17:429-436 (1999); DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip: Tools and Technology, Schena (ed.), Eaton Publishing Company/BioTechniques Books Division (2000) (ISBN: 1881299376).

In some embodiments, a polynucleotide of the present disclosure comprises or consists of a nucleic acid sequence encoding up to 100 amino acids of SEQ ID NO: 159 or SEQ ID NO: 160. In some embodiments, a polynucleotide of the present disclosure comprises or consists of a sequence that encodes a polypeptide of SEQ ID NOs: 1-158 or 177 or 161-176 and/or as set forth in Tables 1B, 1C, and/or 2A or a portion or functional variant thereof. In some such embodiments, a polynucleotide encodes a polypeptide, such as those set forth in SEQ ID NOs: 1-158 or 177 or 161-176 and/or as set forth in Tables 1B, 1C, and/or 2A or a portion or functional variant thereof, that binds to a target represented by SEQ ID NOs 159 or 160.

In some embodiments, a polynucleotide of the present disclosure comprises or consists of a nucleic acid sequence encoding a polypeptide that is or comprises a miniprotein that binds To Nectin-4 or any portion, fragment, or variant thereof. In some embodiments, the polynucleotide encodes a polypeptide that comprises or consists of the amino acid sequence set forth in any one of SEQ ID NOs: 1-158 or 177 or 161-176 and/or as set forth in Tables 1B, 1C, and/or 2A or a portion or functional variant thereof. In some embodiments, the polynucleotide encodes a polypeptide sequence having at least 70%, 80%, 85%, 90%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, or more identity to the amino acid sequences provided in a polypeptide according to those set forth in Tables 1B and/or 1C or a polypeptide of a compound of Table 2A.

In some embodiments, a miniprotein comprises one or more disulfide bridges. In some embodiments, a miniprotein is represented by a nucleic acid sequence encoding a polypeptide that, when folded, comprises one or more disulfide bridges.

In some embodiments, the present disclosure provides nucleic acid molecules comprising or consisting of a sequence as set forth in SEQ ID NO: 159 or SEQ ID NO: 160, or variations (e.g., codon optimized) thereof.

In some embodiments, for example, a nucleic acid molecule (i.e., a polynucleotide) may be non-identical to a reference sequence as provided herein, but still encode a miniprotein or close analog as provided by the present disclosure (e.g., a miniprotein in accordance with any one of SEQ ID NOs: 1-158 or 177 or 161-176 and/or as set forth in Tables 1B, 1C, and/or 2A or a portion or functional variant thereof as provided for herein). In some such embodiments, such as provided polynucleotide (i.e., encoding a miniprotein or analog thereto) hybridizes under stringent conditions as disclosed herein.

In some embodiments, the present disclosure provides nucleic acid molecules comprising a fragment of any polynucleotide as provided herein. In some embodiments, a polynucleotide fragment comprises or consists of a portion of contiguous nucleic acid residues identical to that of a polynucleotide of any of SEQ ID NOs: 1-158 or 177 or 161-176 and/or as set forth in Tables 1B, 1C, and/or 2A or a portion or functional variant thereof. For instance, in some embodiments, a polynucleotide fragment comprises or consists of 20, 25, 30, 35, 40, 45, 50, 55, 60, 65, 70, 75, 80, 85, 100 or more nucleic acid residues encoding some or all of a polypeptide or fragment thereof as set forth in any one of SEQ ID NOs: 1-158 or 177 or 161-176 and/or as set forth in Tables 1B, 1C, and/or 2A or a portion or functional variant thereof.

One of skill in the art will appreciate that the nucleic acid fragments of the present disclosure may be used in a wide variety of techniques capture and/or detection techniques not specifically described herein.

### Vectors

Also provided herein are vectors, including expression vectors, which comprise, among other things, nucleic acids comprising or consisting of sequences encoding miniproteins that specifically bind to Nectin-4. In some embodiments, a vector is used to produce a polypeptide encoding a binder that binds to Nectin-4. In some embodiments, the Nectin-4 is murine or human Nectin-4. In some embodiments, given appropriate contexts, a miniprotein is represented by an amino acid sequence with a corresponding nucleic acid sequence that has been codon optimized. In some such embodiments, one of skill in the art is capable of designing and optimizing polynucleotides that correspond to amino acids of miniproteins that bind to a target (e.g., Nectin-4), for which exemplary amino acid sequences are set forth in Table 1A. In some embodiments, a vector comprises a nucleic acid sequence that comprises or consists of a sequence encoding Nectin-4.

In some embodiments, the vector comprises a nucleic acid sequence encoding Nectin-4 or a fragment or variant thereof, wherein the polynucleotide is codon-optimized (i.e., the nucleic acid sequence is codon optimized). In some embodiments, the vector comprises a nucleic acid sequence encoding up to 100 amino acids. In some embodiments, a vector of the present disclosure comprises or consists of a nucleic acid sequence that encodes an amino acid sequence of a miniprotein. In some embodiments, the vectors of the present disclosure further comprise a nucleic acid sequence as provided herein operably linked to one or more expression control sequences.

Also provided herein are vectors, including expression vectors, which comprise, among other things, nucleic acids comprising or consisting of those described herein. In some embodiments, a vector is used to produce a polypeptide encoding a miniprotein that binds to Nectin-4. In some embodiments, the Nectin-4 is murine or human Nectin-4. In some embodiments, given appropriate contexts, a Nectin-4 miniprotein (e.g., as provided in Table 2A) has a corresponding nucleic acid sequence that has been codon optimized. In some such embodiments, one of skill in the art is capable of designing and optimizing polynucleotides that correspond to amino acids of particular Nectin-4 miniproteins such as, for example, polynucleotides comprising nucleic acid sequences that correspond to amino acid sequences of Table 2A. In some embodiments, a vector comprises a nucleic acid sequence encoding the amino acid sequence set forth in SEQ ID NO: 159. In some embodiments, a vector comprises a nucleic acid sequence encoding the amino acid sequence set forth in SEQ ID NO: 160. In some embodiments, a vector comprises a nucleic acid sequence that comprises or consists of a sequence having 70%, 80%, 85%, 90%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, or more identity to wild type Nectin-4. In some embodiments, the vector comprises a nucleic acid sequence encoding Nectin-4 or a fragment or variant thereof, wherein the polynucleotide is codon-optimized (i.e., the nucleic acid sequence is codon optimized). In some embodiments, the vector comprises a nucleic acid sequence encoding up to 50, 55, 60, 65, 70, 75, 80, 85, 90, 95, or 100 amino acids of any one of SEQ ID NOs: 1-158 or 177 or 161-176 and/or as set forth in Tables 1B, 1C, and/or 2A or a portion or functional variant thereof. In some embodiments, a vector of the present disclosure comprises or consists of a nucleic acid sequence, wherein the nucleic acid sequence encodes an amino acid sequence comprising those as set forth in in Tables 1B, 1C, and/or 2A or a portion or functional variant thereof. In some embodiments, the nucleic acid sequence is a codon optimized nucleic acid sequence. In some embodiments, the vectors of the present disclosure further comprise a nucleic acid sequence as provided herein operably linked to one or more expression control sequences.

### Host Cell Transformants

In some embodiments, the present disclosure provides host cells transformed with polynucleotides, polypeptides, and/or vectors of the present disclosure, and any combinations as well as any descendants thereof. In some embodiments host cells comprise and carry nucleic acid sequences of the present disclosure on vectors. In some embodiments, a host cell is a cell line. In some embodiments, a host cell is a primary cell, such as an immune cell. In some embodiments, such a primary cell is derived from or made compatible with a subject. In some embodiments, a subject is a mammal. In some embodiments, a mammal is a human. In some embodiments, a human is at risk of having or has been diagnosed as having cancer.

In some such embodiments, such vectors may but need not be freely replicating vectors. In some embodiments, nucleic acid sequences or polynucleotides provided by the present disclosure have been integrated into a genome of a host cell.

In some embodiments, host cells of the present disclosure can be mutated by recombination with a disruption, deletion, or mutation of the isolated nucleic acid of the present disclosure so that the activity of one or more functional activities in the host cell is reduced or eliminated compared to a host cell lacking the mutation.

Without limitation, and as will be appreciated by those of skill in the art, a wide variety of host cells is contemplated in various embodiments in order to express binders of the present disclosure (via use of, e.g., nucleic acid sequences, amino acid sequences, and/or additional components as provided here).

### Pharmaceutical Compositions

The present disclosure provides, among other things, pharmaceutical compositions comprise a polypeptide, polynucleotide, vector and/or host cell encoding a miniprotein (e.g., CDP, knottin, binder, affibody, engineered Kunitz domain, monobody, anticalin, designed ankyrin repeat domain (DARPin), avimer) as provided herein. It is to be understood that a pharmaceutical composition comprising a miniprotein (e.g., CDP, knottin, binder, affibody, engineered Kunitz domain, monobody, anticalin, designed ankyrin repeat domain (DARPin), avimer) is interpreted as a pharmaceutical composition comprising a miniprotein (e.g., CDP, knottin, binder, affibody, engineered Kunitz domain, monobody, anticalin, designed ankyrin repeat domain (DARPin), avimer) per se and/or one or more components encoding a miniprotein (e.g., CDP, knottin, binder, affibody, engineered Kunitz domain, monobody, anticalin, designed ankyrin repeat domain (DARPin), avimer) (e.g., a vector, e.g., a host cell). In some embodiments, a pharmaceutical composition comprises a linker and a chelator. In some embodiments, a pharmaceutical composition comprises a linker, chelator, and radionuclide. In some embodiments, a composition comprises a miniprotein, optional linker, and chelator. In some embodiments, a composition comprises a miniprotein, optional linker, chelator, and radionuclide. In some embodiments, a pharmaceutical composition provided by the present disclosure comprises a miniprotein (e.g., CDP, knottin, binder, affibody, engineered Kunitz domain, monobody, anticalin, designed ankyrin repeat domain (DARPin), avimer) that selectively binds to Nectin-4. In some embodiments, the Nectin-4 is human Nectin-4.

In certain embodiments, a pharmaceutical composition comprises a miniprotein (e.g., CDP, knottin, binder, affibody, engineered Kunitz domain, monobody, anticalin, designed ankyrin repeat domain (DARPin), avimer) comprising one or more cysteine-rich domains. In some embodiments, the pharmaceutical composition comprises a miniprotein (e.g., CDP, knottin, binder, affibody, engineered Kunitz domain, monobody, anticalin, designed ankyrin repeat domain (DARPin), avimer) having one or more disulfide bonds. In some embodiments, the pharmaceutical composition comprises a miniprotein (e.g., CDP, knottin, binder, affibody, engineered Kunitz domain, monobody, anticalin, designed ankyrin repeat domain (DARPin), avimer) represented or encoded by an amino acid sequence having <100AAs, <90AAs, <80AAs, <85AAs, <75AAs, <70AAs, <65AAs, <60AAs, <55AAs, <50AAs, <45AAs, <40AAs, <35AAs, <30AAs, <25AAs, <20AAs, <15AAs, <10AAs, or <5AAs.

In some embodiments, a pharmaceutical composition comprising a miniprotein (e.g., CDP, knottin, binder, affibody, engineered Kunitz domain, monobody, anticalin, designed ankyrin repeat domain (DARPin), avimer) as provided herein is characterized as having a molecule weight equal to or less than 12 kDa.

In some embodiments, a pharmaceutical composition comprising a miniprotein (e.g., CDP, knottin, binder, affibody, engineered Kunitz domain, monobody, anticalin, designed ankyrin repeat domain (DARPin), avimer) does not elicit an undesirable immune response or elicits a tolerable immune response. In some embodiments, a pharmaceutical composition of the present disclosure comprises a miniprotein (e.g., CDP, knottin, binder, affibody, engineered Kunitz domain, monobody, anticalin, designed ankyrin repeat domain (DARPin), avimer) having high tissue penetrating properties.

In some embodiments, a pharmaceutical composition comprising a miniprotein comprises acceptable half-life and/or stability. In some such embodiments, acceptable stability is between about 30 minutes to 48 hours in serum and 1-4 days or more in a tumor or tumor microenvironment. By way of non-limiting example, for instance, in some embodiments, a miniprotein of the present disclosure has stability of about 2.5 hours in serum. In some embodiments, stability of a miniprotein is about 30 minutes, 60 minutes, 1, 2,3,4,5,6,7,8,9,10,11,12,13,14,15,16,1718,19, 20, 21, 22, 23, 24, 30, 36, 40, or48 hours in serum. In some embodiments, stability in a tumor or tumor microenvironment is 24, 36, 48, 60, 72, 84, 96 hours or more.

In some embodiments, the pharmaceutical composition is characterized as stable in vivo. In some embodiments, a pharmaceutical composition provided herein is not taken up in kidney or liver. In some embodiments, a pharmaceutical composition provided herein, when taken up in kidney or liver, clears kidney and/or liver faster than a pharmaceutical composition not comprising a polypeptide as provided herein. In some embodiments, biodistribution of a pharmaceutical composition as provided herein is measured at four hours after administration and is between about 1 and about 3 (in % ID/g) in a tumor in a subject (e.g., a murine subject, e.g., a human subject). In some embodiments, concentration in a tumor is greater than concentration in a kidney or liver of a subject to whom a pharmaceutical composition comprising a polypeptide provided herein is administered.

In some embodiments, a pharmaceutical composition of the present disclosure exhibits solubility of >0.05 mg/mL, >0.1 mg/mL, >0.2 mg/mL, >0.3 mg/mL, >0.4 mg/mL, >0.5 mg/mL, >0.6 mg/mL, >0.7 mg/mL, >0.8 mg/mL, >0.9 mg/mL, >1 mg/mL, >2 mg/mL, >3 mg/mL, >4 mg/mL, >5 mg/mL, >6 mg/mL, >7 mg/mL, >8 mg/mL, >9 mg/mL, or >10 mg/mL.

In some embodiments, a pharmaceutical composition provided by the present disclosure exhibits stability of >80%, >81%, >82%, >83%, >84%, 85%, >86%, >87%, >88%, >89%, >90%, >91%, >92%, >93%, >94%, 95%, >96%, >97%, >98%, or >99%.

In some embodiments, a pharmaceutical composition of the present disclosure is characterized as comprising a certain purity, represented as a percentage of parent molecule still intact. In some embodiments, a pharmaceutical composition of the present disclosure comprises about 85% purity or greater at 5 days at room temperature. In some embodiments, a pharmaceutical composition of the present disclosure is characterized as having about 90% purity or greater at 40° C. for 4 hr. In some embodiments, a pharmaceutical composition of the present disclosure comprises cyclic or acyclic sequence.

In some embodiments, a pharmaceutical composition in accordance with the present disclosure comprises a miniprotein (e.g., CDP, knottin, binder, affibody, engineered Kunitz domain, monobody, anticalin, designed ankyrin repeat domain (DARPin), avimer) and one or more additional components. For example, in some embodiments, one or more additional components may be a linker and/or a conjugate such as a cytotoxic payload or detectable moiety for use in diagnosis and/or imaging. In some such embodiments, a pharmaceutical composition comprises a linker, chelator, and/or radionuclide as provided herein.

In some embodiments, pharmaceutical compositions modulating, binding, or inhibiting human Nectin-4 (or any related activity thereto) are provided. In some embodiments, a pharmaceutical composition is or comprises a therapeutic. In some embodiments, a pharmaceutical composition is or comprises a detectable moiety (e.g., as used for imaging such as MRI, CT, PET, etc.).

In preferred embodiments, one or more characteristics of the pharmaceutical compositions are identified for optimized administration parameters including but not limited to dose, effective dose, dose rate, tumor penetration profile, intracellular localization profile, binding specificity, etc. See Sofou S. Radionuclide carriers for targeting of cancer. IntJ Nanomedicine. 2008; 3(2):181-199. doi:10.2147/ijn.s2736.

In some embodiments, a pharmaceutical composition of the present disclosure does not present toxicity or presents less toxicity than a composition comprising one or more different components such as a larger targeting peptide, or a different radionuclide (e.g., beta emitter, etc.).

In some embodiments, a pharmaceutical composition of the present disclosure does not accumulate in the liver, spleen, and/or pancreas and is cleared rapidly. For instance, the biodistribution in some embodiments, after administration to a subject, biodistribution in the kidney is >10% of the injected dose (ID) at 24 hrs and in tumors is >3% ID at 24 hrs. In some embodiments, after administration to a subject, t1/2 is shorter than that of, e.g., a Nectin-4 antibody, e.g., enfortumab vedotin.

### Theranostic Compositions

In some embodiments, theranostic compositions are provided. In some embodiments, the present disclosure provides a diagnostic or a screening to detect the presence or absence, and/or the level of Nectin-4 in a subject or sample. In some embodiments, the subject is a mammal. In some embodiments, the subject is a rodent (e.g., mouse) subject and the Nectin-4 is a rodent (e.g., murine) Nectin-4. In some embodiments, the subject is a human subject and the Nectin-4 is a human Nectin-4. In some embodiments, presence of Nectin-4 in a subject is related to a risk of developing a disease, disorder, or condition. In some embodiments, presence of a particular level of Nectin-4 indicates an increased risk of developing or diagnosis of a disease, disorder, or condition. In some embodiments, a reduction in a level of Nectin-4 (e.g., as compared to a prior measurement) is associated with treatment of a diagnosed disease.

In certain aspects, theranostic compositions are provided. In some embodiments, the present disclosure provides a diagnostic or a screening to detect the presence or absence, and/or the level of human Nectin-4 in a subject or sample.

In certain aspects, the present disclosure provides methods for defining the structure activity relationship of a pharmaceutical composition comprising: (i) a Nectin-4-specific miniprotein; (ii) an optional linker; (iii) a chelator; and (iv) a radioactive molecule, wherein the modified polypeptide sequence modulates human Nectin-4 activity.

Methods of Screening and Development

In some embodiments, directed evolution and computational folding algorithms can be combined for de novo creation of miniproteins (e.g., CDPs, knottins, binders, affibodies, engineered Kunitz domains, monobodies, anticalins, designed ankyrin repeat domains (DARPins), avimers). For example, in some embodiments, hundreds of miniprotein backbones with various secondary structure elements, orientations, and loop lengths can be matched with hotspot binding motifs on a protein target or antigen of interest (e.g., Nectin-4). In some such embodiments, if the binding motifs of the miniprotein do not clash with the backbone of the target, the monomer and interaction energies are optimized with programs known to those of skill in the art such as, e.g., AlphaFold, Rosetta combinatorial sequence optimization, etc.

In some embodiments, oligonucleotide pools encoding design sequences selected through the computational approach can be synthesized, amplified, and co-transformed into yeast. The resulting yeast libraries displaying the design sequences can be incubated with fluorescently labeled target protein or antigen. Cells that display the designs that bind the target can be retrieved by fluorescence-activated cell sorting (FACS) and deep sequenced. Once miniproteins (e.g., CDPs, knottins, binders, affibodies, engineered Kunitz domains, monobodies, anticalins, designed ankyrin repeat domains (DARPins), avimers) are identified either through affinity-maturation or original designs, they can be chemically synthesized or expressed, e.g., in Escherichia coli, and purified, and characterized in solution.

In some embodiments, libraries of stable miniproteins (e.g., CDPs, knottins, binders, affibodies, engineered Kunitz domains, monobodies, anticalins, designed ankyrin repeat domains (DARPins), avimers) may be developed to allow for screening against specific chosen targets. Such a library designs a hydrophobic core to the miniprotein (e.g., CDP, knottin, binder, affibody, engineered Kunitz domain, monobody, anticalin, designed ankyrin repeat domain (DARPin), avimer) to enable folding in addition to cysteine crosslinking, improving the number of folded structures in a library.

In some embodiments, once miniprotein (e.g., CDPs, knottins, binders, affibodies, engineered Kunitz domains, monobodies, anticalins, designed ankyrin repeat domains (DARPins), avimers) are identified or engineered, they may be produced via chemical synthesis or recombinant expression. In some embodiments, a miniprotein (e.g., CDP, knottin, binder, affibody, engineered Kunitz domain, monobody, anticalin, designed ankyrin repeat domain (DARPin), avimer) may be produced by solid phase peptide synthesis followed by in vitro folding. Standard 9-fluorenylmethyloxycarbonyl (Fmoc)-based solid phase peptide chemistry may be employed. In some such embodiments, the linear peptide may then be folded under conditions that promote oxidation of cysteine side chain thiols to form disulfide bonds, followed by purification, e.g., by reversed-phase high-performance liquid chromatography (RP-HPLC). An approach using recombinant DNA may also be employed to produce a miniprotein (e.g., CDP, knottin, binder, affibody, engineered Kunitz domain, monobody, anticalin, designed ankyrin repeat domain (DARPin), avimer) as provided herein.

Iterations between data-driven model improvement and experimental testing with miniproteins (e.g., CDPs, knottins, binders, affibodies, engineered Kunitz domains, monobodies, anticalins, designed ankyrin repeat domains (DARPins), avimers) is likely to optimize the folding and binding abilities of miniproteins (e.g., CDPs, knottins, binders, affibodies, engineered Kunitz domains, monobodies, anticalins, designed ankyrin repeat domains (DARPins), avimers), to develop pharmaceutically superior specific molecules.

### Characterization, Analysis & Synthesis

In some embodiments, a miniprotein (e.g., CDP, knottin, binder, affibody, engineered Kunitz domain, monobody, anticalin, designed ankyrin repeat domain (DARPin), avimer) of the present disclosure is characterized. For example, in some embodiments, binding specificity, binding affinity, binding localization, etc. are performed using methods known to those of skill in the art. For instance, in some embodiments, binding localization is performed using one or more techniques such as immunohistochemistry/immunocytochemistry (e.g., using cell lines or tissue biopsy samples). In some embodiments, binding affinity is performed using surface plasmon resonance measurements. In some such embodiments, binding affinity (e.g., dissociation constant expressed as K_{D}) is measured in one or more assays (e.g., a yeast-based assay where the target is recombinantly expressed in yeast and exposed to a miniprotein (e.g., CDP, knottin, binder, affibody, engineered Kunitz domain, monobody, anticalin, designed ankyrin repeat domain (DARPin), avimer) provided by the present disclosure).

In some embodiments, synthesis and analysis techniques including, without limitation, HPLC, LCMS, CD, quantitative thin layer chromatography and others known to those of skill in the art are used to efficiently synthesize via solid phase peptide synthesis methods, characterize miniproteins (e.g., CDP, knottin, binder, affibody, engineered Kunitz domain, monobody, anticalin, designed ankyrin repeat domain (DARPin), avimer) and fully optimized clinical pharmaceutical composition candidates.

In some embodiments, internalization assays can be performed. For example, in some embodiments, labeled miniproteins (e.g., radiolabeled, e.g., fluorescently labeled, etc.) can be used to assay internalization of Nectin-4 miniproteins. In some embodiments, miniproteins may be coupled to detectable labels (e.g., pH-sensitive fluorescent dye, e.g., radiolabels). Cells can then be examined. In some embodiments, cells can be labeled with an antibody for the target and/or one or more antibodies to endocytic or lysosomal markers (e.g., clathrin, LAMP-1, etc.) after fixation and permeabilization, followed by microscopic analysis.

In some embodiments, internalization assays may be conducted using other methods known to those of skill in the art and provided herein such as, e.g., in Example 13A.

### Methods of Screening and Development of Miniproteins

Directed evolution and computational folding algorithms can be combined for de novo creation of miniproteins as provided herein. In some embodiments, hundreds of miniprotein backbones with various secondary structure elements, orientations, and loop lengths can be matched with hotspot binding motifs on a target of interest (e.g., Nectin-4). In some such embodiments, if binding motifs of the miniprotein do not clash with the backbone of the target, the monomer and interaction energies are optimized with Rosetta combinatorial sequence optimization.

In some embodiments, oligo pools encoding the design sequences selected through the computational approach can be synthesized, amplified, and co-transformed into yeast. In some embodiments, resulting yeast libraries displaying the design sequences can be incubated with fluorescently labeled target. In some embodiments, cells that display the designs that bind the target can be retrieved by fluorescence-activated cell sorting (FACS) and deep sequenced. In some embodiments, once miniproteins are identified either through affinity-maturation or original designs, miniproteins can be chemically synthesized or expressed in Escherichia coli, purified, and characterized in solution.

In some embodiments, libraries of stable CDPs or knottin peptides may be developed to allow for screening against specific chosen targets. The library designs a hydrophobic core to the miniproteins to enable folding in addition to cystine crosslinking, improving the number of folded structures in a library.

In some embodiments, once miniproteins are identified or engineered, they may be produced via chemical synthesis or recombinant expression. In some embodiments, a miniprotein peptide may be produced by solid phase peptide synthesis followed by in vitro folding. Standard 9-fluorenylmethyloxycarbonyl (Fmoc)-based solid phase peptide chemistry may be employed. In some such embodiments, a linear peptide may then be folded under conditions that promote oxidation of cysteine side chain thiols to form disulfide bonds, followed by purification, e.g., by reversed-phase high-performance liquid chromatography (RP-HPLC). In some embodiments, an approach using recombinant DNA may also be employed to produce a desired miniprotein.

In some embodiments, iterations between data-driven model improvement and experimental testing with miniproteins is likely to optimize the folding and binding abilities of the miniproteins, in order to develop pharmaceutically superior specific molecules. In some embodiments, the miniprotein or a portion thereof is engineered at the DNA level (e.g., degenerate codons can be introduced by oligonucleotide assembly using overlap extension PCR; or the genetic material can be amplified using flanking primers with sufficient overlap with the yeast display vector for homologous recombination).

### Modifications to Miniproteins

In some embodiments, the present disclosure further provides one or more modifying components. In some embodiments, a modifying component comprises or consists of an inducible or repressible promoter that is operably linked to the coding sequence of a miniprotein as provided herein. In some embodiments, expression profile of a miniprotein or its underlying amino acid sequence can be altered via the promoter of a nucleic acid sequence encoding it. In some aspects, the expression profile of the miniprotein can be temporally altered or controlled by temporally altering or controlling promoter function. In some embodiments, a promoter may be spatially and/or environmentally controlled. In some embodiments, a modifying component comprises or consists of an enhancer. In some such embodiment, an enhancer is used to modify expression profile of a binder but not necessarily operably linked to the coding sequence of the binder; rather, in some embodiments, an enhancer is located upstream or downstream of a coding sequence of a binder of the present disclosure. In some embodiments, an enhancer may be temporally controlled. In some embodiments, an enhancer may be spatially and/or environmentally controlled.

In some embodiments, an expression profile of a binder and/or a sequence encoding it (e.g., a nucleic acid sequence, e.g., an amino acid sequence such as, e.g., a gene or portion thereof) of the present disclosure can be altered via one or more modifications. In some such embodiments, the one or more modifications comprise one or more mutations in a sequence (e.g., nucleic acid sequence, e.g., amino acid sequence) provided by the present disclosure. In some aspects, a sequence of the present disclosure comprises a deletion relative to a parental sequence or portion thereof.

Modifications may also be made using changes to amino acid sequences and bonds. For example, chemical crosslinking can be used to improve binding ability or affinity of a miniprotein for a target. In some embodiments, changes such as amino acid residues (e.g., lysine, etc., e.g., non-natural amino acids, etc.) fusion proteins, or other chemical moieties can be used to generate miniproteins with enhanced binding and functional activity, e.g., as compared to those without modifications. In some embodiments, miniproteins can be characterized as having small disulfide-rich peptide scaffolds and can have difficulties folding. For example, in some embodiments, miniproteins can form various isomers (e.g., a miniprotein with three core disulfide bonds can, in some embodiments, form at least 15 different isomers). In some such embodiments, such a variety of isomers can impact yield. In some embodiments, miniproteins without cysteine residues (e.g., two or more cysteines, e.g., at least one disulfide bridge) may be modified to improve stability without need for additional chemical crosslinking by increased numbers of disulfide bonds.

In some embodiments, for example, binding affinity of miniproteins can be improved using an SAR approach. For instance, various amino acid residues within the miniprotein structures can be replaced with optimal substitutions, resulting in improved binding affinity. In some embodiments, these substitutions can include natural and/or non-natural amino acids, conjugated chemical moieties, and/or other small molecule attachments.

In some embodiments, chemical crosslinking can be used to provide proper structural conformation and stability. Proper structural conformation can be critical to retention of certain binding affinity (e.g., in an improved Nectin-4 miniprotein, e.g., as compared to a miniprotein that has not been improved using an SAR or other approach). In some embodiments, miniproteins have small disulfide-rich peptide scaffolds and difficulties folding. In some such embodiments, using techniques and approaches known to those of ordinary skill in the art, optimized conditions for folding and purification via reverse-phase HPLC can be used to ensure final (e.g., optimized) compounds (comprising miniproteins as provided herein) have correct structure, conformation, and purity.

### Constraints

Modifications, such as constraints, can be introduced (e.g., engineered) into proteins, such as any miniprotein provided herein. A constraint can alter function (e.g., binding) and/or structure (e.g., folding) of a protein. For example, constraints can assist in maintaining secondary structure of a given protein (e.g., a miniprotein as provided herein, e.g., a conjugate as provided herein). A common protein secondary structure is an α-helix. These helices can play key roles in not only structure of a protein, but also function, impacting how a particular protein can interact with a binding partner. For example, alpha helices can mediate protein-protein interactions (PPIs) by serving as recognition motifs. Introducing a constraint in a protein, such as a miniprotein, on an alpha-helix can, in some embodiments, change one or more features of a protein such as affinity of a protein for a target (e.g., increase affinity), cell penetration (e.g., increased cell penetration), resistance to proteolysis (e.g., increased resistance to proteolytic degradation). Examples of constraints (e.g., α-helix constraints) can include, but are not limited to disulfide bridges/bonds, staples (e.g., hydrocarbon staples), salt bridges between charged amino acid side chain residues, lactam bridges, disulfide bridges, hydrogen bond surrogates, hydrophobic interactions, metal ligation, triazole staples synthesized from alkenyl and azido side chain residues, photocontrollable macrocycles, and introduction amino acids, such as, e.g., α,α-disubstituted amino acids. As will be known to those of skill in the art, a staple can refer to a synthetic constraint (e.g., a brace) between two previously independent entities. For example, a staple can be formed via covalent linkage between two previously independent entities such as, for example, amino acid side-chains (e.g., forming, for example, a peptide macrocycle).

In some embodiments, miniproteins provided herein comprise one or more constraints (e.g., disulfide bridge, e.g., staple). In some embodiments, a miniprotein of the present disclosure comprises one or more disulfide bridges. In some embodiments, a miniprotein provided by the present disclosure comprises two or more disulfide bridges. For example, miniproteins of the present disclosure herein may contain a set of amino acids that together support formation of or are part of a constraint (e.g., a disulfide bridge, e.g., a staple). In some embodiments, a structural feature of a miniprotein is having at least two cysteine residues, positioned relative to one another so that disulfide bridge can be formed (e.g., as provided herein, see, e.g., Table 2A). In some embodiments, a miniprotein provided by the present disclosure has at least three, or at least four cysteine residues, which form one or two disulfide bridges. In some such embodiments, such a miniprotein further comprises one or more additional constraints (e.g., staples).

In some embodiments, a miniprotein of the present disclosure comprises a staple. In some embodiments, the present disclosure provides stapled miniproteins that bind to Nectin-4, wherein presence of the staple changes at least one binding characteristic relative to the miniprotein without the staple (e.g., increased binding affinity for Nectin-4, e.g., a change in avidity for Nectin-4). Those skilled in the art, reading the present disclosure, will appreciate that, a stapled miniprotein may be prepared using any desired stapling technology.

### Binding Assays

In some embodiments, binding assays are used to determine binding affinities and/or binding/dissociation constants or a composition or one or more components thereof (e.g., of a miniprotein with or without one or more additional components as provided herein). For example, in some embodiments, an equilibrium dissociation constant (Kd) is determined using fluorescent labeling and detection methods. In some embodiments, a cell population (e.g., yeast cells) engineered to express a library of miniproteins (e.g., CDPs, knottins, binders, affibodies, engineered Kunitz domains, monobodies, anticalins, designed ankyrin repeat domains (DARPins), avimers) (e.g., binders that bind to a target) is produced. In some embodiments, the cells express a target. Depending on whether a set of cells expresses targets or miniproteins (e.g., CDPs, knottins, binders, affibodies, engineered Kunitz domains, monobodies, anticalins, designed ankyrin repeat domains (DARPins), avimers), in some embodiments, a cell library is incubated with a target (e.g., Nectin-4) or with a miniprotein (e.g., CDP, knottin, binder, affibody, engineered Kunitz domain, monobody, anticalin, designed ankyrin repeat domain (DARPin), avimer) or set of miniproteins (e.g., CDPs, knottins, binders, affibodies, engineered Kunitz domains, monobodies, anticalins, designed ankyrin repeat domains (DARPins), avimers) as provided herein. In some such embodiments, cells and miniproteins (e.g., CDPs, knottins, binders, affibodies, engineered Kunitz domains, monobodies, anticalins, designed ankyrin repeat domains (DARPins), avimers) are assessed using flow cytometry and/or FACS analysis to determine binding affinity using methods known to those of skill in the art. (See, e.g., “Chapter Nine —Engineering CDPs as Novel Binding Agents.” Methods in Enzymology, edited by K. Dane Wittrup and Gregory L. Verdine, vol. 503, Academic Press, 2012, pp. 223-51. ScienceDirect, doi:10.1016/B978-O-12-396962-0.00009-4.).

### Affinity Maturation

In some embodiments, a miniprotein (e.g., CDP, knottin, binder, affibody, engineered Kunitz domain, monobody, anticalin, designed ankyrin repeat domain (DARPin), avimer) of the present disclosure comprises or consists of a sequence that exhibits certain desired affinity ranges for a target. In some embodiments, affinity maturation is performed on a sequence as provided by the present disclosure wherein the affinity matured sequence displays the same or better selectivity and/or affinity for Nectin-4 as compared to the starting sequence or another sequence with “less” affinity as compared to the affinity matured sequence. In some embodiments, affinity maturation is performed using a Nectin-4 antigen and a sequence that binds to the antigen is that of a miniprotein (e.g., CDP, knottin, binder, affibody, engineered Kunitz domain, monobody, anticalin, designed ankyrin repeat domain (DARPin), avimer) as provided herein.

In some embodiments, affinity maturation is performed on a sequence that selectively binds to Nectin-4. In some embodiments, the Nectin-4 is human Nectin-4.

In preferred aspects of the present disclosure, the modified polypeptide sequence of the pharmaceutical composition comprises nM or sub-nM binding affinity to a target on a cell line expressing human Nectin-4, binding potency on protein target or in a cell-based assay.

In certain embodiments, the modified polypeptide sequence comprises a binding affinity of 900, 800, 700, 600, 500, 400, 300. 200, 100, 90, 80, 70, 60, 50, 40, 30 20, 10, 9, 8, 7, 6, 5, 4, 3, 2, 1 nM binding affinity to the human Nectin-4.

In one embodiment, the modified polypeptide sequence comprises a binding affinity of 500 nM binding affinity to the human Nectin-4.

In more preferred embodiments, the modified polypeptide sequence comprises a picomolar binding affinity.

In other preferred embodiments, the modified polypeptide sequence exhibits selectivity with which the sequence binds to only the Nectin-4 protein target. In some embodiments, affinity maturation is performed using magnetic-based assays. In some embodiments, affinity maturation is performed using flow cytometry/FACS-based assays in accordance with procedures known to those of skill in the art.

### Methods of Miniprotein Manufacturing

In some embodiments, synthesis of a miniprotein comprises solid phase synthesis. In some embodiments, miniproteins are synthesized using standard solid phase peptide synthesis as is known to those of skill in the art. (See, e.g., Johannes Meienhofer, Hormonal Proteins and Peptides, Volume II, 1973, Pages 45-267). In some embodiments, SPS comprises synthesis using methods known to those of skill in the art including, for example, Fmoc or Boc amino protecting groups. In some embodiments, synthesis comprises protection from reaction with incoming N-protected amino acids. In some embodiments, synthesized polypeptides are analyzed to determine sequence, structure, and related properties using HPLC/LC-MS.

In some embodiments, a miniprotein is manufactured using recombinant production methods as are known to those of skill in the art including, for example, yeast-based approaches and chemical synthesis.

### Methods of Conjugation

In some embodiments, conjugates of the present disclosure comprise a linker and a chelator. In some embodiments, the chelator has a bound radionuclide. In some embodiments, a linker and chelator, or a linker, chelator, and radionuclide are conjugated to a miniprotein. In some embodiments, a chelator and/or radionuclide are conjugated to a miniprotein. In some embodiments, a miniprotein is conjugated to a chelator either directly or through a linker (e.g., a linker described herein). Any known conjugation chemistry can be utilized to conjugate a miniprotein directly to a chelator or to conjugate a linker to the miniprotein and to the chelator.

In some embodiments, a miniprotein comprises a surface exposed functional group to allow for site specific conjugation. In some embodiments, a miniprotein comprises a surface exposed lysine or cysteine residue that can serve for site specific conjugation. In some embodiments, a miniprotein conjugate comprises one or more non-naturally occurring amino acids that can serve for site specific conjugation.

A person of ordinary skill in the art will recognize that numerous chemical conjugation strategies provide ready access to present technology, whereby exposed amino acid residues on a protein undergo well-known reactions with reactive moieties on a chelator.

A person of ordinary skill in the art will recognize that cysteine coupling reactions may be employed to conjugate chelators with thiol-reactive termini to protein surfaces through exposed thiol side chains on cysteine residues on the protein surface. (See generally Tsuchikama & An, supra, at 36-37; see also, e.g., Pierre Adumeau et al., Thiol-Reactive Bifunctional Chelators for the Creation of Site-Selectively Modified Radioimmuno conjugates with Improved Stability, 29 Bioconjugate Chem. 1364 (2018)). In some embodiments, because cysteine residues readily form disulfide linkages with nearby cysteine residues under physiological conditions, rather than existing as free thiols, some cysteine coupling strategies may rely upon selective reduction of disulfides to generate a higher number of reactive free thiols. Cysteine coupling techniques known in the art include, but are not limited to, cys alkylation reactions, cysteine rebridging reactions, and cys-aryl coupling using organometallic palladium reagents. See, e.g., C.R. Behrens et al., Antibody-Drug Conjugates (ADCs) Derived from Interchain Cysteine Cross-Linking Demonstrates Improved Homogeneity and Other Pharmacological Properties Over Conventional Heterogeneous ADCs, 12 Mol. Pharm. 3986 (2015); Vinogradova et al., Organometallic Palladium Reagents for Cysteine Bioconjugation, 526 Nature 687 (2015); see also Tsuchikama, supra, at 37.

Protein conjugation strategies using non-natural amino acid side chains are also well known in the art. For example, in some embodiments, “click chemistries” provide access to conjugated proteins, by rapid and selective chemical transformations under a diverse range of reaction conditions. In some embodiments, click chemistries are known to yield peptide conjugates with limited by-product formation, despite the presence of unprotected functional groups, in aqueous conditions. For instance, in some embodiments, a click reaction in the formation of conjugated peptides is the copper(I)-catalyzed azide-alkyne 1,3-dipolar cycloaddition reaction (CuAAC). (See Liyuan Liang & Didier Astruc, The Copper(I)-CatalysedAlkyne-Azide Cycloaddition (CuAAC) “Click” Reaction and Its Applications: An Overview, 255 COORD. CHEM. Rev 2933 (2011); see also, e.g., Herman S. Gill & Jan Marik, Preparation of 18F-labeled Peptides using the Copper(I)-Catalyzed Azide-Alkyne 1,3-Dipolar Cycloaddition, 6 Nature Protocols 1718 (2011)). In some embodiments, a CuAAC click reaction may be carried out in the presence of ligands to enhance reaction rates. In some such embodiments, such ligands may include, for example, polydentate nitrogen donors, including amines (e.g., tris(triazolyl)methyl amines) and pyridines. (See Liang & Astruc, supra, at 2934 (collecting examples); P. L. Goias et al., 39 Macromolecules 6451 (2006)). In some embodiments, other widely-utilized click reactions include, but are not limited to, thiol-ene, oxime, Diels-Alder, Michael addition, and pyridyl sulfide reactions.

In some embodiments, copper-free (Cu-free) click methods are also known in the art for delivery of therapeutic and/or diagnostic agents, such as radionuclides (e.g., 18F), chemotherapeutic agents, dyes, contrast agents, fluorescent labels, chemiluminescent labels, or other labels, to protein surfaces. In some embodiments, Cu-free click methods may permit stable covalent linkage between target molecules and prosthetic groups. In some embodiments, Cu-free click chemistry may include reacting an antibody or antigen binding fragment, which has been modified with a non-natural amino acid side chain that includes an activating moiety such as a cyclooctyne (e.g., dibenzocyclooctyne (DBCO)), a nitrone or an azide group, with a prosthetic group that presents a corresponding or complementary reactive moiety, such as an azide, nitrone or cyclooctyne (e.g., DBCO). (See, e.g., David. J. Donnelly et al., Synthesis and Biologic Evaluation of a Novel 18F-Labeled Adnectin as a PET Radio ligand for Imaging PD-L1 Expression, 59 J. NUCL. MED. 529 (2018)). For instance, in some embodiments, where a targeting molecule comprises a cyclooctyne, the prosthetic group may include an azide, nitrone, or similar reactive moiety. In some embodiments, where a targeting molecule includes an azide or nitrone, the prosthetic group may present a complementary cyclooctyne, alkyne, or similar reactive moiety. In some embodiments, Cu-free click reactions may be carried out at room temperature, in aqueous solution, in the presence of phosphate-buffered saline (PBS). In some such embodiments, prosthetic group may be radiolabeled (e.g., with 18F) or may be conjugated to any alternative diagnostic and/or therapeutic agent (e.g., a chelator). (See id. at 531.)

In some embodiments, conjugation chemistries such as the Huisgen cyclo-addition (“click” reaction) are available for synthesis of chelates and peptides. In some embodiments, an efficient, high-yielding three-step synthesis of a versatile monofluoro-substituted cyclooctyne (MFCO) has been shown to facilitate a variety of bioconjugation processes (M. Martin et al., 2013). In some embodiments, MFCO can be utilized to prepare a DOTA derivative for copper-free click chemical addition at an internal azide-modified lysine residue of the CDP or knottin peptide.

In some embodiments, a miniprotein conjugate provided herein has a lysine at a specific position (e.g., in a cysteine knot, or cysteine-dense region) and can be replaced with an azide derivative for “click” chemistry with DOTA-MFCO.

In some embodiments, a DOTA-MFCO-CDP conjugate can be prepared by first coupling an amine-modified DOTA to MFCO, then conjugating the DOTA-MFCO to the azide on the desired lysine of the miniprotein.

In some embodiments, the chelator and miniprotein are joined together by a cycloaddition reaction in the presence of a transition metal catalyst. In some embodiments, a metal catalyst is based on Cu or Rh.

In some embodiments, utilizing solution phase conjugation, a chelator (DOTA) and a miniprotein are joined with 1-ethyl-3-[3-(dimethylamino)propyl](EDC) and N-hydroxysulfonosuccinimide (SNHS) in water (pH 5.5) for 40 min at room temperature using a 1:1:1 molar ratio of DOTA:EDC:SNHS. In some such embodiments, peptides are dissolved in sodium phosphate buffer and added to the above sulfosuccinimidyl ester of DOTA (DOTA-OSSu). In some such embodiments, a molar excess of DOTA-OSSu is used to drive the conjugation on the N-termini of the peptide (See, e.g., Kimura, Richard H et al. “Engineered knottin peptides: a new class of agents for imaging integrin expression in living subjects.” Cancer research vol. 69,6 (2009): 2435-42. doi:10.1158/0008-5472.CAN-08-2495).

In some embodiments, a new DOTA derivative, μ-amino-DOTA is prepared with the objective of attaching DOTA to the C-terminus of a peptide, since in some scenarios, the peptide function might be compromised because of DOTA conjugation to the N-terminus or to lysine side chains.

In some embodiments, a miniprotein is generated by solid-phase peptide synthesis (SPPS). The tris-tert-butyl ester of DOTA, a bifunctional ligand (in the salt free, zwitterionic form), is readily soluble in most organic solvents and the tert-butyl ester protection is fully compatible with standard SPPS techniques. The most convenient way of conjugation comprises the addition of DOTA to the N-terminus of the protected peptide chain as the last amino acid in an automated peptide synthesizer followed by cleavage from the resin and removal of the acid-labile protecting groups. It can also be attached to Lys side chains. The preformed activated NHS ester of DOTA-tris(tert-butyl ester) has also been synthesized, and this reagent does not require a coupling agent to couple DOTA to free amino groups. The DOTA unit is linked to peptides through one of the acetate sidearms, and the conjugate has four amino, three carboxylates, and one amide group available for metal binding. See, e.g., De Le6n-Rodriguez L M, Kovacs Z. The synthesis and chelation chemistry of DOTA-peptide conjugates. Bioconjug Chem. 2008 February; 19(2):391-402. doi: 10.1021/bc700328s. Epub 2007 Dec 12. PMID: 18072717.

In some embodiments, a more general method involves the use of preformed DOTA-amino acid derivatives which allows the introduction of a DOTA unit into any desired position in the peptide sequence without the need of orthogonal protection. Protected DOTA-Lys and DOTA-Phe derivatives that are fully compatible with standard SPPS conditions (N—R-Fmoc protection, free carboxyl for the coupling, and acid-labile tert-butyl protection of the remaining acetate sidearms of the DOTA unit) have been synthesized. These DOTA-amino acids can be used in SPPS to build peptides that incorporate the DOTA moiety in any desired position. See, e.g., De Le6n-Rodriguez L M, Kovacs Z. The synthesis and chelation chemistry of DOTA-peptide conjugates. Bioconjug Chem. 2008 February; 19(2):391-402. doi: 10.1021/bc700328s. Epub 2007 Dec 12. PMID: 18072717.

General methods for coupling DOTA-type macrocycles to targeting groups through a linker (e.g., by activation of one of the carboxylates of the DOTA to form an active ester, which is then reacted with an amino group on the linker to form a stable amide bond), are known to those skilled in the art. See, e.g., Tweedle et al. U.S. Pat. No. 4,885,363.

A linker may be incorporated between the chelator and the targeting vector to influence the pharmacokinetic properties of the conjugate. Hydrocarbon, PEG, or polypeptide linkers can alter the pharmacokinetics and biodistribution by changing the overall charge and hydrophilicity of the radiopharmaceutical. See, e.g., De Le6n-Rodriguez L M, Kovacs Z. The synthesis and chelation chemistry of DOTA-peptide conjugates. Bioconjug Chem. 2008 Feb; 19(2):391-402. doi: 10.1021/bc700328s. Epub 2007 Dec 12. PMID: 18072717.

### Miniprotein Conjugate Orientation

In some embodiments, a conjugate has the following orientation: linker-chelator, linker-chelator-radionuclide, linker-radionuclide, chelator-radionuclide. In some such embodiments a conjugate has the following orientation: miniprotein-linker-radionuclide, miniprotein-chelator, chelator-miniprotein, miniprotein-linker-chelator, chelator-linker-miniprotein, miniprotein-chelator-radionuclide, radionuclide-chelator-miniprotein, miniprotein-linker-chelator-radionuclide, or radionuclide-chelator-linker-miniprotein.

In some embodiments, a conjugate provided by the present disclosure comprises a miniprotein. In some such embodiments, the miniprotein functions as a targeting moiety, e.g., specifically binding to a target, e.g., a protein expressed on the surface of a target tumor cell. Accordingly, in some embodiments, the miniprotein in the conjugates of the present disclosure may vary depending on the target of interest.

### Exemplary Miniprotein Conjugates

The following provides exemplary embodiments of miniprotein conjugates as provided herein. In some such embodiments, such miniproteins specifically bind to Nectin-4 expressed on the surface of a cancer cell (e.g., a solid tumor cell). In some embodiments, a conjugate comprises a linker, a chelator, and/or a radionuclide.

In some embodiments, a conjugate comprises a miniprotein, optional linker, chelator, and radionuclide. In some embodiments, a miniprotein comprises or consists of a binder, CDP, knottin, affibody, engineered Kunitz domain, monobody, anticalin, designed ankyrin repeat domain (DARPin), or avimer. In some embodiments, a chelator comprises or consists of DOTA, Crown, NOPO, or Macropa. In some embodiments, a radionuclide comprises or consists of an alpha emitter. In some embodiments, a radionuclide comprises or consists of a beta emitter. In some embodiments, a radionuclide comprises or consists of Ac-225, In-111, Ga-68, Pb-212, Lu-177, Cu-67, Cu-64, La-132, La-135, or Ce-134.

In some embodiments, a conjugate comprises or consists of a miniprotein that specifically binds to Nectin-4, an optional PEG linker, a DOTA chelator, and Ac-225.

In some embodiments, a conjugate comprises or consists of a miniprotein that specifically binds to Nectin-4, an optional PEG linker, a DOTA chelator, and In-111.

In some embodiments, a conjugate comprises or consists of a miniprotein that specifically binds to Nectin-4, an optional PEG linker, a DOTA chelator, and Lu-177.

In some embodiments, a conjugate comprises or consists of a miniprotein that specifically binds to Nectin-4, an optional PEG linker, a DOTA chelator, and Ga-68.

In some embodiments, a conjugate comprises or consists of a miniprotein that specifically binds to Nectin-4, an optional PEG linker, a DOTA chelator, and La-132.

In some embodiments, a conjugate comprises or consists of a miniprotein that specifically binds to Nectin-4, an optional PEG linker, a DOTA chelator, and La-135.

In some embodiments, a conjugate comprises or consists of a miniprotein that specifically binds to Nectin-4, an optional PEG linker, a DOTA chelator, and Cu-64.

In some embodiments, a conjugate comprises or consists of a miniprotein that specifically binds to Nectin-4, an optional PEG linker, a DOTA chelator and Cu-67.

In some embodiments, a conjugate comprises or consists of a miniprotein that specifically binds to Nectin-4, an optional PEG linker, a DOTA chelator, and Ce-134.

In some embodiments, a conjugate comprises or consists of a miniprotein that specifically binds to Nectin-4, an optional PEG linker, a Crown chelator, and Ac-225.

In some embodiments, a conjugate comprises or consists of a miniprotein that specifically binds to Nectin-4, an optional PEG linker, a Crown chelator, and In-111.

In some embodiments, a conjugate comprises or consists of a miniprotein that specifically binds to Nectin-4, an optional PEG linker, a Crown chelator, and Lu-177.

In some embodiments, a conjugate comprises or consists of a miniprotein that specifically binds to Nectin-4, an optional PEG linker, a Crown chelator, and Ga-68.

In some embodiments, a conjugate comprises or consists of a miniprotein that specifically binds to Nectin-4, an optional PEG linker, a Crown chelator, and La-132.

In some embodiments, a conjugate comprises or consists of a miniprotein that specifically binds to Nectin-4, an optional PEG linker, a Crown chelator, and La-135.

In some embodiments, a conjugate comprises or consists of a miniprotein that specifically binds to Nectin-4, an optional PEG linker, a Crown chelator, and Cu-64.

In some embodiments, a conjugate comprises or consists of a miniprotein that specifically binds to Nectin-4, an optional PEG linker, a Crown chelator, and Cu-67.

In some embodiments, a conjugate comprises or consists of a miniprotein that specifically binds to Nectin-4, an optional PEG linker, a Crown chelator, and Ce-134.

In some embodiments, a conjugate comprises or consists of a miniprotein that specifically binds to Nectin-4, an optional PEG linker, a NOPO chelator, and Ac-225.

In some embodiments, a conjugate comprises or consists of a miniprotein that specifically binds to Nectin-4, an optional PEG linker, a NOPO chelator, and In-111.

In some embodiments, a conjugate comprises or consists of a miniprotein that specifically binds to Nectin-4, an optional PEG linker, a NOPO chelator, and Ga-68.

In some embodiments, a conjugate comprises or consists of a miniprotein that specifically binds to Nectin-4, an optional PEG linker, a NOPO chelator, and La-132.

In some embodiments, a conjugate comprises or consists of a miniprotein that specifically binds to Nectin-4, an optional PEG linker, a NOPO chelator, and La-135.

In some embodiments, a conjugate comprises or consists of a miniprotein that specifically binds to Nectin-4, an optional PEG linker, a NOPO chelator, and Cu-64.

In some embodiments, a conjugate comprises or consists of a miniprotein that specifically binds to Nectin-4, an optional PEG linker, a NOPO chelator, and Cu-67.

In some embodiments, a conjugate comprises or consists of a miniprotein that specifically binds to Nectin-4, an optional PEG linker, a NOPO chelator, and Ce-134.

In some embodiments, a conjugate comprises or consists of a miniprotein that specifically binds to Nectin-4, an optional PEG linker, a Macropa chelator, and Ac-225.

In some embodiments, a conjugate comprises or consists of a miniprotein that specifically binds to Nectin-4, an optional PEG linker, a Macropa chelator, and In-111.

In some embodiments, a conjugate comprises or consists of a miniprotein that specifically binds to Nectin-4, an optional PEG linker, a Macropa chelator, and Lu-177.

In some embodiments, a conjugate comprises or consists of a miniprotein that specifically binds to Nectin-4, an optional PEG linker, a Macropa chelator, and Ga-68.

In some embodiments, a conjugate comprises or consists of a miniprotein that specifically binds to Nectin-4, an optional PEG linker, a Macropa chelator, and La-132.

In some embodiments, a conjugate comprises or consists of a miniprotein that specifically binds to Nectin-4, an optional PEG linker, a Macropa chelator, and La-135.

In some embodiments, a conjugate comprises or consists of a miniprotein that specifically binds to Nectin-4, an optional PEG linker, a Macropa chelator, and Cu-64.

In some embodiments, a conjugate comprises or consists of a miniprotein that specifically binds to Nectin-4, an optional PEG linker, a Macropa chelator, and Cu-67.

In some embodiments, a conjugate comprises or consists of a miniprotein that specifically binds to Nectin-4, an optional PEG linker, a Macropa chelator, and Ce-134.

In some embodiments, an exemplary imaging/diagnostic miniprotein conjugate described herein comprises a miniprotein that specifically binds to Nectin-4 or a fragment or portion thereof (e.g., expressed on the surface of a solid tumor cell), a PEG linker, a DOTA chelator, and Ac-225 or a cold-metal surrogate thereof.

In some embodiments, an exemplary imaging/diagnostic miniprotein conjugate described herein comprises a miniprotein that specifically binds to Nectin-4 or a fragment or portion thereof (e.g., expressed on the surface of a solid tumor cell), a PEG linker, a DOTA chelator, and Indium-111 or a cold-metal surrogate thereof.

In some embodiments, an exemplary imaging/diagnostic miniprotein conjugate described herein comprises a miniprotein that specifically binds to Nectin-4 or a fragment or portion thereof (e.g., expressed on the surface of a solid tumor cell), a PEG linker, a DOTA chelator, and Gallium-68 (or a cold-metal surrogate thereof).

In some embodiments, an exemplary imaging/diagnostic miniprotein conjugate described herein comprises a miniprotein that specifically binds to Nectin-4 or a fragment or portion thereof (e.g., expressed on the surface of a solid tumor cell), a PEG linker, a DOTA chelator, and Copper-64 (or a cold-metal surrogate thereof).

In some embodiments, an exemplary imaging/diagnostic miniprotein conjugate described herein comprises a miniprotein that specifically binds to Nectin-4 or a fragment or portion thereof (e.g., expressed on the surface of a solid tumor cell), a PEG linker, a DOTA chelator, and Lutetium-177 (or a cold-metal surrogate thereof).

In some embodiments, exemplary imaging/diagnostic CDP conjugate described herein comprises a miniprotein that specifically binds to Nectin-4 or a fragment or portion thereof (e.g., expressed on the surface of a solid tumor cell), a PEG linker, a DOTA chelator, and Lead-212 (or a cold-metal surrogate thereof).

In some embodiments, exemplary imaging/diagnostic CDP conjugate described herein comprises a miniprotein that specifically binds to Nectin-4 or a fragment or portion thereof (e.g., expressed on the surface of a solid tumor cell), a PEG linker, a DOTA chelator, and Cerium-134 (or a cold-metal surrogate thereof).

In some embodiments, a pharmaceutical composition comprising the radionuclide is employed in imaging scans to detect or diagnosis one or more diseases. Further embodiments include use as companion diagnostics.

In some embodiments, one or more different linkers and different chelators for different radionuclides and their respective chelators are operably linked to the same miniprotein.

In some embodiments, one or more different linkers and different chelators for Ga-68 (e.g., NOPO) and Ac-225 (e.g., Crown or DOTA) for both are operably linked to the same miniprotein.

### Methods of Use

In some embodiments, the present disclosure provides methods of treating or preventing disease or disorder in a subject, the method comprising administering to the subject the pharmaceutical composition in an amount effective that modulates, binds, or inhibits human Nectin-4 to treat or prevent disease or disorder in the subject. In preferred embodiments, the disease or disorder associated with Nectin-4 is treated (e.g., prevented, progression is slowed, symptoms are relieved, tumor size reduced to improve overall survival, etc.).

In some embodiments, subjects, e.g., patient or patients inclusion criteria include, without limitation, Nectin-4 positive candidates shown via imaging (e.g., DOTA PET/CT), candidates with progressive disease, advanced or metastatic disease, candidates who are not candidates for surgery, refractory or relapsed candidates.

In some embodiments, possible certain side effects including nausea, suppression of blood cell counts are managed through one or more medications. In some embodiments, side effects may include renal toxicity, myelodysplastic syndrome, however, it is contemplated that the pharmaceutical compositions are manageable, and treatment is generally well-tolerated.

In some embodiments, the present disclosure comprises a method for treatment, comprising administering a pharmaceutical composition as provided herein in the absence of administering targeted conditioning or pre-conditioning regimens where conditioning is necessary prior to administration of therapies, e.g., adoptive cell therapies and gene therapies to ablate certain cells.

In some embodiments, methods and compositions of the present disclosure include multistep or pretargeting approaches. For instance, in some embodiments, a radionuclide can be decoupled to a provided composition and may be subsequently administered after an initial step of administering a miniprotein or an antibody (e.g., a first ligand binding moiety). In such embodiments, the first ligand binding moiety is not conjugated to a radionuclide and has the desired affinity and specificity for the tumor cells. The first ligand binding moiety is then targeted by a second moiety carrying the radionuclide. For instance, the first ligand binding moiety may comprise an antibody to the Nectin-4 and the second moiety may be the pharmaceutical composition comprising the miniprotein, linker, chelator and the radionuclide wherein the miniprotein may exhibit a desired avidity to the first ligand binding moiety.

In some embodiments, the present disclosure provides methods of use (e.g., treatment, manufacture, etc.) of miniproteins (e.g., CDPs, knottins, binders, affibodies, engineered Kunitz domains, monobodies, anticalins, designed ankyrin repeat domains (DARPins), avimers) as provided herein.

In some embodiments, anti-Nectin-4 compositions and pharmaceutical compositions are produced, e.g., using miniproteins (e.g., CDPs, knottins, binders, affibodies, engineered Kunitz domains, monobodies, anticalins, designed ankyrin repeat domains (DARPins), avimers), as provided herein.

In some embodiments, Nectin-4 miniproteins (e.g., CDPs, knottins, binders, affibodies, engineered Kunitz domains, monobodies, anticalins, designed ankyrin repeat domains (DARPins), avimers) provided by the present disclosure are formatted to generate peptides, antibodies, antibody and antibody fragments, ADCs, BiTEs, cAR-Ts, and TRuCs, Fc-domain components, portions, or modifications, bispecific antibodies etc. In some such embodiments, such compositions and pharmaceutical compositions are used in treatment of a disease, disorder, or condition wherein Nectin-4 expression is suspected or detected. In some such embodiments, the disease, disorder, or condition is related to overexpression and/or aberrant expression of Nectin-4. In some embodiments, the disease, disorder and/or condition is cancer. Accordingly, the present disclosure provides various anti-Nectin-4 compositions and pharmaceutical compositions for the treatment of disease related to Nectin-4.

Among other things, the present disclosure provides methods of treating a subject in need thereof by administering a composition as provided herein. In some such embodiments, a composition is or comprises a miniprotein (e.g., CDP, knottin, binder, affibody, engineered Kunitz domain, monobody, anticalin, designed ankyrin repeat domain (DARPin), avimer). In some embodiments, the composition is a miniprotein conjugate comprising a miniprotein and one or more of a chelator and radionuclide, as well as, optionally, a linker (e.g., linking the chelator to the miniprotein).

In some embodiments, a subject treated herein is at risk of having or has been diagnosed as having a cancer. In some embodiments, the subject is a mammal. In some embodiments, the mammal is a human. In some embodiments, the human is a fetus, infant, child, adolescent, adult, or elderly adult. In some embodiments, a human subject having a cancer is treated by administering a miniprotein (e.g., CDP, knottin, binder, affibody, engineered Kunitz domain, monobody, anticalin, designed ankyrin repeat domain (DARPin), avimer) as described herein.

In some embodiments, the cancer expresses a target protein (e.g., Nectin-4) specifically bound by a miniprotein (e.g., CDP, knottin, binder, affibody, engineered Kunitz domain, monobody, anticalin, designed ankyrin repeat domain (DARPin), avimer) of the present disclosure. In some embodiments, the Nectin-4 or portion thereof is expressed on the surface of a cancer cell of the subject. In some such embodiments, the Nectin-4 is expressed on the cancer cell and has lower or non-detectable expression on cells of normal tissues, and/or is expressed at much higher density on cancer cells versus normal cells.

In some embodiments, the cancer is a solid tumor. In some embodiments, the cancer is metastatic. In some embodiments, the cancer is recurrent. In some embodiments, the cancer is remitting. In some embodiments, the cancer is selected from the group consisting of bladder, breast, pancreas, ovary, stomach, gastrointestinal tract, liver, lung, prostate, skin, colon, rectum, colon and rectum, skin.

In some embodiments, miniproteins (e.g., CDPs, knottins, binders, affibodies, engineered Kunitz domains, monobodies, anticalins, designed ankyrin repeat domains (DARPins), avimers) provided herein can be used in conjunction with one or more additional components. For example, in some embodiments, a miniprotein (e.g., CDP, knottin, binder affibody, engineered Kunitz domain, monobody, anticalin, designed ankyrin repeat domain (DARPin), avimer) may be combined with one or more other components for use in imaging, diagnosis, prognosis/monitoring and/or treating a disease, disorder or condition. In some embodiments, the disease is cancer. In some embodiments, a miniprotein (e.g., CDP, knottin, binder affibody, engineered Kunitz domain, monobody, anticalin, designed ankyrin repeat domain (DARPin), avimer) of the present disclosure may be used in a wide variety of cancers, including, but not limited to, breast cancer, ovarian cancer, melanoma, pancreatic cancer, peripheral neuroma, glioblastoma, adrenocortical carcinoma, AIDS-related lymphoma, anal cancer, bladder cancer, meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, male breast cancer, malignant mesothelioma, medulloblastoma, Merkel cell carcinoma, metastatic squamous neck cell carcinoma, multiple myeloma and other plasma cell neoplasms, mycosis fimgoides and the Sezary syndrome, myelodysplastic syndromes, nasopharyngeal cancer, neuroblastoma, non-small cell lung cancer, small cell lung cancer, head and neck cancer, skin cancer, oropharyngeal cancer, bone cancers, including osteosarcoma and malignant fibrous histiocytoma of bone, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

In some embodiments, treatment (e.g., including with a miniprotein (e.g., CDP, knottin, binder affibody, engineered Kunitz domain, monobody, anticalin, designed ankyrin repeat domain (DARPin), avimer) of the present disclosure), diagnosis, prognosis/monitoring, or imaging is in a subject who does not exhibit signs or symptoms of a disease, disorder, and/or condition. In some embodiments, treatment is in a subject who exhibits one or more signs or symptoms of a disease, disorder, or condition even if, for example, such signs or symptoms are not objectively observable without further testing such as laboratory diagnostics. In some embodiments, a subject is susceptible to having or at risk of developing a disease, disorder, or condition (e.g., cancer), based on one or more factors (e.g., level of Nectin-4, etc.) that are related to increased risk of developing of the disease, disorder or condition. In some embodiments, a subject has been diagnosed as having a disease, disorder, or condition (e.g., cancer).

In some embodiments, present disclosure provides methods of treating or preventing disease or disorder in a subject, the method comprising administering to the subject a pharmaceutical composition comprising a miniprotein (e.g., CDP, knottin, binder affibody, engineered Kunitz domain, monobody, anticalin, designed ankyrin repeat domain (DARPin), avimer) of the present disclosure in an amount effective that modulates, binds or inhibits human Nectin-4 to treat or prevent disease or disorder in the subject. In some embodiments, the disease or disorder is cancer. In some embodiments, the cancer is treated (e.g., resolved, prevented, progression is slowed, symptoms are relieved, tumor size reduced to improve overall survival, etc.).

### Formulation and Administration

In various aspects formulations of the pharmaceutical compositions of the present disclosure include parenteral e.g., subcutaneous, intravenous, intraarterial, intramuscular, intradermal, intraperitoneal, intraurethral, interperitoneal, and intrathecal administration. See for instance, Remington: The Science and Practice of Pharmacy, Lippincott Williams & Wilkins; 22nd ed. (2013) described in more detail.

In some embodiments, a pharmaceutical composition comprising a miniprotein (e.g., CDP, knottin, binder affibody, engineered Kunitz domain, monobody, anticalin, designed ankyrin repeat domain (DARPin), avimer) of the present disclosure is administered to a subject in need thereof. In some embodiments, the subject has or is at risk of having cancer. By way of non-limiting example, in some embodiments, the cancer is selected from breast cancer, ovarian cancer, melanoma, pancreatic cancer, peripheral neuroma, glioblastoma, adrenocortical carcinoma, AIDS-related lymphoma, anal cancer, bladder cancer (e.g., early stage bladder cancer), meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, male breast cancer, malignant mesothelioma, medulloblastoma, Merkel cell carcinoma, metastatic squamous neck cell carcinoma, multiple myeloma and other plasma cell neoplasms, mycosis fimgoides and the Sezary syndrome, myelodysplastic syndromes, nasopharyngeal cancer, neuroblastoma, non-small cell lung cancer, small cell lung cancer, head and neck cancer, skin cancer, oropharyngeal cancer, bone cancers, including osteosarcoma and malignant fibrous histiocytoma of bone, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

In some embodiments, determination of an appropriate dose and regimen of a miniprotein (e.g., CDP, knottin, binder, affibody, engineered Kunitz domain, monobody, anticalin, designed ankyrin repeat domain (DARPin), avimer) of the present disclosure can be made by the clinician, e.g., using parameters or factors known or suspected in the art to affect treatment or predicted to affect treatment. Actual dosage levels of the active ingredients (e.g., miniproteins (e.g., CDPs, knottins, binders, affibodies, engineered Kunitz domains, monobodies, anticalins, designed ankyrin repeat domains (DARPins), avimers) as provided by compositions of the present disclosure may be varied so as to obtain an amount of the active ingredient which is effective to achieve the desired therapeutic response for a particular patient, composition, and mode of administration, without being toxic to the patient. In some embodiments, the selected dosage level will depend upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors known in the medical arts.

In some embodiments, administration is by one or more routes including, but not limited to bronchial, buccal, enteral, interdermal, intra-arterial, intradermal, intragastric, intramedullary, intramuscular, intranasal, intraperitoneal, intrathecal, intraurethral, intravenous, intraventricular, within a specific organ and/or tissue, mucosal, nasal, oral, rectal, subcutaneous, sublingual, topical, tracheal (including by intratracheal instillation), transdermal, vaginal and vitreal.

In some embodiments, administration may comprise or consist of continuous dosing (e.g., intravenous administration) for a period of time.

In some embodiments, administration may comprise or consist of intermittent dosing.

In some embodiments, administration may comprise or consist of dosing separated by a selected period of time and with one or more doses, based on clinical response and/or activity following one or more doses.

In some embodiments, administration is to a subject is suffering from a relevant disease, disorder or condition. In some embodiments, administration is to a subject susceptible to or at risk of developing a disease, disorder, or condition. In some such embodiments, a subject displays one or more symptoms or characteristics of a disease, disorder or condition. In some embodiments, a subject does not display any symptom or characteristic of a disease, disorder, or condition. In some such embodiments, a subject is someone with one or more features characteristic of susceptibility to or at risk of developing a disease, disorder, or condition. In some embodiments, a subject has received a diagnosis of a disease, disorder, or condition.

In some embodiments, the present disclosure provides a method for modulating biological activity of Nectin-4 in a subject. In some such embodiments, the method comprises administering a pharmaceutical composition provided by the disclosure to the subject in an amount effective to modulate the biological activity of Nectin-4 in the subject.

In some embodiments, the present disclosure provides a method for treating or preventing cancer in a subject. In some embodiments, the method comprises administering to the subject a pharmaceutical composition provided by the present disclosure, wherein the miniprotein (e.g., CDP, knottin, binder, affibody, engineered Kunitz domain, monobody, anticalin, designed ankyrin repeat domain (DARPin), avimer) of the pharmaceutical composition selectively binds to Nectin-4 in an amount effective to treat or prevent the cancer in the subject.

In some embodiments, the present disclosure provides methods and compositions that bind target (e.g., Nectin-4) and are capable of activating or inhibiting immune cell response. In some embodiments, compositions are administered for the treatment of non-small-cell lung cancer (NSCLC), cutaneous squamous cell carcinoma, pancreatic cancer, primary hepatocellular carcinoma, colorectal carcinoma, clear cell renal carcinoma, breast cancer and prostate cancer. (Yang, S et al., Int J of Bio Sci 2020 Mar 25 (16): 11; 1767-1773).

In some embodiments, the present disclosure provides a method for detecting the presence or extent of a cancer in a subject. In some such embodiments, the method comprises measuring a level of Nectin-4 in a sample comprising one or more cells from the subject; wherein detection of the level of Nectin-4 in the subject relative to the levels of the Nectin-4 in one or more control subjects is indicative of the presence or extent of the cancer.

In some embodiments, a composition provided by the present disclosure is used to downregulate an inhibitory immune response in a subject. For example, in some embodiments, a miniprotein (e.g., CDP, knottin, binder, affibody, engineered Kunitz domain, monobody, anticalin, designed ankyrin repeat domain (DARPin), avimer) of the present disclosure specifically binds such that it may be used to deliver a cytotoxic payload and promote cellular cytotoxicity of T cells for specific Nectin-4-expressing (e.g., tumor) cells. (See Goodman A, Patel S P, Kurzrock R Nat Rev Clin Oncol. 2017 April; 14(4):203-220.) In some such embodiments, the miniprotein (e.g., CDP, knottin, binder, affibody, engineered Kunitz domain, monobody, anticalin, designed ankyrin repeat domain (DARPin), avimer) modulates IFN-γ IL-2, IL-10, and IL-13 production during T-cell activation.

### Treatment, Imaging, and Diagnostic/Prognostic Methods of Use

In some embodiments, the present disclosure provides methods of treating cancer in a human subject by administering a miniprotein conjugate described herein. In some embodiments, the cancer expresses the target (e.g., Nectin-4) specifically bound by the miniprotein of the conjugate. In some embodiments, the target protein is expressed on the surface of malignant cells with limited expression on cells of normal tissues, and/or expressed at much higher density on malignant versus normal cells.

In some embodiments, the cancer is a solid tumor. In some embodiments, the cancer is metastatic. In some embodiments, the cancer is recurrent. In some embodiments, the cancer is remitting. In some embodiments, the cancer is selected from the group consisting of bladder, breast, pancreas, ovary, stomach, gastrointestinal tract, liver, lung, prostate, skin, colon, rectum, colon and rectum, skin.

In some embodiment, miniprotein conjugates provided herein can be used for imaging and treating a wide variety of cancers, including, but not limited to, breast cancer, ovarian cancer, melanoma, pancreatic cancer, peripheral neuroma, glioblastoma, adrenocortical carcinoma, AIDS-related lymphoma, anal cancer, bladder cancer, meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, male breast cancer, malignant mesothelioma, medulloblastoma, Merkel cell carcinoma, metastatic squamous neck cell carcinoma, multiple myeloma and other plasma cell neoplasms, mycosis fimgoides and the Sezary syndrome, myelodysplastic syndromes, nasopharyngeal cancer, neuroblastoma, non-small cell lung cancer, small cell lung cancer, head and neck cancer, skin cancer, oropharyngeal cancer, bone cancers, including osteosarcoma and malignant fibrous histiocytoma of bone, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

As will be known to those of skill in the art, determination of the appropriate dose and regimen of a composition provided by the present disclosure can be made by the clinician, e.g., using parameters or factors known or suspected in the art to affect treatment or predicted to affect treatment. In some embodiments, actual dosage levels of the active ingredients in compositions provided by the present disclosure may be varied so as to obtain an amount of the active ingredient which is effective to achieve the desired therapeutic response for a particular patient, composition, and mode of administration, without being toxic to the patient. In some embodiments, the selected dosage level will depend upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors known in the medical arts.

In some embodiments, the present disclosure provides methods of imaging, diagnosing and/or monitoring (including determining prognosis) of presence of a target in a subject. In some embodiments, a conjugate (e.g., a miniprotein conjugate comprising, e.g., a chelator and/or radionuclide) of the present disclosure is useful for PET, SPECT, or MRI imaging.

In some embodiments, conjugates (e.g., miniprotein conjugates comprising a chelator and/or radionuclide) of the present disclosure can be used in image-guided surgery. For example, in some embodiments, tissue of interest suspected of containing cancerous cells or a tumor can be contacted with a Nectin-4-targeted miniprotein (e.g., CDP, knottin, binder, affibody, engineered Kunitz domain, monobody, anticalin, designed ankyrin repeat domain (DARPin), avimer), such that the Nectin-4-targeted miniprotein (e.g., CDP, knottin, binder, affibody, engineered Kunitz domain, monobody, anticalin, designed ankyrin repeat domain (DARPin), avimer) or component(s) thereof (chelator, radionuclide) accumulates in metastatic cancerous cells. Imaging of tissues labeled with the Nectin-4 miniprotein conjugate wherein the conjugate comprises more additional detectable components (e.g., chelator, e.g., radionuclide, e.g., other detectable imaging moiety) can be used, for example, for detection of metastatic cells, tumor margin delineation, evaluation of the completeness of resection, and evaluation of the efficacy of treatment.

In some embodiments, the present disclosure provides methods of imaging a cancer in a subject. In some embodiments, miniprotein conjugates of the present disclosure are useful for PET, SPECT, or MRI imaging. In some embodiments, a detectably effective amount of a miniprotein conjugate is administered to a subject; that is, an amount that is sufficient to yield an acceptable image using the imaging equipment that is available for clinical use. In some embodiments, a detectably effective amount of a miniprotein conjugate may be administered in more than one injection if needed. In some such embodiments, a detectably effective amount of miniprotein conjugate needed for an individual may vary according to factors such as the degree of uptake of miniprotein conjugates into cancerous tissue, the age, sex, and weight of the individual, and the particular medical imaging method used. Optimization of such factors is within the level of skill in the art.

In some embodiments, imaging with miniprotein conjugates can be used in assessing efficacy of therapeutic drugs in treating cancer. For example, images can be acquired after treatment with an anti-cancer therapy to determine if the individual is responding to treatment. In some embodiments, in a subject with cancer, imaging with miniprotein conjugate can be used to evaluate whether a tumor is shrinking or growing. Further, the extent of cancerous disease (how far and where the cancer has spread) can be determined to aid in determining prognosis and evaluating optimal strategies for treatment (e.g., surgery, radiation, or chemotherapy).

In some embodiments, miniprotein conjugates can be used in image-guided surgery. Tissue of interest suspected of containing cancerous cells or a tumor can be contacted with a miniprotein conjugate, such that the miniproteins or components thereof (e.g., chelator, e.g., radionuclide) accumulate in metastatic cancerous cells. In some embodiments, imaging of tissues labeled with miniprotein conjugate in this way can be used, for example, for detection of metastatic cells, tumor margin delineation, evaluation of the completeness of resection, and evaluation of the efficacy of treatment.

### Kits

In one aspect, provided herein are kits comprising a pharmaceutical composition described herein for therapeutic, imaging, or diagnostic uses. In some embodiments, kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a cancer, the kit comprising: (a) a dosage of pharmaceutical composition described herein and (b) instructions for using the in methods of therapy methods disclosed herein. In certain embodiments for treating human patients, the kit comprises a pharmaceutical composition described herein comprising a miniprotein conjugate described herein.

In some embodiments, a kit comprises a cold miniprotein conjugate as provided herein (i.e., a miniprotein conjugate that does not contain a radionuclide), and instructions for chelation of the miniprotein conjugate to the radionuclide. In some embodiments, the kit comprises a hot miniprotein conjugate as provided herein (i.e., a miniprotein conjugate described herein comprising the radionuclide), with instructions for administration to a subject.

In some embodiments, the present disclosure provides kits comprising a miniprotein (e.g., CDP, knottin, binder, affibody, engineered Kunitz domain, monobody, anticalin, designed ankyrin repeat domain (DARPin), avimer) as provided herein. In some embodiments, the kit comprises compositions for detecting the Nectin-4 miniprotein (e.g., CDP, knottin, binder, affibody, engineered Kunitz domain, monobody, anticalin, designed ankyrin repeat domain (DARPin), avimer) (e.g., conjugation to one or more detectable moieties).

In some embodiments, a kit comprises a label indicating the intended use of the contents of the kit and instructions for use. The term label includes any writing, or recorded material supplied on or with the kit, or which otherwise accompanies the kit.

In some embodiments, the present disclosure provides a kit for treating, monitoring, or diagnosing a subject having or suspected of having cells overexpressing Nectin-4, the kit comprising: (a) a unit of a pharmaceutical composition described herein and (b) instructions for using the in methods of administration disclosed herein. In certain embodiments for treating human patients, the kit comprises a pharmaceutical composition described herein comprising miniprotein (e.g., CDP, knottin, binder, affibody, engineered Kunitz domain, monobody, anticalin, designed ankyrin repeat domain (DARPin), avimer) as provided herein.

### Certain Exemplary Embodiments

In some embodiments, the present disclosure provides compositions comprising a miniprotein (M), an optional linker (L), and one or both of a chelator (C) and a radionuclide (R), represented a formula selected from M)x-L-C-R, (M)x-L-C, (M)x-C-R, (M)x-L-R, (M)x-C, (M)ₓ-L, and (M)ₓ-R. In some embodiments, the miniprotein comprises or consists of a cysteine-dense polypeptide, a knottin-protein, a binder, an affibody, an engineered Kunitz domain, a monobody, an anticalin, a designed ankyrin repeat domain (DARPin), or an avimer. In some embodiments, the binder comprises or consists of a linear polypeptide and/or a non-disulfide sequence. In some embodiments, M is characterized in that it comprises (i) no more than 1,000 amino acids, 900 amino acids, 800 amino acids, 700 amino acids, 600 amino acids, 500 amino acids, 400 amino acids, 300 amino acids, 200 amino acids, or 100 amino acids. In some preferred embodiments, the miniprotein is characterized in that it comprises (i) no more than 100 amino acids and/or 12 kDa; (ii) at least one secondary structure elements; (iii) a sequestered hydrophobic core; and/or displays cooperative folding. In some embodiments, the miniprotein comprises no more than about 100 amino acids or less, 90 amino acids, 85 amino acids, 80 amino acids, 75 amino acids, 70 amino acids, 65 amino acids, 60 amino acids, 55 amino acids, 50 amino acids, 45 amino acids, 40 amino acids, 35 amino acids, 30 amino acids, 25 amino acids, 20 amino acids, 15 amino acids, 10 amino acids, or 5 amino acids. In some embodiments, the miniprotein comprises at least one disulfide bridge.

In some embodiments, the present disclosure provides compositions represented by the formula L-C, wherein L comprises or consists of a linker, C comprises or consists of a chelator, and wherein the linker is designed to be conjugated to a polypeptide. In some embodiments, the present disclosure provides compositions represented by the formula L-C-R, wherein L comprises or consists of a linker, C comprises or consists of a chelator, and R comprises or consists of a radionuclide, and wherein the composition is capable of being conjugated to a miniprotein. In some embodiments, L comprises or consists of a polyethylene glycol (PEG) linker of PEG4, PEG2, PEG6, PEG8, PEG12, PEG24, an ester linker, an amide linker, a maleimide linker, a succinimidyl-4-(N-maleimidomethyl) cyclohexane-1-carboxylate (SMCC) linker, a propanoic acid linker, a caproleic acid linker, or (Gly)n-(γGlu)n- or (PEG)n, wherein n is from 1 to 10, (Gly)1-10, or any fragment or combination via covalent bond thereof. In some embodiments, C comprises or consists of tetrazacyclododecane-1,4,7,10-tetraacetic acid (DOTA), Crown, NOPO, or Macropa as set forth as follows:

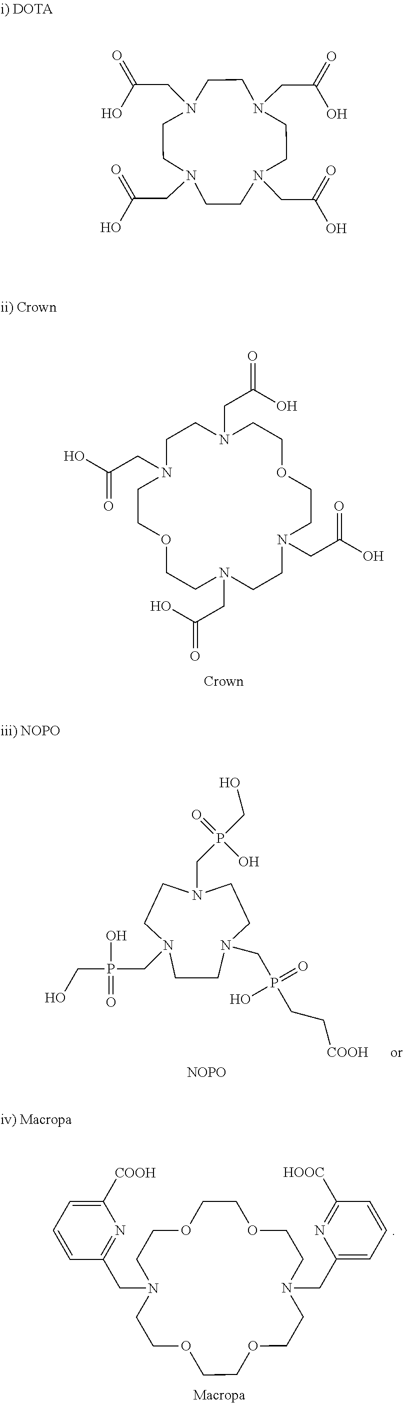 [SMILES: O=C(O)CN1CCN(CC(=O)O)CCN(CC(=O)O)CCN(CC(=O)O)CC1 ; O=C(O)CN1CCOCCN(CC(=O)O)CCN(CC(=O)O)CCOCCN(CC(=O)O)CC1 ; O=C(O)CCP(=O)(O)CN1CCN(CP(=O)(O)CO)CCN(CP(=O)(O)CO)CC1 ; "NOPO" ; O=C(O)c1cccc(CN2CCOCCOCCN(Cc3cccc(C(=O)O)n3)CCOCCOCC2)n1]

In some embodiments, C comprises or consists of tetrazacyclododecane-1,4,7,10-tetraacetic acid (DOTA), Crown, NOPO, or Macropa, or a derivative thereof.

In some embodiments, the present disclosure provides isolated constructs or pharmaceutically acceptable salts thereof comprising a miniprotein, optional linker, and at least one of a chelator or radionuclide. In some embodiments, R comprises or consists of Ac-225, ln-111, Ga-68, Pb-212, Lu-177, Cu-67, Cu-64, La-132, La-135, or Ce-134. In some embodiments, the composition comprises at least one additional component. In some embodiments, the composition can penetrate tumor tissue. In some embodiments, the miniprotein specifically binds to a target. In some embodiments, the composition displays m or nM binding affinity to the target (e.g., in an in vitro assay, e.g., in a cell isolated from a tumor e.g., in vivo, e.g., to a tumor.) In some embodiments, the composition displays m or nM binding affinity to the target, e.g., in an in vitro assay. In some embodiments, the composition binds to the target with an affinity of 1 pM to 100 nM e.g., as measured by an in vitro binding assay. In some embodiments, the composition binds to the target with an affinity of 100 pM to 10 nM, e.g., as measured by an in vitro binding assay. In some embodiments, the miniprotein binding to the target modulates biological function. In some embodiments, the miniprotein binding to the target does not elicit an immune response. In some embodiments, the immune response includes a systemic immune response or a local immune response. In some embodiments, the target is located in, on, or near a cell. In some embodiments, the target is a protein expressed on the surface of the cell. In some embodiments, the cell is a tumor cell. In some embodiments, the tumor cell is a solid tumor cell. In some embodiments, the cell is a human cell. In some embodiments, the target is Nectin-4. In some embodiments, the miniprotein selectively binds to Nectin-4 or a portion thereof.

In some embodiments, the present disclosure provides pharmaceutical compositions comprising a miniprotein, (M) an optional linker (L) and one or both of a chelator (C) and a nuclide. In some embodiments, when L is present, L comprises or consists of a polyethylene glycol (PEG) linker, an ester linker, an amide linker, a maleimide linker, a valine-citrulline linker, a hydrazone linker, a N-succinimidyl-4-(2-pyridyldithio)butyrate (SPDB) linker, a succinimidyl-4-(N-maleimidomethyl)cyclohexane-1-carboxylate (SMCC) linker, a vinylsulfone-based linker, a propanoic acid linker, a caproleic acid linker, or any fragment or combination thereof. In some embodiments, when C is present, C comprises or consists of tetrazacyclododecane-1,4,7,10-tetraacetic acid (DOTA), Crown, NOPO, or Macropa as set forth as follows:

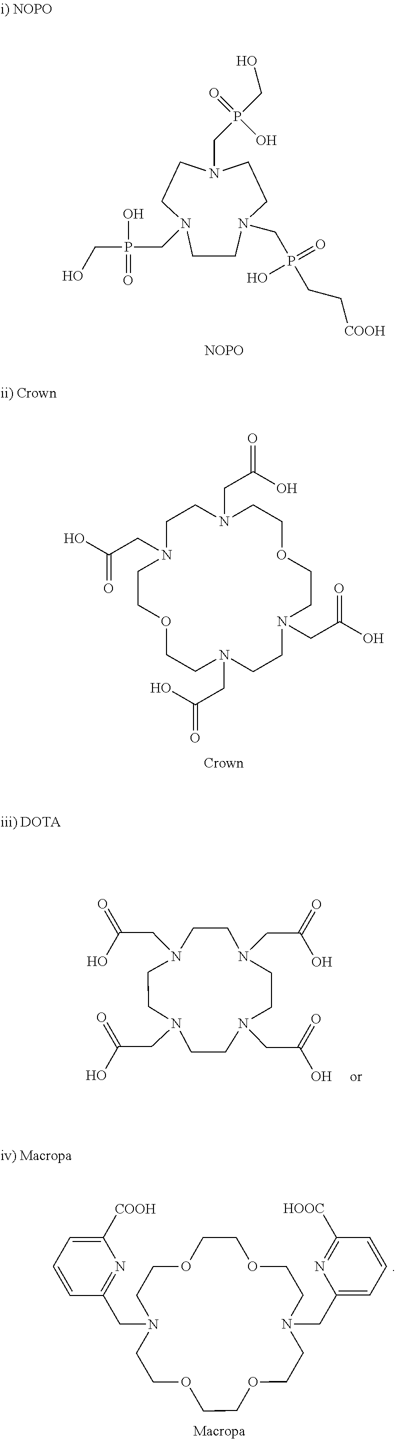 [SMILES: O=C(O)CCP(=O)(O)CN1CCN(CP(=O)(O)CO)CCN(CP(=O)(O)CO)CC1 ; "NOPO" ; O=C(O)CN1CCOCCN(CC(=O)O)CCN(CC(=O)O)CCOCCN(CC(=O)O)CC1 ; O=C(O)CN1CCN(CC(=O)O)CCN(CC(=O)O)CCN(CC(=O)O)CC1 ; O=C(O)c1cccc(CN2CCOCCOCCN(Cc3cccc(C(=O)O)n3)CCOCCOCC2)n1]

In some embodiments, the chelator covalently attaches to the miniprotein. In some embodiments, the chelation efficiency is >90%. In some embodiments, when R is present, the radionuclide is an alpha-emitter. In some embodiments, the radionuclide is Ac-225, In-111, Ga-68, Pb-212, Lu-177, Cu-67, Cu-64, La-132, La-135, or Ce-134. In some embodiments, the miniprotein specifically binds to a target. In some embodiments, the target is Nectin-4. In some embodiments, the Nectin-4 is expressed on a cell. In some embodiments, the cell is a human cell. In some embodiments, the human cell is a tumor cell. In some embodiments, the tumor cell is a solid tumor cell. In some embodiments, the miniprotein comprises or consists of a cysteine-dense polypeptide, a knottin-protein, a binder, an affibody, an engineered Kunitz domain, a monobody, an anticalin, a designed ankyrin repeat domain (DARPin), or an avimer. In some embodiments, the miniprotein comprises one or more disulfide bonds. In some embodiments, the miniprotein is characterized in that it has nM or sub-nM binding affinity on the target in vivo or in a cell-based assay. In some embodiments, the miniprotein a binding affinity of 1 pM to 100 nM to Nectin-4 on a cell line expressing human Nectin-4. In some embodiments, the miniprotein a binding affinity of 100 pM to 10 nM to Nectin-4 on a cell line expressing human Nectin-4. In some embodiments, the miniprotein has an amino acid sequence no more than about 100 amino acids or less, 90 amino acids, 85 amino acids, 80 amino acids, 75 amino acids, 70 amino acids, 65 amino acids, 60 amino acids, 55 amino acids, 50 amino acids, 45 amino acids, 40 amino acids, 35 amino acids, 30 amino acids, 25 amino acids, 20 amino acids, 15 amino acids, 10 amino acids, or 5 amino acids. In some embodiments, the miniprotein does not elicit an immune response or wherein the immune response elicited is tolerable. In some embodiments, the pharmaceutical composition comprises high tumor tissue penetration. In some embodiments, the pharmaceutical composition is not taken up and/or retained in the kidney or liver. In some embodiments, the pharmaceutical composition is internalized in a cell expressing human Nectin-4.

In some embodiments, the present disclosure provides methods of treating a subject in need thereof comprising administering a composition comprising a miniprotein (M), an optional linker (L), and one or both of a chelator (C) and a radionuclide (R). In some embodiments, when L is present, L comprises or consists of a polyethylene glycol (PEG) linker, an ester linker, an amide linker, a maleimide linker, a valine-citrulline linker, a hydrazone linker, a N-succinimidyl-4-(2-pyridyldithio)butyrate (SPDB) linker, a succinimidyl-4-(N-maleimidomethyl)cyclohexane-1-carboxylate (SMCC) linker, a vinylsulfone-based linker, a propanoic acid linker, a caproleic acid linker, or any fragment or combination thereof. In some embodiments, when C is present, C comprises or consists of NOPO, Crown, Macropa or tetrazacyclododecane-1,4,7,10-tetraacetic acid (DOTA) as set forth as follows:

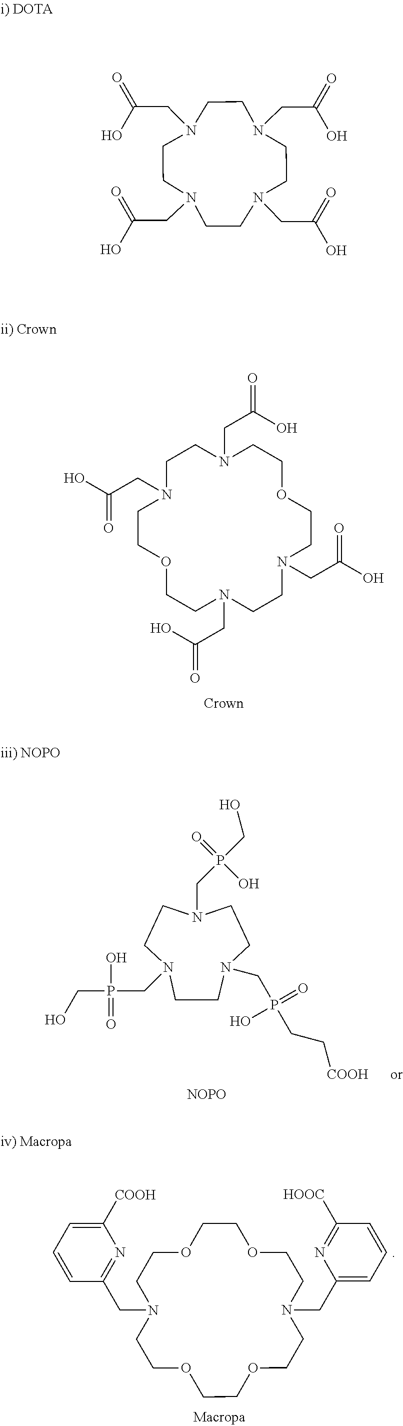 [SMILES: O=C(O)CN1CCN(CC(=O)O)CCN(CC(=O)O)CCN(CC(=O)O)CC1 ; O=C(O)CN1CCOCCN(CC(=O)O)CCN(CC(=O)O)CCOCCN(CC(=O)O)CC1 ; O=C(O)CCP(=O)(O)CN1CCN(CP(=O)(O)CO)CCN(CP(=O)(O)CO)CC1 ; "NOPO" ; O=C(O)c1cccc(CN2CCOCCOCCN(Cc3cccc(C(=O)O)n3)CCOCCOCC2)n1]

In some embodiments, when R is present, R comprises or consists of Ac-225, In-111, Ga-68, Pb-212, Lu-177, Cu-67, Cu-64, La-132, La-135, or Ce-134. In some embodiments, M comprises or consists of a cysteine-dense peptide, a knottin peptide, a binder, an affibody, an engineered Kunitz domain, a monobody, an anticalin, a designed ankyrin repeat domain (DARPin), or an avimer. In some embodiments, M is characterized in that it comprises (i) no more than 1,000 amino acids, 900 amino acids, 800 amino acids, 700 amino acids, 600 amino acids, 500 amino acids, 400 amino acids, 300 amino acids, 200 amino acids, or 100 amino acids. In some preferred embodiments, M is characterized in that it comprises (i) no more than 100 amino acids and/or 12 kDa; (ii) at least two secondary structure elements; (iii) a sequestered hydrophobic core; and/or displays cooperative folding. In some embodiments, a composition comprising M, optional L, and one or both of C and R, for use in a method of the present disclosure, comprises at least one additional component. In some embodiments, the composition can penetrate tumor tissue. In some embodiments, the miniprotein comprises no more than about 100 amino acids or less, 90 amino acids, 85 amino acids, 80 amino acids, 75 amino acids, 70 amino acids, 65 amino acids, 60 amino acids, 55 amino acids, 50 amino acids, 45 amino acids, 40 amino acids, 35 amino acids, 30 amino acids, 25 amino acids, 20 amino acids, 15 amino acids, 10 amino acids, or 5 amino acids. In some embodiment, the miniprotein comprises at least one disulfide bridge. In some embodiments, the miniprotein specifically binds to a target. In some embodiments, the composition displays mm or nM binding affinity to the target in an in vitro assay. In some embodiments, the miniprotein a binding affinity of 1 pM to 100 nM to Nectin-4 on a cell line expressing human Nectin-4. In some embodiments, the composition binds to the target with an affinity of 100 pM to 10 nM, e.g., as measured by an in vitro binding assay. In some embodiments, the composition is characterized in that it has high tissue penetrating properties relative to a composition comprising a full-size protein that binds to the same target. In some embodiments, the miniprotein binding to the target modulates biological function. In some embodiments, administration of the composition does not elicit an immune response. In some embodiments, the immune response includes a systemic immune response or a local immune response. In some embodiments, the target is located in, on, or near a cell. In some embodiments, the target is a protein expressed on the surface of the cell. In some embodiments, the cell is a tumor cell. In some embodiments, the tumor cell is a solid tumor cell. In some embodiments, the cell is a human cell. In some embodiments, the target is Nectin-4. In some embodiments, the subject has or is at risk of having cancer. In some embodiments, the cancer is selected from breast cancer, ovarian cancer, melanoma, pancreatic cancer, peripheral neuroma, glioblastoma, adrenocortical carcinoma, AIDS-related lymphoma, anal cancer, bladder cancer, meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, male breast cancer, malignant mesothelioma, medulloblastoma, Merkel cell carcinoma, metastatic squamous neck cell carcinoma, multiple myeloma and other plasma cell neoplasms, mycosis fimgoides and the Sezary syndrome, myelodysplastic syndromes, nasopharyngeal cancer, neuroblastoma, non-small cell lung cancer, small cell lung cancer, head and neck cancer, skin cancer, oropharyngeal cancer, bone cancers, including osteosarcoma and malignant fibrous histiocytoma of bone, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors. In some embodiments, after administration of the composition, the cancer is treated. In some embodiments, the composition is administered intravenously or subcutaneously.

In some embodiments, the present disclosure provides methods of characterizing miniprotein conjugates comprising contacting a population of cells expressing Nectin-4 with a miniprotein conjugate and measuring one or more of: (i) binding affinity; (ii) internalization; (iii) binding specificity; (iv) immune response as characterize by secretion or expression of one or more cytokines.

In some embodiments, the present disclosure provides methods of detecting cancer comprising administering to a subject a composition comprising a Nectin-4-specific miniprotein, further comprising a detectable moiety, and detecting the presence and/or quantity of the composition in the subject, wherein detection of the miniprotein is associated with risk of developing or having cancer. In some embodiments, the miniprotein of the composition is designed for conjugation to one or more additional components. In some embodiments, the composition penetrates tumor tissue. In some embodiments, the miniprotein of the composition comprises less than 12 kDa. In some embodiments, the miniprotein of the composition comprises no more than about 100 amino acids or less, 90 amino acids, 85 amino acids, 80 amino acids, 75 amino acids, 70 amino acids, 65 amino acids, 60 amino acids, 55 amino acids, 50 amino acids, 45 amino acids, 40 amino acids, 35 amino acids, 30 amino acids, 25 amino acids, 20 amino acids, 15 amino acids, 10 amino acids, or 5 amino acids. In some embodiments, the miniprotein of the composition comprises at least one disulfide bond. In some embodiments, the miniprotein that does not comprise multiple cysteine residues such as, for example, a miniprotein comprising a single cysteine residue. In some such embodiments, the miniprotein may form a dimer, such as with another miniprotein (e.g., self-dimerization). In some embodiments, two miniproteins are linked together to form a dimer. In other embodiments, two miniproteins are each linked to a linker to form a dimer. In other embodiments, two different miniproteins are each linked to a linker to form a dimer

In some embodiments, the method identifies a subject as having a cancer. In some embodiments, the cancer is selected from breast cancer, ovarian cancer, melanoma, pancreatic cancer, peripheral neuroma, glioblastoma, adrenocortical carcinoma, AIDS-related lymphoma, anal cancer, bladder cancer, meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, male breast cancer, malignant mesothelioma, medulloblastoma, Merkel cell carcinoma, metastatic squamous neck cell carcinoma, multiple myeloma and other plasma cell neoplasms, mycosis fimgoides and the Sezary syndrome, myelodysplastic syndromes, nasopharyngeal cancer, neuroblastoma, non-small cell lung cancer, small cell lung cancer, head and neck cancer, skin cancer, oropharyngeal cancer, bone cancers, including osteosarcoma and malignant fibrous histiocytoma of bone, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors. In some embodiments, a cancer cell from the subject expresses Nectin-4, or a portion thereof. In some embodiments, the expression of the target is higher in the cancer cell than in a non-cancer cell.

In some embodiments, the present disclosure provides methods of targeting a population of cancer cells expressing Nectin-4, the method comprising: (i) determining a level of expression of a target in a population of cancer cells; (ii) administering to a subject in need thereof a composition according to the present disclosure or a pharmaceutical composition according to the present disclosure, wherein the composition specifically binds Nectin-4; and (iii) wherein the composition targets the Nectin-4-expressing cells and is internalized into the Nectin-4 expressing cells; (iv) wherein the patient is treated after the administering as compared to prior to the administering and (v) wherein the treatment preferentially damages cells expressing Nectin-4.

In some embodiments, a composition provided herein preferentially damages (e.g., to a greater extent, e.g., kills) cells expressing Nectin-4 as compared to cells not expressing Nectin-4. In some embodiments, a composition provided herein does not damage cells not expressing Nectin-4.

A miniprotein conjugate comprising: (i) miniprotein (M) that specifically binds to Nectin-4; (ii) a chelator (C) conjugated to (M) through an optional linker (L), wherein (C) comprises DOTA, and (L), when present, comprises PEG; and (ii) a radionuclide (R) chelated to (C), wherein (R) is Actinium-225.

A miniprotein conjugate comprising: (i) miniprotein (M) that specifically binds to Nectin-4; (ii) a chelator (C) conjugated to (M) through an optional linker (L), wherein (C) comprises DOTA, and (L), when present, comprises PEG; and (ii) a radionuclide (R) chelated to (C), wherein (R) is Indium-111.

A miniprotein conjugate comprising: (i) miniprotein (M) that specifically binds to Nectin-4; (ii) a chelator (C) conjugated to (M) through an optional linker (L), wherein (C) comprises DOTA, and (L), when present, comprises PEG; and (ii) a radionuclide (R) chelated to (C), wherein (R) is Lead-212.

A miniprotein conjugate comprising: (i) miniprotein (M) that specifically binds to Nectin-4; (ii) a chelator (C) conjugated to (M) through an optional linker (L), wherein (C) comprises DOTA, and (L), when present, comprises PEG; and (ii) a radionuclide (R) chelated to (C), wherein (R) is Lutetium-177.

A miniprotein conjugate comprising: (i) miniprotein (M) that specifically binds to Nectin-4; (ii) a chelator (C) conjugated to (M) through an optional linker (L), wherein (C) comprises DOTA, and (L), when present, comprises PEG; and (ii) a radionuclide (R) chelated to (C), wherein (R) is Gallium-68.

In some embodiments, the PEG linker is PEG (2-24).

In some embodiments, the present disclosure provides pharmaceutical compositions comprising a conjugate in accordance with the present disclosure and a pharmaceutically acceptable excipient. In some embodiments, the composition is formulated for parenteral or oral administration.

In some embodiments, the present disclosure provides methods of imaging a cell or population of cells in a subject having or suspected of having cancer comprising administering a pharmaceutical composition in accordance with the present disclosure and detecting a presence of the pharmaceutical composition in the subject.

In some embodiments, the present disclosure provides methods of treating cancer in a subject in need thereof comprising administering a pharmaceutical composition in accordance with the present disclosure to the subject, wherein the subject is treated and non-cancer cells of the subject are not killed.

The present disclosure is further illustrated by the following examples which should not be construed as further limiting. The contents of all references cited throughout this application are expressly incorporated herein by reference.

### Examples

### Example 1: Screening Scaffold Libraries to Identify Nectin-4 Specific Miniproteins

This Example describes exemplary library screening for initial identification of miniproteins that bind to Nectin-4 or a portion or fragment thereof.

### 1.]Using a Phage Display Library

A panning experiment was carried out to bind a phage displayed miniprotein scaffold to a Nectin-4 target linked to a solid support. Tissue culture grade plates were coated with recombinant Nectin-4 and a buffer. Through the first round of panning, colonies were allowed to bind on the plate for 2 hours at 37° C. The plate was washed ˜20 times, and the bound phage were eluted with HCl. The bound phage was used to infect E. coli cells to produce additional phage used in additional rounds of panning. To measure the binding of the phage to Nectin-4, an ELISA assay was performed. The positive clones were sequenced.

### 1.2 Using Yeast Surface Display

A miniprotein polypeptide library was generated and genetically fused to the yeast mating agglutinin protein Aga2p. It attaches by two disulfide bonds to Aga1p, a yeast cell wall protein. Expression of this construct was put under the control of a galactose-inducible promoter and the N-terminal of the miniprotein polypeptide was fused to Aga2.

### 1.3 Library Construction

To construct a miniprotein library, mutants were created by varying disulfide-constrained CDP loop regions. The library included variations in the loop length along with sequence diversity. In some cases, a larger surface contact area was necessary to achieve high-affinity binding to the Nectin-4 target. To mutate any miniprotein loop regions at the DNA level, degenerate codons were introduced by oligonucleotide assembly using overlap extension PCR. The degenerate portion was flanked by sufficient sequence homology with neighboring oligonucleotides to design primers to allow for specific annealing. The genetic material was amplified with flanking primers that had sufficient overlap with the yeast display vector for homologous recombination in yeast.

Two-color FACS was used for yeast library screening, using APC and FITC fluorophores. The c-myc epitope was measured using APC and the other (FITC) measured the interaction of the miniprotein mutant against the Nectin-4 target. Nectin-4 is incubated with the yeast library and allowed to come to equilibrium prior to FACS sorting.

### Example 2: Engineering Miniproteins for Improved Nectin-4 Binding

This Example describes exemplary engineering of miniproteins that bind to Nectin-4 to achieve, among other things, improved binding affinity to Nectin-4 or a portion or fragment thereof as compared to proteins that are not engineered as provided herein.

To improve the binding affinity of the engineered miniproteins to Nectin-4, an SAR approach was used, wherein, various amino acid residues within the engineered structure were replaced with optimal substitutions, resulting in improved binding affinity. These substitutions included natural and non-natural amino acids, conjugated chemical moieties, and other small molecule attachments. Chemical crosslinking was used to provide proper structural conformation and stability, which are key features to retention of binding affinity. Miniproteins in accordance with the present disclosure can be generally characterized by having small disulfide-rich peptide scaffolds and can have difficulty folding. Using techniques known to those of ordinary skill in the art, optimized conditions for folding and purification via reverse-phase HPLC were developed to ensure final compounds with correct structure, conformation, and purity. Improved miniproteins were generated and are provided herein (see, e.g., Tables 2A and 2B).

### Example 3: Direct Binding Assay Against Nectin-4

This Example describes exemplary binding assays of miniproteins that bind to Nectin-4.

A direct binding assay was utilized to measure the affinity of miniproteins to the Nectin-4 expressed on the surface of mammalian cells. An equilibrium binding constant was measured (KD) using a miniprotein conjugated to a fluorophore or radioisotope. A miniprotein that possessed an N- or C- terminal epitope tag for detection by a labeled antibody was also used in an assay such as performed herein.

For the direct binding assay utilizing fluorescence, aliquots of mammalian cells expressing Nectin-4 were pelleted in polypropylene tubes. The cells were resuspended in an appropriate binding buffer with a range of miniprotein concentration of at least 100-fold above and below the expected KD (e.g., one set of dilutions for each miniprotein being tested). The culture media contained BSA to prevent nonspecific binding, and binding was measured at several volumes ranging from 50 L to 5 mL. Cells expressing Nectin-4 were incubated with Nectin-4-specific miniproteins until equilibrium was reached (several hours). Miniproteins labeled with an N- or C-terminal epitope tag were washed and resuspended in fluorescently labeled anti-epitope tag antibody. After incubation, cells were analyzed with a flow cytometer and the mean fluorescence signal at each miniprotein concentration was determined using data analysis software. For the negative control, the assay was performed with cells that did not express Nectin-4. Expression of Nectin-4 was confirmed using commercially available antibodies. In order to differentiate between live and dead cells to properly analyze the data, instrument scatter patterns or propidium iodide were used to prevent nonspecific binding of dead cells. Moore, Sarah J., and Jennifer R. Cochran. “Chapter Nine - Engineering CDPs as Novel Binding Agents.” Methods in Enzymology, edited by K. Dane Wittrup and Gregory L. Verdine, vol. 503, Academic Press, 2012, pp. 223-51. ScienceDirect, doi:10.1016/B978-O-12-396962-0.00009-4.

### Example 4: Affinity Maturation of Miniproteins

This Example describes exemplary affinity maturation of miniproteins that bind to Nectin-4, including to enrich for and produce proteins that bind to Nectin-4 with higher affinity than proteins prior to affinity maturation.

Yeast codon optimized DNA encoding for a Nectin-4 miniprotein (e.g., cysteine-dense polypeptide, knottin-protein, binder, affibody, engineered Kunitz domain, monobody, anticalin, designed ankyrin repeat domain (DARPin), or avimer) sequences was amplified using an error prone DNA polymerase to introduce random mutations. This amplified pool of DNA was transformed into yeast for cell surface display of the mutated Nectin-4 miniprotein(s).

The yeast library displaying the miniprotein variants was then subjected to cell sorting. Cell sorting was first performed using magnetic based approaches and then subsequently by fluorescent-based sorting using flow cytometry/FACs analysis.

In each round, the yeast was incubated with Nectin-4 protein at decreasing concentrations and the yeast bound to Nectin-4 were isolated. Subsequent rounds of sorting were performed to enrich for higher affinity miniproteins. Individual yeast clones harboring a single peptide miniprotein sequence were isolated after multiple round of selection. Plasmid DNA was isolated and Sanger sequencing was performed to determine the affinity matured miniprotein sequence.

Resulting engineered Nectin-4 miniproteins developed using this assay were synthesized and have amino acid sequences as set forth in SEQ ID NOs: 1-158 or 177 (see Table 2A).

### Example 5: Synthesis of Nectin-4 Miniprotein-Peg-Dota Conjugates

This Example describes synthesis of exemplary Nectin-4 miniprotein-PEG-DOTA conjugates, which can also be labeled with radiolabels or cold-metal surrogates thereof.

### Synthesis of Polypeptide

Polypeptides were synthesized on a peptide synthesizer, such as a Prelude peptide synthesizer (Protein Technologies Inc., Tucson, AZ)) by solid-phase methods using Fmoc strategy with N-[(dimethylamino)-1H-1,2,3-triazolo-[4,5-b]pyridin-1-ylmethylene]-N-methylmethanaminium hexafluorophosphate N-oxide (HATU) or 2-(6-chloro-l —H-benzotriazole-1-yl)-1,1, 3,3-tetramethylaminium hexafluorophosphate (HCTU) activation (5-fold molar excess to amino acid) in N,N-dimethylformamide (DMF), and N′N-diisopropylethylamine (DIEA) was used as a base. A 20% piperidine/DMF solution was used for Fmoc deprotection. The resin was Rink Amide MBHA LL (Novabiochem) with loading of (0.30-0.40) mmol/g on a (20-400) pmol scale. Final deprotection and cleavage of the peptide from the solid support was performed by treatment of the resin with (92.5% TFA, 2.5% phenol, 2.5% water and 2.5% triisopropylsilane) for 2-3 hours.

The cleaved peptide was precipitated using cold diethyl ether. The diethyl ether was decanted, and the solids were triturated with cold diethyl ether and pelleted by centrifugation. The crude solids were dissolved in a 1:1 solution of ACN/water, 0.01% TFA. Disulfide bridge formation was accomplished via orthogonally protected cysteine residues and after selective deprotection and/or natural folding cyclization, the crude product solution was lyophilized in preparation for final purification. The lyophilized solid was re-dissolved in a 1:1 solution of acetonitrile/water, with 0.1% TFA (10⁻¹⁵ mL), purified via reverse phase HPLC on a Waters XBridge™ BEH 130, CIS, 10 μm, 130 A, 30×250 mm ID column, using a 30 gradient within the ranges of 5-75% acetonitrile/water with 0.1% TFA over 30-45 minutes at a flow rate of 30 mL/min, k −215 nm.

### Conjugation of Chelator to Polypeptide

Metal-chelating motifs were covalently attached to polypeptide chains at the end of the linear SPPS. First, orthogonally protected linkers (e.g., amino-discrete polyethylene glycols, amino-hexanoic acids, and amino acids;) were coupled to either the N-terminal amine, or alternatively the &-amino side chain of a lysine. After an appropriate deprotection step (20% piperidine in dimethylformamide for FMOC), metal chelators were coupled to the linker-polypeptide chain with conventional HATU activation in DMF containing 2% N,N-diisopropylethylamine (DIEA). Chelators such as tri-t-butyl-DOTA, bis-t-butyl-NOTA, tetra-t-butyl-DTPA, or CROWN were coupled to the linker also using HATU activation in DMF containing 2% N,N-diisopropylethylamine (DIEA). Finally, polypeptides were cleaved from solid support and acid-labile protecting groups were removed with a cocktail of TFA.

Formation of Metal Complex with Polypeptide

Peptides with covalently attached metal chelators were complexed with either their cold-metal surrogates (e.g., indium, lanthanum, gallium, copper, or europium) or radionuclides (e.g., 111-In or 225-Ac). See, e.g., Table 2A. Metalation reactions were carried out in slightly alkaline aqueous buffers. Peptides were dissolved in buffers comprised of 100 mM ammonium acetate, pH 7-8 with 3 molar equivalents of metal. Additional weakly coordinating formulants were also used to avoid oxidation of some metals. Metalation reactions were monitored by RP-HPLC under neutral mobile phases such as 10 mM triethylammonium acetate as a modifier. Ultra-violet signals were used to identify analytes and their shifting retention times were indicative of metalated peptides. Elevated temperatures of 60-90° C. were used, as appropriate, for efficient complexation. After the completion of the metal complexation, excess metal was separated from metalated peptide by semi-prep HPLC under neutral conditions. Mass spectroscopy was used to confirm the metalated peptide species. Radioactive species were characterized for radiochemical yield and purity with either radio-HPLC or radio-TLC.

### EXAMPLE 6A: RADIOLABELING OF NECTIN-4-PEG-DOTA with Ac²²⁵

This Example describes radiolabeling of exemplary Nectin-4 miniprotein-PEG-DOTA conjugates with Ac-225.

For radiolabeling reaction, Nectin-4 miniprotein-PEG-DOTA conjugates (see, e.g., Table 2A) were incubated with 1 to 1 molar ratio of Ac225 (in the form of a nitrate salt) in 0.1 N sodium acetate (pH 5) for 5 hrs at 70° C. The reaction was terminated with the addition of EDTA. Ac225-CDP-PEG-DOTA was purified using a PD-10 column (Amersham Biosciences/GE Healthcare) and eluted with PBS (pH 7.4), and passed through a 0.22 μm filter. The radiochemical purity was determined as the ratio of the main product peak to other peaks. The radiochemical yield was determined as the ratio of final activity of the product over the starting activity used for the reaction adjusted for the radioactive decay.

### Example 6B: Radiolabeling of Nectin-4-Peg-Dota with In¹¹¹

This Example describes radiolabeling of exemplary Nectin-4 miniprotein-PEG-DOTA conjugates with In-111 (see, e.g., Table 2A for exemplary PEG-DOTA conjugates; note that In-PEG-DOTA conjugates disclosed in Table 2A, such as, e.g., C110, etc. are labeled using natural abundance Indium, which can be a combination of 113-In and 115-In, and may be seen, for example, as ^{null}In. Radioactive Indium is used to label PEG-DOTA conjugates such as, e.g., C109, etc.)

For radiolabeling reaction, Nectin-4 miniprotein-PEG-DOTA conjugates were incubated with 1 to 1 molar ratio of In-111 (see, e.g., “PEG-DOTA” compounds set forth in Table 2A). The reaction was terminated with the addition of EDTA. The conjugate was purified using a PD-10 column (Amersham Biosciences/GE Healthcare) and eluted with PBS (pH 7.4), and passed through a 0.22 μm filter. The radiochemical purity was determined as the ratio of the main product peak to other peaks. The radiochemical yield was determined as the ratio of final activity of the product over the starting activity used for the reaction adjusted for the radioactive decay.

### Example 7A: Peptide Binding Assay to Nectin-4 Via Surface Plasma Resonance (Spr)

This Example describes SPR analysis to measure Nectin-4 miniprotein binding affinity.

Peptide binding affinity to the target protein was measured by SPR using a Biacore T200 instrument. The target protein was covalently coupled to a Biacore chip surface using free amine-coupling chemistry (NHS/EDC) and all uncoupled sites were blocked with ethanolamine. Free peptides were flowed over the target protein surface in increasing concentrations to measure association (K_{A}) and dissociation (K_{D}) to calculate the equilibrium dissociation constant (K_{D}) using an appropriate binding model (see, e.g., Nikolas Stroth, J Biol Methods. 2016; 3 (1)).

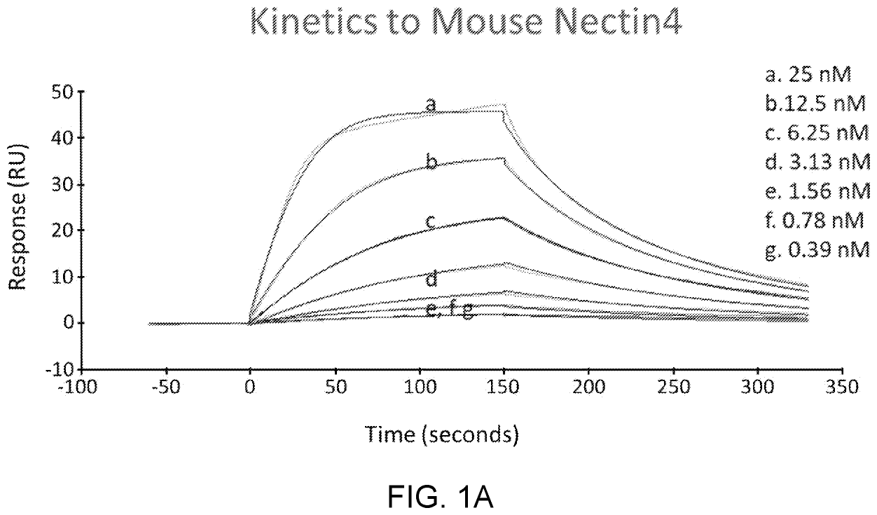

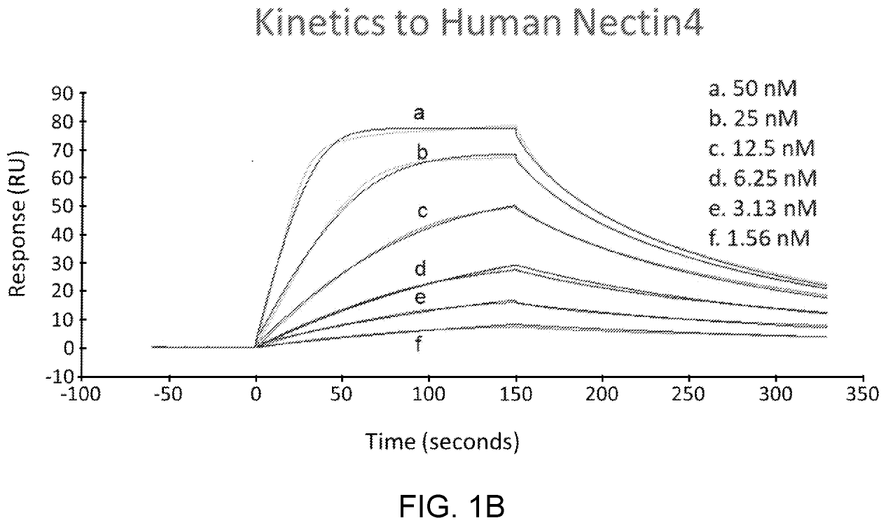

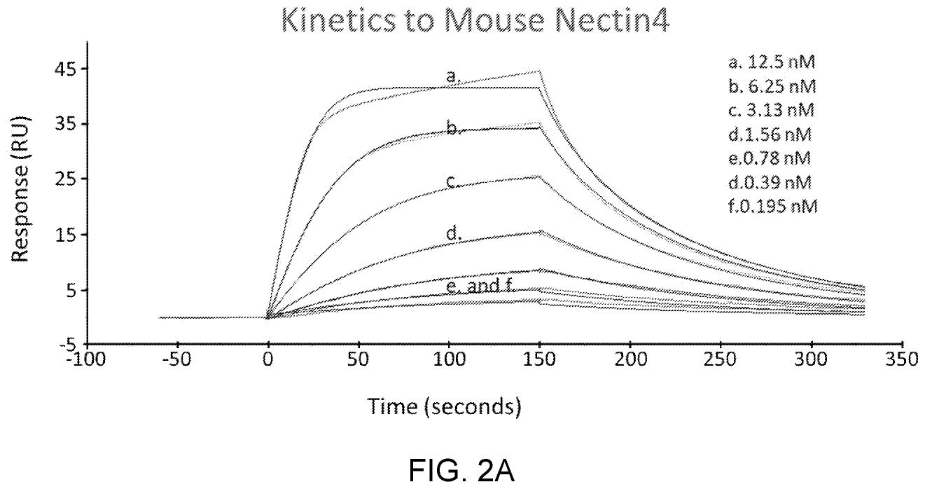

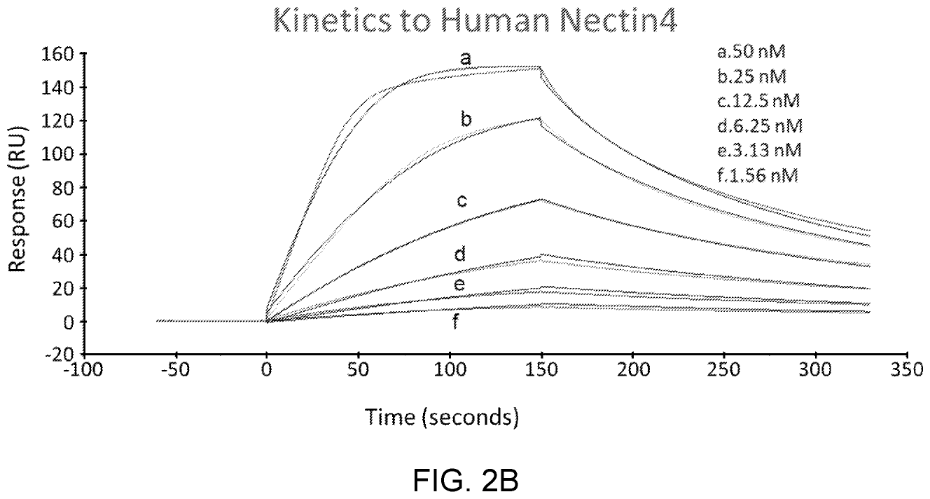

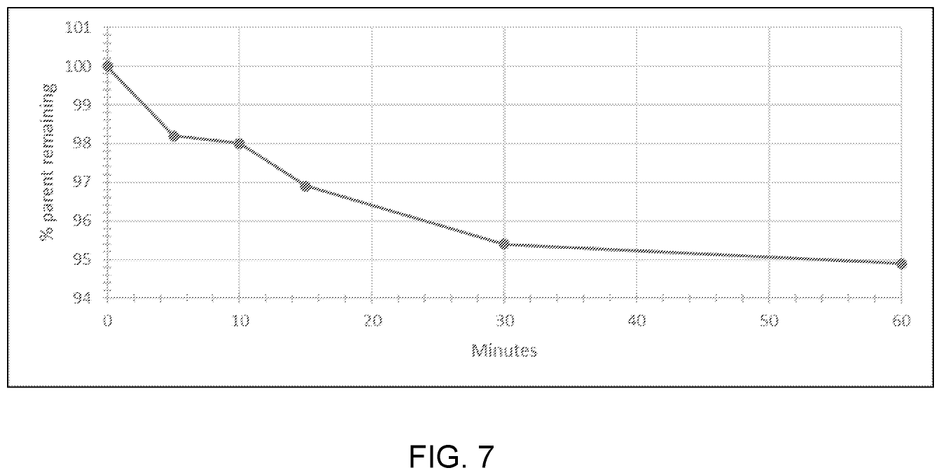

FIGS. 1A and 1B show BIACORE data demonstrating the affinity of an exemplary Nectin-4 compound, C9, with a miniprotein with an amino acid sequence according to SEQ ID NO: 5 and Table 2A. Table 2B shows BIACORE data demonstrating the affinity of an exemplary Nectin-4 compound, C12, with a miniprotein with an amino acid sequence according to SEQ ID NO: 5. In this example, affinity for mouse Nectin-4 (FIGS. 1A and 2A) and human Nectin-4 (FIGS. 1B and 2B) were measured using a 1:1 binding model at twenty-five degrees Celsius. In all figures (FIGS. 1A, 1B, 2A, and 2B), lines on graphs are labeled from top to bottom, beginning with letter “a,” and the corresponding concentration is noted to the right of the figure, next to the letter designating each line. As demonstrated by the BIACORE measurements, C9 had a K_{D} of 7.825 nM (Chi²=0.130; k_{d}=0.02134 s⁻¹) for mouse Nectin-4 (FIG. 1A) and 7.496 nM (Chi²=0.560; k_{d}=0.02271 s⁻¹) for human Nectin-4 (FIG. 1B) and C12, had a K_{D} of 2.942 nM (Chi²=0.230; k_{d}=0.02625 s⁻¹) for mouse Nectin-4 (FIG. 2A) and 10.5 nM (Chi²=2.68; k_{d}=0.01697 s⁻¹) for human Nectin-4 (FIG. 2B).

### EXAMPLE 7B: PEPTIDE BINDING ASSAYS

This Example describes peptide binding assays using Nectin-4 miniproteins and conjugates thereof.

Surface plasmon resonance (SPR) analyses were performed using Biacore T200 (GE Healthcare). Target protein (“ligand”) was immobilized to a CM5 Series S sensor chip (Cytiva) using amine-coupling chemistry. Running buffer was 0.05% Surfactant P20 in HEPES buffered saline (HBS-P), pH 7.4 (Cytiva). Immobilization was performed at 25° C. Carboxyl groups in each flow cell were activated using a 1:1 mixture of 0.4 M 1-ethyl-3-(3-dimethylaminopropyl)-carbodiimide in water (EDC) and 0.1 M N-hydroxysuccinimide in water (NHS) for 7 minutes. The ligand was prepared in 10 mM Sodium acetate pH 5 (Cytiva). Pulsatile injections were made to target a specific immobilization level. Any remaining amine-reactive esters on the chip surface were blocked with a 7-minute injection of 1 M ethanolamine, pH 8.5. Three 10-second pulses of 50 mM Sodium hydroxide were then made to remove any unbound ligand. Reference cells were modified using amine coupling chemistry, omitting ligand injection.

Exemplary compounds set forth in Table 2A (“analyte”) were injected to the prepared ligand surface and blank reference surface in increasing concentrations to cover 0.1-10× the expected KD range at 25° C. Increase in signal (RU) is proportional to an increase in binding events between the analyte and ligand surface. The injection was then stopped, and dissociation was measured. The resulting sensorgram has reference surface and blank injection subtracted prior to curve fitting. Measured association (kₐ, 1/M^{s-1}) and dissociation (k_{d}, s⁻¹) were fit to a 1:1 binding model to calculate an equilibrium dissociation constant, K_{D}(M) for each analyte. Equilibrium binding constants (KD) and binding inhibition constants (Ki) estimated from surface plasmon resonance (SPR) or DELFIA® binding assays for exemplary compounds as set forth in Table 2A.

### Example 8A: Binding Affinity Assay of Radiolabeled Conjugates

This Example describes measurements of binding affinity using radiolabeled Nectin-4 miniproteins, cold-labeled surrogates, and conjugates thereof.

To measure binding affinity of miniproteins conjugated to Ac225, La138, and DOTA to the N-terminus to determine if the miniprotein affects Nectin-4 binding specificity, a binding assay as provided in Example 3 was performed. The positive control was a Nectin-4 miniprotein not conjugated to DOTA and Ac²²⁵ (or its cold metal surrogate). It was expected that the miniprotein alone would bind to Nectin-4. Binding affinity with cells that do not express Nectin-4 was also performed, as a negative control.

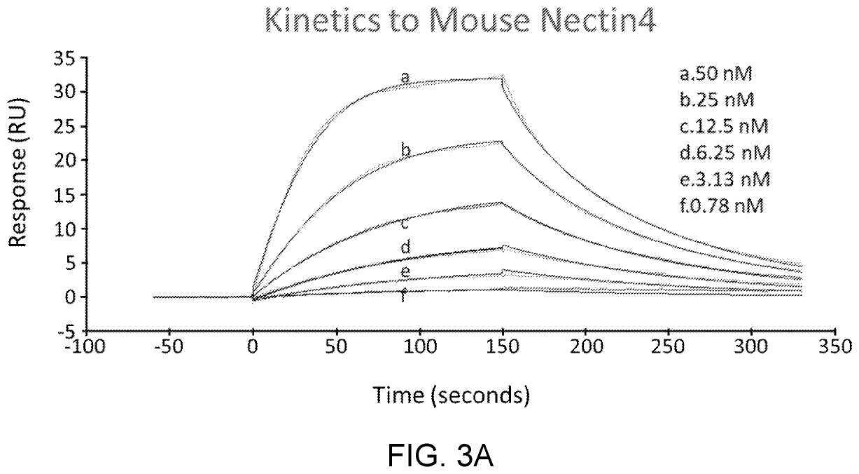

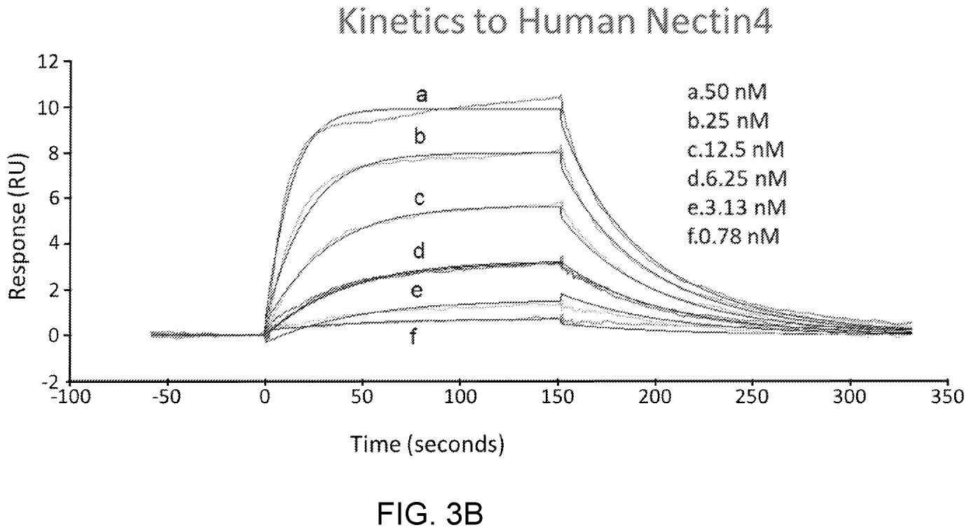

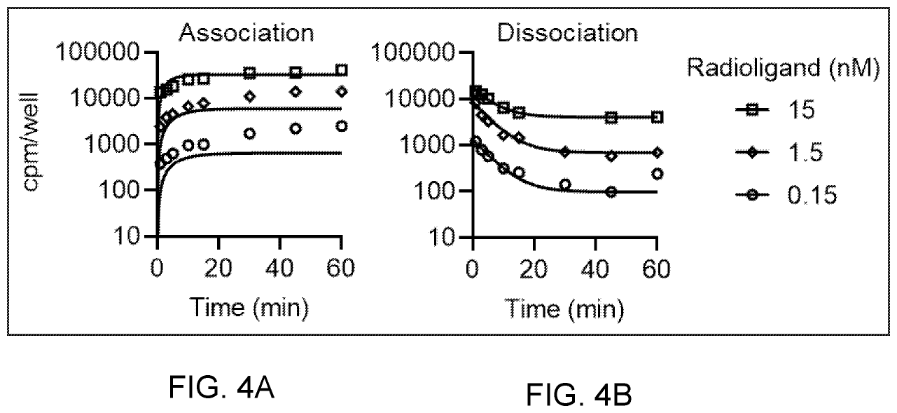

FIGS. 3A and 4B show BIACORE data demonstrating the affinity of an exemplary Nectin-4 compound, C91, (i.e., a conjugate comprising a Nectin-4 peptide of SEQ ID NO: 5 and arranged having an N-terminus comprising ¹³⁸La-DOTA-PEG4 and a C-terminus comprising OH). In this example, affinity for mouse Nectin-4 (FIG. 3A) and human Nectin-4 (FIG. 3B) were measured using a 1:1 binding model at twenty-five degrees Celsius. In both of FIGS. 3A and 3B, lines on graphs were labeled from top to bottom, beginning with letter a, and the corresponding concentration was noted to the right of the figure. As demonstrated by the BIACORE measurements, C91 had a K_{D} of 24.65 nM (Chi²=0.0634; k_{d}=0.02817 s⁻¹) for mouse Nectin-4 (FIG. 3A) and 17.91 nM (Chi²=0.0281; k_{d}=0.02966 s⁻¹) for human Nectin-4 (FIG. 3B).

### Example 8B: Radioligand Binding Assays

Radioligand binding was performed in HT1376 urothelial cancer cells by method of saturation. Cells were seeded at 20,000 cells per well in 96-well format 48 hours prior to assay. Lutetium-177-labeled radioconjugates of C215 and C117 were incubated with cells at 4 deg C. for 1 h at various increasing concentrations of radioligand. At the end of incubation cells were washed three times with PBS and radioactivity was quantified by gamma counting as shown in Table 3.

TABLE 3

| Compound | Kd (nM) | Confidence Interval (CI) |
|---|---|---|
| C215 | 4.24 | 3.64 to 4.93 |
| C117 | 0.89 | 0.79 to 1.03 |

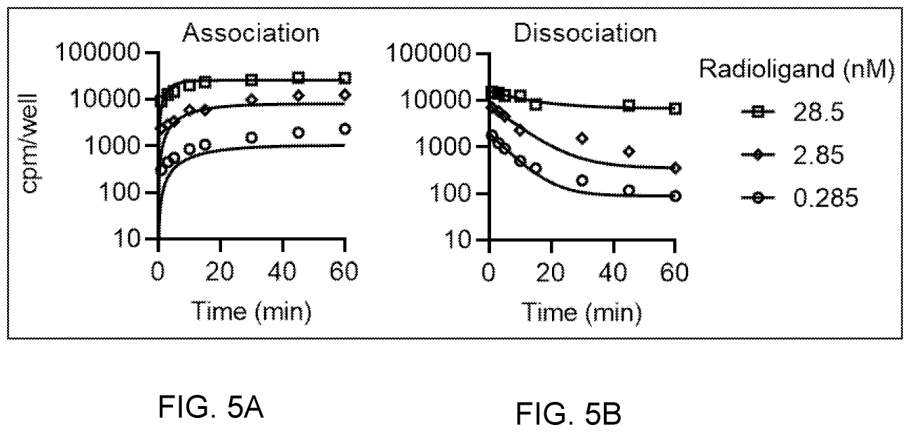

Radioligand binding was performed in HT1376 urothelial cancer cells by method of kinetic analysis. Cells were seeded at 20,000 cells per well in 96-well format 48 hours prior to assay. On the day of assay culture medium was removed and PBS was added to cells. Association kinetics were measured at three concentrations of radioligand and this was performed by the addition of lutetium-177 radioconjugates added to cells at various time points from 1 to 60 minutes. Dissociation kinetics were measured by method of forced dissociation at three concentrations of radioligand by first incubating cells with radioligand until equilibrium was reached at 1 hour, followed by the addition of 1000 times excess non-radioactive competitor ligand at time points from 1 to 60 minutes. Data (as shown in Table 4) were fitted for association (FIG. 4A and FIG. 5A) and dissociation (FIGS. 4B and 5B) by global fit for all concentrations evaluated and the association rate, dissociation rate, and constant were determined.

TABLE 4

| Compound (PEG-DOTA prior to | | Confidence |
|---|---|---|
| radiolabeling) | Kd (nM) | Interval (CI) |
| C215 | 5.01 | 4.17 to 6.08 |
| C82 | 14.08 | 0.78 to 1.03 |
| C214 | 3.03 | 2.42 to 3.77 |
| C109 | 8.72 | 5.2 to 15.86 |
| C117 | 2.64 | 1.73 to 3.9 |

### Example 9: ¹¹¹In Labeling of Miniproteins for Use in Biodistribution Assays

This Example describes radiolabeling of exemplary compounds for use in biodistribution assays.

In-111 labeling of miniproteins with MES buffer: In-111 was neutralized with 0.5 M MES buffer pH 5.5. This mixture was added to miniproteins prepared at 2 mg/mL in water with an equivalent amount of 0.5 M MES buffer pH 5.5, in a 1.5 mL Eppendorf vial and heated at 37° C. for 60 minutes. After the reaction, an HPLC sample was taken and an equivalent amount of 10 mM DTPA in 0.1 M ammonium acetate pH 5 was added and incubated for at least 15 minutes. The sample was then used for HPLC analysis.

In-111 labeling of miniproteins with Sodium Acetate buffer: In-111 was neutralized with 0.1 M sodium acetate buffer pH 5. This mixture was added to a vessel with an exemplary compound, C188 (which has a miniprotein of SEQ ID NO: 136), prepared at 5 mg/mL in sodium acetate buffer, with various amounts of 0.1 M sodium acetate buffer pH 5, in a 1.5 mL Eppendorf vial and heated at 60° C. for 45 minutes. After the reaction, an HPLC sample was taken and an equivalent amount of 10 mM DTPA in 0.1 M ammonium acetate pH 5 was added and incubated for at least 15 minutes. The sample was then used for HPLC analysis.

Purification of ¹¹¹In-miniproteins: Purification was done on two of the crude reaction mixtures mixed with 10 mM DTPA in 0.1 M ammonium acetate pH 5 and incubated for 15 minutes. A 3 kDa 0.5 mL Amicon filter was used for purification and the filter was spun at 15,000 RCF for 9 minutes. Saline was used as a formulation buffer.

### Example 10: In Vivo Biodistribution Involving ¹¹¹In-Labeled Miniproteins

This Example describes biodistribution assays using radiolabeled miniproteins and conjugates thereof as provided herein.

Animals: Female athymic nude mice (6-8 weeks of age) were purchased from Charles River Laboratories and housed according to IACUC guidelines with ad libitum feeding. Biodistribution imaging experiments were performed on both null and tumor bearing athymic nude mice. Tumor xenograft models were generated by inoculating mice subcutaneously with 3×10⁶ HT1376 cells, in 200 μL (50:50 PBS/Matrigel) in the right shoulder or right flank. Tumors were monitored for 14 days prior to SPECT/CT imaging dates. Mice with tumor volumes between 150 mm³ and 250 mm³ were selected for study inclusion and randomized to treatment arms. An excess of 60% of required imaging study mice were inoculated with tumor cells to ensure enough mice with appropriate tumor ranges were generated.

Animal grouping and Treatment: Animals were monitored for body weight bi-weekly and at the time of experimentation, grouped into groups of n=3. Animals were administered test-agents (TAs) that were prepared as described above. Animals received one of two standard injected dose activates and mass dose injections, depending at which site imaging studies were performed.
Site-1: 350 μCi of activity (¹¹¹In) at approximately 3-5 g of total peptide per mouse.
Site-2: 30MBq (810 Ci) of activity (¹¹¹In) at approximately 1 nM (4-6 g) of total peptide per mouse.

Animal imaging: Animals were sedated using isoflurane gas and imaged in a 3 bed hotel using a NanoScan SPECT/CT scanner. Animals were imaged at timepoints between 10 mins and 72 h post-dosing via SPECT scan, followed by CT scans. Details of the image process and analysis are described in Example 11.

Humane endpoints: All animals were euthanized following the final imaging time point and carcasses were discarded according to IACUC protocols.

Results of the Biodistribution (BioD) assay with exemplary miniproteins are shown in Table 5. A relationship between target affinity and tumor uptake was observed, confirming that Nectin-4 miniprotein compounds specifically target and bind to tumors with high affinity and do not accumulate in non-target organs as compared to Nectin-4 miniproteins with less binding affinity.

TABLE 5
BioD Data for Exemplary Nectin-4 Miniprotein Compounds

| Compound Name | Data of BioD (+4 h Tumor - % ID/g) |
|---|---|
| C101 | 1.62 |
| C105 | 1.55 |
| C109 | 2.25 |
| C113 | 1.65 |
| C117 | 2.29 |
| C121 | 2.03 |
| C129 | 1.77 |
| C133 | 1.29 |
| C137 | 1.12 |

### Example 11: Image Analysis Methods

This Example describes imaging analysis methods for use with biodistribution assays such as in Example 10.

Image Processing: Images were generated as SPECT/CT pair with the SPECT reported in units of activity. Namely, the values assigned to the voxels (volume elements) comprising the SPECT images were in units of μCi. SPECT images were co-registered to the CT scan for anatomical reference, resampled to 0.2 mm³ voxels, masked to remove the CT background, and cropped to a uniform size prior to analysis.

Estimating tissue uptake: Regions of interest (ROIs) were defined using VivoQuant software. The kidneys and bladder were segmented as fixed volume phantoms and registered using the CT for anatomical reference. Two fixed volume spheres were used to create the liver ROI. Spheres were placed at appropriate anatomical locations based on CT and SPECT signal. Group and individual master spreadsheets were generated which included the volume, activity, and concentration (Activity/Volume) at each time point for each ROI generated. Additionally, plots of activity were generated using Matplotlib based python tools to highlight trends in the data. Outputs of each region were plotted and reported in percent injected dose per gram (% ID/g) and regions which were fully segmented were additionally presented in percent injected dose (% ID). Plots of body weights and tumor volumes measured manually in the lab were also created in the same manner.

Uptake unit definitions: Results were presented in units of percent injected dose (% ID) and percent injected dose per gram (% ID/g). The definition of these units can be found in the equations below: The % ID for each analyzed region from the in vivo imaging data can be defined as stated in Equation 1:

$$\% I D = \frac{Upta k e}{Injected Dose} * 100$$

where Uptake=Radioactivity (μCi) in a particular ROI, decay-corrected to the time of injection and imaging timepoint; and Injected dose=Radioactivity (μCi) injected into the subject.

The % ID/g for each analyzed region from the in vivo imaging data can be defined as stated in Equation 2:

$$\frac{ID}{g} = \frac{\frac{U p t a k e}{Injected Dose} * 100}{ROI weight}$$

where Uptake=Radioactivity (μCi) in a particular ROI, decay-corrected to the time of injection and imaging timepoint; Injected dose=Radioactivity (μCi) injected into the subject; and Weight=For in vivo, this is the volume of the particular ROI in mL.

Image generation: After the preprocessing described in A, individual maximum intensity projections (MIPs) were created with VivoQuant software for each subject at each time point and scaled in percent injected dose per gram (% ID/g). The CT for each image was windowed from −500 to 4500 Hounsfield Units (HU). The SPECT was windowed at various ranges to highlight different regions of uptake. Images were then stitched together using Image Magick based python tools to create montages of subjects over time and time points over groups.

ROIs for quantitative analysis were generated from CT scans in order to quantify the injected dose per gram (% ID/g) in tissues of mice as shown in Table 5 and Example 10. A relationship between binding affinity and tumor uptake was observed, showing that increased affinity for Nectin-4 resulted in increased tumor uptake. This supports specificity and improvement in targeting over miniproteins with lower binding affinity.

### Example 12: Plasma Pharmacokinetic Analysis in Sprague Dawley Rats

This Example describes plasma pharmacokinetic analysis of compounds provided herein using plasma analysis in Sprague Dawley rats.

To begin, double jugular vein-cannulated Sprague Dawley rats are dosed with bolus intravenous injections of miniproteins (0.03-0.3 mg/kg) and FITC-sinistrin. Nine blood collection timepoints are taken from 2-24 hours, processed to plasma with K2EDTA, and are frozen for subsequent bioanalysis. Plasma miniprotein concentrations are measured by LC-MS/MS. Plasma FITC-sinistrin is measured fluorometrically. All unknown sample measurements are interpolated against known authentic standards spiked into normal rat plasma to calculate concentration. Finally, non-compartmental analysis is performed to estimate plasma pharmacokinetic parameters.

Exemplary pK data for exemplary Nectin-4 miniprotein monomers were tested in vivo (t1/2 mins), showing an average pK for these exemplary compounds. Data for pK of a GFR control and previously published (see Challita-Eid et al Cancer Res (2016) 76 (10):3003-3013) measurements for enfortumab vedotin are also shown in Table 6.

TABLE 6

| Compound | PK DATA (t½ - min) |
|---|---|
| C102 | 28.9 |
| C110 | 26.8 |
| C217 | 31.4 |
| C216 | 31.4 |
| C97 | 27.3 |
| Compound Average | 29.2 |
| GFR Control | 26.7 |
| enfortumab vedotin | 2,476.8 (1.72 days) |

### Example 13A: Internalization Assay of Radiolabeled Conjugate

This Example describes internalization assays of radiolabeled conjugates of Nectin-4 miniproteins.

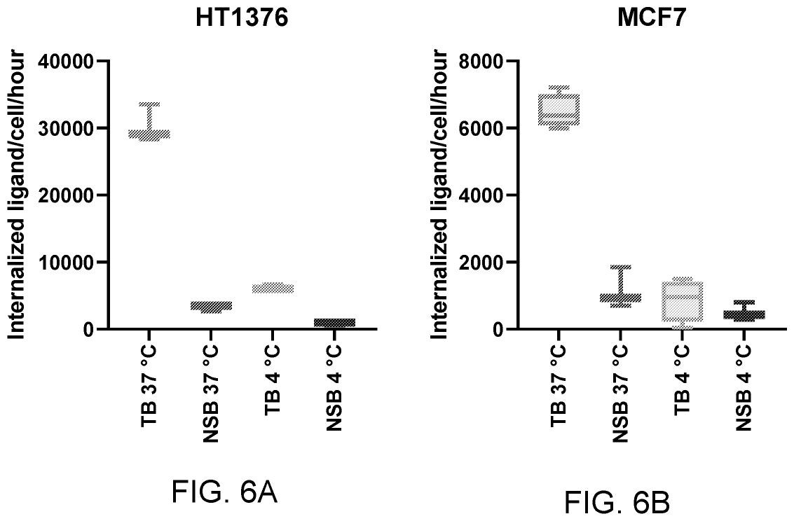

Internalization was assessed for indium-111 radiolabeled C215 in HT1376 and MCF7 cells. Cells were incubated in the presence of radioligand or radioligand plus 1000 times excess unlabeled compound (C215) at 37 deg C. or 4 deg C. for 1 h. At the end of incubation cells were washed four times with PBS and membrane bound radioligand was removed by three 0.5% trypsin washes. Cell suspensions were washed three times by method of centrifugation and removal of supernatant. Internalized radioactivity was measured by gamma counting cell pellets and cell number was determined by counting cells on an automatic cell counter. The binding assay confirmed that radiolabeled conjugates were successfully internalized into cells, as shown in FIGS. 6A and 6B, which depict internalization of an exemplary indium-111 radioconjugate, C215, in HT1376 (FIG. 6A) and MCF7 cell lines (FIG. 6B).

### Example 13B: In Vitro Cell Cytotoxicity Assay

Cytotoxicity to actinium-225 radioconjugates was assessed in HT1376 urothelial cancer cells. Cells were seeded in 96-well plates at densities of 250 to 1000 cells per well two days prior to treatment. On the day of treatment actinium-225 radioconjugates were diluted in culture medium to concentrations ranging from 0.0001 to 100 uCi/mL and treatment solutions were incubated with cells for 24 hours. At the end of the treatment period, the treatment solution was removed, and fresh culture medium was replenished for the regrowth period of the assay that lasted 4-5 days. At the end of the regrowth period the culture medium was removed and viability was assessed by CellTiter-Glo (Promega, Waltham MA) luminescent assay and the plates were read on a Perkin Elmer Enspire multimode plate reader. Data were normalized to treatment controls and plotted as non-linear sigmoidal dose response curves to determine the effective concentration for 50% reduction in cell viability. EC50 values for these exemplary radioconjugates are shown in Table 7.

TABLE 7

| Compound radiolabeled | EC50 (microcurie/milliliter) |
|---|---|
| with ²²⁵Ac | for Ac-225 radioconjugates |
| C215 | 0.086 |
| C214 | 0.014 |
| C109 | 0.029 |
| C113 | 0.042 |
| C117 | 0.014 |

This example shows thermal stability of an exemplary Nectin-4 miniprotein.

The thermal stability of an exemplary compound, C215, was analyzed at a concentration of 1 mg/mL at 75° C. in 0.9% Saline solution. A heating plate was set to 75° C. and the sample was heated for a period of one hour. The vial was shaken prior to collecting L aliquots at 5, 10, 15, 30 and 60 minute time intervals. The % parent remaining was analyzed by HPLC using the following system and method: Analytical Agilent 1100 HPLC system using a 4.6×250 mm, 300 Angstrom, BEH300, 5 m, Xbridge C18 column at 1.0 vmL/min. The solvent system consisted of A=Water+0.1% TFA, B=Acetonitrile+0.1% TFA. The gradient consisted of 5% to 70% B over 15 minutes, then the column was washed and equilibrated to initial conditions. C215 was determined to remain stable over 60 minutes (see FIG. 7).

### Example 15: In Vitro Cell Killing Assay

This Example describes in vitro cell killing assays using Nectin-4 miniproteins and conjugates thereof.

T47D cells, a breast cancer cell line, NCI-H358 cells, a human non-small cell lung cancer cell line, or SW780 cells, a human non-small cell lung cancer cell line, are incubated with serially diluted unconjugated miniprotein, miniprotein-Ac225 conjugate, or miniprotein-Lu177 conjugate. Cells are plated in black, 96-well Coming Costar 3603 plates at densities to allow for Log phase growth throughout the experiment (On Day 0, cells are allowed to settle and attach overnight). Cells are plated in triplicate for each concentration and controls examined. On Day 1, media is removed and replaced with RPMI-1640+10% Dialyzed FBS (Gibco 26400-044) with the indicated concentration of control non-conjugated miniprotein, miniprotein-Ac225 conjugate, or miniprotein-Lul77 conjugate (starting concentration 100 nM, serially diluted 1:3 to a final of concentration of 0.045 nM), 0.01% DMS0 or 1 μM Staurosporine as positive control for cell death. On Day 4 (72 hours after treatment) cells are analyzed by Cell Titer Glo (Promega G7573) per manufacturer's instructions. Plates are read in luminescence mode on a PheraStarFS plate reader. Data are collected, replicates averaged, and standard deviations were calculated and analyzed through nonlinear regression analysis with 4 parameters to generate IC50s for each cell line tested.

### Example 16: Comparison of Miniprotein Conjugate to Control

This Example describes comparisons of Nectin-4 miniproteins and conjugates versus known (control) conjugates that do or do not bind to Nectin-4.

PC-3 cells engineered to express Nectin-4 of human, monkey, or rat origin or SW780 urothelial carcinoma cells are incubated with serially diluted compounds as provided herein such as a miniprotein-Ac225 conjugate, a miniprotein-Lul77 conjugate, or MGC018 or enfortumab vedotin at 37C in 5% C02. Both MGC018 (which does not target Nectin-4) and enfortumab vedotin (which does bind to Nectin-4) serve as controls, as do non-radiolabeled Nectin-4 miniproteins as provided herein. The PC-3 cells at the different surface expression levels of 100,000, 30,000, 10,000, 3,000, 1,000, 300 copies of miniprotein per cell are used. Surface receptor density is determined by FACS analysis. Cell viability is measured after 5 days using AlamarBlue (Life Technologies). Percent survival is calculated as the number of live cells in treated wells divided by the number of live cells in control wells. The IC50 is derived from the survival curve using the sigmoid Emax nonlinear regression analysis function in GraphPad Prism (GraphPad).

### Example 17: Potency of Radiolabeled Conjugates

This Example describes measurement of potency of radiolabeled conjugates of Nectin-4 miniproteins.

### Measuring Potency of CDP-Ac Conjugates

The alkaline comet assay involves measurement of DNA damage in SSB and DSB. Similar to the binding assay of Example 3, aliquots of mammalian tumor cells expressing Nectin-4 are pelleted in polypropylene tubes. The cells are resuspended in an appropriate binding buffer with a range of miniprotein concentration of at least 100-fold above and below the expected KD. The culture media contains BSA to prevent nonspecific binding, and binding should be measured at several volumes ranging from 50 μL to 5 mL. The cells expressing Nectin-4 are incubated with the miniprotein-Actinium conjugate until equilibrium is reached, which will take several hours.

The cells incubated with the conjugate are embedded in a thin layer of agarose on a thin glass slide. The cells are lysed in a solution containing detergent and NaCl, releasing the DNA from the proteins bound to it, but leaving DNA fragments still attached to the nuclear membrane. Then, the plate is incubated in an alkaline solution, an electrophoresis is run and DNA is stained with ethidium bromide. DNA fragments travel to the anode forming a comet-like image when viewed by fluorescence microscopy. The image of the comet head denotes DNA content and the tail denotes frequency of DNA breaks. Software programs designed to analyze the comet image allow measurement of DNA content and tail length. The length of the comet tail correlates with the level of DNA damage. Tumor cells that do not express Nectin-4 are used for a negative control to measure DNA damage.

### Measuring DNA Damage Via H2AX Chromatin Staining

Cells expressing Nectin-4 are incubated with the miniprotein-Ac conjugate. After incubation, cells are fixed at different time points to study γ-H2AX induction and loss kinetics. Cells are fixed in ice cold 50% CH3OH and 50% (CH₃)₂CO for 20 minutes at room temperature. After fixation cells are permeabilized with 0.5% Triton X100:PBS and then blocked with 0.2% skimmed milk, 0.1% TritonX - 100, 5% FBS in Phosphate Saline Buffer (PBS). Cells are then stained with anti-γ-H2AX antibody (Upstate) and anti-mouse AlexaFluor-488 secondary antibody (Molecular Probes) for the kinetics experiments and with anti-mouse AlexaFluor 568 (Molecular Probes) for the 53BP1/γ-H2AX colocalization experiments. Coverslips are mounted with VECTASHIELD® Mounting Medium containing DAPI, to counterstain cellular nuclei. γ-H2AX foci are scored manually by the same operator throughout the cell nuclei using a Zeiss Apotome fluorescence microscope with 63× objective and the average number of foci per cell are calculated from a minimum of 250 cells per dose/time point.

### Example 18: In Vivo Radiotherapy Assay

This Example describes in vivo radiotherapy assays using Nectin-4 miniproteins and conjugates thereof, including comparison to known control conjugates that bind to the same or different target.

CD-1 nude female mice (Charles River) are implanted with NCI-H358 cells (2×10⁶ cells/mouse diluted with matrigel 1:1) injected subcutaneously on the right flank and allowed to grow to an average volume of 200 mm³ as monitored by caliper measurements. At this point, animals are randomized into groups of 10. Mice are between 6-10 weeks old. All animals receive LabDiet 5053 chow ad libitum. Animals are treated with either a control, which includes either no therapeutic agent or a known ADC if one is available for the target, or non-miniprotein conjugate therapeutic, a non-conjugated miniprotein, a miniprotein-Ac225 conjugate, or a miniprotein-Lul77 conjugate as provided herein. The peptides are administered intravenously via the tail vein at dose level 1 or dose level 2 respectively once a week for the duration of the study. Tumor volumes are measured twice a week by caliper and body weight and clinical signs are monitored for the duration of the studies. Tumor volume is calculated using the formula: V=l²*L/2 (1 length; L width).

CD-1 nude female mice (Charles River) are implanted with SW2780 cells (2×10⁶ cells/mouse diluted with matrigel 1:1) injected subcutaneously on the right flank and allowed to grow to an average volume of 200 mm³ as monitored by caliper measurements. At this point, animals are randomized into groups of 10. Mice are between 6-10 weeks old. All animals receive LabDiet 5053 chow ad libitum. Animals are treated with either a control, which includes either no therapeutic agent or a known ADC if one is available for the target, or non-miniprotein conjugate therapeutic, a non-conjugated miniprotein, a miniprotein-Ac225 conjugate, or a miniprotein-Lul77 conjugate as provided herein. The peptides are administered intravenously via the tail vein at dose level 1 or dose level 2 respectively once a week for the duration of the study. Tumor volumes are measured twice a week by caliper and body weight and clinical signs are monitored for the duration of the studies. Tumor volume is calculated using the formula: V=l²*L/2 (1 length; L width).

### Example 19: Xenograft Model Assay

This Example describes a xenograft assay and treatment using Nectin-4 miniproteins and conjugates thereof, including as compared to known conjugates that bind to the same or different target.

The AG-Br7 xenograft model is derived from a specimen of a patient with triple-negative breast cancer. AG-Panc4 is established from a patient with moderately differentiated adenocarcinoma of the pancreas. NCI-H322M lung adenocarcinoma cell line is used as a lung cancer model. Nectin-4 expression in the xenografts is monitored and determined by qPCR, FACS, and IHC. Cell populations with different expressions of Nectin-4 on the cell surface are isolated via FACS. Five- to 6-week old ICR-SCID mice are obtained from Taconic BioSciences. For experiments with subcutaneous models of AG-B1, AG-Br7, and AG-Panc4, xenograft tumor fragments (5-6 pieces, 1 mm³ each) are implanted subcutaneously into the flank of each mouse. For experiments using the orthotopic breast tumor model AG-Br7, tumor pieces are enzymatically digested to single-cell suspension using Liberase Blendzyme (Roche Applied Science), and 3×10⁶ cells are injected into the mammary fat pad of individual female SCID mice. For experiments with NCI-H322M lung adenocarcinoma-derived xenografts, mice are injected with 2.5×10⁶ tumor cells subcutaneously into the flank of the mice. Various concentrations of Nectin-4 surface expression in the xenografts are utilized: 100,000, 30,000, 10,000, 3,000, 1,000, 300 copies of Nectin-4 per cell. Treatment with the miniprotein-Ac conjugate, miniprotein-Lul77, or appropriate control (e.g., e.g., MGC018, e.g., Enfortumab vedotin or another ADC or therapeutic) by intravenous administration is initiated when tumors reach approximately 200 mm³ in size. Tumor length (L) and width (W) are measured with a caliper, and tumor volume is calculated using the formula W2 L/2. Percent tumor regression is calculated as the tumor volume in a mouse treated with CDP-Ac conjugates relative to the tumor volume in mice treated with control ADC or therapeutic (e.g., Enfortumab vedotin, e.g., MGC018 or another ADC or non-ADC therapeutic), or miniprotein-Lul77. Percent tumor regression at the end of the study is calculated as the tumor volume in each mouse one day before sacrificing relative to the tumor volume in the same mouse on day 1 of the study. A statistical analysis of the tumor volumes at the start of treatment and one day before animal sacrifice is performed using the nonparametric Kruskal-Wallis test. Pairwise comparisons are made using the Tukey-Kramer method (two sided) on the ranks of the data to protect experiment-wise error rate.

### Example 20: In Vivo Assay of Radiolabeled Conjugate

This Example describes in vivo assays using radiolabeled conjugates of Nectin-4 miniproteins and conjugates thereof.

PC-3 ells expressing Nectin-4 are obtained. Their identity is confirmed by DNA testing (STR profiling). To determine Nectin-4 surface expression, cells are incubated with 1 mg/mL AMA in FACS buffer (3% FBS in PBS) on ice for 1 hour followed by Alexa488 anti-human antibody (Invitrogen 11013) and are analyzed using a FACS Calibur Flow Cytometer (Becton Dickinson) and FlowJo (v8.4.5) software. Scatchard analysis is used to determine surface Nectin-4 copy numbers with 125I-AMA.

Female mice are acclimated at least 1 week prior to study start and are 4 to 5 months old. For all experiments, mice are age-matched and tumors are the same size (250-400 mm³) for tumor growth inhibition studies and imaging studies. Cells are prepared for inoculation in HBSS (Gibco) or HBSS and 50% v/v Matrigel (BD Biosciences) and 200 mL are injected into the right dorsal flank. The number of cells inoculated to establish similar-sized tumors are OVCAR-32.1, HPAF-II, and MSTO-211H: 10 million cells; Capan-2, HPAC, and AsPC1: 5 million cells. All are in immunocompromised CB17.SCID. bg mice (Charles River) except HPAF-II (CB17.SCID) and HPAC (Taconic NCR).

Tumor growth inhibition studies are conducted using a miniprotein-Ac 225 conjugate in tumor-bearing mice. The conjugate is administered intravenously via the tail vein (n %4 5-8/group). Tumor length (1, the longest dimension) and width (w, perpendicular to the length) are measured by calipers; tumor volume V was approximated as V1/4 lw2/2.

### Example 21: In Vivo Assay of Radiolabeled Conjugate and Cpi

This Example describes in vivo assays using radiolabeled conjugates of Nectin-4 miniproteins and conjugates thereof in combination with a checkpoint inhibitor.

The mouse melanoma cell line B16F10 is maintained in DMEM containing 10% FCS. B16F10 cells (1×10⁷) are transfected with the pcDNA3 vector (Invitrogen) expressing Myc-tagged human Nectin-4 by electroporation, selected in medium in the presence of G418 (0.5 mg/ml), and then cloned using a limiting dilution procedure. Nectin-4 expression can be verified via IHC. Once tumors reach a certain volume, the mice are treated with the miniprotein-Ac conjugate and μ-mouse PD1. Tumor volume is measured three times a week, and are analyzed via FACS. Animals are euthanized 10 days after treatments, or when tumors reach a volume of 30 mm³ suitable for tumor harvest and cell processing.

1.-135. (canceled)

136. A polypeptide, comprising: an amino acid sequence, wherein the amino acid sequence comprises an amino acid sequence represented by Formula I:
(SEQ ID NO: 161)
CX1YDX2X3FFTALX4X5LRGX6DICX7YIX8X9X10FX11X12X13
X14X15X16CIX17EILX18X19LGCX20
wherein X20 is an optional amino acid or carboxy terminus comprising an —OH and wherein X1 is E or D; X2 is E or G; X3 is E or Q; X4 is K, A, or S; X5 is R, A, Q, K, S, or Cit; X6 is G, A, D, or S; X7 is Y, D, Q, E, L, or S; X8 is Q, L, or S; X9 is A, Q, E, K; X10 is S, A, Q, K, Y, OH-Norleu, or Norleu; X1I is Q, A, N, or S; X12 is Y, N, or T; X13 is L, Y, or V; X14 is P or E; X15 is G, A, D, Q, or K; X16 is L, D, Q, E, or I; X17 is E or Q; X18 is D, Q, or E; X19 is N or Q; and X20, when present as an amino acid is S.

137. The polypeptide of claim 136, wherein X1 is E; X2 is E; X3 is E; X4 is K; X5 is R; X6 is G; X7 is Y; X8 is Q; X9 is A; X10 is S; X1I is Q; X12 is Y; X13 is L; X14 is P; X15 is G; X16 is L; X17 is E; X18 is D; X19 is N; and X20 is S.

138. The polypeptide of claim 136, further comprising one or more of a linker, a chelator, and a radionuclide.

139. The polypeptide of claim 138, wherein (i) the linker, when present, is attached to the C- or N-terminal end of the polypeptide and comprises a polyethylene glycol (PEG) linker comprising PEG4, PEG2, PEG, PEG6, PEG8, PEG12, or PEG24, an ester linker, an amide linker, a maleimide linker, a succinimidyl-4-(N-maleimidomethyl) cyclohexane-1-carboxylate (SMCC) linker, a propanoic acid linker, a caproleic acid linker, or (Gly)n-(gGlu)n- or (PEG)n, wherein n is from 0 to 10, (Gly)₁-10, or any fragment or combination via covalent bond thereof, (ii) the chelator, when present, is attached to either the polypeptide or the linker and comprises DOTA, DOPA, Macropa, or Crown; and/or (iii) the radionuclide, when present, is attached to the chelator, and is selected from the group consisting of Ac-225, Ga-68, Cu-64, In-111, Pb-212, Lu-177, Cu-67, La-132, La-135, and Ce-134.

140. A polypeptide having amino acid sequence comprising an amino acid sequence with (i) at least 90% sequence identity to SEQ ID NO: 78 or (ii) at least 90% sequence identity to an amino acid sequence selected from SEQ ID NO: 78, or any one of SEQ ID NOS: 3, 4, 6-47, 49-77, 79-158, 177, and 161-176 (including amino acid substitutions as set forth in Table 1C).

141. A composition represented by a formula selected from one or more of (M)x-L-C-R, (M)x-L-C, (M)x-C-R, (M)x-L-R, (M)x-C, (M)x-L, and (M)x-R, wherein M comprises a miniprotein (M), L comprises a linker (L), C comprises a chelator (C), R comprises a radionuclide (R), and x is 1, 2, 3, or 4, wherein M comprises the polypeptide of claim 140.

142. The composition of claim 141, wherein, (a) when L is present, L comprises a polyethylene glycol (PEG) linker comprising PEG4, PEG, PEG2, PEG6, PEG8, PEG12, or PEG24, an ester linker, an amide linker, a maleimide linker a valine-citrulline linker, a hydrazone linker, a N-succinimidyl-4-(2-pyridyldithio)butyrate (SPDB) linker, a succinimidyl-4-(N-maleimidomethyl)cyclohexane-1-carboxylate (SMCC) linker, a vinylsulfone-based linker, a propanoic acid linker, a caproleic acid linker, or (Gly)n-(gGlu)n- or (PEG)n, wherein n is from 0 to 10, (Gly)1-10, or any fragment or combination via covalent bond thereof;
(b) when C is present, C comprises 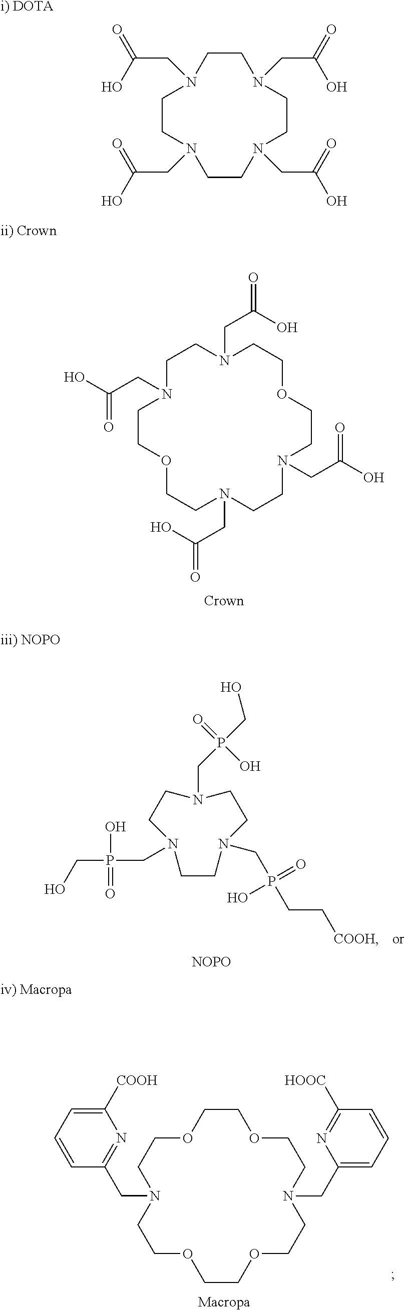
(c) when R is present, R is selected from the group consisting of Ac-225, Ga-68, Cu-64, In-111, Pb-212, Lu-177, Cu-67, La-132, La-135, and Ce-134.

143. A composition comprising a miniprotein-drug conjugate, comprising a miniprotein and at least one drug moiety, wherein the miniprotein comprises the polypeptide of claim 140.

144. The composition of claim 143, wherein the drug moiety is selected from a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder, a DHFR inhibitor, a topoisomerase inhibitor, an auristatin (e.g., monomethyl auristatin E), and an immunotoxin.

145. A pharmaceutical composition, comprising the polypeptide of claim 140 and at least one pharmaceutically acceptable excipient.

146. A miniprotein conjugate comprising: (i) miniprotein (M) that specifically binds to Nectin-4, comprising the polypeptide of claim 140, (ii) a chelator (C) conjugated to (M) through an optional linker (L).

147. The miniprotein conjugate of claim 146, wherein (C) comprises DOTA, and (L), when present, comprises a PEG4.

148. The miniprotein conjugate of claim 147, further comprising a radionuclide (R), chelated to (C), wherein (R) is selected from the group consisting of Ac-225, Ga-68, Cu-64, In-111, Pb-212, Lu-177, Cu-67, La-132, La-135, and Ce-134.

149. A method of treating cancer in a subject in need thereof comprising administering a composition comprising the composition of claim 141.

150. The method of claim 149, wherein the cancer is selected from the group consisting of breast cancer, ovarian cancer, melanoma, pancreatic cancer, peripheral neuroma, glioblastoma, adrenocortical carcinoma, AIDS-related lymphoma, anal cancer, bladder cancer, meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, male breast cancer, malignant mesothelioma, medulloblastoma, Merkel cell carcinoma, metastatic squamous neck cell carcinoma, multiple myeloma and other plasma cell neoplasms, mycosis fimgoides and the Sezary syndrome, myelodysplastic syndromes, nasopharyngeal cancer, neuroblastoma, non-small cell lung cancer, small cell lung cancer, head and neck cancer, skin cancer, oropharyngeal cancer, bone cancers, including osteosarcoma and malignant fibrous histiocytoma of bone, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, Wilm's tumor or other childhood kidney tumors and combinations thereof.

151. A method of targeting a population of cancer cells expressing Nectin-4, the method comprising:
(i) determining a level of expression of Nectin-4 in a population of cancer cells;
(ii) administering to a subject in need thereof a composition according to claim 141, wherein the composition comprises at least one component that specifically binds Nectin-4, wherein the composition targets the Nectin-4-expressing cells and is internalized into the Nectin-4 expressing cells; and
(iii) wherein the patient is treated after the administering as compared to prior to the administering; and the treatment preferentially damages cells expressing Nectin-4.

152. A method of targeting a population of cancer cells expressing Nectin-4, the method comprising:
(i) determining a level of expression of Nectin-4 in a population of cancer cells;
(ii) administering to a subject in need thereof a composition according to claim 141, wherein the composition comprises at least one component that specifically binds Nectin-4, wherein the composition targets the Nectin-4-expressing cells and is internalized into the Nectin-4 expressing cells; and
(iii) wherein the patient is treated after the administering as compared to prior to the administering; and the treatment preferentially damages cells expressing Nectin-4.

153. A method of manufacturing a composition of claim 141.

154. A method of producing a composition comprising a miniprotein (M), optional linker (L), and chelator (C), the method comprising obtaining or synthesizing the miniprotein (M), wherein the miniprotein, after synthesis, has an amino acid sequence comprising an amino acid sequence with at least 90% sequence identity to an amino acid sequence selected from SEQ ID NO: 78, or any one of SEQ ID NOS: 3, 4, 6-47, 49-77, 79-158, 177, and 161-176 (including amino acid substitutions as set forth in Table 1C); and attaching the miniprotein, on its N- or C-terminal end, to an optional linker (L); and/or (b) to the chelator (C).

155. The method of claim 154, wherein the producing further comprises chelating a radionuclide to the composition, wherein the radionuclide is selected from the group consisting of Ac-225, Ga-68, Cu-64, In-111, Pb-212, Lu-177, Cu-67, La-132, La-135, and Ce-134.
